(19) United States
(12) Patent Application Publication
(10) Pub. No.: US 2024/0383878 A1
(43) Pub. Date: Nov. 21, 2024

(54) ALLOSTERIC CHROMENONE INHIBITORS OF PHOSPHOINOSITIDE 3-KINASE (PI3K) FOR THE TREATMENT OF DISEASE

(71) Applicant: Petra Pharma Corporation, New York, NY (US)

(72) Inventors: Erin Danielle ANDERSON, Boulder, CO (US); Sean Douglas ARONOW, Boulder, CO (US); Nicholas A. BOYLES, Hillsboro, OR (US); Xiaohong CHEN, Broomfield, CO (US); Surendra DAWADI, Longmont, CO (US); Eugene R. HICKEY, Danbury, CT (US); Thomas Combs IRVIN, Erie, CO (US); Edward A. KESICKI, New York, NY (US); Gabrielle R. KOLAKOWSKI, Durango, CO (US); Jennifer Lynn KNIGHT, Jersey City, NJ (US); Manoj KUMAR, Boulder, CO (US); Katelyn Frances LONG, Lafayette, CO (US); Christopher Glenn MAYNE, Boulder, CO (US); Alfredo PICADO, Superior, CO (US); Gerit Maria POTOTSCHNIG, San Diego, CA (US); Hua-Yu WANG, San Diego, CA (US); Michael Brian WELCH, Westminster, CO (US); Tien WIDJAJA, Lafayette, CO (US); Nathan Edward WRIGHT, San Diego, CA (US)

(21) Appl. No.: 18/766,351

(22) Filed: Jul. 8, 2024

Related U.S. Application Data
(63) Continuation of application No. 17/734,745, filed on May 2, 2022.
(60) Provisional application No. 63/183,366, filed on May 3, 2021.
(60) Provisional application No. 63/227,652, filed on Jul. 30, 2021.
(60) Provisional application No. 63/250,564, filed on Sep. 30, 2021.
(60) Provisional application No. 63/253,282, filed on Oct. 7, 2021.
(60) Provisional application No. 63/253,412, filed on Oct. 7, 2021.

(51) Int. Cl. C07D 405/04; A61P 35/00; C07D 405/14; C07D 417/04

(52) U.S. Cl. CPC C07D 405/04; A61P 35/00; C07D 405/14; C07D 417/04

(57) ABSTRACT

The disclosure relates to compounds of Formula (I) as allosteric chromenone inhibitors of phosphoinositide 3-kinase (PI3K) useful in the treatment of diseases or disorders associated with PI3K modulation, Formula (I):

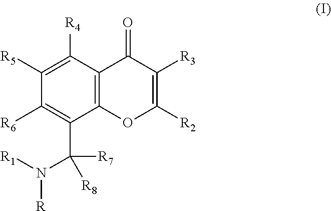

or pharmaceutically acceptable salts thereof wherein R, R₁, R₂, R₃, R₄, R₅, R₆, R₇, and R₈, are as defined herein. The disclosure also relates to methods of making and using compounds of Formula (I) or pharmaceutically acceptable salts thereof.

### FIELD

The present invention is directed to allosteric chromenone inhibitors of phosphoinositide 3-kinase (PI3K) useful in the treatment of diseases, or disorders associated with PI3K modulation. The invention is directed toward compounds, and compositions which inhibit PI3K, methods of (or uses for) treating a disease, or disorder associated with PI3K (e.g., CLOVES syndrome (congenital lipomatous overgrowth, vascular malformations, epidermal naevi, scoliosis/skeletal and spinal syndrome), PIK3CA-related overgrowth syndrome (PROS), breast cancer, brain cancer, prostate cancer, endometrial cancer, gastric cancer, leukemia, lymphoma, sarcoma, colorectal cancer, lung cancer, ovarian cancer, skin cancer, or head and neck cancer), and using, or methods of using, PI3K inhibitors in combination with one or more additional cancer therapies.

### BACKGROUND

The activity of cells can be regulated by external signals that stimulate, or inhibit intracellular events. The process by which stimulatory, or inhibitory signals are transmitted into, and within a cell to elicit an intracellular response is referred to as signal transduction. Over the past decades, cascades of signal transduction events have been elucidated, and found to play a central role in a variety of biological responses. Defects in various components of signal transduction pathways have been found to account for a vast number of diseases, including numerous forms of cancer, inflammatory disorders, metabolic disorders, vascular, and neuronal diseases (Gaestel et al. Current Medicinal Chemistry (2007) 14:2214-2234).

Kinases represent a class of important signaling molecules. Kinases can generally be classified into protein kinases, lipid kinases, and certain kinases exhibiting dual specificities. Protein kinases are enzymes that phosphorylate other proteins and/or themselves (i.e., autophosphorylation). Protein kinases can be generally classified into three major groups based upon their substrate utilization: tyrosine kinases which predominantly phosphorylate substrates on tyrosine residues (e.g., erb2, PDGF receptor, EGF receptor, VEGF receptor, src, abl), serine/threonine kinases which predominantly phosphorylate substrates on serine and/or threonine residues (e.g., mTorC1, mTorC2, ATM, ATR, DNA-PK, Akt), and dual-specificity kinases which phosphorylate substrates on tyrosine, serine and/or threonine residues.

Lipid kinases are enzymes that catalyze the phosphorylation of lipids within cells. These enzymes, and the resulting phosphorylated lipids, and lipid-derived biologically active organic molecules, play a role in many different physiological processes, including cell proliferation, migration, adhesion, and differentiation. A particular group of lipid kinases comprises membrane lipid kinases, i.e., kinases that catalyze the phosphorylation of lipids contained in, or associated with cell membranes. Examples of such enzymes include phosphoinositide(s) kinases (such as PI3-kinases, PI4-Kinases), diacylglycerol kinases, and sphingosine kinases.

The phosphoinositide 3-kinases (PI3Ks) signaling pathway is one of the most highly mutated systems in human cancers. PI3K signaling is involved in many other disease states including allergic contact dermatitis, rheumatoid arthritis, osteoarthritis, inflammatory bowel diseases, chronic obstructive pulmonary disorder, psoriasis, multiple sclerosis, asthma, disorders related to diabetic complications, and inflammatory complications of the cardiovascular system such as acute coronary syndrome.

PI3Ks are members of a unique, and conserved family of intracellular lipid kinases that phosphorylate the 3′-OH group on phosphatidylinositols, or phosphoinositides. The PI3K family comprises 15 kinases with distinct substrate specificities, expression patterns, and modes of regulation (Katso et al., Annu Rev Cell Dev Biol. 2001; 17:615-75). The class I PI3Ks (p110α, p110β, p110δ, and p110γ) are typically activated by tyrosine kinases, or G-protein coupled receptors to generate PIP3, which engages downstream effectors such as those in the pathways of Akt/PDK1, mTOR, the Tec family kinases, and the Rho family GTPases. The class II, and III PI3Ks play a key role in intracellular trafficking through the synthesis of PI(3)P, and PI(3,4)P2.

The PI3K isoforms have been implicated, for example, in a variety of human cancers, and disorders. Mutations in the gene coding for PI3K isoforms, or mutations which lead to upregulation of a PI3K isoform are believed to occur in many human cancers. Mutations in the gene coding for a PI3K isoform are point mutations clustered within several hotspots in helical, and kinase domains. Because of the high rate of PI3K mutations, targeting of this pathway may provide valuable therapeutic opportunities.

Genetic alterations in genes in PI3K signaling are believed to be involved in a range of cancers such as endometrial cancer, breast cancer, esophageal squamous-cell cancer, cervical squamous-cell carcinoma, cervical adenocarcinoma, colorectal adenocarcinoma, bladder urothelial carcinoma, glioblastoma, ovarian cancer, non-small-cell lung cancer, esophagogastric cancer, nerve-sheath tumor, head and neck squamous-cell carcinoma, melanoma, esophagogastric adenocarcinoma, soft-tissue sarcoma, prostate cancer, fibrolamellar carcinoma, hepatocellular carcinoma, diffuse glioma, colorectal cancer, pancreatic cancer, cholangiocarcinoma, B-cell lymphoma, mesothelioma, adrenocortical carcinoma, renal non-clear-cell carcinoma, renal clear-cell carcinoma, germ-cell carcinoma, thymic tumor, pheochromocytoma, miscellaneous neuroepithelial tumor, thyroid cancer, leukemia, and encapsulated glioma (Goncalves M D, Hopkins B D, Cantley L C. Phosphatidylinositol 3-Kinase, Growth Disorders, and Cancer. N Engl J Med. 2018 Nov. 22; 379(21):2052-2062).

The alpha (α) isoform of PI3K has been implicated, for example, in a variety of human cancers. Angiogenesis has been shown to selectively require the α isoform of PI3K in the control of endothelial cell migration. (Graupera et al, Nature 2008; 453; 662-6). Mutations in the gene coding for PI3Kα, or mutations which lead to upregulation of PI3Kα are believed to occur in many human cancers such as lung, stomach, endometrial, ovarian, bladder, breast, colon, brain, prostate, and skin cancers. Mutations in the gene coding for PI3Kα are point mutations clustered within several hotspots in helical, and kinase domains, such as E542K, E545K, and H1047R. Many of these mutations have been shown to be oncogenic gain-of-function mutations. Because of the high rate of PI3Kα mutations, targeting of this pathway may provide valuable therapeutic opportunities. While other PI3K isoforms such as PI3Kδ, or PI3Kγ are expressed primarily in hematopoietic cells, PI3Kα, along with PI3Kβ, is expressed constitutively.

Mutated PI3Kα has been implicated in brain metastases in HR+/HER2− metastatic breast cancers. Development of brain-penetrant PI3Kα inhibitors may provide improved therapeutic benefit over current PI3Kα inhibitors. (Fitzgerald et al., Association between PIK3CA mutation status and development of brain metastases in HR+/HER2− metastatic breast cancer. Ann Oncol 30:v110, 2019 (suppl 5)).

Due to the central role of PI3Kα in regulating organismal glucose homeostasis, PI3K inhibition in patients often gives rise to hyperglycemia and/or hyperinsulinemia (Busaidy N L, et al, Management of metabolic effects associated with anticancer agents targeting the PI3K-Akt-mTOR pathway. J Clin Oncol 2012; 30:2919-28). High levels of circulating insulin could potentially be mitogenic and/or antiapoptotic for cancer cells, and thus negate the antiproliferative effects of PI3K inhibitors (Blouin M-J, et al, Abstract 4615: the hyperinsulinemia caused by PI3K inhibitors attenuates their antineoplastic efficacy, but can be minimized by co-administration of metformin. Cancer Res 2013; 73:4615).

In the setting of cancer with mutated PI3Kα, one way to overcome the problem of compensatory production of insulin and/or glucose upon systemic PI3Kα inhibition would be to develop inhibitors with enhanced selectivity for mutant PI3Kα over wild-type PI3Kα. This would create an increased window for drug dosing to selectively inhibit the pathologic signaling of mutant PI3Kα in the cancer cells without affecting the wild-type PI3Kα in the host tissues that control systemic metabolism (Okkenhaug K, Graupera M, Vanhaesebroeck B. Targeting PI3K in Cancer: Impact on Tumor Cells, Their Protective Stroma, Angiogenesis, and Immunotherapy. Cancer Discov. 2016 October; 6(10):1090-1105), thus limiting toxicities, and permitting higher doses, and more complete inhibition of the drug target (Ariella B. Hanker, et al, Challenges for the clinical development of PI3K inhibitors: Strategies to improve their impact in solid tumors. Cancer Discov. 2019 April; 9(4): 482-491).

Currently PI3Kα inhibitors are nearly equipotent to wild-type, and mutant PI3Kα. Mutant selective inhibitors have been elusive due to the PI3Kα mutations location far from the active site. As such, inhibitors which target a second, peripheral binding pocket near a known mutation (e.g., H1047R) may provide a route to selective PI3Kα inhibition. Thus, targeting a mutated, peripheral binding pocket of PI3Kα, provides a valuable therapeutic target for drug development.

As such, kinases, for example lipid kinases such as PI3Ks, are prime targets for drug development. The present invention provides a new class of kinase inhibitors.

### SUMMARY

In one aspect, the present invention relates to compounds of Formula (I):

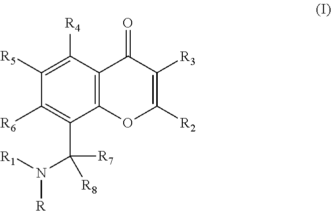

or pharmaceutically acceptable salts thereof, wherein:
   R is —H or C₁-C₃ alkyl;
   R₁ is a group of the formula:

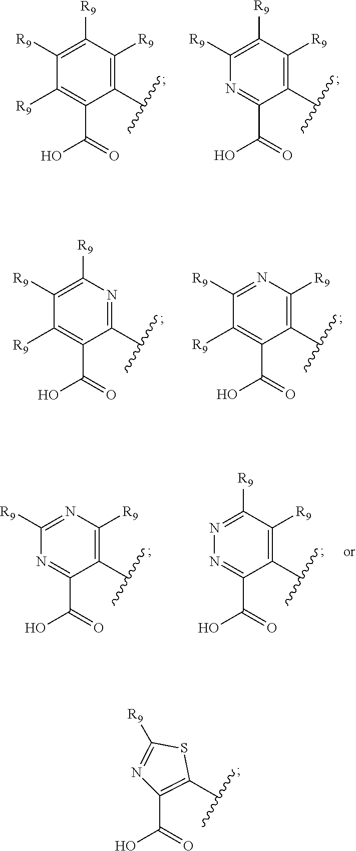

R₂ is a group of the formula:

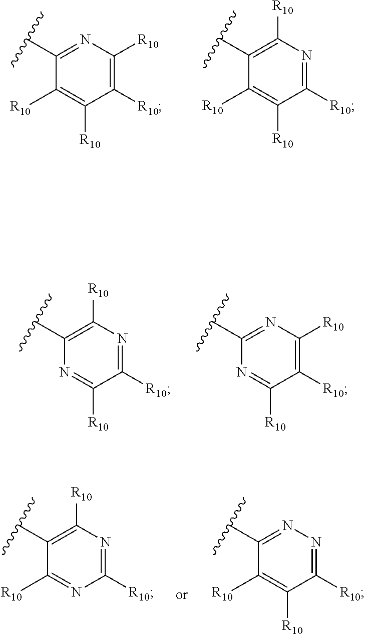

or
R₂ is an optionally substituted 5-member ring heteroaryl selected from pyrrole, furan, thiophene, pyrazole, isoxazole, isothiazole, imidazole, oxazole, thiazole, triazole, tetrazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —C(O)OC₁—C₃ alkyl, —CONR₁₁R₁₁, —NR₁₁R₁₁, —NR₁₁CO₂R₁₁, —OH, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyridine, pyrimidine, pyridazine, pyrazine, pyrazole, isoxazole, isothiazole, imidazole, oxazole, or thiazole; wherein the optionally substituted C₁-C₆ alkyl, C₂-C₆ alkenyl, or C₂-C₆ alkynyl is each optionally substituted with a —CN, —OH, oxetanyl, C₁-C₃ alkoxy, or —CONR₁₁R₁₁; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN;
R₃ is —H, halogen, —CN, —N(H)(C₁-C₃ alkyl), —N(C₁-C₃ alkyl)₂, —N(H)(CH₂CH₂CO₂H), —C(O)C₁-C₃ alkyl, C₁-C₆ alkyl, C₁-C₆ haloalkyl, C₁-C₆ hydroxyalkyl, C₃-C₅ cycloalkyl, an optionally substituted heterocycle of 3 to 5 ring atoms containing 1, 2, or 3 ring heteroatoms independently selected from N, O, or S, or an optionally substituted heteroaryl of 5 or 6 ring atoms containing 1, 2, or 3 ring heteroatoms independently selected from N, O, or S; wherein the optionally substituted heterocycle or heteroaryl is each optionally substituted with one to three substituents each independently selected from halogen, C₁-C₃ alkyl, or C₁-C₃ haloalkyl;
each of R₄, R₅ and R₆ is independently —H, halogen, C₁-C₆ alkyl or C₁-C₆ haloalkyl;
R₇ is —CN, C₁-C₆ alkyl or C₁-C₆ haloalkyl;
R₈ is —H or C₁-C₆ alkyl;
each R₉ is independently —H, halogen, —CN, C₁-C₆ alkyl, C₁-C₆ haloalkyl, C₁-C₆ alkoxy, or C₃-C₅ cycloalkyl;
each R₁₀ is independently —H, —CN, halogen, C₁-C₆ haloalkyl, C₁-C₆ alkoxy, C₁-C₆ haloalkoxy, —SO₂R₁₁, —C(O)OC₁—C₃ alkyl, —CONR₁₁R₁₁, —NR₁₁R₁₁, —NR₁₁—CO₂R₁, —OH, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyrazole, isoxazole, isothiazole, imidazole, oxazole, or thiazole; wherein the optionally substituted C₁-C₆ alkyl, C₂-C₆ alkenyl, or C₂-C₆ alkynyl is each optionally substituted with a —CN, —OH, oxetanyl, C₁-C₃ alkoxy, or —CONR₁₁R₁₁; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN; and
each R₁₁ is independently —H or C₁-C₃ alkyl.

In another aspect, the present invention provides a pharmaceutical composition comprising a compound of Formula (I), (II), or (III), or a pharmaceutically acceptable salt thereof, and a pharmaceutically acceptable diluent, or carrier.

In another aspect, the present invention provides a method of modulating PI3K (e.g., PI3Kα) activity (e.g., in vitro, or in vivo), comprising contacting a cell with a therapeutically effective amount of a compound of Formula (I), (II), or (III), or a pharmaceutically acceptable salt thereof.

In some aspects, the present invention provides a method of treating, or preventing a disease, or disorder disclosed herein in a subject in need thereof, comprising administering to the subject a therapeutically effective amount of a compound of Formula (I), (II), or (III), or a pharmaceutically acceptable salt thereof.

In some aspects, the present invention provides a method of treating, or preventing a disease, or disorder disclosed herein in a subject in need thereof, comprising administering to the subject a therapeutically effective amount of a pharmaceutical composition of a compound of Formula (I), (II), or (III), or a pharmaceutically acceptable salt thereof.

In some aspects, the present invention provides a method of treating a disease, or disorder disclosed herein in a subject in need thereof, comprising administering to the subject a therapeutically effective amount of a compound of Formula (I), (II), or (III), or a pharmaceutically acceptable salt thereof.

In some aspects, the present invention provides a method of treating a disease, or disorder disclosed herein in a subject in need thereof, comprising administering to the subject a therapeutically effective amount of a pharmaceutical composition of a compound of Formula (I), (II), or (III), or a pharmaceutically acceptable salt thereof.

In another aspect, the present invention provides a compound of Formula (I), (II), or (III), or a pharmaceutically acceptable salt thereof for use in therapy.

In another aspect, the present invention provides a compound of Formula (I), (II), or (III), or a pharmaceutically acceptable salt thereof for use in modulating PI3K (e.g., PI3Kα) activity (e.g., in vitro, or in vivo).

In another aspect, the present invention provides a compound of Formula (I), (II), or (III), or a pharmaceutically acceptable salt thereof, for use in selective inhibition for mutant PI3Kα over wild-type PI3Kα.

In another aspect, the present invention provides a compound of Formula (I), (II), or (III), or a pharmaceutically acceptable salt thereof, for use in treating, or preventing a disease, or disorder disclosed herein.

In another aspect, the present invention provides a compound of Formula (I), (II), or (III), or a pharmaceutically acceptable salt thereof, for use in treating a disease, or disorder disclosed herein.

In another aspect, the present invention provides use of a compound of Formula (I), (II), or (III), or a pharmaceutically acceptable salt thereof, in the manufacture of a medicament for modulating PI3K (e.g., PI3Kα) activity (e.g., in vitro, or in vivo).

In another aspect, the present invention provides use of a compound of Formula (I), (II), or (III), or a pharmaceutically acceptable salt thereof, in the manufacture of a medicament for treating, or preventing a disease, or disorder disclosed herein.

In another aspect, the present invention provides use of a compound of Formula (I), (II), or (III), or a pharmaceutically acceptable salt thereof, in the manufacture of a medicament for treating a disease, or disorder disclosed herein.

In another aspect, the present invention provides a method of preparing a compound of Formula (I), (II), or (III), or a pharmaceutically acceptable salt thereof.

In another aspect, the present invention provides a method of preparing a compound, comprising one, or more steps described herein.

In another aspect, the present invention provides a compound obtainable by, or obtained by, a method for preparing a compound as described herein (e.g., a method comprising one, or more steps described in the Schemes).

In another aspect, the present invention provides an intermediate as described herein, being suitable for use in a method for preparing a compound as described herein (e.g., the intermediate is selected from the intermediates described in the Examples).

Other features, and advantages of the invention will be apparent from the following detailed description, and claims.

### DETAILED DESCRIPTION

The present invention provides methods of treating, preventing, or ameliorating a disease, or disorder, (or uses in the treatment, prevention, or amelioration of a disease, or disorder), in which PI3K plays a role by administering to a patient in need thereof a therapeutically effective amount of a PI3K inhibitor of the present invention. The methods (or uses) of the present invention can be used in the treatment of a variety of PI3K-dependent diseases, and disorders.

In some embodiments, the disease, or disorder is a cancer (e.g., breast cancer, brain cancers, prostate cancer, endometrial cancer, gastric cancer, leukemia, lymphoma, sarcoma, colorectal cancer, lung cancer, ovarian cancer, skin cancer, or head and neck cancer). In some embodiments, the disease, or disorder associated with PI3K includes, but is not limited to, CLOVES syndrome (congenital lipomatous overgrowth, vascular malformations, epidermal naevi, scoliosis/skeletal and spinal syndrome), PIK3CA-related overgrowth syndrome (PROS), endometrial cancer, breast cancer, esophageal squamous-cell cancer, cervical squamous-cell carcinoma, cervical adenocarcinoma, colorectal adenocarcinoma, bladder urothelial carcinoma, glioblastoma, ovarian cancer, non-small-cell lung cancer, esophagogastric cancer, nerve-sheath tumor, head and neck squamous-cell carcinoma, melanoma, esophagogastric adenocarcinoma, soft-tissue sarcoma, prostate cancer, fibrolamellar carcinoma, hepatocellular carcinoma, diffuse glioma, colorectal cancer, pancreatic cancer, cholangiocarcinoma, B-cell lymphoma, mesothelioma, adrenocortical carcinoma, renal non-clear-cell carcinoma, renal clear-cell carcinoma, germ-cell carcinoma, thymic tumor, pheochromocytoma, miscellaneous neuroepithelial tumor, thyroid cancer, leukemia, and encapsulated glioma.

The details of the invention are set forth in the accompanying description below. Although methods, and materials similar, or equivalent to those described herein can be used in the practice, or testing of the present disclosure, illustrative methods, and materials are now described. Other features, objects, and advantages of the invention will be apparent from the description, and from the claims. In the specification, and the appended claims, the singular forms also include the plural unless the context clearly dictates otherwise. Unless defined otherwise, all technical, and scientific terms used herein have the same meaning as commonly understood by one of ordinary skill in the art to which this invention belongs. All patents, and publications cited in this specification are incorporated herein by reference in their entireties.

### Definitions

The articles “a”, and “an” refer to one, or more than one (i.e., to at least one) of the grammatical object of the article. By way of example, “an element” means one element, or more than one element.

The term “and/or” means either “and”, or “or” unless indicated otherwise.

The term “administer”, “administering”, or “administration” refers to either directly administering a disclosed compound, or pharmaceutically acceptable salt of the disclosed compound, or a composition to a subject.

The term “alkenyl” refers to a straight, or branched chain unsaturated hydrocarbon containing 2-12 carbon atoms. The “alkenyl” group contains at least one double bond in the chain. The double bond of an alkenyl group can be unconjugated, or conjugated to another unsaturated group. Examples of alkenyl groups include ethenyl, propenyl, n-butenyl, iso-butenyl, pentenyl, or hexenyl.

The term “alkoxy” refers to a straight, or branched chain saturated hydrocarbon containing 1-12 carbon atoms containing a terminal “O” in the chain, i.e., —O(alkyl). Examples of alkoxy groups include without limitation, methoxy, ethoxy, propoxy, butoxy, t-butoxy, or pentoxy groups.

The term “alkyl” refers to a straight, or branched chain saturated hydrocarbon containing 1-12 carbon atoms, preferably 1-6 carbon atoms. Examples of a (C₁-C₆) alkyl group include, but are not limited to, methyl, ethyl, propyl, butyl, pentyl, hexyl, isopropyl, isobutyl, sec-butyl, tert-butyl, isopentyl, neopentyl, and isohexyl.

The term “alkynyl” refers to a straight, or branched chain unsaturated hydrocarbon containing 2-12 carbon atoms. The “alkynyl” group contains at least one triple bond in the chain. Examples of alkynyl groups include ethynyl, propargyl, n-butynyl, iso-butynyl, pentynyl, or hexynyl.

The term “aromatic” means a planar ring having 4n+2 electrons in a conjugated system. As used herein, “conjugated system” means a system of connected p-orbitals with delocalized electrons, and the system may include lone electron pairs.

The term “aryl” unless otherwise specifically defined refers to cyclic, aromatic hydrocarbon groups that have 1 to 3 aromatic rings, including monocyclic, or bicyclic groups such as phenyl, biphenyl, or naphthyl. Where containing two aromatic rings (bicyclic, etc.), the aromatic rings of the aryl group may be joined at a single point (e.g., biphenyl), or fused (e.g., naphthyl). Furthermore, when containing two fused rings the aryl groups herein defined may have one, or more saturated, or partially unsaturated ring fused with a fully unsaturated aromatic ring. Exemplary ring systems of these aryl groups include, but are not limited to, phenyl, biphenyl, naphthyl, anthracenyl, phenalenyl, phenanthrenyl, indanyl, indenyl, tetrahydronaphthalenyl, and tetrahydrobenzoannulenyl.

The term “carrier” encompasses carriers, excipients, and diluents, and means a material, composition, or vehicle, such as a liquid, or solid filler, diluent, excipient, solvent, or encapsulating material, involved in carrying, or transporting a pharmaceutical agent from one organ, or portion of the body, to another organ, or portion of the body of a subject.

The term “cyano” means a substituent having a carbon atom joined to a nitrogen atom by a triple bond, i.e., C≡N.

The term “cycloalkyl” means mono, or polycyclic saturated carbon rings containing 3-18 carbon atoms, preferably 3-10 carbon atoms. Examples of cycloalkyl groups include, without limitation, cyclopropyl, cyclobutyl, cyclopentyl, cyclohexyl, cycloheptanyl, cyclooctanyl, norbornyl, norborenyl, bicyclo[2.2.2]octanyl, and bicyclo[2.2.2]octenyl.

The term “disorder” means, and is used interchangeably with, the terms disease, condition, or illness, unless otherwise indicated.

The term “haloalkoxy” refers to an alkoxy group, as defined herein, which is substituted with one, or more halogen. Examples of haloalkoxy groups include, but are not limited to, trifluoromethoxy, difluoromethoxy, pentafluoroethoxy, and trichloromethoxy.

The term “haloalkyl” refers to an alkyl group, as defined herein, which is substituted with one, or more halogen. Examples of haloalkyl groups include, but are not limited to, trifluoromethyl, difluoromethyl, pentafluoroethyl, and trichloromethyl.

The term “halogen” or “halo” refers to fluorine, chlorine, bromine, or iodine.

The term “heteroaryl” unless otherwise specifically defined means a monovalent monocyclic, or a polycyclic aromatic radical of 5 to 24 ring atoms, preferably 5 to 10 ring atoms, containing one, or more ring heteroatoms selected from N, O, S, P, or B, preferably 1, 2, 3, or 4 ring heteroatoms selected from N, O, or S, the remaining ring atoms being C. A polycyclic aromatic radical includes two, or more fused rings, and may further include two, or more spiro-fused rings, e.g., bicyclic, tricyclic, tetracyclic, and the like. Unless otherwise specifically defined, “fused” means two rings sharing two ring atoms. Unless otherwise specifically defined, “spiro-fused” means two rings sharing one ring atom. Heteroaryl as herein defined also means a bicyclic heteroaromatic group wherein the heteroatom is selected from N, O, S, P, or B, preferably N, O, or S. Heteroaryl as herein defined also means a tricyclic heteroaromatic group containing one, or more ring heteroatoms selected from N, O, S, P, or B, preferably N, O, or S. Heteroaryl as herein defined also means a tetracyclic heteroaromatic group containing one, or more ring heteroatoms selected from N, O, S, P, or B, preferably N, O, or S. Examples of heteroaromatic groups include, but are not limited to, furyl, thienyl, pyrrolyl, pyridyl, pyrazolyl, pyrimidinyl, imidazolyl, isoxazolyl, oxazolyl, oxadiazolyl, pyrazinyl, indolyl, thiophen-2-yl, quinolyl, benzopyranyl, isothiazolyl, thiazolyl, thiadiazole, indazole, benzimidazolyl, thieno[3,2-b]thiophene, triazolyl, triazinyl, imidazo[1,2-b]pyrazolyl, furo[2,3-c]pyridinyl, imidazo[1,2-a]pyridinyl, indazolyl, pyrrolo[2,3-c]pyridinyl, pyrrolo[3,2-c]pyridinyl, pyrazolo[3,4-c]pyridinyl, thieno[3,2-c]pyridinyl, thieno[2,3-c]pyridinyl, thieno[2,3-b]pyridinyl, benzothiazolyl, indolinyl, indolinonyl, dihydrobenzothiophenyl, dihydrobenzofuranyl, benzofuranyl, chromanyl, thiochromanyl, tetrahydroquinolinyl, dihydrobenzothiazinyl, quinolinyl, isoquinolinyl, 1,6-naphthyridinyl, benzo[de]isoquinolinyl, pyrido[4,3-b][1,6]naphthyridinyl, thieno[2,3-b]pyrazinyl, quinazolinyl, tetrazolo[1,5-a]pyridinyl, [1,2,4]triazolo[4,3-a]pyridinyl, isoindolyl, pyrrolo[2,3-b]pyridinyl, pyrrolo[3,4-b]pyridinyl, pyrrolo[3,2-b]pyridinyl, imidazo[5,4-b]pyridinyl, pyrrolo[1,2-a]pyrimidinyl, tetrahydro pyrrolo[1,2-a]pyrimidinyl, 3,4-dihydro-2H-1-pyrrolo[2,1-b]pyrimidine, dibenzo[b,d]thiophene, pyridin-2-one, furo[3,2-c]pyridinyl, furo[2,3-c]pyridinyl, 1H-pyrido[3,4-b][1,4]thiazinyl, benzooxazolyl, benzoisoxazolyl, furo[2,3-b]pyridinyl, benzothiophenyl, 1,5-naphthyridinyl, furo[3,2-b]pyridine, [1,2,4]triazolo[1,5-a]pyridinyl, benzo[1,2,3]triazolyl, imidazo[1,2-a]pyrimidinyl, [1,2,4]triazolo[4,3-b]pyridazinyl, benzo[c][1,2,5]thiadiazolyl, benzo[c][1,2,5]oxadiazole, 1,3-dihydro-2H-benzo[d]imidazol-2-one, 3,4-dihydro-2H-pyrazolo[1,5-b][1,2]oxazinyl, 4,5,6,7-tetrahydropyrazolo[1,5-a]pyridinyl, thiazolo[5,4-d]thiazolyl, imidazo[2,1-b][1,3,4]thiadiazolyl, thieno[2,3-b]pyrrolyl, and 3H-indolyl. Furthermore, when containing two, or more fused rings, the heteroaryl groups defined herein may have one, or more saturated, or partially unsaturated ring fused with one, or more fully unsaturated aromatic ring. In heteroaryl ring systems containing more than two fused rings, a saturated, or partially unsaturated ring may further be fused with a saturated, or partially unsaturated ring described herein. Furthermore, when containing three, or more fused rings, the heteroaryl groups defined herein may have one, or more saturated, or partially unsaturated ring spiro-fused. Any saturated, or partially unsaturated ring described herein is optionally substituted with one, or more oxo. Exemplary ring systems of these heteroaryl groups include, for example, indolinyl, indolinonyl, dihydrobenzothiophenyl, dihydrobenzofuran, chromanyl, thiochromanyl, tetrahydroquinolinyl, dihydrobenzothiazine, 3,4-dihydro-1H-isoquinolinyl, 2,3-dihydrobenzofuranyl, benzofuranonyl, oxindolyl, indolyl, 1,6-dihydro-7H-pyrazolo[3,4-c]pyridin-7-onyl, 7,8-dihydro-6H-pyrido[3,2-b]pyrrolizinyl, 8H-pyrido[3,2-b]pyrrolizinyl, 1,5,6,7-tetrahydrocyclopenta[b]pyrazolo[4,3-e]pyridinyl, 7,8-dihydro-6H-pyrido[3,2-b]pyrrolizinyl, pyrazolo[1,5-a]pyrimidin-7(4H)-onyl, 3,4-dihydropyrazino[1,2-a]indol-1(2H)-onyl, benzo[c][1,2]oxaborol-1(3H)-olyl, 6,6a,7,8-tetrahydro-9H-pyrido[2,3-b]pyrrolo[1,2-d][1,4]oxazin-9-onyl, and 6a′,7′-dihydro-6′H,9′H-spiro[cyclopropane-1,8′-pyrido[2,3-b]pyrrolo[1,2-d][1,4]oxazin]-9′-onyl.

The term “heterocyclyl”, “heterocycle”, or “heterocycloalkyl” means mono, or polycyclic rings containing 3-24 atoms, preferably 3-10 atoms, which include carbon, and one, or more heteroatoms selected from N, O, S, P, or B, preferably 1, 2, 3, or 4 heteroatoms selected from N, O, and S, and wherein the rings are not aromatic. Examples of heterocyclyl rings include, but are not limited to, oxetanyl, azetidinyl, tetrahydrofuranyl, tetrahydropyranyl, pyrrolidinyl, oxazolinyl, oxazolidinyl, thiazolinyl, thiazolidinyl, pyranyl, thiopyranyl, tetrahydropyranyl, dioxalinyl, piperidinyl, morpholinyl, thiomorpholinyl, thiomorpholinyl S-oxide, thiomorpholinyl S-dioxide, piperazinyl, azepinyl, oxepinyl, diazepinyl, tropanyl, oxazolidinonyl, and homotropanyl.

The term “hydroxyalkyl” refers to an alkyl group, as defined herein, which is substituted with a hydroxy group.

The term “isomers” refers to compounds that have the same molecular formula but differ in the nature or sequence of bonding of their atoms or the arrangement of their atoms in space. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomers or as a mixture thereof. A mixture containing equal proportions of the enantiomers is called a “racemic mixture”.

The term “modulate”, “modulation”, or “modulating” refers to a biological activity of a compound, or substrate that inhibits and/or activates PI3K.

The term “patient”, or “subject” is a mammal, e.g., a human, mouse, rat, guinea pig, dog, cat, horse, cow, pig, or non-human primate, such as a monkey, chimpanzee, baboon, or rhesus. Preferably, the mammal is human.

The term “therapeutically effective amount” when used in connection with a compound refers to the amount or dose of the compound which upon single or multiple dose administration to the patient, provides the desired effect in the patient under diagnosis or treatment. An effective amount can be determined by one skilled in the art by the use of known techniques and by observing results obtained under analogous circumstances. In determining the effective amount for a patient, a number of factors are considered by the attending diagnostician, including, but not limited to: the species of patient; its size, age, and general health; the specific disease or disorder involved; the degree of or involvement or the severity of the disease or disorder; the response of the individual patient; the particular compound administered; the mode of administration; the bioavailability characteristics of the preparation administered; the dose regimen selected; the use of concomitant medication; and other relevant circumstances.

The term “treating” with regard to a subject, includes restraining, slowing, stopping, or reversing the progression or severity of an existing symptom or disorder.

### Compounds of the Present Invention

In one aspect, the present invention provides compounds of Formula (I), or pharmaceutically acceptable salts thereof:

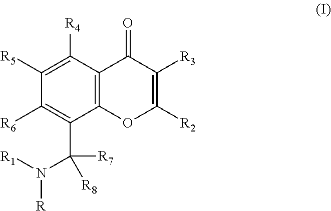

wherein R, R₁, R₂, R₃, R₄, R₅, R₆, R₇, and R₈, are as defined in the Summary for Formula (I).

In a further aspect, compounds of Formula (I) wherein R₈ is —H have Formula (II), or pharmaceutically acceptable salts thereof:

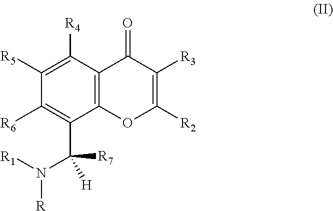

wherein R, R₁, R₂, R₃, R₄, R₅, R₆, and R₇, are as defined in the Summary for Formula (I).

In a compound of Formula (I), or pharmaceutically acceptable salts thereof,
R is —H or C₁-C₃ alkyl;
R₁ is a group of the formula:

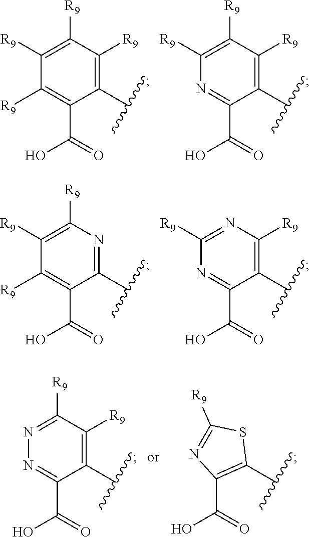

R₂ is a group of the formula:

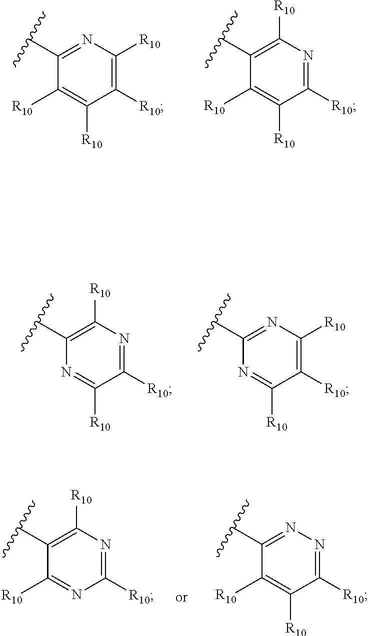

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyridine, pyrimidine, pyridazine, pyrazine, pyrazole, isoxazole, isothiazole, imidazole, oxazole, or thiazole; wherein the optionally substituted C₁-C₆ alkyl, C₂-C₆ alkenyl, or C₂-C₆ alkynyl is each optionally substituted with a —CN, —OH, oxetanyl, or C₁-C₃ alkoxy; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN;
R₃ is —H, halogen, —CN, C₁-C₆ alkyl, C₁-C₆ haloalkyl, C₃-C₅ cycloalkyl, a heterocycle of 3 to 5 ring atoms containing 1, 2, or 3 ring heteroatoms independently selected from N, O, or S, or a heteroaryl of 5 ring atoms containing 1, 2, or 3 ring heteroatoms independently selected from N, O, or S;
each of R₄, R₅ and R₆ is independently —H, halogen, C₁-C₆ alkyl or C₁-C₆ haloalkyl;
R₇ is —CN, C₁-C₆ alkyl or C₁-C₆ haloalkyl;
R₈ is —H or C₁-C₆ alkyl;
each R₉ is independently —H, halogen, C₁-C₆ alkyl, C₁-C₆ haloalkyl, C₁-C₆ alkoxy, or C₃-C₅ cycloalkyl;
each R₁₀ is independently —H, —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁—CO₂R₁₁, —OH, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyrazole, isoxazole, isothiazole, imidazole, oxazole, or thiazole; wherein the optionally substituted C₁-C₆ alkyl, C₂-C₆ alkenyl, or C₂-C₆ alkynyl is each optionally substituted with a —CN, —OH, oxetanyl, or C₁-C₃ alkoxy; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN; and
each R₁₁ is independently —H or C₁-C₃ alkyl.

In a compound of Formula (I), or pharmaceutically acceptable salts thereof,
R is —H or C₁-C₃ alkyl;
R₁ is a group of the formula:

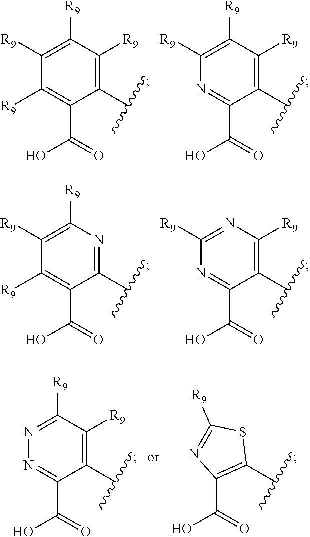

R₂ is a group of the formula:

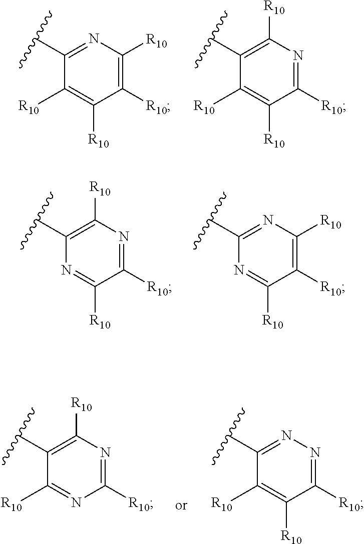

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN;
R₃ is —H, —CN, C₁-C₆ alkyl or C₁-C₆ haloalkyl;
each of R₄, R₅ and R₆ is independently —H, halogen, C₁-C₆ alkyl or C₁-C₆ haloalkyl;
R₇ is —CN, C₁-C₆ alkyl or C₁-C₆ haloalkyl;
R₈ is —H or C₁-C₆ alkyl;
each R₉ is independently —H, halogen, C₁-C₆ alkyl, C₁-C₆ haloalkyl, C₁-C₆ alkoxy, or C₃-C₅ cycloalkyl;
each R₁₀ is independently —H, —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁—CO₂R₁₁, —OH, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN; and
each R₁₁ is independently —H or C₁-C₃ alkyl

In a compound of Formula (I), or pharmaceutically acceptable salts thereof,
R is independently —H or C₁-C₃ alkyl;
R₁ is a group of the formula:

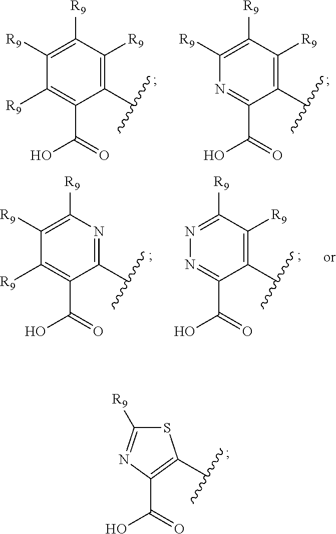

R₂ is a group of the formula:

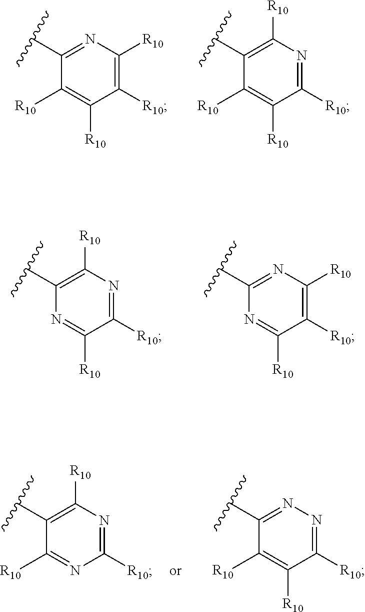

R₃ is —H, —CN, C₁-C₆ alkyl or C₁-C₆ haloalkyl;
each of R₄, R₅ and R₆ is independently —H, halogen, C₁-C₆ alkyl or C₁-C₆ haloalkyl;
R₇ is —CN, C₁-C₆ alkyl or C₁-C₆ haloalkyl;
R₈ is —H or C₁-C₆ alkyl;
each R₉ is independently —H, halogen, C₁-C₆ alkyl, C₁-C₆ haloalkyl, C₁-C₆ alkoxy, or C₃-C₅ cycloalkyl;
each R₁₀ is independently —H, —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁—CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN; and
each R₁₁ is independently —H or C₁-C₃ alkyl.

In a compound of Formula (I), or (II), or pharmaceutically acceptable salts thereof, R₂ is a group of the formula:

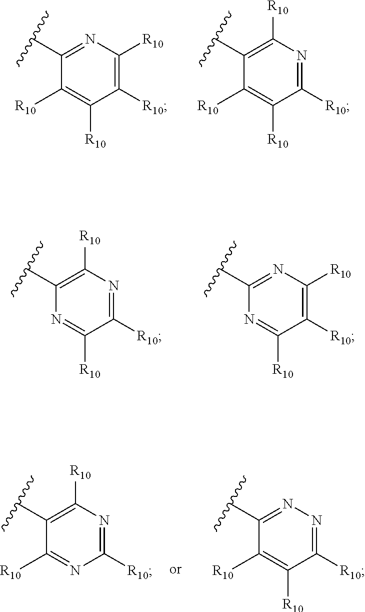

and
each R₁₀ is independently —H, —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁—CO₂R₁₁, —OH, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine or an optionally substituted heteroaryl selected from pyrazole, isoxazole, isothiazole, imidazole, oxazole, or thiazole; wherein the optionally substituted C₁-C₆ alkyl, C₂-C₆ alkenyl, or C₂-C₆ alkynyl is each optionally substituted with a —CN, —OH, oxetanyl, or C₁-C₃ alkoxy; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN.

In a compound of Formula (I), or (II), or pharmaceutically acceptable salts thereof, R₂ is a group of the formula:

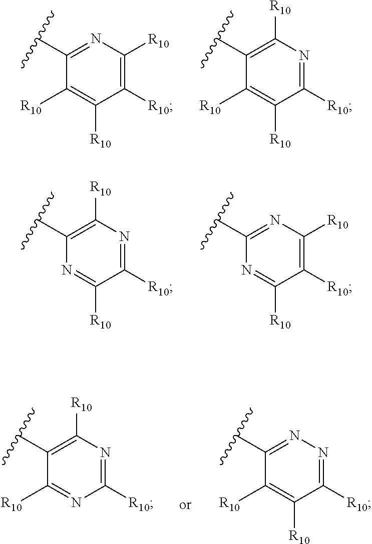

and
each R₁₀ is independently —H, —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁—CO₂R₁₁, —OH, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN; and each R₁₁ is independently —H or C₁-C₃ alkyl.

In a compound of Formula (I), or (II), or pharmaceutically acceptable salts thereof, R₂ is a group of the formula:

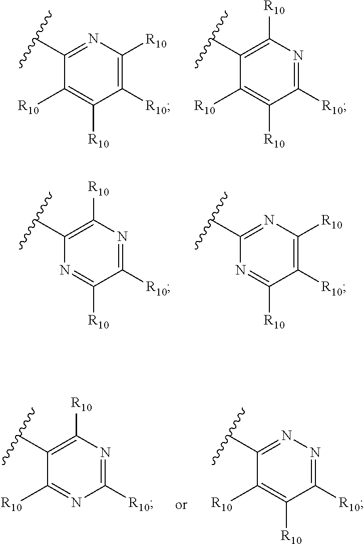

and
each R₁₀ is independently —H, —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁—CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN; and each R₁₁ is independently —H or C₁-C₃ alkyl.

In a compound of Formula (I), or (II), or pharmaceutically acceptable salts thereof, R₂ is a group of the formula:

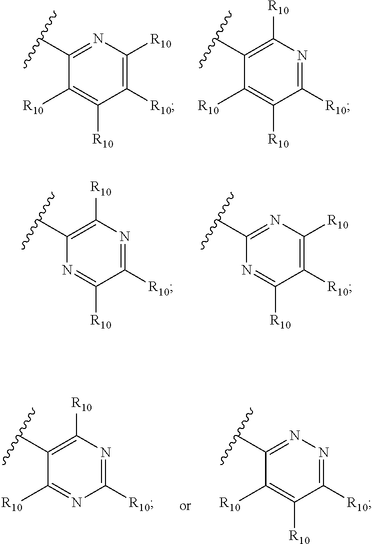

and
each R₁₀ is independently —H, —CN, halogen, C₁-C₆ haloalkyl, C₁-C₆ alkoxy, —SO₂R₁₁, an optionally substituted C₁-C₆ alkyl, an optionally substituted C₂-C₆ alkynyl, an optionally substituted C₃-C₅ cycloalkyl, or an optionally substituted heteroaryl selected from pyrazole, isoxazole, isothiazole, imidazole, oxazole, or thiazole; wherein the optionally substituted C₁-C₆ alkyl or C₂-C₆ alkynyl is optionally substituted with a —CN, —OH, or C₁-C₃ alkoxy; and the optionally substituted C₃-C₅ cycloalkyl or heteroaryl is optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN.

In a compound of Formula (I), or (II), or pharmaceutically acceptable salts thereof, R₂ is a group of the formula:

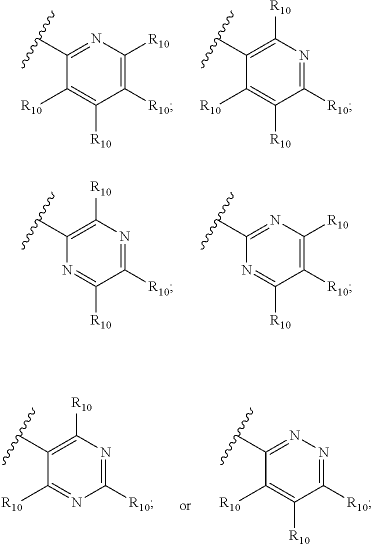

and
each R₁₀ is independently —H, —CN, halogen, C₁-C₆ haloalkyl, C₁-C₆ alkoxy, —SO₂R₁₁, an optionally substituted C₁-C₆ alkyl, an optionally substituted C₂-C₆ alkynyl, or an optionally substituted C₃-C₅ cycloalkyl; wherein the optionally substituted C₁-C₆ alkyl or C₂-C₆ alkynyl is optionally substituted with a —CN, —OH, or C₁-C₃ alkoxy; and the optionally substituted C₃-C₅ cycloalkyl is optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN.

In a compound of Formula (I), or (II), or pharmaceutically acceptable salts thereof, R₂ is a group of the formula:

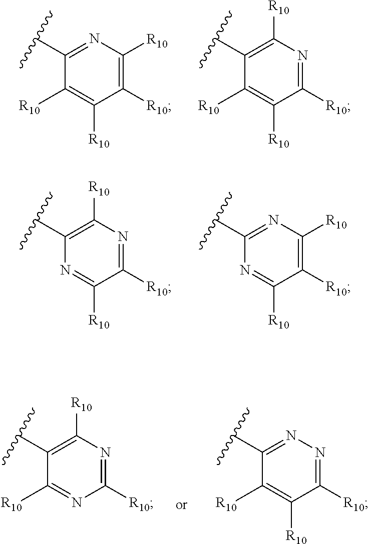

and
each R₁₀ is independently —H, —CN, halogen, C₁-C₆ haloalkyl, C₁-C₆ alkoxy, —SO₂R₁₁, a C₁-C₆ alkyl, a C₂-C₆ alkynyl optionally substituted with —OH, a C₃ cycloalkyl optionally substituted with —CN, or a heteroaryl selected from pyrazole optionally substituted with one to three substituents each independently selected from C₁-C₃ alkyl.

In a compound of Formula (I), or (II), or pharmaceutically acceptable salts thereof, R₂ is a group of the formula:

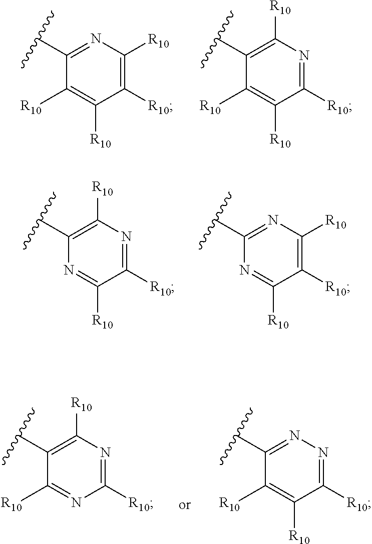

and
each R₁₀ is independently —H, —CN, halogen, C₁-C₆ haloalkyl, C₁-C₆ alkoxy, —SO₂R₁₁, a C₁-C₆ alkyl, a C₂-C₆ alkynyl optionally substituted with —OH, or a C₃ cycloalkyl optionally substituted with —CN.

In a compound of Formula (I), or (II), or pharmaceutically acceptable salts thereof, R₂ is a group of the formula:

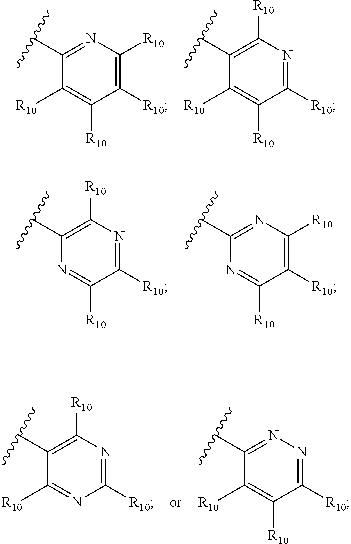

and
each R₁₀ is independently:

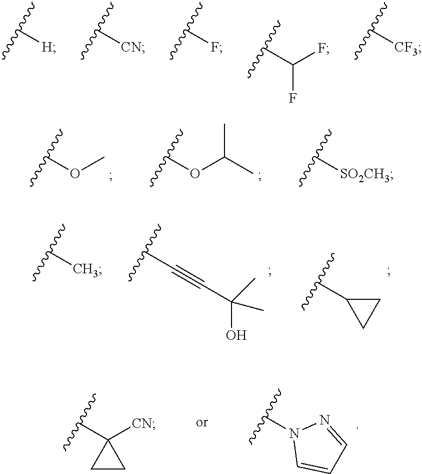

In a compound of Formula (I), or (II), or pharmaceutically acceptable salts thereof, R₂ is a group of the formula:

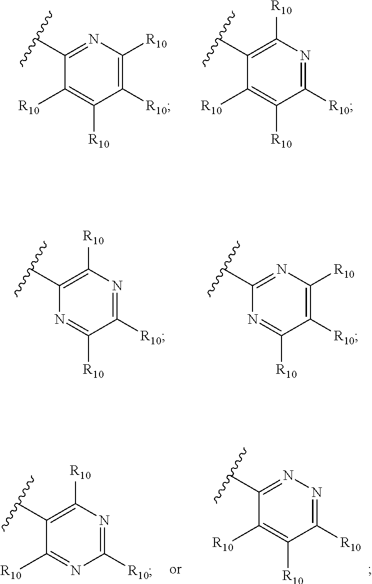

and
each R₁₀ is independently:

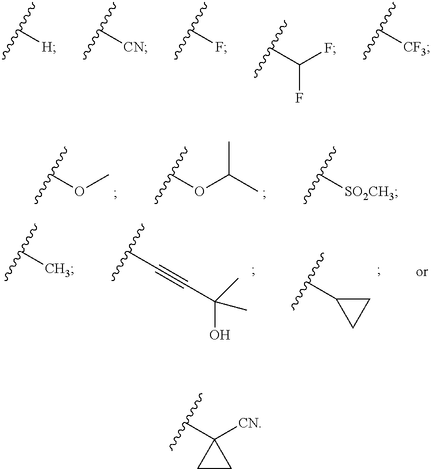

In a compound of Formula (I), or (II), or pharmaceutically acceptable salts thereof, R₂ is a group of the formula:

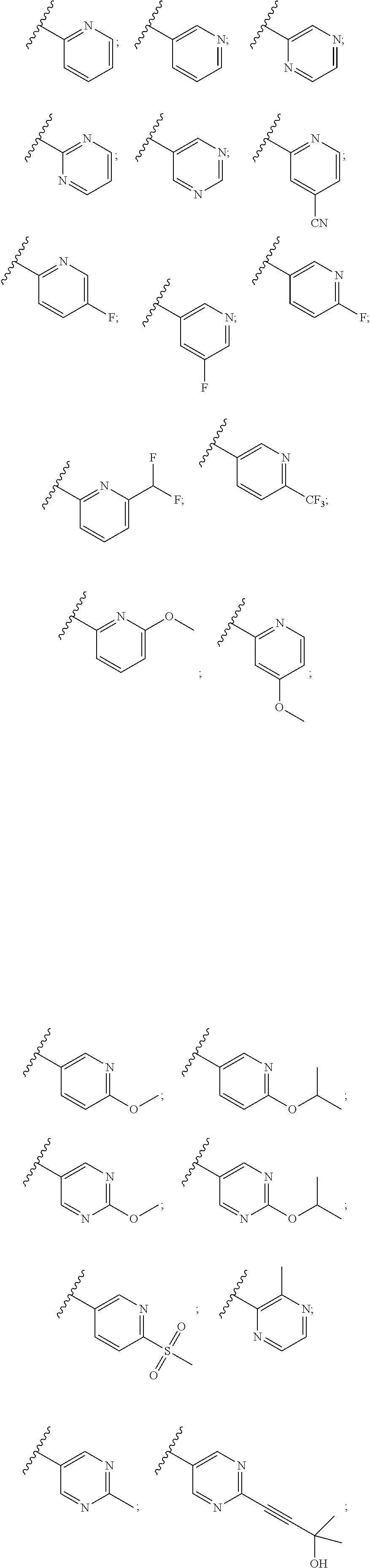

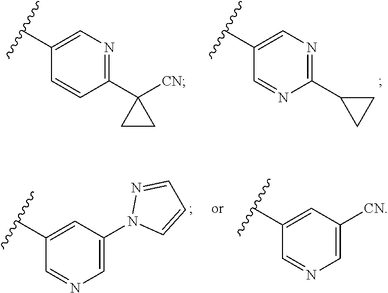 [SMILES: *c1ccc(C2(C#N)CC2)nc1 ; *c1cnc(C2CC2)nc1 ; *c1cncc(-n2cccn2)c1 ; *c1cncc(C#N)c1]

In a compound of Formula (I), or (II), or pharmaceutically acceptable salts thereof, R₂ is a group of the formula:

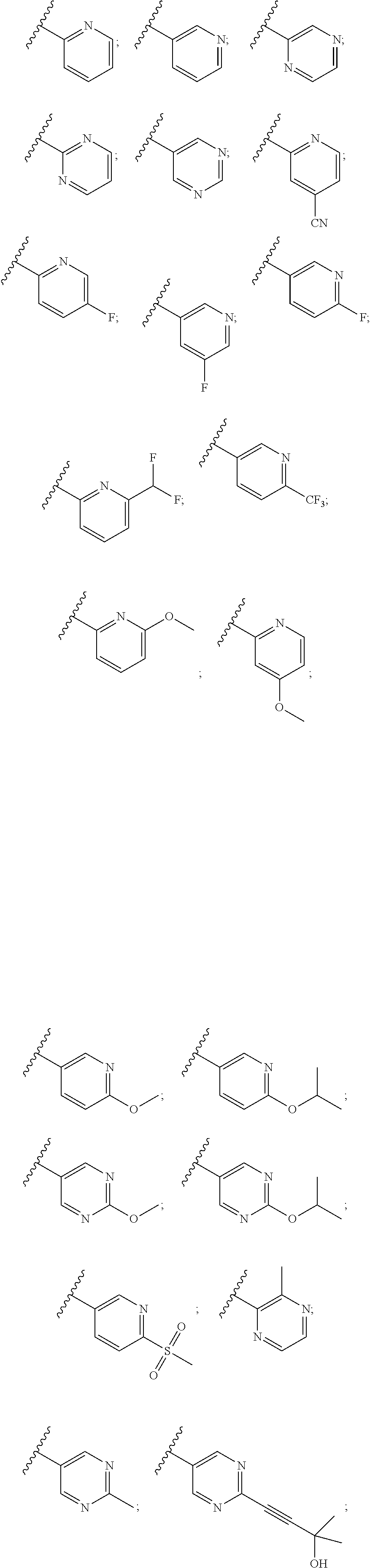

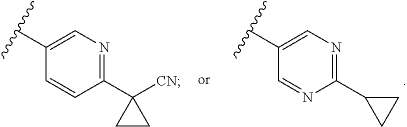 [SMILES: *c1ccc(C2(C#N)CC2)nc1 ; "or" ; *c1cnc(C2CC2)nc1]

In a compound of Formula (I), or (II), or pharmaceutically acceptable salts thereof, R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyridine, pyrimidine, pyridazine, pyrazine, pyrazole, isoxazole, isothiazole, imidazole, oxazole, or thiazole; wherein the optionally substituted C₁-C₆ alkyl, C₂-C₆ alkenyl, or C₂-C₆ alkynyl is each optionally substituted with a —CN, —OH, oxetanyl, or C₁-C₃ alkoxy; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN.

In a compound of Formula (I), or (II), or pharmaceutically acceptable salts thereof, R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN; and each R₁₁ is independently —H or C₁-C₃ alkyl.

In a compound of Formula (I), or (II), or pharmaceutically acceptable salts thereof, R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from C₁-C₆ haloalkyl, an optionally substituted C₁-C₆ alkyl, an optionally substituted phenyl, an optionally substituted 1,3-benzodioxole, or an optionally substituted heteroaryl selected from pyridine, pyrimidine, pyridazine, pyrazine, pyrazole, isoxazole, isothiazole, imidazole, oxazole, or thiazole; wherein the optionally substituted C₁-C₆ alkyl, is optionally substituted with a —CN, —OH, oxetanyl, or C₁-C₃ alkoxy; and the optionally substituted phenyl, 1,3-benzodioxole, or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN; and each R₁₁ is independently —H or C₁-C₃ alkyl.

In a compound of Formula (I), or (II), or pharmaceutically acceptable salts thereof, R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from C₁-C₆ haloalkyl, C₁-C₆ alkyl, an optionally substituted phenyl, an optionally substituted 1,3-benzodioxole, or an optionally substituted heteroaryl selected from pyridine or pyrimidine; wherein the optionally substituted phenyl, 1,3-benzodioxole, or heteroaryl is each optionally substituted with one to three substituents each independently selected from halogen, C₁-C₃ haloalkyl, C₁-C₃ alkoxy, C₁-C₃ haloalkoxy, —SO₂R₁₁, or —CN; and each R₁₁ is independently —H or C₁-C₃ alkyl.

In a compound of Formula (I), or (II), or pharmaceutically acceptable salts thereof, R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from:

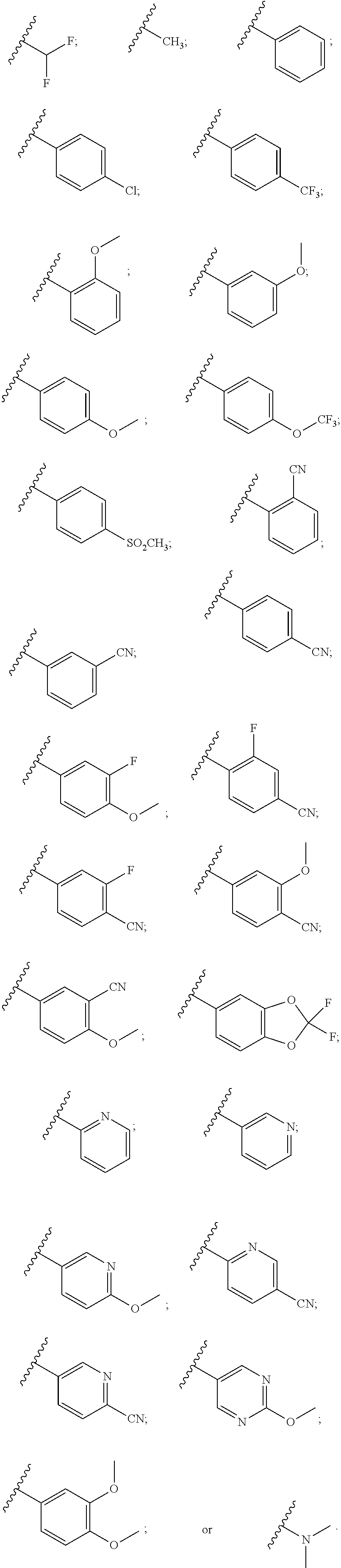

In a compound of Formula (I), or (II), or pharmaceutically acceptable salts thereof, R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from:

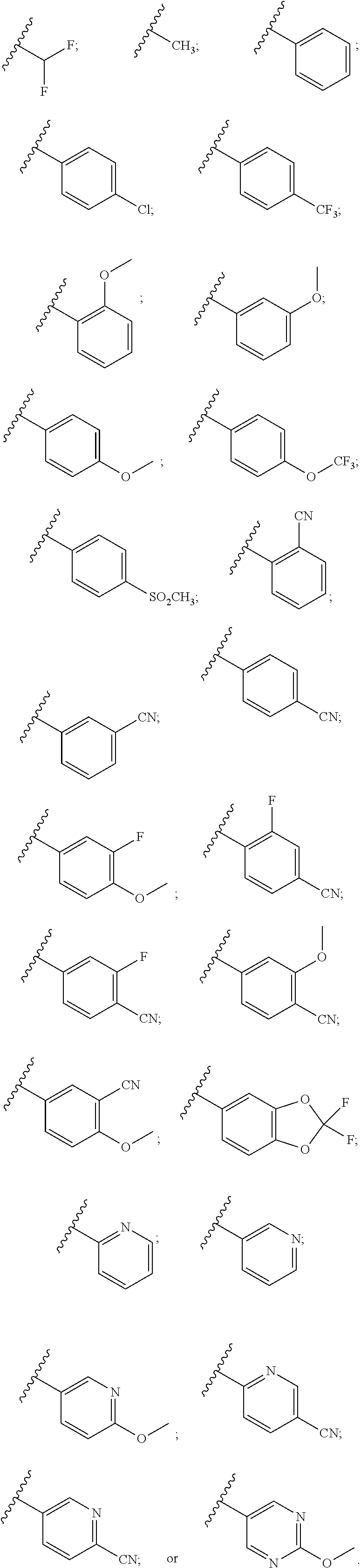

In a compound of Formula (I), or (II), or pharmaceutically acceptable salts thereof, R₂ is a group of the formula:

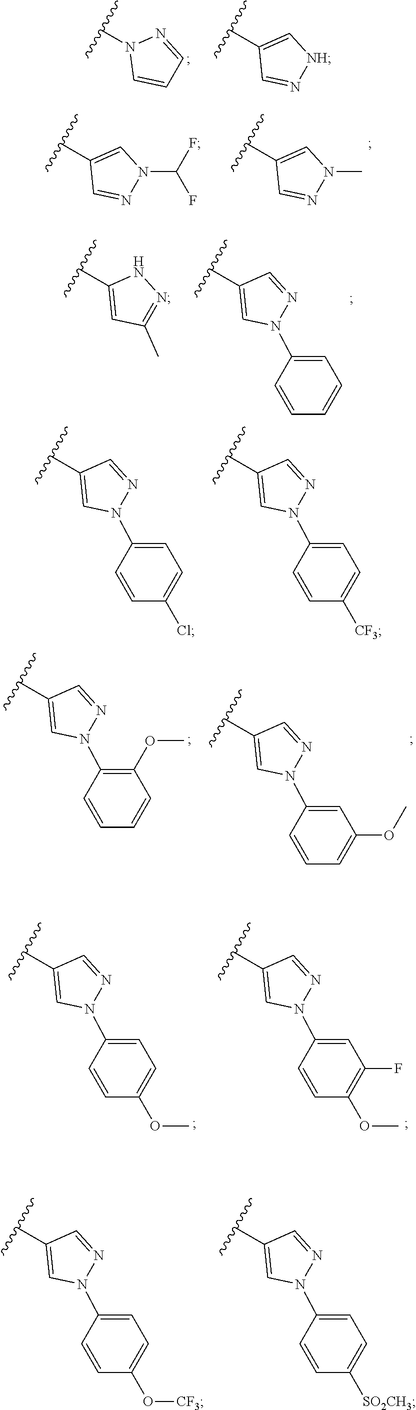

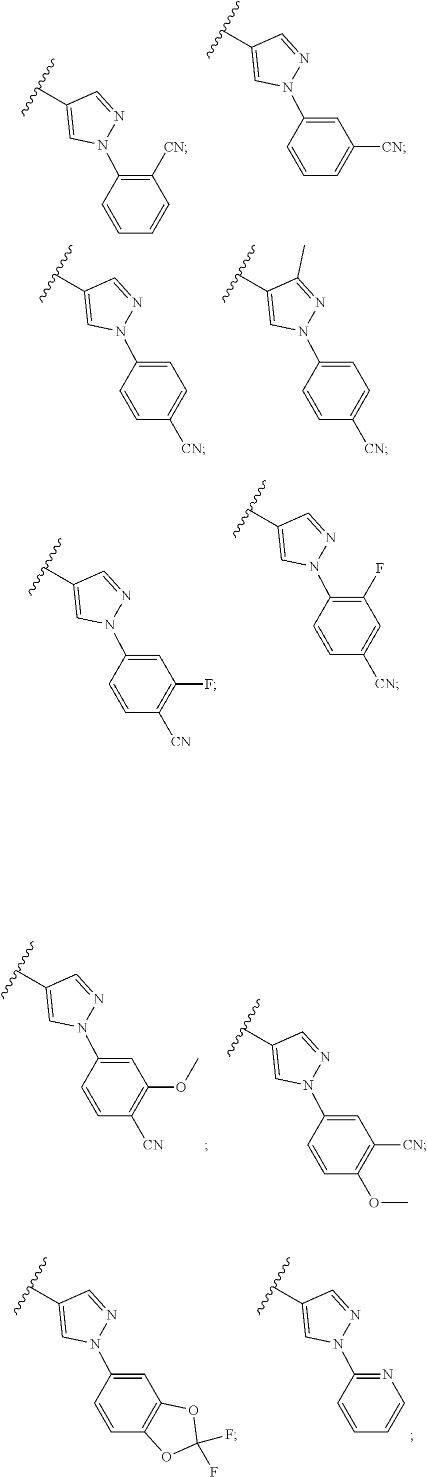

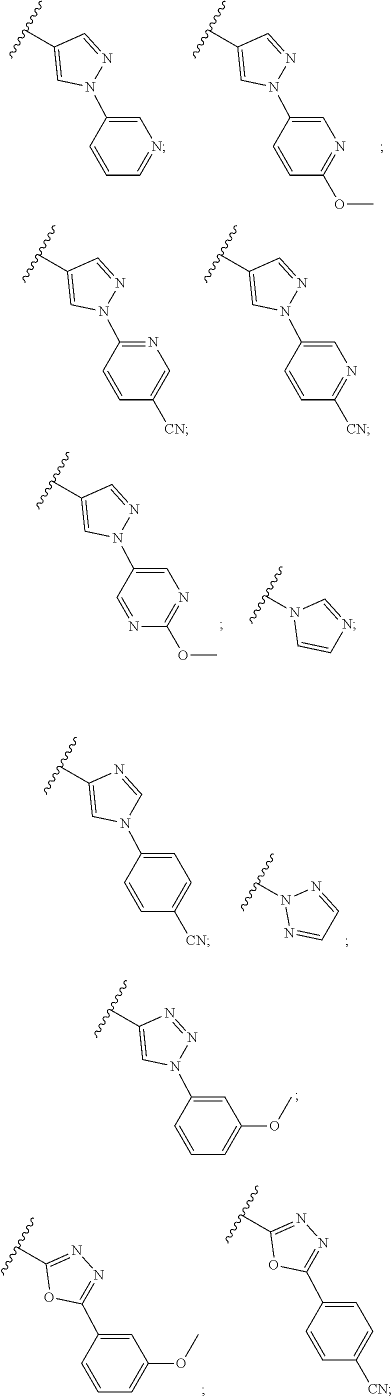

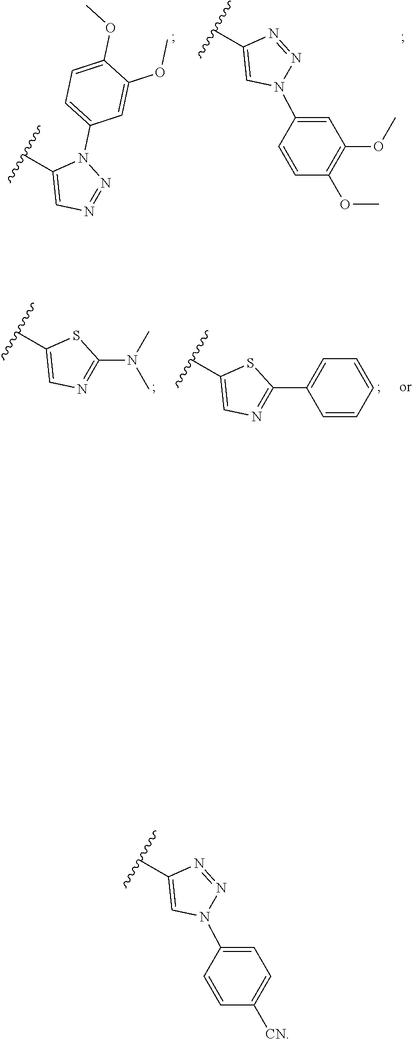

In a compound of Formula (I), or (II), or pharmaceutically acceptable salts thereof, R₂ is a group of the formula:

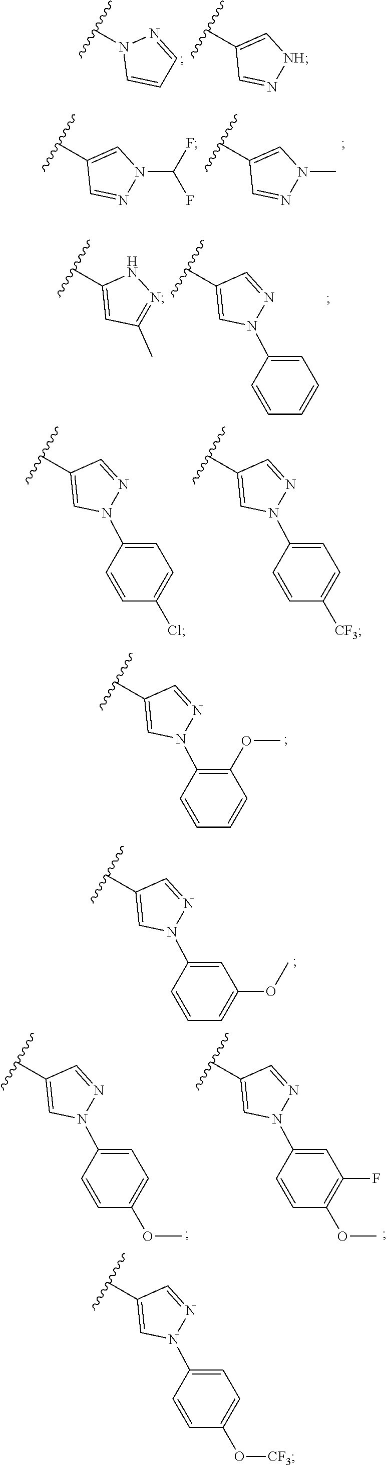

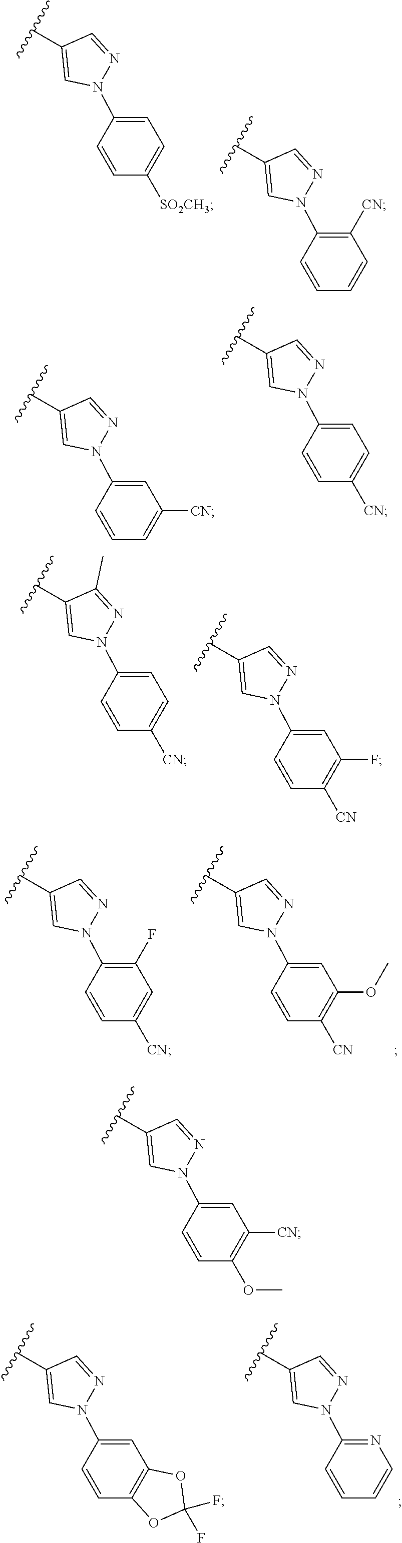

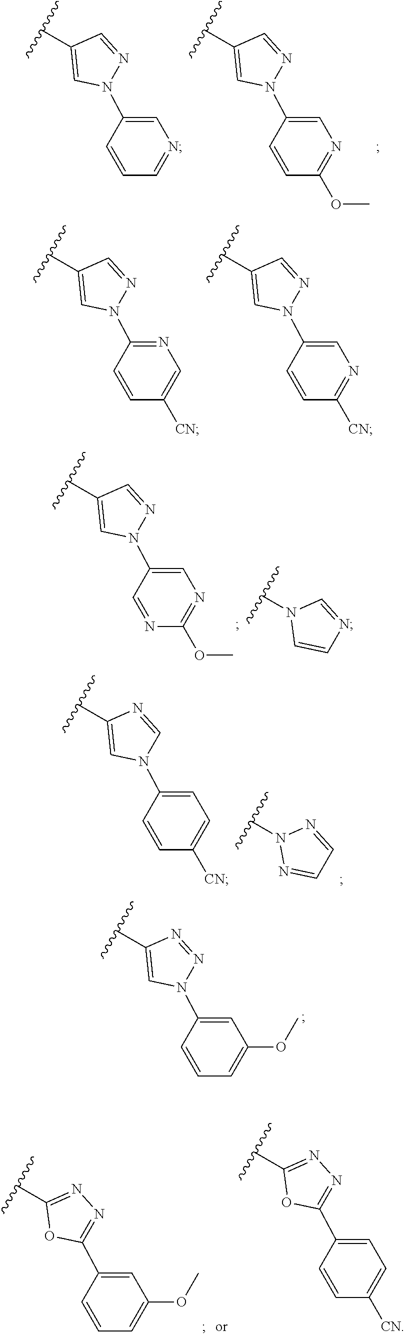

In yet a further compound of Formula (I), or (II), or pharmaceutically acceptable salts thereof, R₃ is —H, halogen, —CN, —N(H)(CH₂CH₂CO₂H), —C(O)C₁-C₃ alkyl, C₁-C₆ alkyl, C₁-C₆ haloalkyl, oxetane, isoxazole, or pyridine (preferably 3-pyridine). In yet a further compound of Formula (I), or (II), or pharmaceutically acceptable salts thereof, R₃ is —H, halogen, —CN, C₁-C₆ alkyl, C₁-C₆ haloalkyl, C₃-C₅ cycloalkyl, a heterocycle of 3 to 5 ring atoms containing 1, 2, or 3 ring heteroatoms independently selected from N, O, or S, or a heteroaryl of 5 ring atoms containing 1, 2, or 3 ring heteroatoms independently selected from N, O, or S. In yet a further compound of Formula (I), or (II), or pharmaceutically acceptable salts thereof, R₃ is —H, halogen, —CN, C₁-C₆ alkyl, C₁-C₆ haloalkyl, oxetane, or isoxazole. In yet a further compound of Formula (I), or (II), or pharmaceutically acceptable salts thereof, R₃ is —H, —CN, C₁-C₆ alkyl, or C₁-C₆ haloalkyl. In yet a further compound of Formula (I), or (II), or pharmaceutically acceptable salts thereof, R₃ is —H, —CN, C₁-C₃ alkyl or C₁-C₃ haloalkyl (preferably R₃ is —H, —CN, or C₁-C₃ alkyl); most preferably R₃ is —H, or methyl. Also preferably R₃ is —H, methyl, or trifluoromethyl.

In yet a further compound of Formula (I), or (II), or pharmaceutically acceptable salts thereof, R₄ is —H or halogen, preferably R₄ is —H.

In yet a further compound of Formula (I), or (II), or pharmaceutically acceptable salts thereof, R₅ is —H, halogen, C₁-C₆ alkyl, or C₁-C₆ haloalkyl; preferably R₅ is —H, halogen, C₁-C₃ alkyl or C₁-C₃ haloalkyl; more preferably R₅ is —H, halogen, methyl, or trifluoromethyl.

In yet a further compound of Formula (I), or (II), or pharmaceutically acceptable salts thereof, R₆ is —H or halogen.

In yet a further compound of Formula (I), or (II), or pharmaceutically acceptable salts thereof, R₃ is —H, —CN, C₁-C₃ alkyl, or C₁-C₃ haloalkyl (preferably R₃ is —H, —CN, or C₁-C₃ alkyl), and R₂ is a group of the formula:

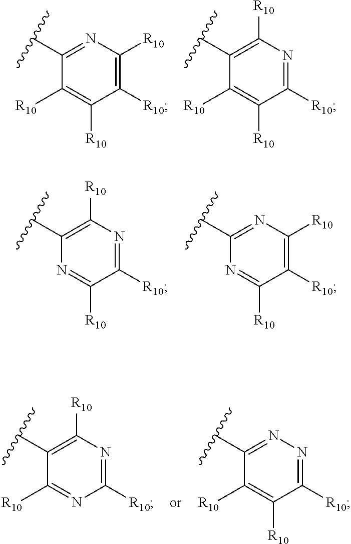

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN;
wherein each R₁₀ is independently —H, —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁—CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN; and each R₁₁ is independently —H or C₁-C₃ alkyl.

In yet a further compound of Formula (I), or (II), or pharmaceutically acceptable salts thereof, R₃ is —H, —CN, C₁-C₃ alkyl, or C₁-C₃ haloalkyl (preferably R₃ is —H, —CN, or C₁-C₃ alkyl), and R₂ is a group of the formula:

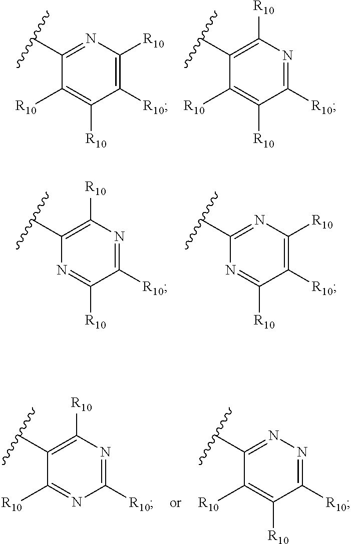

wherein each R₁₀ is independently —H, —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁—CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN; and each R₁₁ is independently —H or C₁-C₃ alkyl.

In yet a further compound of Formula (I), or (II), or pharmaceutically acceptable salts thereof, R₄ is —H or halogen (preferably R₄ is H), and R₂ is a group of the formula:

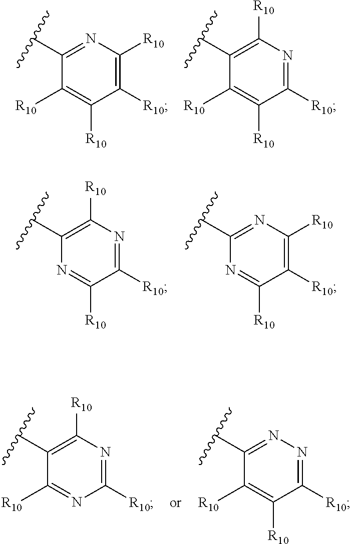

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN;
wherein each R₁₀ is independently —H, —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁—CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN; and each R₁₁ is independently —H or C₁-C₃ alkyl.

In yet a further compound of Formula (I), or (II), or pharmaceutically acceptable salts thereof, R₄ is —H or halogen (preferably R₄ is H), and R₂ is a group of the formula:

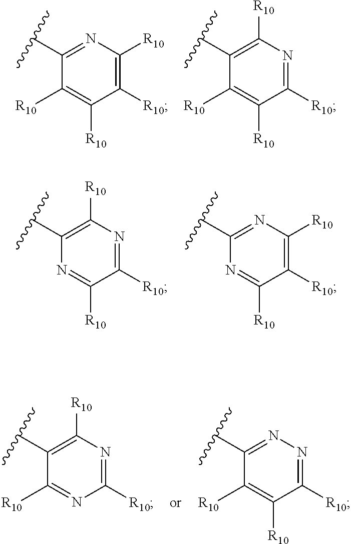

wherein each R₁₀ is independently —H, —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁—CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN; and each R₁₁ is independently —H or C₁-C₃ alkyl.

In yet a further compound of Formula (I), or (II), or pharmaceutically acceptable salts thereof, R₅ is —H, halogen, C₁-C₆ alkyl, or C₁-C₆ haloalkyl, and R₂ is a group of the formula:

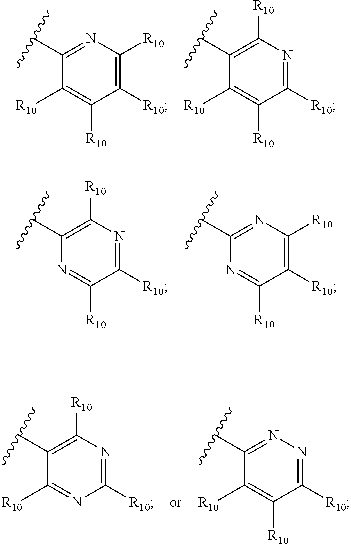

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN;
wherein each R₁₀ is independently —H, —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁—CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN; and each R₁₁ is independently —H or C₁-C₃ alkyl.

In yet a further compound of Formula (I), or (II), or pharmaceutically acceptable salts thereof, R₅ is —H, halogen, C₁-C₆ alkyl, or C₁-C₆ haloalkyl, and R₂ is a group of the formula:

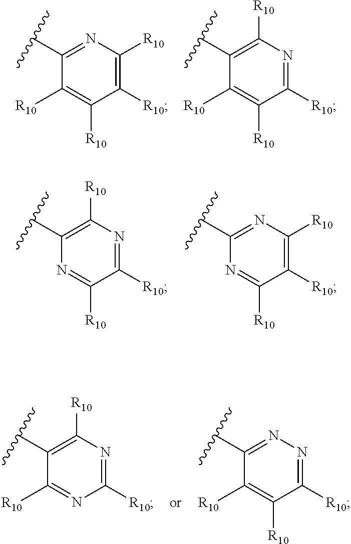

wherein each R₁₀ is independently —H, —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁—CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN; and each R₁₁ is independently —H or C₁-C₃ alkyl.

In yet a further compound of Formula (I), or (II), or pharmaceutically acceptable salts thereof, R₆ is —H or halogen, and R₂ is a group of the formula:

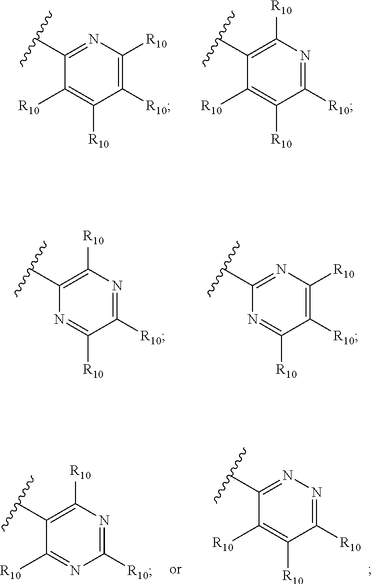

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN;
wherein each R₁₀ is independently —H, —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁—CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN; and each R₁₁ is independently —H or C₁-C₃ alkyl.

In yet a further compound of Formula (I), or (II), or pharmaceutically acceptable salts thereof, R₆ is —H or halogen, and R₂ is a group of the formula:

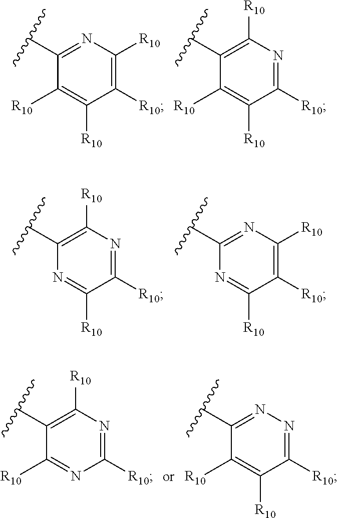

wherein each R₁₀ is independently —H, —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁—CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN; and each R₁₁ is independently —H or C₁-C₃ alkyl.

In yet a further compound of Formula (I), or (II), or pharmaceutically acceptable salts thereof, R₃ is —H, —CN, C₁-C₃ alkyl or C₁-C₃ haloalkyl, and R₄ is —H or halogen; more preferably R₃ is —H, —CN, or C₁-C₃ alkyl, and R₄ is H; most preferably R₃ is —H, or methyl, and R₄ is —H.

In yet a further compound of Formula (I), or (II), or pharmaceutically acceptable salts thereof, R₃ is —H, —CN, C₁-C₃ alkyl or C₁-C₃ haloalkyl, (preferably R₃ is —H, —CN, or C₁-C₃ alkyl), and R₅ is —H, halogen, C₁-C₃ alkyl or C₁-C₃ haloalkyl; more preferably R₃ is —H, or methyl, and R₅ is —H, halogen, methyl, or trifluoromethyl.

In yet a further compound of Formula (I), or (II), or pharmaceutically acceptable salts thereof, R₃ is —H, —CN, C₁-C₃ alkyl or C₁-C₃ haloalkyl, (preferably R₃ is —H, —CN, or C₁-C₃ alkyl), and R₆ is —H or halogen; more preferably R₃ is —H, or methyl, and R₆ is —H.

In yet a further compound of Formula (I), or (II), or pharmaceutically acceptable salts thereof, R₄ is —H or halogen (preferably R₄ is H), and R₅ is —H, halogen, C₁-C₃ alkyl or C₁-C₃ haloalkyl; preferably R₅ is —H, halogen, methyl, or trifluoromethyl.

In yet a further compound of Formula (I), or (II), or pharmaceutically acceptable salts thereof, R₄ is —H or halogen (preferably R₄ is —H) and R₆ is —H or halogen.

In yet a further compound of Formula (I), or (II), or pharmaceutically acceptable salts thereof, R₅ is —H, halogen, C₁-C₃ alkyl or C₁-C₃ haloalkyl, and R₆ is —H or halogen; preferably R₅ is —H, halogen, methyl, or trifluoromethyl, and R₆ is —H.

In yet a further compound of Formula (I), or (II), or pharmaceutically acceptable salts thereof, R₃ is —H, —CN, C₁-C₃ alkyl or C₁-C₃ haloalkyl, (preferably R₃ is —H, —CN, or C₁-C₃ alkyl), R₄ is —H or halogen (preferably R₄ is H), and R₂ is a group of the formula:

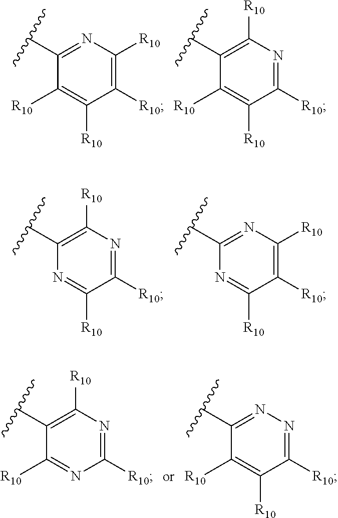

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN;
more preferably R₃ is —H, or methyl, and R₄ is —H.

In yet a further compound of Formula (I), or (II), or pharmaceutically acceptable salts thereof, R₃ is —H, —CN, C₁-C₃ alkyl or C₁-C₃ haloalkyl, (preferably R₃ is —H, —CN, or C₁-C₃ alkyl), R₄ is —H or halogen (preferably R₄ is H), and R₂ is a group of the formula:

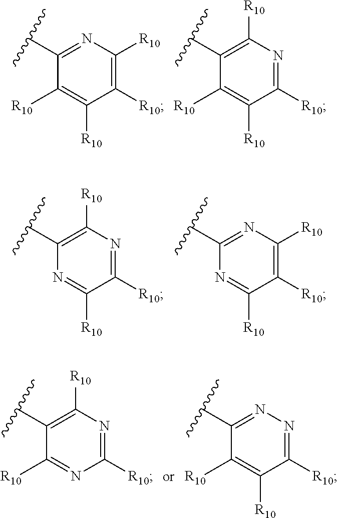

more preferably R₃ is —H, or methyl, and R₄ is —H.

In yet a further compound of Formula (I), or (II), or pharmaceutically acceptable salts thereof, R₃ is —H, —CN, C₁-C₃ alkyl or C₁-C₃ haloalkyl, (preferably R₃ is —H, —CN, or C₁-C₃ alkyl), R₅ is —H, halogen, C₁-C₆ alkyl, or C₁-C₆ haloalkyl, and R₂ is a group of the formula:

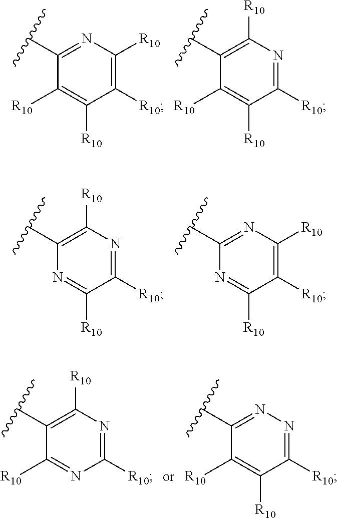

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN;
more preferably R₃ is —H, or methyl, and R₅ is —H, halogen, methyl, or trifluoromethyl.

In yet a further compound of Formula (I), or (II), or pharmaceutically acceptable salts thereof, R₃ is —H, —CN, C₁-C₃ alkyl or C₁-C₃ haloalkyl, (preferably R₃ is —H, —CN, or C₁-C₃ alkyl), R₅ is —H, halogen, C₁-C₆ alkyl, or C₁-C₆ haloalkyl, and R₂ is a group of the formula:

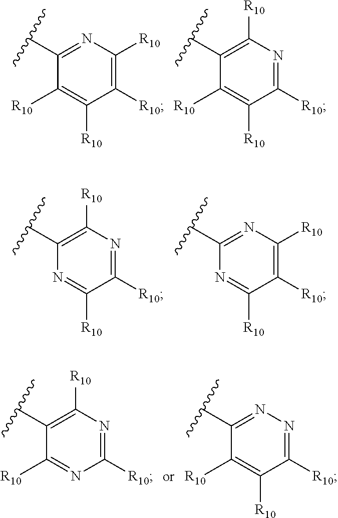

more preferably R₃ is —H, or methyl, and R₅ is —H, halogen, methyl, or trifluoromethyl.

In yet a further compound of Formula (I), or (II), or pharmaceutically acceptable salts thereof, R₃ is —H, —CN, C₁-C₃ alkyl or C₁-C₃ haloalkyl, (preferably R₃ is —H, —CN, or C₁-C₃ alkyl), R₆ is —H or halogen, and R₂ is a group of the formula:

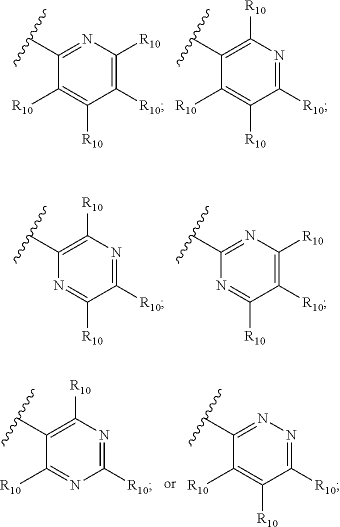

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN;
more preferably R₃ is —H, or methyl, and R₆ is —H.

In yet a further compound of Formula (I), or (II), or pharmaceutically acceptable salts thereof, R₃ is —H, —CN, C₁-C₃ alkyl or C₁-C₃ haloalkyl, (preferably R₃ is —H, —CN, or C₁-C₃ alkyl), R₆ is —H or halogen, and R₂ is a group of the formula:

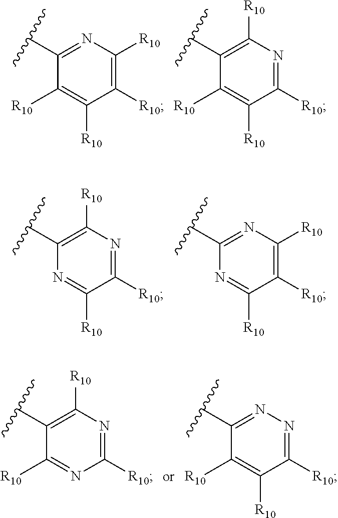

more preferably R₃ is —H, or methyl, and R₆ is —H.

In yet a further compound of Formula (I), or (II), or pharmaceutically acceptable salts thereof, R₄ is —H or halogen (preferably R₄ is H), R₅ is —H, halogen, C₁-C₃ alkyl or C₁-C₃ haloalkyl, and R₂ is a group of the formula:

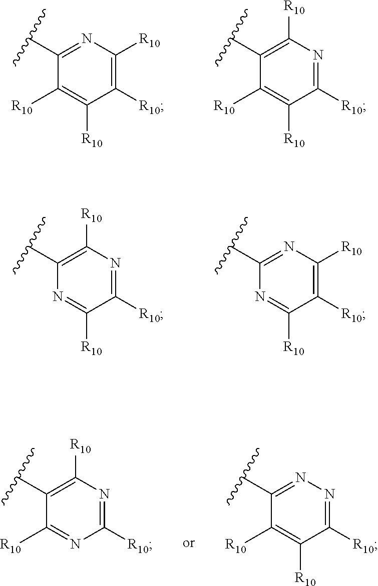

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN; more preferably R₅ is —H, halogen, methyl, or trifluoromethyl.

In yet a further compound of Formula (I), or (II), or pharmaceutically acceptable salts thereof, R₄ is —H or halogen (preferably R₄ is H), R₅ is —H, halogen, C₁-C₃ alkyl or C₁-C₃ haloalkyl, and R₂ is a group of the formula:

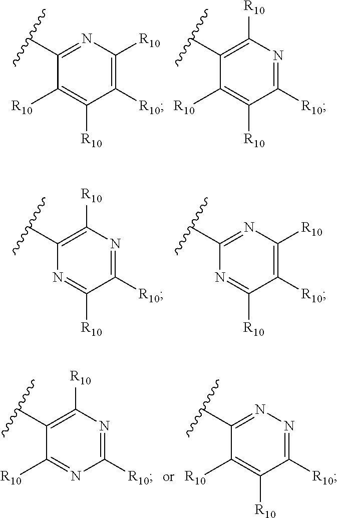

more preferably R₅ is —H, halogen, methyl, or trifluoromethyl.

In a compound of Formula (I), or (II), or pharmaceutically acceptable salts thereof, R₄ is —H or halogen (preferably R₄ is H), R₆ is —H or halogen, and R₂ is a group of the formula:

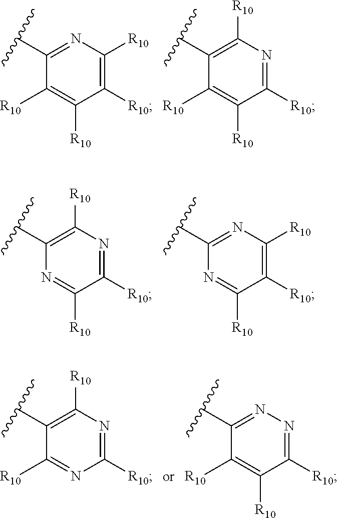

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN;
more preferably R₄ and R₆ are each —H.

In a compound of Formula (I), or (II), or pharmaceutically acceptable salts thereof, R₄ is —H or halogen (preferably R₄ is H), R₆ is —H or halogen, and R₂ is a group of the formula:

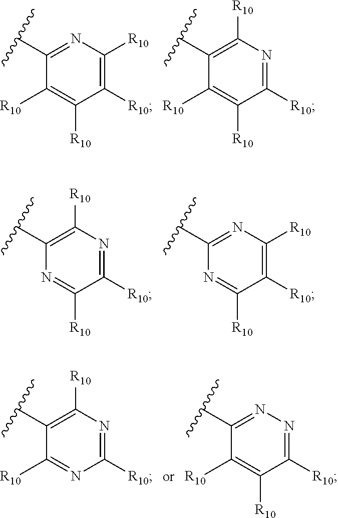

more preferably R₄ and R₆ are each —H.

In yet a further compound of Formula (I), or (II), or pharmaceutically acceptable salts thereof, R₅ is —H, halogen, C₁-C₃ alkyl or C₁-C₃ haloalkyl, R₆ is —H or halogen, and R₂ is a group of the formula:

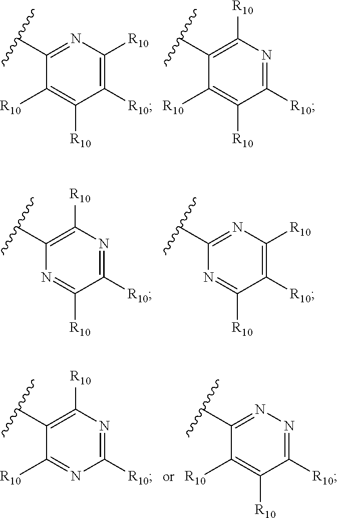

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN;
preferably R₅ is —H, halogen, methyl, or trifluoromethyl, and R₆ is —H.

In yet a further compound of Formula (I), or (II), or pharmaceutically acceptable salts thereof, R₅ is —H, halogen, C₁-C₃ alkyl or C₁-C₃ haloalkyl, R₆ is —H or halogen, and R₂ is a group of the formula:

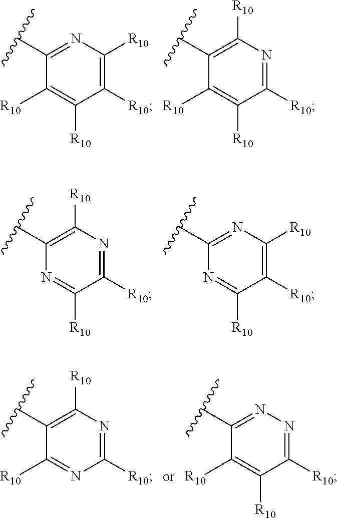

preferably R₅ is —H, halogen, methyl, or trifluoromethyl, and R₆ is —H.

In yet a further compound of Formula (I), or (II), or pharmaceutically acceptable salts thereof, R₃ is —H, —CN, C₁-C₃ alkyl, or C₁-C₃ haloalkyl (preferably R₃ is —H, —CN, or C₁-C₃ alkyl), R₄ is —H or halogen (preferably R₄ is H), and R₅ is —H, halogen, C₁-C₃ alkyl or C₁-C₃ haloalkyl; more preferably R₃ is —H, or methyl, R₄ is —H, and R₅ is —H, halogen, methyl, or trifluoromethyl.

In yet a further compound of Formula (I), or (II), or pharmaceutically acceptable salts thereof, R₃ is —H, —CN, C₁-C₃ alkyl, or C₁-C₃ haloalkyl (preferably R₃ is —H, —CN, or C₁-C₃ alkyl), R₄ is —H or halogen (preferably R₄ is H), and R₆ is —H or halogen; more preferably R₃ is —H, or methyl, and R₄ and R₆ are each —H.

In yet a further compound of Formula (I), or (II), or pharmaceutically acceptable salts thereof, R₃ is —H, —CN, C₁-C₃ alkyl, or C₁-C₃ haloalkyl (preferably R₃ is —H, —CN, or C₁-C₃ alkyl), R₅ is —H, halogen, C₁-C₃ alkyl or C₁-C₃ haloalkyl, and R₆ is —H or halogen; more preferably R₃ is —H, or methyl, R₅ is —H, halogen, methyl, or trifluoromethyl, and R₆ is —H.

In yet a further compound of Formula (I), or (II), or pharmaceutically acceptable salts thereof, R₅ is —H, halogen, C₁-C₃ alkyl or C₁-C₃ haloalkyl, R₄ is —H or halogen (preferably R₄ is H), and R₆ is —H or halogen; more preferably R₅ is —H, halogen, methyl, or trifluoromethyl, and R₄ and R₆ are each —H.

In yet a further compound of Formula (I), or (II), or pharmaceutically acceptable salts thereof, R₃ is —H, —CN, C₁-C₃ alkyl, or C₁-C₃ haloalkyl (preferably R₃ is —H, —CN, or C₁-C₃ alkyl), R₄ is —H or halogen (preferably R₄ is H), R₅ is —H, halogen, C₁-C₆ alkyl, C₁-C₆ haloalkyl, or C₁-C₆ alkoxy, and R₂ is a group of the formula:

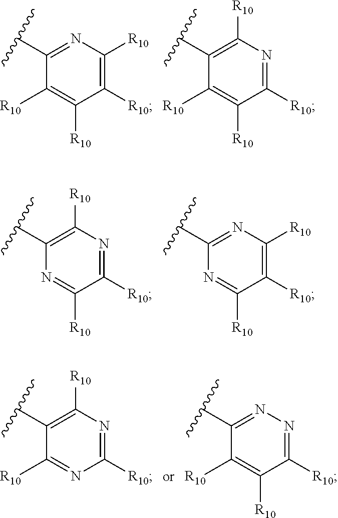

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN;
more preferably R₃ is —H, or methyl, R₄ is —H, and R₅ is —H, halogen, methyl, or trifluoromethyl.

In yet a further compound of Formula (I), or (II), or pharmaceutically acceptable salts thereof, R₃ is —H, —CN, C₁-C₃ alkyl, or C₁-C₃ haloalkyl (preferably R₃ is —H, —CN, or C₁-C₃ alkyl), R₄ is —H or halogen (preferably R₄ is H), R₅ is —H, halogen, C₁-C₆ alkyl, C₁-C₆ haloalkyl, or C₁-C₆ alkoxy, and R₂ is a group of the formula:

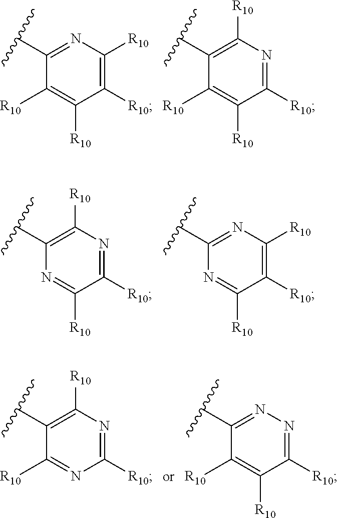

more preferably R₃ is —H, or methyl, R₄ is —H, and R₅ is —H, halogen, methyl, or trifluoromethyl.

In yet a further compound of Formula (I), or (II), or pharmaceutically acceptable salts thereof, R₃ is —H, —CN, C₁-C₃ alkyl, or C₁-C₃ haloalkyl (preferably R₃ is —H, —CN, or C₁-C₃ alkyl), R₄ is —H or halogen (preferably R₄ is —H), R₆ is —H, and R₂ is a group of the formula:

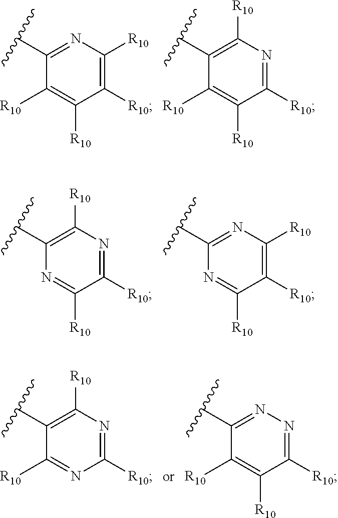

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN;
more preferably R₃ is —H, or methyl, and R₄ and R₆ are each —H.

In yet a further compound of Formula (I), or (II), or pharmaceutically acceptable salts thereof, R₃ is —H, —CN, C₁-C₃ alkyl, or C₁-C₃ haloalkyl (preferably R₃ is —H, —CN, or C₁-C₃ alkyl), R₄ is —H or halogen (preferably R₄ is —H), R₆ is —H, and R₂ is a group of the formula:

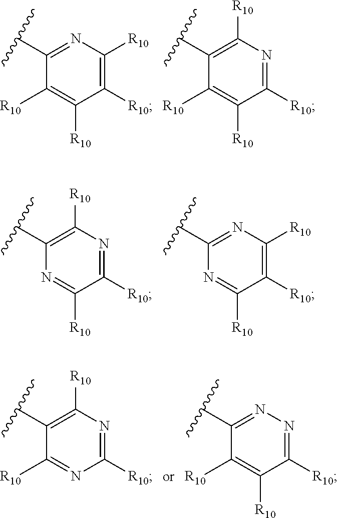

more preferably R₃ is —H, or methyl, and R₄ and R₆ are each —H.

In yet a further compound of Formula (I), or (II), or pharmaceutically acceptable salts thereof, R₃ is —H, —CN, C₁-C₃ alkyl, or C₁-C₃ haloalkyl (preferably R₃ is —H, —CN, or C₁-C₃ alkyl), R₅ is —H, halogen, C₁-C₆ alkyl, or C₁-C₆ haloalkyl, R₆ is —H or halogen, and R₂ is a group of the formula:

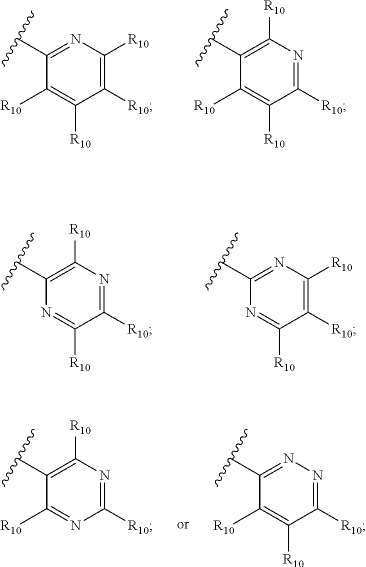

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN;
more preferably R₃ is —H, or methyl, R₅ is —H, halogen, methyl, or trifluoromethyl, and R₆ is —H.

In yet a further compound of Formula (I), or (II), or pharmaceutically acceptable salts thereof, R₃ is —H, —CN, C₁-C₃ alkyl, or C₁-C₃ haloalkyl (preferably R₃ is —H, —CN, or C₁-C₃ alkyl), R₅ is —H, halogen, C₁-C₆ alkyl, or C₁-C₆ haloalkyl, R₆ is —H or halogen, and R₂ is a group of the formula:

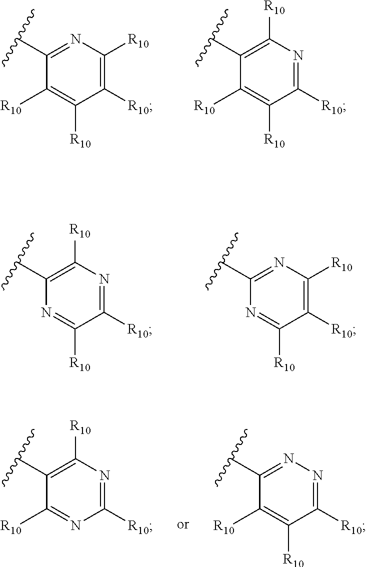

more preferably R₃ is —H, or methyl, R₅ is —H, halogen, methyl, or trifluoromethyl, and R₆ is —H.

In yet a further compound of Formula (I), or (II), or pharmaceutically acceptable salts thereof, R₅ is —H, halogen, C₁-C₃ alkyl or C₁-C₃ haloalkyl, R₄ is —H or halogen (preferably R₄ is H), R₆ is —H or halogen, and R₂ is a group of the formula:

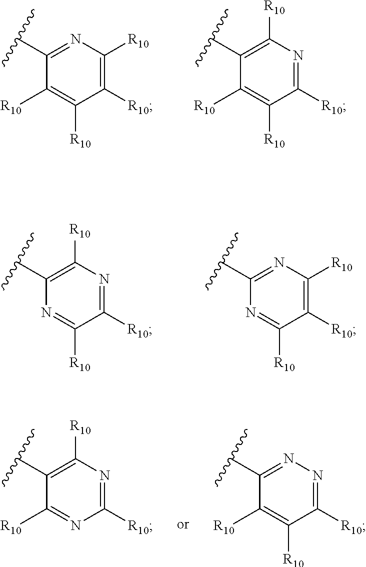

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN;
more preferably R₅ is —H, halogen, methyl, or trifluoromethyl, and R₄ and R₆ are each —H.

In yet a further compound of Formula (I), or (II), or pharmaceutically acceptable salts thereof, R₅ is —H, halogen, C₁-C₃ alkyl or C₁-C₃ haloalkyl, R₄ is —H or halogen (preferably R₄ is H), R₆ is —H or halogen, and R₂ is a group of the formula:

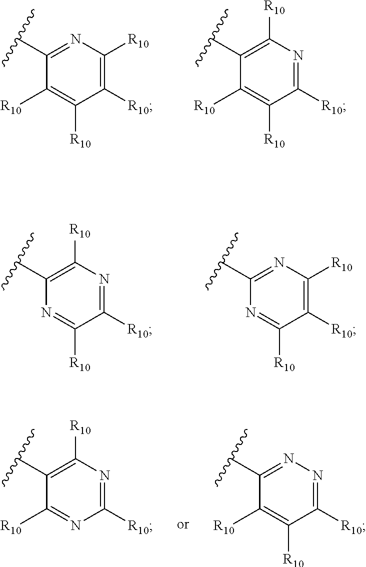

more preferably R₅ is —H, halogen, methyl, or trifluoromethyl, and R₄ and R₆ are each —H.

In yet a further compound of Formula (I), or (II), or pharmaceutically acceptable salts thereof, R₃ is —H, —CN, C₁-C₃ alkyl, or C₁-C₃ haloalkyl (preferably R₃ is —H, —CN, or C₁-C₃ alkyl), R₄ is —H or halogen (preferably R₄ is H), R₆ is —H or halogen, and R₅ is —H, halogen, C₁-C₃ alkyl or C₁-C₃ haloalkyl; preferably R₃ is —H, or methyl, R₄ and R₆ are each —H, and R₅ is —H, halogen, methyl, or trifluoromethyl.

In yet a further compound of Formula (I), or (II), or pharmaceutically acceptable salts thereof, R₃ is —H, —CN, C₁-C₃ alkyl, or C₁-C₃ haloalkyl (preferably R₃ is —H, —CN, or C₁-C₃ alkyl), R₄ is —H or halogen (preferably R₄ is H), R₆ is —H or halogen, R₅ is —H, halogen, C₁-C₃ alkyl or C₁-C₃ haloalkyl, and R₂ is a group of the formula:

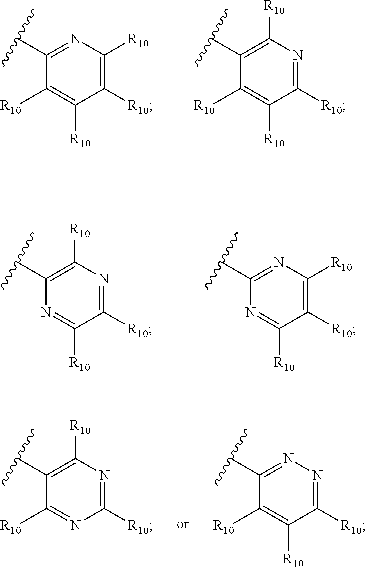

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN;
more preferably R₃ is —H, or methyl, R₄ and R₆ are each —H, and R₅ is —H, halogen, methyl, or trifluoromethyl.

In yet a further compound of Formula (I), or (II), or pharmaceutically acceptable salts thereof, R₃ is —H, —CN, C₁-C₃ alkyl, or C₁-C₃ haloalkyl (preferably R₃ is —H, —CN, or C₁-C₃ alkyl), R₄ is —H or halogen (preferably R₄ is H), R₆ is —H or halogen, R₅ is —H, halogen, C₁-C₃ alkyl or C₁-C₃ haloalkyl, and R₂ is a group of the formula:

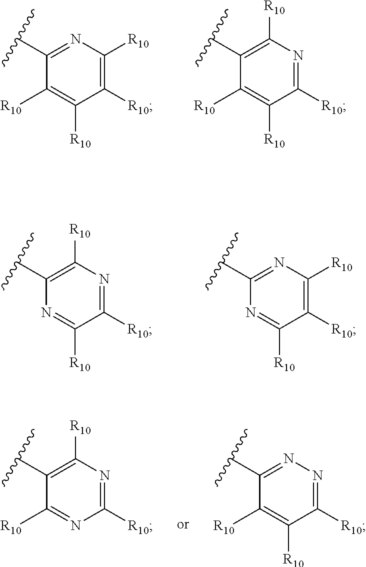

more preferably R₃ is —H, or methyl, R₄ and R₆ are each —H, and R₅ is —H, halogen, methyl, or trifluoromethyl.

In yet a further compound of Formula (I), or (II), or pharmaceutically acceptable salts thereof, R is —H.

In yet a further compound of Formula (I), or (II), or pharmaceutically acceptable salts thereof, R₇ is —CN, C₁-C₆ alkyl, or C₁-C₆ haloalkyl; preferably R₇ is —CN, C₁-C₃ alkyl or C₁-C₃ haloalkyl; more preferably R₇ is —CN, methyl or trifluoromethyl.

In yet a further compound of Formula (I), or (II), or pharmaceutically acceptable salts thereof, R₈ is —H.

In yet a further compound of Formula (I), or (II), or pharmaceutically acceptable salts thereof, R₇ is —CN, C₁-C₃ alkyl or C₁-C₃ haloalkyl, and R is —H. In yet a further compound of Formula (I), or (II), or pharmaceutically acceptable salts thereof, R₇ is C₁-C₃ alkyl (preferably methyl), and R is —H.

In yet a further compound of Formula (I), or (II), or pharmaceutically acceptable salts thereof, R₈ and R are each —H.

In yet a further compound of Formula (I), or (II), or pharmaceutically acceptable salts thereof, R₇ is —CN, C₁-C₃ alkyl or C₁-C₃ haloalkyl, and R₈ is —H. In yet a further compound of Formula (I), or (II), or pharmaceutically acceptable salts thereof, R₇ is C₁-C₃ alkyl (preferably methyl), and R₈ is —H.

In yet a further compound of Formula (I), or (II), or pharmaceutically acceptable salts thereof, R₇ is —CN, C₁-C₃ alkyl or C₁-C₃ haloalkyl, and R₈ and R are each —H. In yet a further compound of Formula (I), or (II), or pharmaceutically acceptable salts thereof, R₇ is C₁-C₃ alkyl (preferably methyl), R₈ and R are H.

In yet a further compound of Formula (I), or (II), or pharmaceutically acceptable salts thereof, R₇ is —CN, C₁-C₃ alkyl or C₁-C₃ haloalkyl, R₈ is —H, R is —H, and R₂ is a group of the formula:

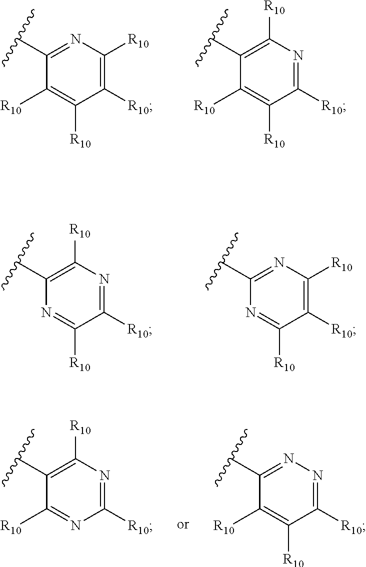

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN.

In yet a further compound of Formula (I), or (II), or pharmaceutically acceptable salts thereof, R₇ is —CN, C₁-C₃ alkyl or C₁-C₃ haloalkyl, R₈ is —H, R is —H, and R₂ is a group of the formula:

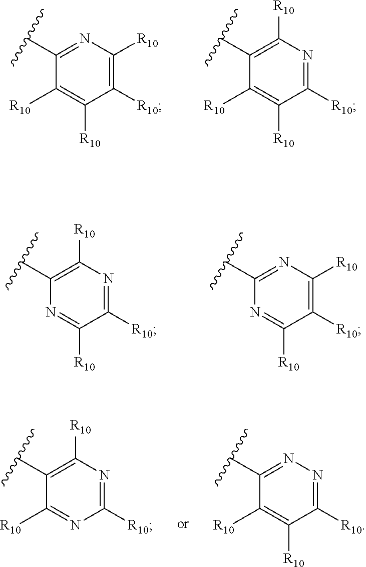

In yet a further compound of Formula (I), or (II), or pharmaceutically acceptable salts thereof, R₇ is C₁-C₃ alkyl (preferably methyl), R₈ is —H, R is —H, and R₂ is a group of the formula:

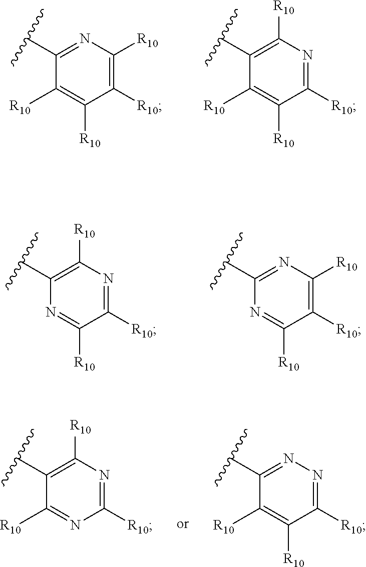

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN.

In yet a further compound of Formula (I), or (II), or pharmaceutically acceptable salts thereof, R₇ is C₁-C₃ alkyl (preferably methyl), R₈ is —H, R is —H, and R₂ is a group of the formula:

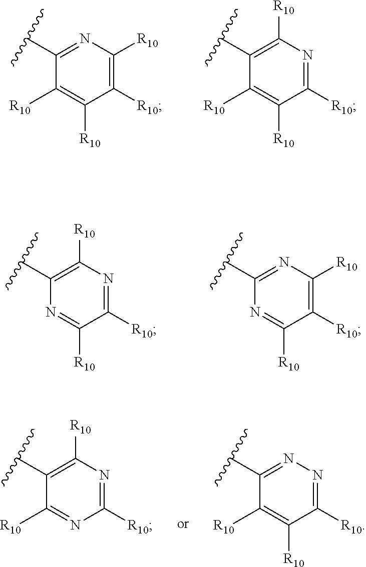

In yet a further compound of Formula (I), or (II), or pharmaceutically acceptable salts thereof, R₃ is —H, —CN, C₁-C₃ alkyl, or C₁-C₃ haloalkyl (preferably R₃ is —H, —CN, or C₁-C₃ alkyl), R₇ is —CN, C₁-C₃ alkyl or C₁-C₃ haloalkyl, and R₈ and R are each —H. In yet a further compound of Formula (I), or (II), or pharmaceutically acceptable salts thereof, R₃ is —H, or methyl, R₇ is C₁-C₃ alkyl (preferably methyl), and R₈ and R are each —H.

In yet a further compound of Formula (I), or (II), or pharmaceutically acceptable salts thereof, R₇ is —CN, C₁-C₃ alkyl or C₁-C₃ haloalkyl, and R₄, R₈ and R are each —H. In yet a further compound of Formula (I), or (II), or pharmaceutically acceptable salts thereof, R₇ is C₁-C₃ alkyl (preferably methyl), and R₄, R₈ and R are each —H.

In yet a further compound of Formula (I), or (II), or pharmaceutically acceptable salts thereof, R₅ is —H, halogen, C₁-C₃ alkyl or C₁-C₃ haloalkyl, R₇ is —CN, methyl or trifluoromethyl, and R₈ and R are each —H. In yet a further compound of Formula (I), or (II), or pharmaceutically acceptable salts thereof, R₅ is —H, halogen, methyl, or trifluoromethyl, R₇ is methyl, and R₈ and R are each —H.

In yet a further compound of Formula (I), or (II), or pharmaceutically acceptable salts thereof, R₃ is —H, —CN, C₁-C₃ alkyl, or C₁-C₃ haloalkyl (preferably R₃ is —H, —CN, or C₁-C₃ alkyl), R₄ is —H or halogen (preferably R₄ is H), R₆ is —H or halogen, R₅ is —H, halogen, C₁-C₆ alkyl, or C₁-C₆ haloalkyl, R₇ is —CN, methyl or trifluoromethyl, R₈ is —H, R is —H, and R₂ is a group of the formula:

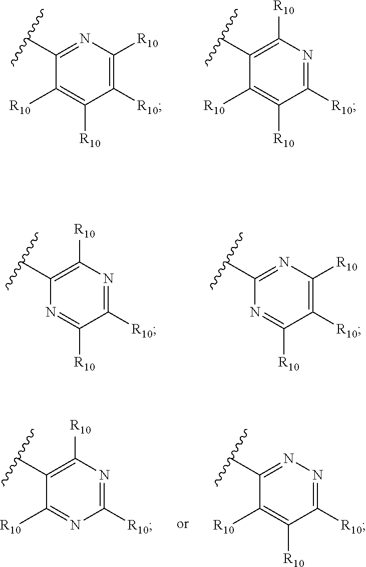

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN;
more preferably R₃ is —H, or methyl, R₄ and R₆ are each —H, R₅ is —H, halogen, methyl, or trifluoromethyl, R₇ is methyl, and R₈ and R are each —H.

In yet a further compound of Formula (I), or (II), or pharmaceutically acceptable salts thereof, R₃ is —H, —CN, C₁-C₃ alkyl, or C₁-C₃ haloalkyl (preferably R₃ is —H, —CN, or C₁-C₃ alkyl), R₄ is —H or halogen (preferably R₄ is H), R₆ is —H or halogen, R₅ is —H, halogen, C₁-C₆ alkyl, or C₁-C₆ haloalkyl, R₇ is —CN, methyl or trifluoromethyl, R₈ is —H, R is —H, and R₂ is a group of the formula:

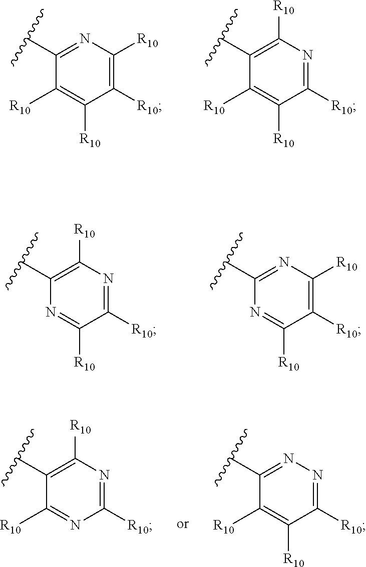

more preferably R₃ is —H, or methyl, R₄ and R₆ are each —H, R₅ is —H, halogen, methyl, or trifluoromethyl, R₇ is methyl, and R₈ and R are each —H.

In yet a further compound of Formula (I), or (II), or pharmaceutically acceptable salts thereif, R₁ is a group of the formula:

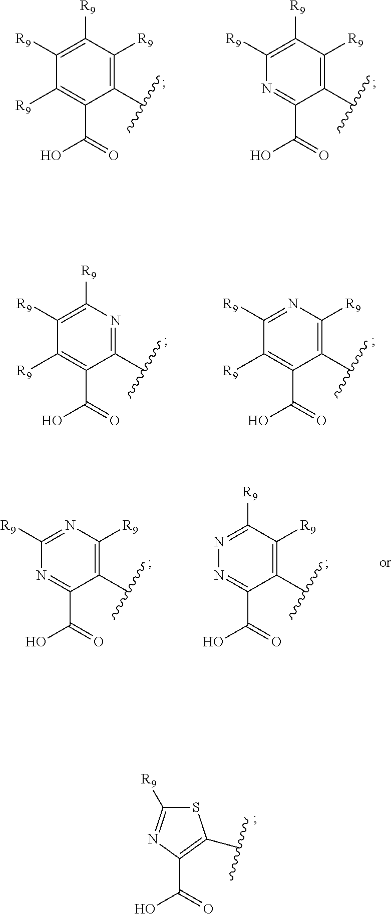

wherein each R₉ is independently —H, halogen, —CN, C₁-C₃ alkyl, C₁-C₃ haloalkyl, C₁-C₃ alkoxy, or C₃-C₅ cycloalkyl; preferably each R₉ is independently —H, halogen, —CN, methyl, trifluoromethyl, methoxy, or cyclopropyl.

In yet a further compound of Formula (I), or (II), or pharmaceutically acceptable salts thereof, R₁ is a group of the formula:

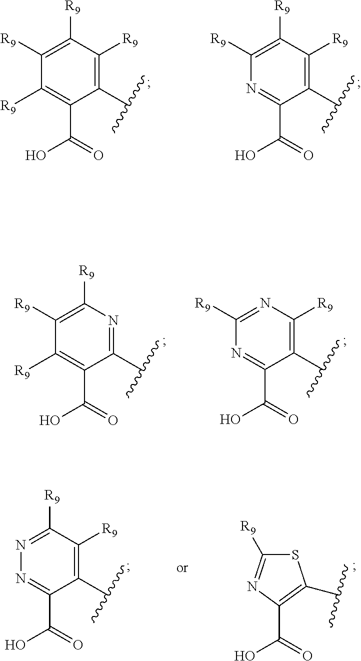

wherein each R₉ is independently —H, halogen, C₁-C₆ alkyl, C₁-C₆ haloalkyl, C₁-C₆ alkoxy, or C₃-C₅ cycloalkyl; preferably each R₉ is independently —H, halogen, C₁-C₃ alkyl, C₁-C₃ haloalkyl, C₁-C₃ alkoxy, or C₃-C₅ cycloalkyl; more preferably each R₉ is independently —H, halogen, methyl, trifluoromethyl, methoxy, or cyclopropyl.

In yet a further compound of Formula (I), or (II), or pharmaceutically acceptable salts thereof, R₁ is a group of the formula:

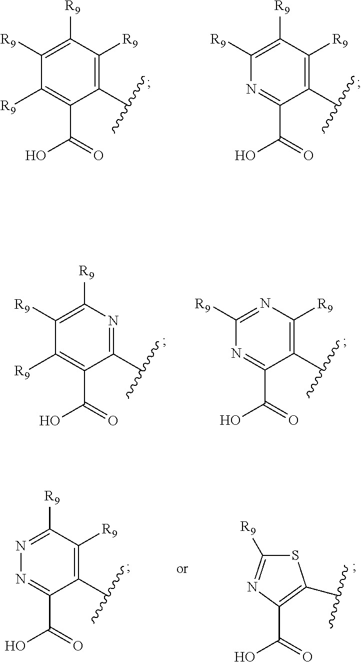

wherein each R₉ is independently —H, halogen, C₁-C₃ alkyl, C₁-C₃ haloalkyl, or C₃-C₅ cycloalkyl; preferably each R₉ is independently —H, halogen, methyl, trifluoromethyl, or cyclopropyl.

In yet a further compound of Formula (I), or (II), or pharmaceutically acceptable salts thereof, R₁ is a group of the formula:

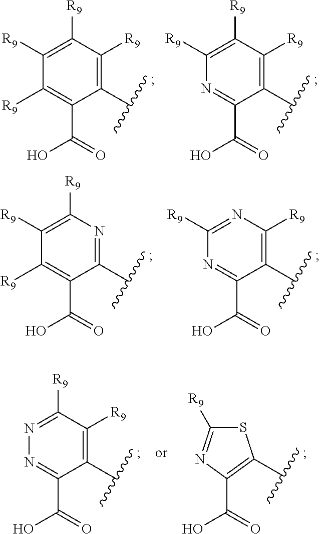

wherein each R₉ is independently —H, halogen, C₁-C₃ alkyl or C₁-C₃ haloalkyl; preferably each R₉ is independently —H, halogen, methyl or trifluoromethyl.

In yet a further compound of Formula (I), or (II), or pharmaceutically acceptable salts thereof, R₁ is a group of the formula:

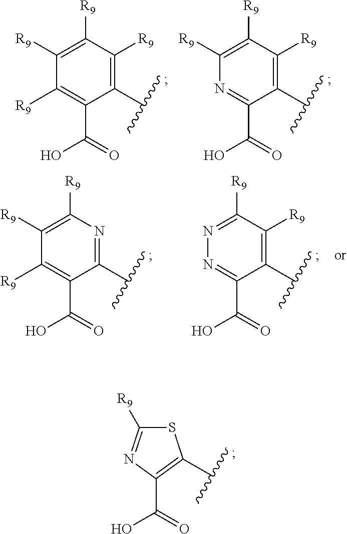

wherein each R₉ is independently —H, halogen, C₁-C₃ alkyl or C₁-C₃ haloalkyl; preferably each R₉ is independently —H, halogen, methyl, or trifluoromethyl.

In yet a further compound of Formula (I), or (II), or pharmaceutically acceptable salts thereof, R₁ is a group of the formula:

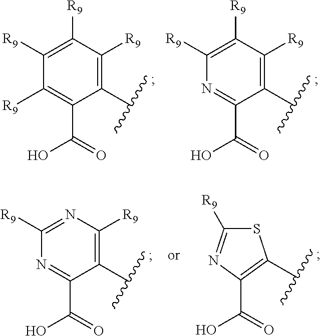

wherein each R₉ is independently —H, halogen, C₁-C₆ alkyl, C₁-C₆ haloalkyl, C₁-C₆ alkoxy, or C₃-C₅ cycloalkyl. Preferably each R₉ is independently —H, halogen, C₁-C₃ alkyl, C₁-C₃ haloalkyl, or C₃-C₅cycloalkyl. More preferably each R₉ is independently —H, halogen, methyl, trifluoromethyl, or cyclopropyl.

In yet a further compound of Formula (I), or (II), or pharmaceutically acceptable salts thereof, R₁ is a group of the formula:

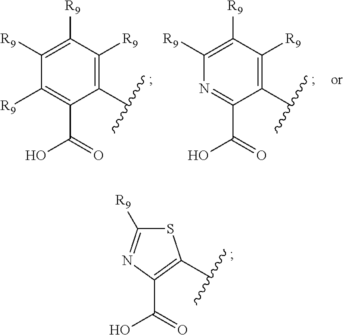

wherein each R₉ is independently —H, halogen, C₁-C₆ alkyl, C₁-C₆ haloalkyl, C₁-C₆ alkoxy, or C₃-C₅ cycloalkyl. Preferably each R₉ is independently —H, halogen, C₁-C₃ alkyl or C₁-C₃ haloalkyl. More preferably each R₉ is independently —H, halogen, or trifluoromethyl.

In yet a further compound of Formula (I), or (II), or pharmaceutically acceptable salts thereof, R₁ is a group of the formula

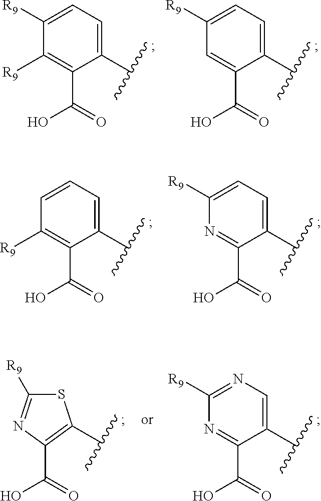

wherein each R₉ is independently —H, halogen, —CN, C₁-C₆ alkyl, C₁-C₆ haloalkyl, C₁-C₆ alkoxy, or C₃-C₅ cycloalkyl; preferably each R₉ is independently —H, halogen, —CN, C₁-C₃ alkyl, C₁-C₃ haloalkyl, C₁-C₃ alkoxy, or C₃-C₅ cycloalkyl; more preferably each R₉ is independently —H, halogen, —CN, methyl, trifluoromethyl, methoxy, or cyclopropyl.

In yet a further compound of Formula (I), or (II), or pharmaceutically acceptable salts thereof, R₁ is a group of the formula

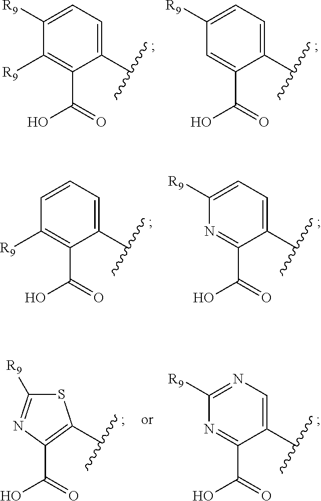

wherein each R₉ is independently —H, halogen, C₁-C₆ alkyl, C₁-C₆ haloalkyl, C₁-C₆ alkoxy, or C₃-C₅ cycloalkyl; preferably each R₉ is independently —H, halogen, C₁-C₃ alkyl, C₁-C₃ haloalkyl, C₁-C₃ alkoxy, or C₃-C₅ cycloalkyl; more preferably each R₉ is independently —H, halogen, methyl, trifluoromethyl, methoxy, or cyclopropyl.

In yet a further compound of Formula (I), or (II), or pharmaceutically acceptable salts thereof, R₁ is a group of the formula:

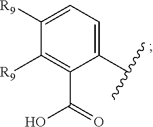

wherein each R₉ is independently —H, halogen, —CN, C₁-C₃ alkyl, C₁-C₃ haloalkyl or C₁-C₃ alkoxy. Preferably each R₉ is independently —H, halogen, C₁-C₃ alkyl or C₁-C₃ haloalkyl. More preferably each R₉ is independently —H, halogen, methyl or trifluoromethyl.

In yet a further compound of Formula (I), or (II), or pharmaceutically acceptable salts thereof, R₁ is a group of the formula:

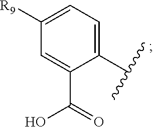

wherein R₉ is —H, halogen, —CN, C₁-C₃ haloalkyl, or C₁-C₃ alkoxy. Preferably R₉ is —H, halogen, or C₁-C₃ haloalkyl. More preferably R₉ is —H, or trifluoromethyl.

In yet a further compound of Formula (I), or (II), or pharmaceutically acceptable salts thereof, R₁ is a group of the formula:

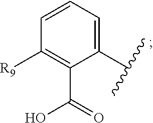

wherein R₉ is —H, halogen, —CN, C₁-C₃ haloalkyl, or C₁-C₃ alkoxy. Preferably R₉ is —H, halogen, or C₁-C₃ haloalkyl. More preferably R₉ is —H, or halogen. Even more preferably, R₉ is —H, or fluoro.

In yet a further compound of Formula (I), or (II), or pharmaceutically acceptable salts thereof, R₁ is a group of the formula:

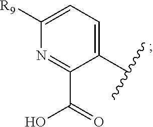

wherein R₉ is —H, halogen, —CN, C₁-C₃ alkyl, C₁-C₃ haloalkyl, C₁-C₃ alkoxy, or C₃-C₅ cycloalkyl. Preferably R₉ is —H, halogen, C₁-C₃ alkyl, C₁-C₃ haloalkyl, or C₃-C₅ cycloalkyl. More preferably R₉ is —H, halogen, methyl, trifluoromethyl, or cyclopropyl.

In yet a further compound of Formula (I), or (II), or pharmaceutically acceptable salts thereof, R₁ is a group of the formula:

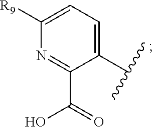

wherein R₉ is —H, halogen, or C₁-C₃ haloalkyl. Preferably R₉ is halogen or trifluoromethyl. More preferably R₉ is chloro or trifluoromethyl.

In yet a further compound of Formula (I), or (II), or pharmaceutically acceptable salts thereof, R₁ is a group of the formula:

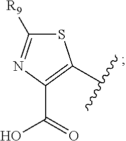

wherein R₉ is —H, halogen, —CN, C₁-C₆ alkyl, C₁-C₆ haloalkyl or C₁-C₆ alkoxy. Preferably R₉ is —H, halogen, C₁-C₆ alkyl or C₁-C₆ haloalkyl. More preferably R₉ is —H, halogen, C₁-C₃ alkyl or C₁-C₃ haloalkyl.

In yet a further compound of Formula (I), or (II), or pharmaceutically acceptable salts thereof, R₁ is a group of the formula:

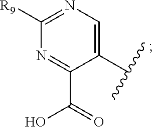

wherein R₉ is —H, halogen, —CN, C₁-C₃ alkyl, C₁-C₃ haloalkyl, C₁-C₃ alkoxy, or C₃-C₅ cycloalkyl. Preferably R₉ is —H, halogen, C₁-C₃ alkyl, C₁-C₃ haloalkyl, or C₃-C₅ cycloalkyl. More preferably R₉ is —H, halogen, methyl, trifluoromethyl, or cyclopropyl.

In yet a further compound of Formula (I), or (II), or pharmaceutically acceptable salts thereof, R₁ is a group of the formula:

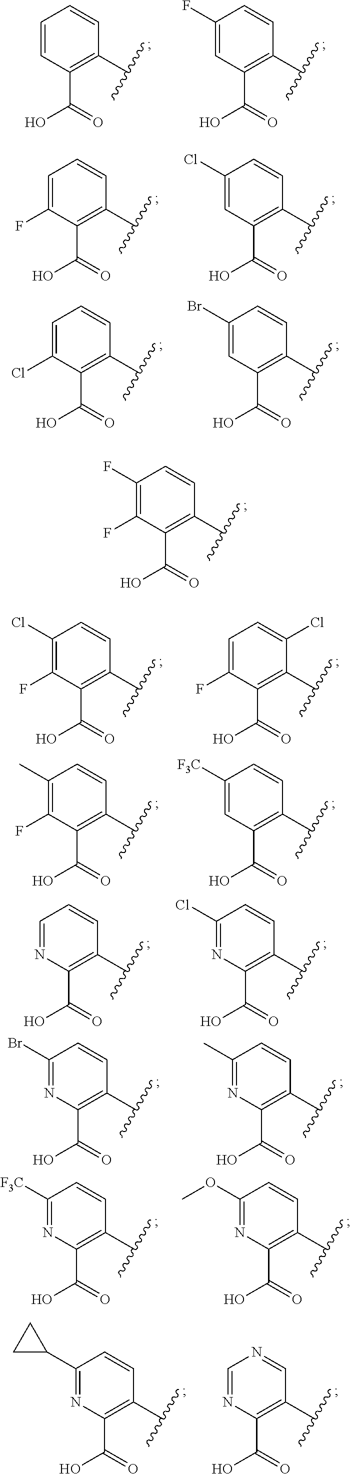

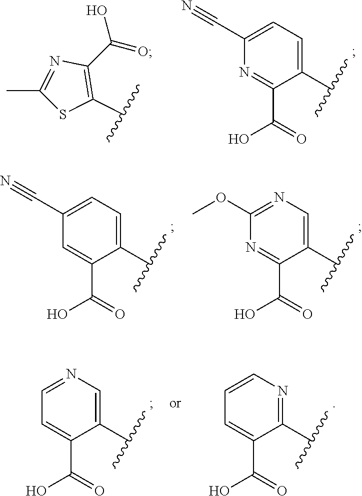

In yet a further compound of Formula (I), or (II), or pharmaceutically acceptable salts thereof, R₁ is a group of the formula:

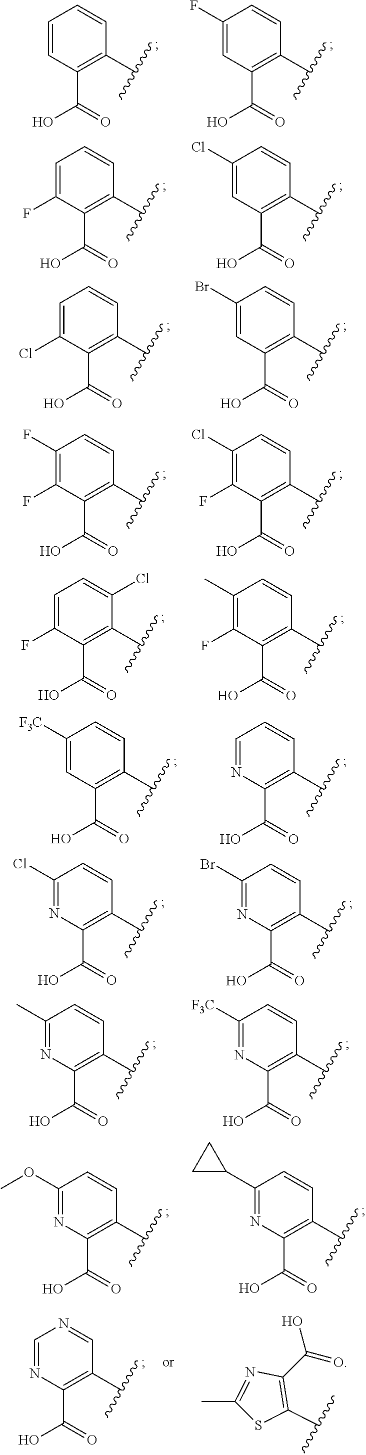

In yet a further compound of Formula (I), or (II), or pharmaceutically acceptable salts thereof, R₁ is a group of the formula:

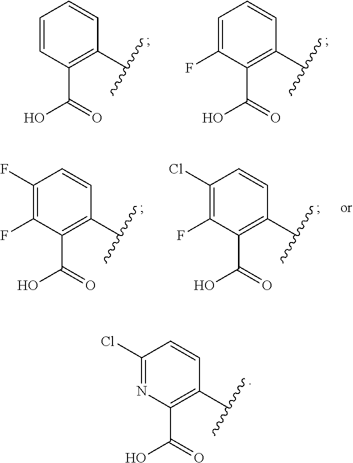 [SMILES: *c1ccccc1C(=O)O ; *c1cccc(F)c1C(=O)O ; *c1ccc(F)c(F)c1C(=O)O ; *c1ccc(Cl)c(F)c1C(=O)O ; *c1ccc(Cl)nc1C(=O)O]

In yet a further compound of Formula (I), or (II), or pharmaceutically acceptable salts thereof, R₃ is —H, —CN, C₁-C₃ alkyl, or C₁-C₃ haloalkyl, R₄ is —H, or halogen, R₆ is —H, or halogen, R₅ is —H, halogen, C₁-C₃ alkyl or C₁-C₃ haloalkyl, and R₁ is a group of the formula:

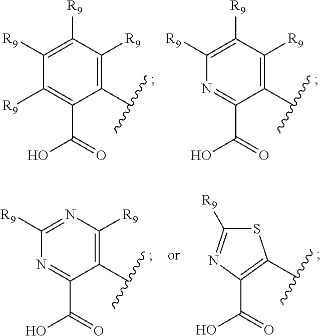

wherein each each R₉ is independently —H, halogen, C₁-C₃ alkyl, C₁-C₃ haloalkyl, or C₃-C₅ cycloalkyl. More preferably each R₉ is independently —H, halogen, methyl, trifluoromethyl, or cyclopropyl. Preferably R₃ is —H, methyl, or trifluoromethyl, R₄ is —H, or halogen, R₆ is —H, or halogen, R₅ is —H, halogen, methyl, or trifluoromethyl, and each R₉ is independently —H, halogen, methyl, trifluoromethyl, or cyclopropyl.

In yet a further compound of Formula (I), or (II), or pharmaceutically acceptable salts thereof, R₃ is —H, —CN, or C₁-C₃ alkyl, R₄ is —H, R₆ is —H or halogen, R₅ is —H, halogen, C₁-C₃ alkyl or C₁-C₃ haloalkyl, and R₁ is a group of the formula:

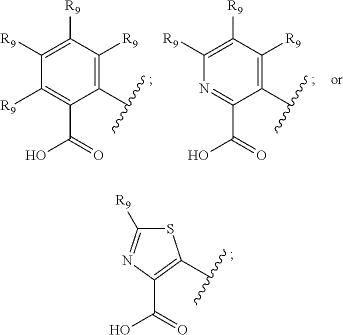

wherein each R₉ is independently —H, halogen, C₁-C₃ alkyl or C₁-C₃ haloalkyl. Preferably R₃ is —H, or methyl, R₄ and R₆ are each —H, R₅ is —H, halogen, methyl, or trifluoromethyl, and each R₉ is independently —H, halogen, methyl, or trifluoromethyl.

In yet a further compound of Formula (I), or (II), or pharmaceutically acceptable salts thereof, R₇ is —CN, methyl or trifluoromethyl, R₈ and R are each —H, and R₁ is a group of the formula:

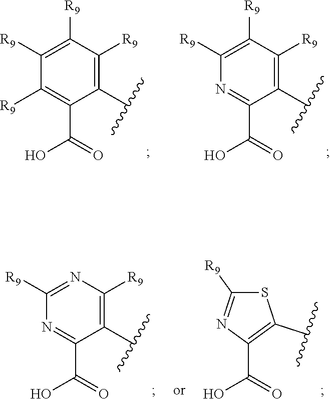

wherein each R₉ is independently —H, halogen, C₁-C₃ alkyl, C₁-C₃ haloalkyl, or C₃-C₅ cycloalkyl; more preferably R₇ is methyl, R₈ and R are each —H, and each R₉ is independently —H, halogen, methyl, trifluoromethyl, or cyclopropyl.

In yet a further compound of Formula (I), or (II), or pharmaceutically acceptable salts thereof, R₇ is —CN, methyl or trifluoromethyl, R₈ and R are each —H, and R₁ is a group of the formula:

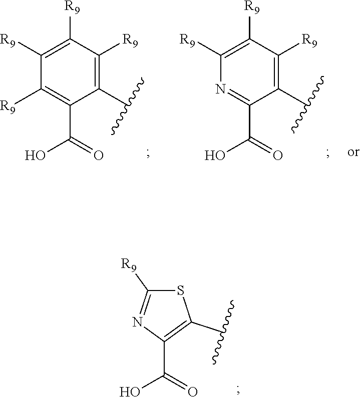

wherein each R₉ is independently —H, halogen, C₁-C₃ alkyl or C₁-C₃ haloalkyl; more preferably R₇ is methyl, R₈ and R are each —H, and each R₉ is independently —H, halogen, methyl, or trifluoromethyl.

In yet a further compound of Formula (I), or (II), or pharmaceutically acceptable salts thereof, R₃ is —H, —CN, C₁-C₃ alkyl, or C₁-C₃ haloalkyl, R₄ is —H, or halogen, R₈ and R are each —H, R₅ is —H, halogen, C₁-C₃ alkyl or C₁-C₃ haloalkyl, R₆ is —H, or halogen, R₇ is —CN, methyl or trifluoromethyl, and R₁ is a group of the formula:

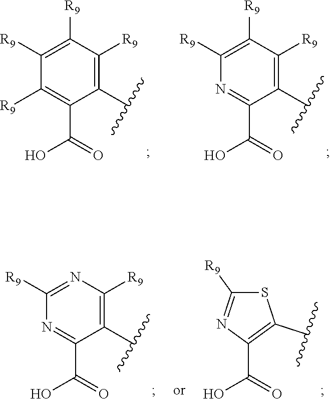

wherein each R₉ is independently —H, halogen, C₁-C₃ alkyl, C₁-C₃ haloalkyl, or C₃-C₅ cycloalkyl; more preferably R₃ is —H, methyl, or trifluoromethyl, R₄ is —H, or halogen, R₆ is —H, or halogen, R₈ and R are each —H, R₅ is —H, halogen, methyl, or trifluoromethyl, R₇ is methyl, and each R₉ is independently —H, halogen, methyl, trifluoromethyl, or cyclopropyl.

In yet a further compound of Formula (I), or (II), or pharmaceutically acceptable salts thereof, R₃ is —H, —CN, or C₁-C₃ alkyl, R₄, R₆, R₈ and R are each —H, R₅ is —H, halogen, C₁-C₃ alkyl or C₁-C₃ haloalkyl, R₇ is —CN, methyl or trifluoromethyl, and R₁ is a group of the formula:

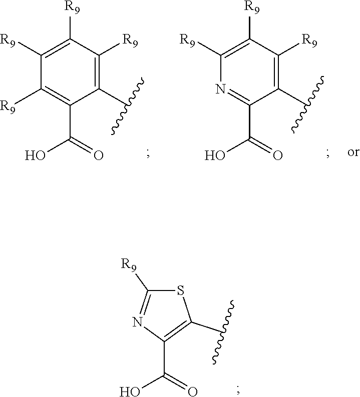

wherein each R₉ is independently —H, halogen, C₁-C₃ alkyl or C₁-C₃ haloalkyl; preferably R₃ is —H, or methyl, R₄, R₆, R₈ and R are each —H, R₅ is —H, halogen, methyl, or trifluoromethyl, R₇ is methyl, and each R₉ is independently —H, halogen, methyl, or trifluoromethyl.

In yet a further compound of Formula (I), or (II), or pharmaceutically acceptable salts thereof, R₃ is —H, —CN, C₁-C₃ alkyl, or C₁-C₃ haloalkyl, R₄ is —H, or halogen, R₈ and R are each —H, R₅ is —H, halogen, C₁-C₃ alkyl or C₁-C₃ haloalkyl, R₆ is —H, or halogen, R₇ is —CN, methyl or trifluoromethyl, and R₁ is a group of the formula:

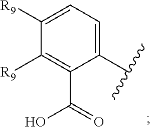

wherein each R₉ is independently —H, halogen, C₁-C₃ alkyl or C₁-C₃ haloalkyl; preferably R₃ is —H, methyl, or trifluoromethyl, R₄ is —H, or halogen, R₆ is —H, or halogen, R₈ and R are each —H, R₅ is —H, halogen, methyl, or trifluoromethyl, R₇ is methyl, and each R₉ is independently —H, halogen, methyl or trifluoromethyl.

In yet a further compound of Formula (I), or (II), or pharmaceutically acceptable salts thereof, R₃ is —H, —CN, or C₁-C₃ alkyl, R₄, R₆, R₈ and R are each —H, R₅ is —H, halogen, C₁-C₃ alkyl or C₁-C₃ haloalkyl, R₇ is —CN, methyl or trifluoromethyl, and R₁ is a group of the formula:

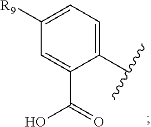

wherein R₉ is —H, halogen, or C₁-C₃ haloalkyl; preferably R₃ is —H, or methyl, R₄, R₆, R₈ and R are each —H, R₅ is —H, halogen, methyl, or trifluoromethyl, R₇ is methyl, and R₉ is —H, or trifluoromethyl.

In yet a further compound of Formula (I), or (II), or pharmaceutically acceptable salts thereof, R₃ is —H, —CN, or C₁-C₃ alkyl, R₄, R₆, R₈ and R are each —H, R₅ is —H, halogen, C₁-C₃ alkyl or C₁-C₃ haloalkyl, R₇ is —CN, methyl or trifluoromethyl, and R₁ is a group of the formula:

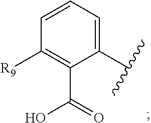

wherein R₉ is —H, halogen, or C₁-C₃ haloalkyl; preferably R₃ is —H, or methyl, R₄, R₆, R₈ and R are each —H, R₅ is —H, halogen, methyl, or trifluoromethyl, R₇ is methyl, and R₉ is —H, or halogen. More preferably, R₉ is —H, or fluoro.

In yet a further compound of Formula (I), or (II), or pharmaceutically acceptable salts thereof, R₃ is —H, —CN, C₁-C₃ alkyl, or C₁-C₃ haloalkyl, R₄ is —H, or halogen, R₈ and R are each —H, R₅ is —H, halogen, C₁-C₃ alkyl or C₁-C₃ haloalkyl, R₆ is —H, or halogen, R₇ is —CN, methyl or trifluoromethyl, and R₁ is a group of the formula:

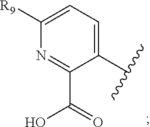

wherein R₉ is —H, halogen, C₁-C₃ alkyl, C₁-C₃ haloalkyl, or C₃-C₅ cycloalkyl; preferably R₃ is —H, methyl, or trifluoromethyl, R₄ is —H, or halogen, R₆ is —H, or halogen, R₈ and R are each —H, R₅ is —H, halogen, methyl, or trifluoromethyl, R₇ is methyl, and R₉ is —H, halogen, methyl, trifluoromethyl, or cyclopropyl.

In yet a further compound of Formula (I), or (II), or pharmaceutically acceptable salts thereof, R₃ is —H, —CN, or C₁-C₃ alkyl, R₄, R₆, R₈ and R are each —H, R₅ is —H, halogen, C₁-C₃ alkyl or C₁-C₃ haloalkyl, R₇ is —CN, methyl or trifluoromethyl, and R₁ is a group of the formula:

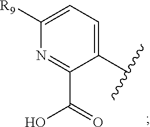

wherein R₉ is —H, halogen, or C₁-C₃ haloalkyl; preferably R₃ is —H, or methyl, it, R₆, R₈ and R are each —H, R₅ is —H, halogen, methyl, or trifluoromethyl, R₇ is methyl, and R₉ is halogen or trifluoromethyl.

In yet a further compound of Formula (I), or (II), or pharmaceutically acceptable salts thereof, R₃ is —H, —CN, or C₁-C₃ alkyl, it, R₆, R₈ and R are each —H, R₅ is —H, halogen, C₁-C₃ alkyl or C₁-C₃ haloalkyl, R₇ is —CN, methyl or trifluoromethyl, and R₁ is a group of the formula:

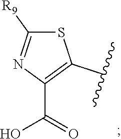

wherein R₉ is —H, halogen, C₁-C₃ alkyl or C₁-C₃ haloalkyl; preferably R₃ is —H, or methyl, it, R₆, R₈ and R are each —H, R₅ is —H, halogen, methyl, or trifluoromethyl, R₇ is methyl, and R₉ is —H, halogen, C₁-C₃ alkyl or C₁-C₃ haloalkyl.

In yet a further compound of Formula (I), or (II), or pharmaceutically acceptable salts thereof, R₃ is —H, —CN, C₁-C₃ alkyl, or C₁-C₃ haloalkyl, R₄ is —H, or halogen, R₈ and R are each —H, R₅ is —H, halogen, C₁-C₃ alkyl or C₁-C₃ haloalkyl, R₆ is —H, or halogen, R₇ is —CN, methyl or trifluoromethyl, and R₁ is a group of the formula:

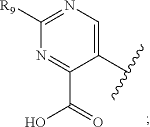

wherein R₉ is —H, halogen, C₁-C₃ alkyl, C₁-C₃ haloalkyl, or C₃-C₅ cycloalkyl; preferably R₃ is —H, methyl, or trifluoromethyl, R₄ is —H, or halogen, R₆ is —H, or halogen, R₈ and R are each —H, R₅ is —H, halogen, methyl, or trifluoromethyl, R₇ is methyl, and R₉ is —H, halogen, methyl, trifluoromethyl, or cyclopropyl.

In yet a further compound of Formula (I), or (II), or pharmaceutically acceptable salts thereof, R₁ is a group of the formula:

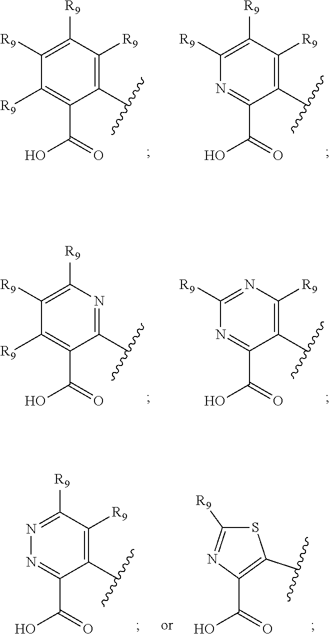

wherein each R₉ is independently —H, halogen, C₁-C₃ alkyl, C₁-C₃ haloalkyl, or C₃-C₅ cycloalkyl, and R₂ is a group of the formula:

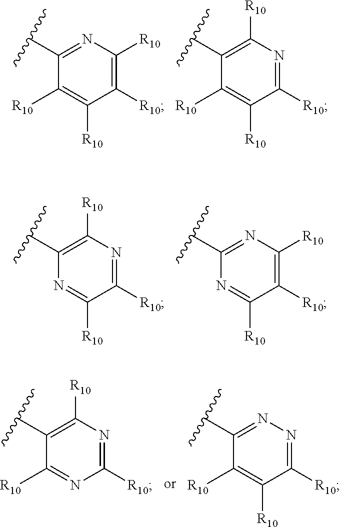

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁—CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN;
wherein each R₁₀ is independently —H, —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁—CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN; and each R₁₁ is independently —H or C₁-C₃ alkyl.

In yet a further compound of Formula (I), or (II), or pharmaceutically acceptable salts thereof, R₁ is a group of the formula:

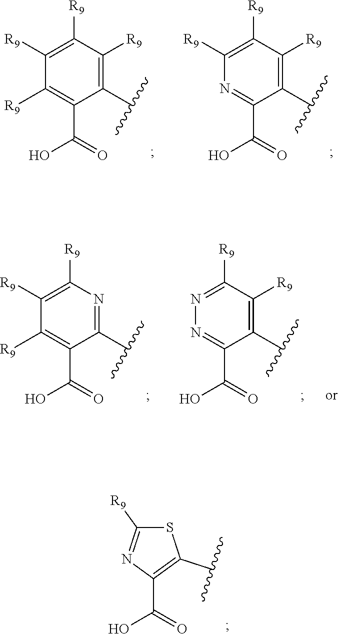

wherein each R₉ is independently —H, halogen, C₁-C₃ alkyl or C₁-C₃ haloalkyl, and R₂ is a group of the formula:

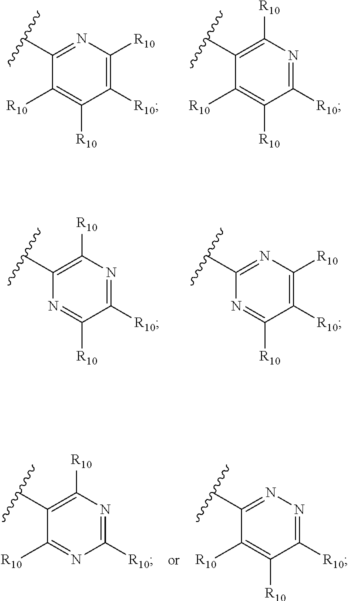

wherein each R₁₀ is independently —H, —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁—CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN; and each R₁₁ is independently —H or C₁-C₃ alkyl.

In yet a further compound of Formula (I), or (II), or pharmaceutically acceptable salts thereof, R₁ is a group of the formula:

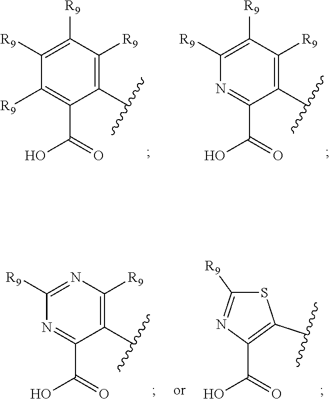

wherein each R₉ is independently —H, halogen, C₁-C₆ alkyl, C₁-C₆ haloalkyl, C₁-C₆ alkoxy, or C₃-C₅ cycloalkyl, and R₂ is a group of the formula:

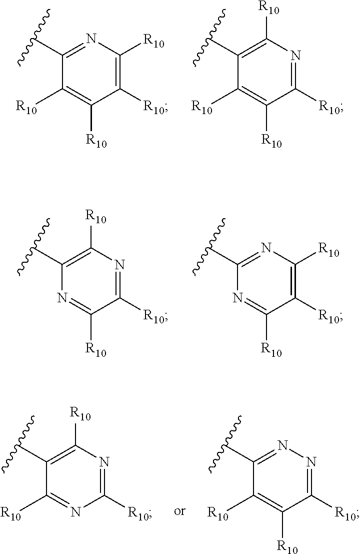

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN; preferably each R₉ is independently —H, halogen, C₁-C₃ alkyl or C₁-C₃ haloalkyl. Most preferably each R₉ is independently —H, halogen, methyl, or trifluoromethyl.

In yet a further compound of Formula (I), or (II), or pharmaceutically acceptable salts thereof, R₁ is a group of the formula:

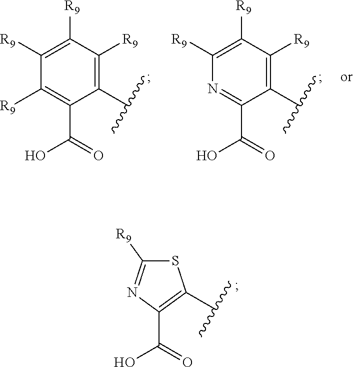

wherein each R₉ is independently —H, halogen, C₁-C₆ alkyl, C₁-C₆ haloalkyl, C₁-C₆ alkoxy, or C₃-C₅ cycloalkyl, and R₂ is a group of the formula:

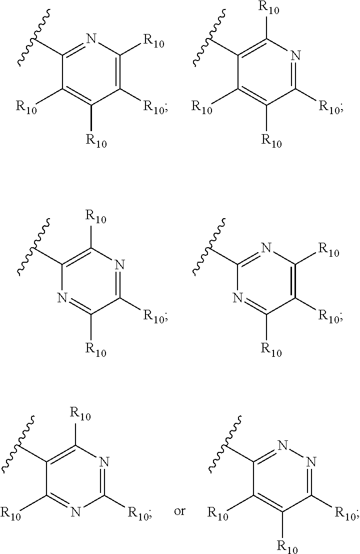

preferably each R₉ is independently —H, halogen, C₁-C₃ alkyl or C₁-C₃ haloalkyl. Most preferably each R₉ is independently —H, halogen, methyl, or trifluoromethyl.

In yet a further compound of Formula (I), or (II), or pharmaceutically acceptable salts thereof, R₁ is a group of the formula:

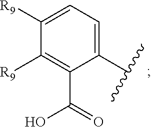

wherein each R₉ is independently —H, halogen, C₁-C₃ alkyl or C₁-C₃ haloalkyl, and R₂ is a group of the formula:

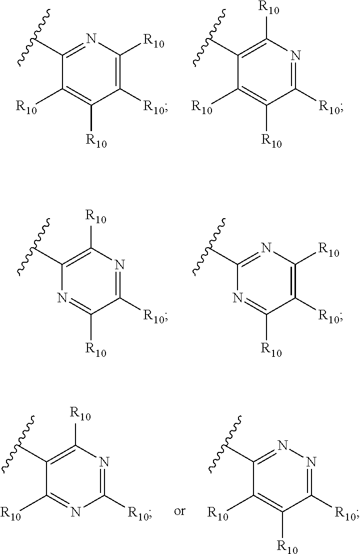

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN;
preferably each R₉ is independently —H, halogen, methyl or trifluoromethyl.

In yet a further compound of Formula (I), or (II), or pharmaceutically acceptable salts thereof, R₁ is a group of the formula:

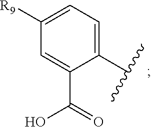

wherein R₉ is —H, halogen, or C₁-C₃ haloalkyl, and R₂ is a group of the formula:

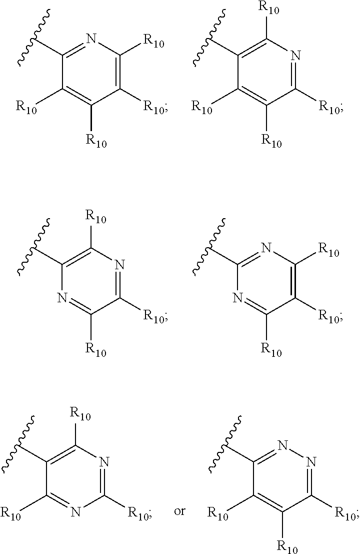

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN;
preferably R₉ is —H, or trifluoromethyl.

In yet a further compound of Formula (I), or (II), or pharmaceutically acceptable salts thereof, R₁ is a group of the formula:

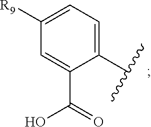

wherein R₉ is —H, halogen, or C₁-C₃ haloalkyl, and R₂ is a group of the formula:

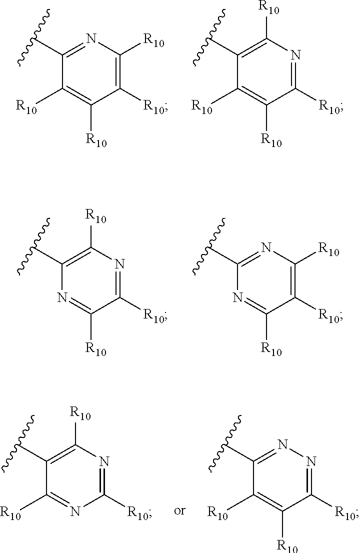

preferably R₉ is —H, or trifluoromethyl.

In yet a further compound of Formula (I), or (II), or pharmaceutically acceptable salts thereof, R₁ is a group of the formula:

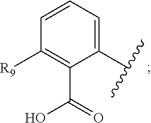

wherein R₉ is —H, halogen, or C₁-C₃ haloalkyl, and R₂ is a group of the formula:

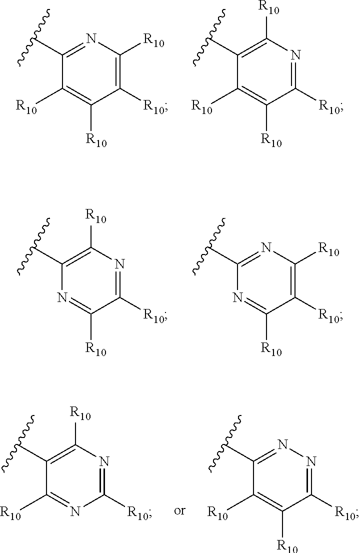

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN;
preferably R₉ is —H, or halogen.

In yet a further compound of Formula (I), or (II), or pharmaceutically acceptable salts thereof, R₁ is a group of the formula:

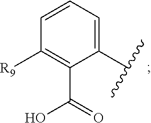

wherein R₉ is —H, halogen, or C₁-C₃ haloalkyl, and R₂ is a group of the formula:

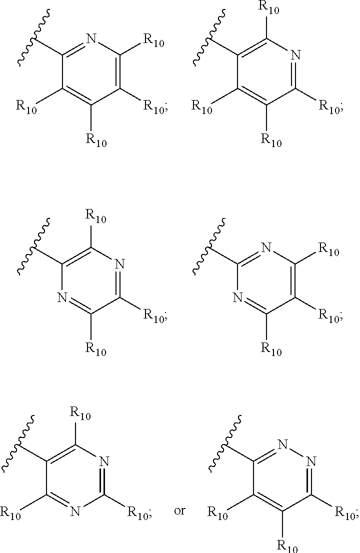

preferably R₉ is —H, or halogen.

In yet a further compound of Formula (I), or (II), or pharmaceutically acceptable salts thereof, R₁ is a group of the formula:

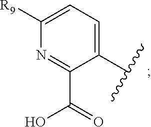

wherein R₉ is —H, halogen, C₁-C₃ alkyl, C₁-C₃ haloalkyl, or C₃-C₅ cycloalkyl, and R₂ is a group of the formula:

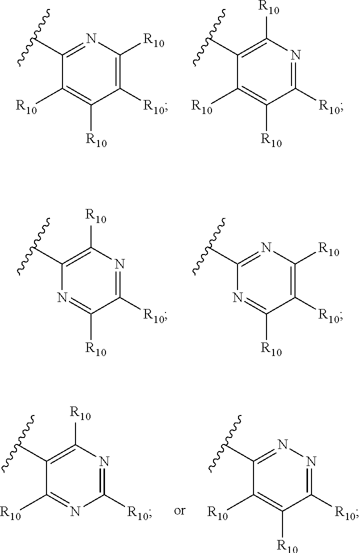

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN;
preferably R₉ is —H, halogen, methyl, trifluoromethyl, or cyclopropyl.

In yet a further compound of Formula (I), or (II), or pharmaceutically acceptable salts thereof, R₁ is a group of the formula:

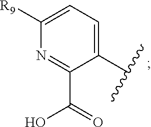

wherein R₉ is halogen, or C₁-C₃ haloalkyl, and R₂ is a group of the formula:

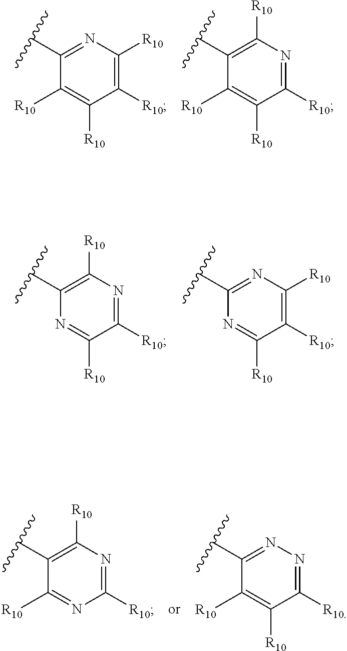

Preferably R₉ is halogen or trifluoromethyl. More preferably R₉ is chloro or trifluoromethyl.

In yet a further compound of Formula (I), or (II), or pharmaceutically acceptable salts thereof, R₁ is a group of the formula:

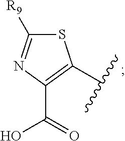

wherein R₉ is —H, halogen, C₁-C₃ alkyl or C₁-C₃ haloalkyl, and R₂ is a group of the formula:

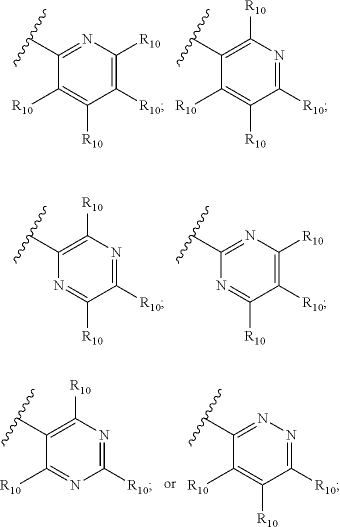

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN.

In yet a further compound of Formula (I), or (II), or pharmaceutically acceptable salts thereof, R₁ is a group of the formula:

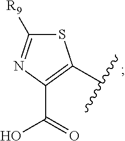

wherein R₉ is —H, halogen, C₁-C₃ alkyl or C₁-C₃ haloalkyl, and R₂ is a group of the formula:

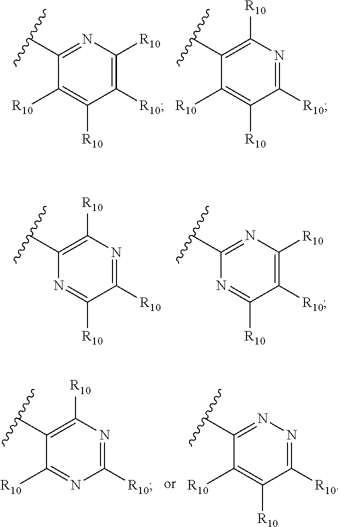

In yet a further compound of Formula (I), or (II), or pharmaceutically acceptable salts thereof, R₁ is a group of the formula:

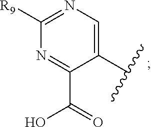

wherein R₉ is —H, halogen, C₁-C₃ alkyl, C₁-C₃ haloalkyl, or C₃-C₅ cycloalkyl, and R₂ is a group of the formula:

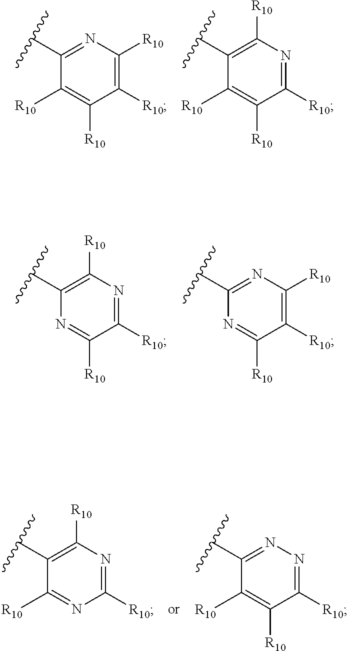

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN.

In yet a further compound of Formula (I), or (II), or pharmaceutically acceptable salts thereof, R₂ is a group of the formula:

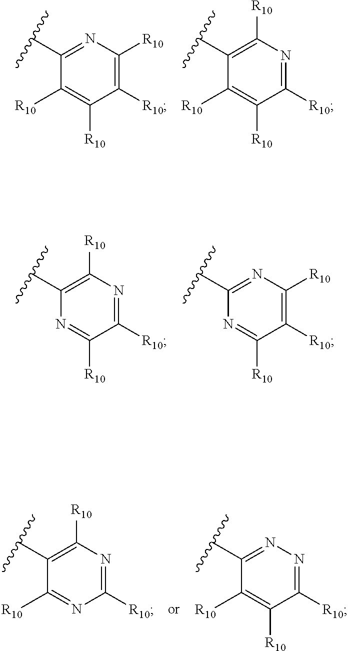

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN; R₃ is —H, —CN, C₁-C₃ alkyl, or C₁-C₃ haloalkyl, R₄ is —H, or halogen, R₆ is —H, or halogen, R₅ is —H, halogen, C₁-C₃ alkyl or C₁-C₃ haloalkyl, and R₁ is a group of the formula:

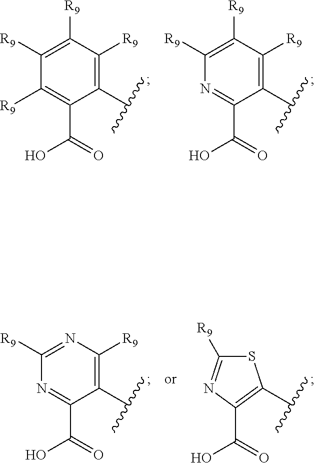

wherein each R₉ is independently —H, halogen, C₁-C₃ alkyl, C₁-C₃ haloalkyl, or C₃-C₅ cycloalkyl. Preferably R₃ is —H, methyl, or trifluoromethyl, R₄ is —H, or halogen, R₆ is —H, or halogen, R₅ is —H, halogen, methyl, or trifluoromethyl, and each R₉ is independently —H, halogen, methyl, trifluoromethyl, or cyclopropyl.

In yet a further compound of Formula (I), or (II), or pharmaceutically acceptable salts thereof, R₂ is a group of the formula:

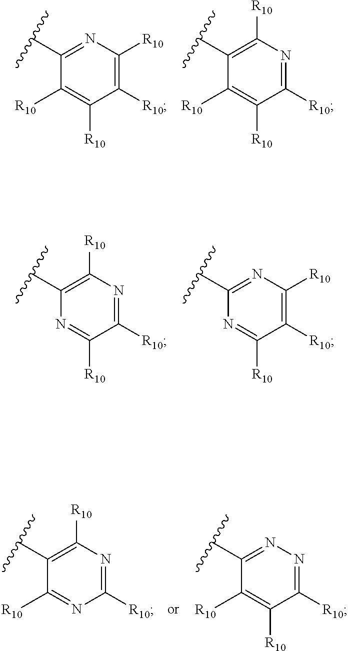

R₃ is —H, —CN, or C₁-C₃ alkyl, R₄ is —H, R₆ is —H or halogen, R₅ is —H, halogen, C₁-C₃ alkyl or C₁-C₃ haloalkyl, and R₁ is a group of the formula:

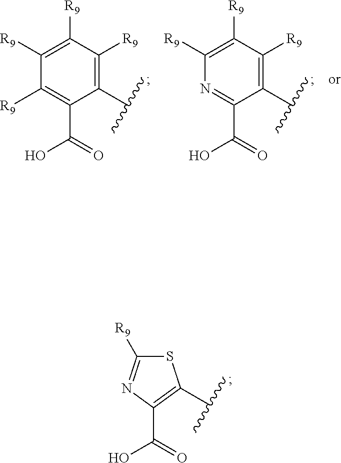

wherein each R₉ is independently —H, halogen, C₁-C₃ alkyl or C₁-C₃ haloalkyl. Preferably R₃ is —H, or methyl, R₄ and R₆ are each —H, R₅ is —H, halogen, methyl, or trifluoromethyl, and each R₉ is independently —H, halogen, methyl, or trifluoromethyl.

In yet a further compound of Formula (I), or (II), or pharmaceutically acceptable salts thereof, R₂ is a group of the formula:

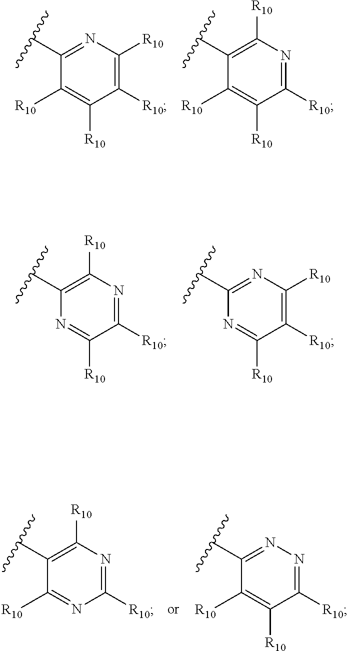

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN; R₇ is —CN, methyl or trifluoromethyl, R₈ and R are each —H, and R₁ is a group of the formula:

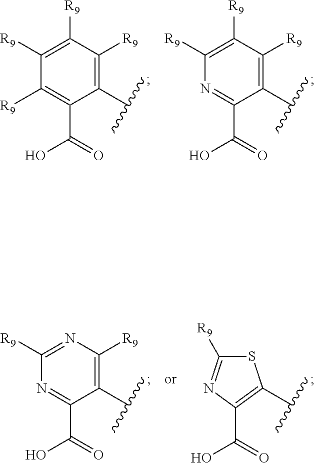

wherein each R₉ is independently —H, halogen, methyl, C₁-C₃ haloalkyl, or cyclopropyl.

In yet a further compound of Formula (I), or (II), or pharmaceutically acceptable salts thereof, R₂ is a group of the formula:

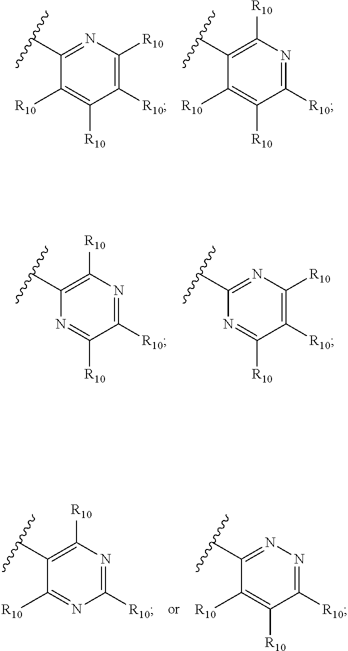

R₇ is —CN, methyl or trifluoromethyl, R₈ and R are each —H, and R₁ is a group of the formula:

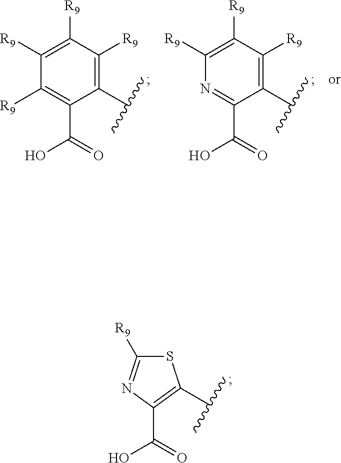

wherein each R₉ is independently —H, halogen, C₁-C₃ alkyl or C₁-C₃ haloalkyl.

In yet a further compound of Formula (I), or (II), or pharmaceutically acceptable salts thereof, R₁ is a group of the formula:

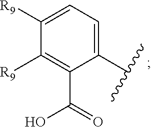

wherein each R₉ is independently —H, halogen, methyl or trifluoromethyl, R₂ is a group of the formula:

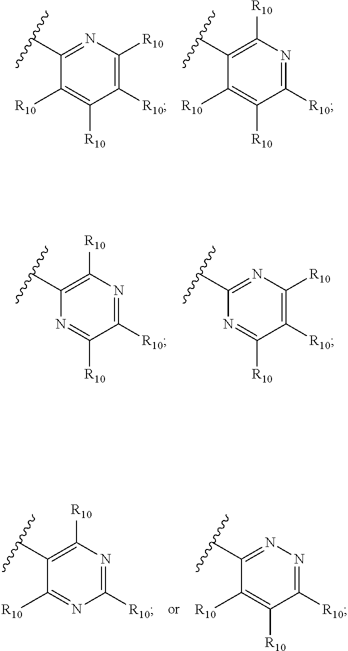

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN; R₇ is C₁-C₃ alkyl (preferably methyl), and R₈ and R are each —H.

In yet a further compound of Formula (I), or (II), or pharmaceutically acceptable salts thereof, R₁ is a group of the formula:

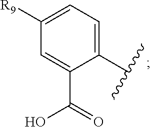

wherein R₉ is —H, halogen, or trifluoromethyl, (preferably R₉ is —H, or trifluoromethyl), R₂ is a group of the formula:

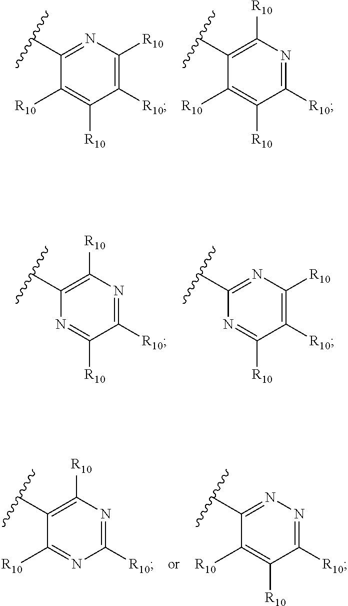

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN; R₇ is C₁-C₃ alkyl (preferably methyl), and R₈ and R are each —H.

In yet a further compound of Formula (I), or (II), or pharmaceutically acceptable salts thereof, R₁ is a group of the formula:

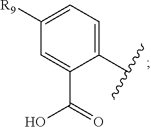

wherein R₉ is —H, halogen, or trifluoromethyl, (preferably R₉ is —H, or trifluoromethyl), R₂ is a group of the formula:

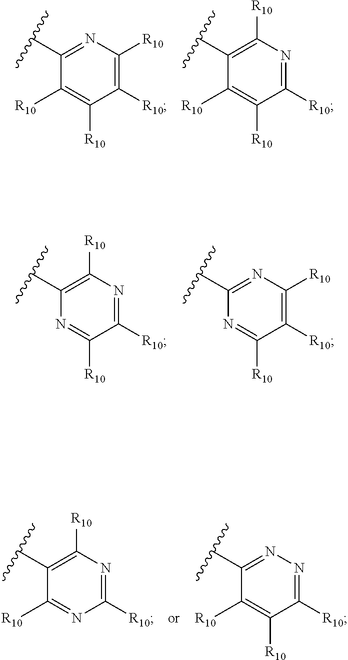

R₇ is C₁-C₃ alkyl (preferably methyl), and R₈ and R are each —H.

In yet a further compound of Formula (I), or (II), or pharmaceutically acceptable salts thereof, R₁ is a group of the formula:

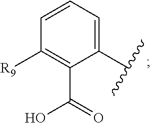

wherein R₉ is —H, halogen, or trifluoromethyl, (preferably R₉ is —H, or halogen), R₂ is a group of the formula:

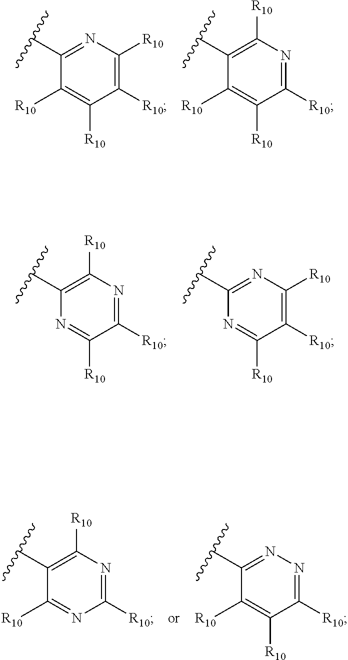

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN; R₇ is C₁-C₃ alkyl (preferably methyl), and R₈ and R are each —H.

In yet a further compound of Formula (I), or (II), or pharmaceutically acceptable salts thereof, R₁ is a group of the formula:

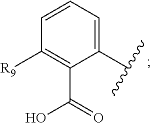

wherein R₉ is —H, halogen, or trifluoromethyl, (preferably R₉ is —H, or halogen), R₂ is a group of the formula:

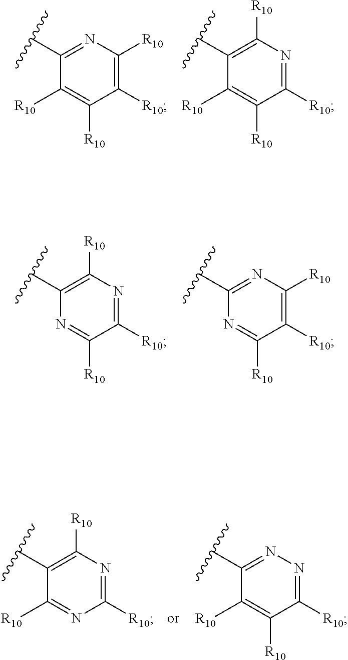

R₇ is C₁-C₃ alkyl (preferably methyl), and R₈ and R are each —H.

In yet a further compound of Formula (I), or (II), or pharmaceutically acceptable salts thereof, R₁ is a group of the formula:

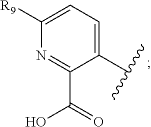

wherein R₉ is —H, halogen, methyl, trifluoromethyl, or cyclopropyl, R₂ is a group of the formula:

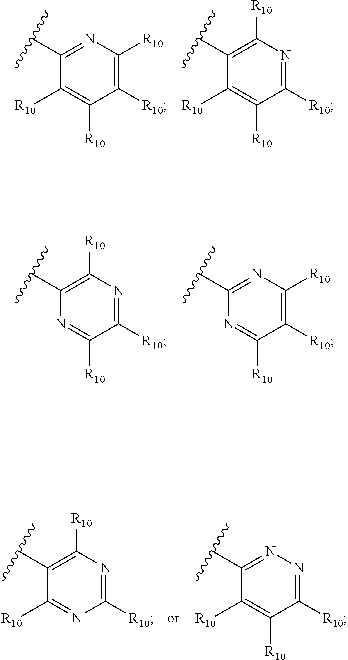

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN; R₇ is C₁-C₃ alkyl (preferably methyl), and R₈ and R are each —H.

In yet a further compound of Formula (I), or (II), or pharmaceutically acceptable salts thereof, R₁ is a group of the formula:

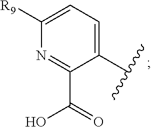

wherein R₉ is —H, halogen, or trifluoromethyl (preferably R₉ is halogen or trifluoromethyl), R₂ is a group of the formula:

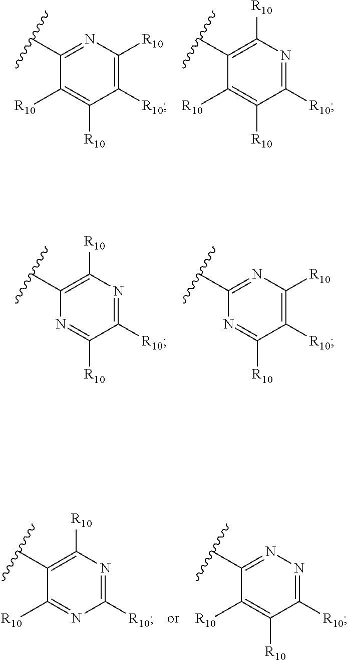

R₇ is C₁-C₃ alkyl (preferably methyl), and R₈ and R are each —H.

In yet a further compound of Formula (I), or (II), or pharmaceutically acceptable salts thereof, R₁ is a group of the formula:

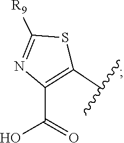

wherein R₉ is —H, halogen, C₁-C₃ alkyl or C₁-C₃ haloalkyl, R₂ is a group of the formula:

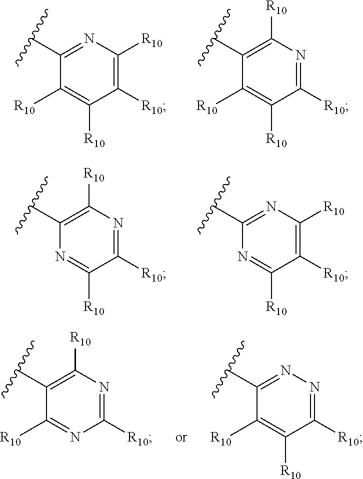

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN; R₇ is C₁-C₃ alkyl (preferably methyl), and R₈ and R are each —H.

In yet a further compound of Formula (I), or (II), or pharmaceutically acceptable salts thereof, R₁ is a group of the formula:

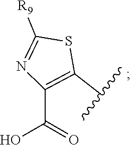

wherein R₉ is —H, halogen, C₁-C₃ alkyl or C₁-C₃ haloalkyl, R₂ is a group of the formula:

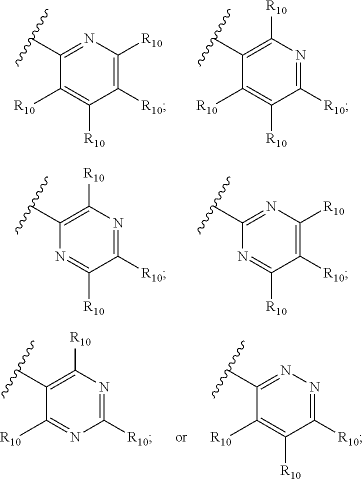

R₇ is C₁-C₃ alkyl (preferably methyl), and R₈ and R are each —H.

In yet a further compound of Formula (I), or (II), or pharmaceutically acceptable salts thereof, R₁ is a group of the formula:

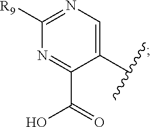

wherein R₉ is —H, halogen, C₁-C₃ alkyl, C₁-C₃ haloalkyl, or C₃-C₅ cycloalkyl, R₂ is a group of the formula:

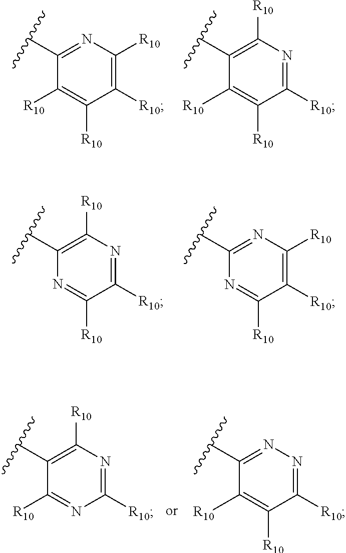

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN; R₇ is C₁-C₃ alkyl (preferably methyl), and R₈ and R are each —H.

In yet a further compound of Formula (I), or (II), or pharmaceutically acceptable salts thereof, R₂ is a group of the formula:

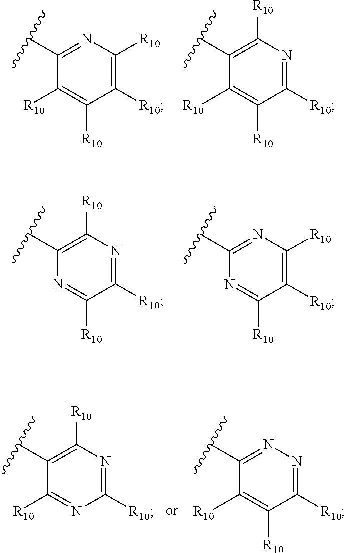

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN; R₃ is —H, —CN, or C₁-C₃ alkyl, R₄, R₆, R₈ and R are each —H, R₅ is —H, halogen, C₁-C₃ alkyl or C₁-C₃ haloalkyl, R₇ is —CN, methyl or trifluoromethyl, and R₁ is a group of the formula:

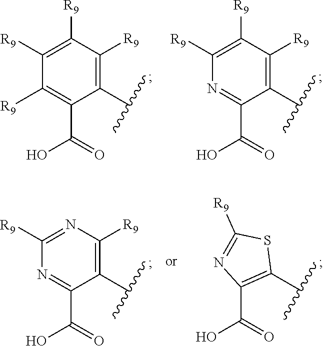

wherein each R₉ is independently —H, halogen, methyl, C₁-C₃ haloalkyl, or cyclopropyl.

In yet a further compound of Formula (I), or (II), or pharmaceutically acceptable salts thereof, R₂ is a group of the formula:

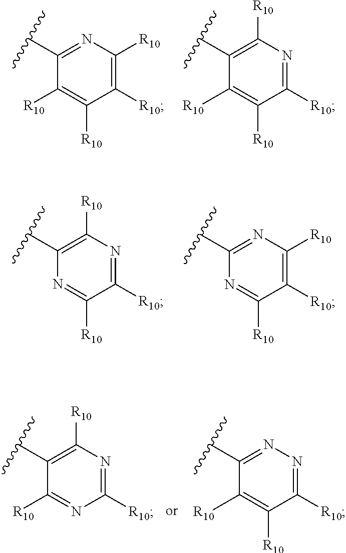

R₃ is —H, —CN, or C₁-C₃ alkyl, R₄, R₆, R₈ and R are each —H, R₅ is —H, halogen, C₁-C₃ alkyl or C₁-C₃ haloalkyl, R₇ is —CN, methyl or trifluoromethyl, and R₁ is a group of the formula:

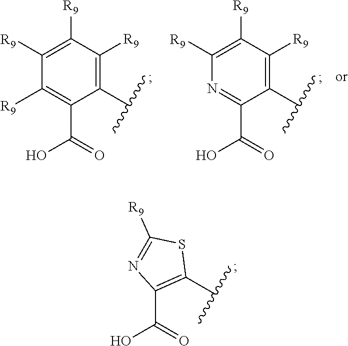

wherein each R₉ is independently —H, halogen, C₁-C₃ alkyl or C₁-C₃ haloalkyl.

In yet a further compound of Formula (I), or (II), or pharmaceutically acceptable salts thereof, R₁ is a group of the formula:

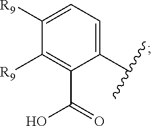

wherein each R₉ is independently —H, halogen, methyl or trifluoromethyl, R₂ is a group of the formula:

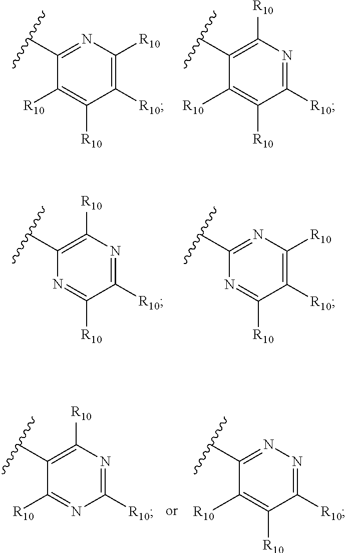

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN; R₃ is —H, methyl, or trifluoromethyl, R₄ is —H, or halogen, R₈ and R are each —H, R₅ is —H, halogen, methyl or trifluoromethyl, R₆ is —H, or halogen, and R₇ is C₁-C₃ alkyl (preferably methyl).

In yet a further compound of Formula (I), or (II), or pharmaceutically acceptable salts thereof, R₁ is a group of the formula:

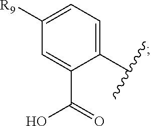

wherein R₉ is —H, halogen, or trifluoromethyl, (preferably R₉ is —H, or trifluoromethyl), R₂ is a group of the formula:

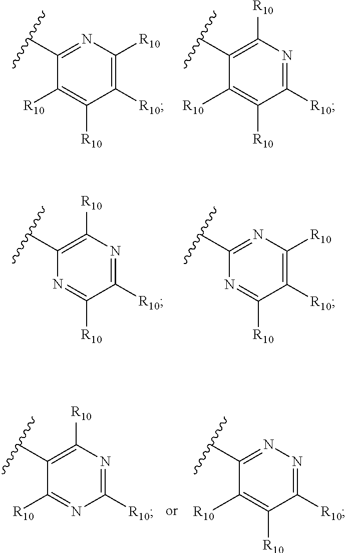

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN; R₃ is —H, or methyl, R₄, R₆, R₈ and R are each —H, R₅ is —H, halogen, methyl, or trifluoromethyl, and R₇ is C₁-C₃ alkyl (preferably methyl).

In yet a further compound of Formula (I), or (II), or pharmaceutically acceptable salts thereof, R₁ is a group of the formula:

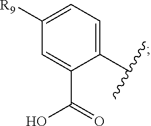

wherein R₉ is —H, halogen, or trifluoromethyl, (preferably R₉ is —H, or trifluoromethyl), R₂ is a group of the formula:

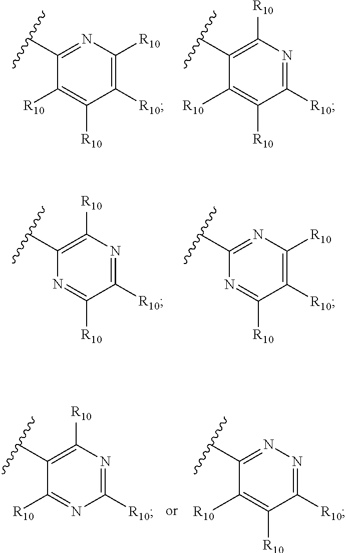

R₃ is —H, or methyl, R₄, R₆, R₈ and R are each —H, R₅ is —H, halogen, methyl, or trifluoromethyl, and R₇ is C₁-C₃ alkyl (preferably methyl).

In yet a further compound of Formula (I), or (II), or pharmaceutically acceptable salts thereof, R₁ is a group of the formula:

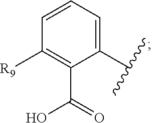

wherein R₉ is —H, halogen, or trifluoromethyl, (preferably R₉ is —H, or halogen), R₂ is a group of the formula:

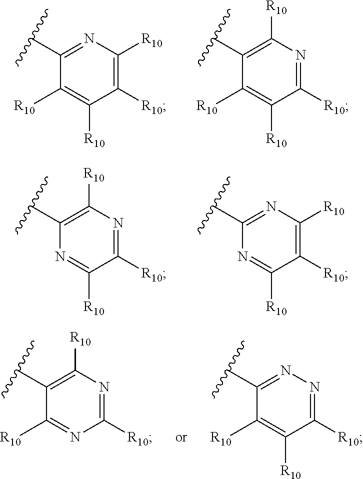

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN; R₃ is —H, or methyl, R₄, R₆, R₈ and R are each —H, R₅ is —H, halogen, methyl, or trifluoromethyl, and R₇ is C₁-C₃ alkyl (preferably methyl).

In yet a further compound of Formula (I), or (II), or pharmaceutically acceptable salts thereof, R₁ is a group of the formula:

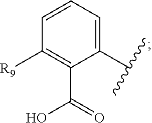

wherein R₉ is —H, halogen, or trifluoromethyl, (preferably R₉ is —H, or halogen), R₂ is a group of the formula:

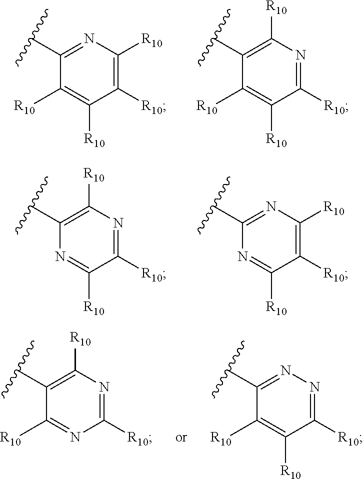

R₃ is —H, or methyl, R₄, R₆, R₈ and R are each —H, R₅ is —H, halogen, methyl, or trifluoromethyl, and R₇ is C₁-C₃ alkyl (preferably methyl).

In yet a further compound of Formula (I), or (II), or pharmaceutically acceptable salts thereof, R₁ is a group of the formula:

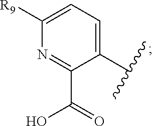

wherein R₉ is independently —H, halogen, methyl, trifluoromethyl, or cyclopropyl, R₂ is a group of the formula:

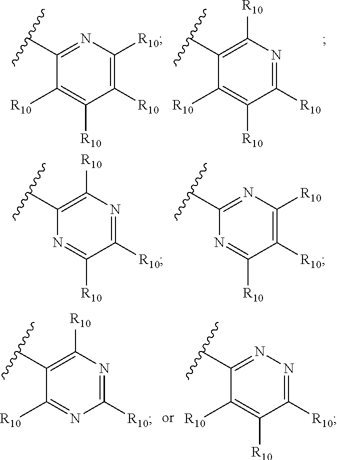

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN; R₃ is —H, methyl, or trifluoromethyl, R₄ is —H, or halogen, R₈ and R are each —H, R₅ is —H, halogen, methyl or trifluoromethyl, R₆ is —H, or halogen, and R₇ is C₁-C₃ alkyl (preferably methyl).

In yet a further compound of Formula (I), or (II), or pharmaceutically acceptable salts thereof, R₁ is a group of the formula:

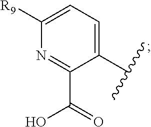

wherein R₉ is —H, halogen, or trifluoromethyl, (preferably R₉ is halogen or trifluoromethyl), R₂ is a group of the formula:

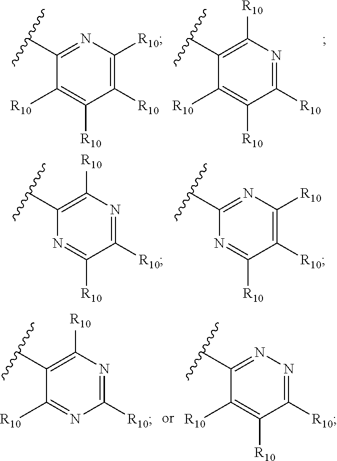

R₃ is —H, or methyl, R₄, R₆, R₈ and R are each —H, R₅ is —H, halogen, methyl, or trifluoromethyl, and R₇ is C₁-C₃ alkyl (preferably methyl).

In yet a further compound of Formula (I), or (II), or pharmaceutically acceptable salts thereof, R₁ is a group of the formula:

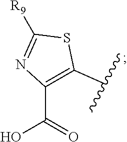

wherein R₉ is —H, halogen, C₁-C₃ alkyl or C₁-C₃ haloalkyl, R₂ is a group of the formula:

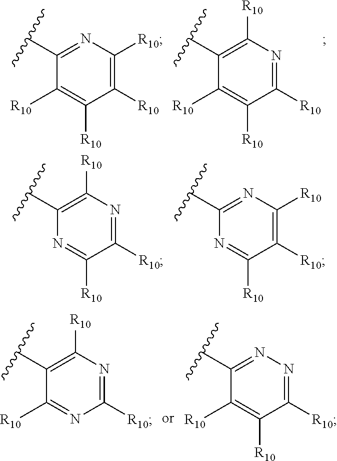

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN; R₃ is —H, or methyl, R₄, R₆, R₈ and R are each —H, R₅ is —H, halogen, methyl, or trifluoromethyl, and R₇ is C₁-C₃ alkyl (preferably methyl).

In yet a further compound of Formula (I), or (II), or pharmaceutically acceptable salts thereof, R₁ is a group of the formula:

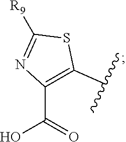

wherein R₉ is —H, halogen, C₁-C₃ alkyl or C₁-C₃ haloalkyl, R₂ is a group of the formula:

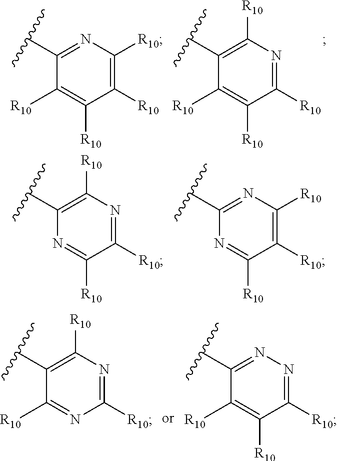

R₃ is —H, or methyl, R₄, R₆, R₈ and R are each —H, R₅ is —H, halogen, methyl, or trifluoromethyl, and R₇ is C₁-C₃ alkyl (preferably methyl).

In yet a further compound of Formula (I), or (II), or pharmaceutically acceptable salts thereof, R₁ is a group of the formula:

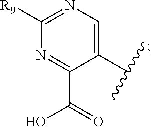

wherein each R₉ is independently —H, halogen, methyl, trifluoromethyl, or cyclopropyl, R₂ is a group of the formula:

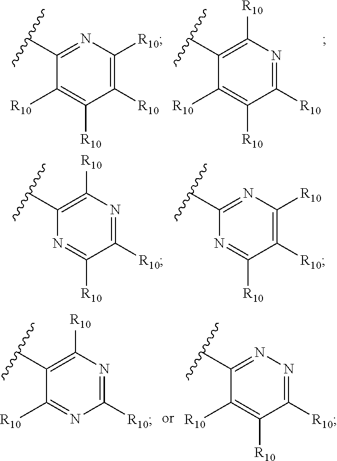

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN; R₃ is —H, methyl, or trifluoromethyl, R₄ is —H, or halogen, R₈ and R are each —H, R₅ is —H, halogen, methyl or trifluoromethyl, R₆ is —H, or halogen, and R₇ is C₁-C₃ alkyl (preferably methyl).

In a further aspect, compounds of Formula (I) or (II) have Formula (III), or pharmaceutically acceptable salts thereof:

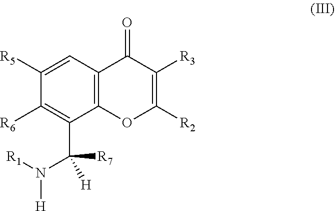

wherein R₁, R₂, R₃, R₅, R₆, and R₇ are as defined in the Summary for Formula (I) above.

In a compound of Formula (III), or pharmaceutically acceptable salts thereof, R₂ is a group of the formula:

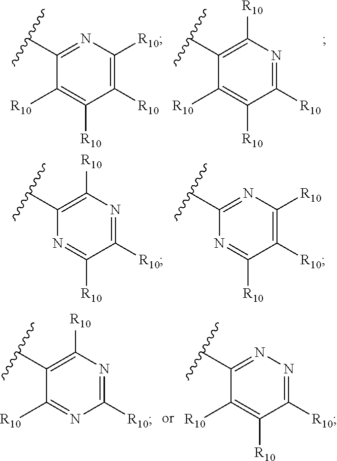

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN.

In a compound of Formula (III), or pharmaceutically acceptable salts thereof, R₂ is a group of the formula:

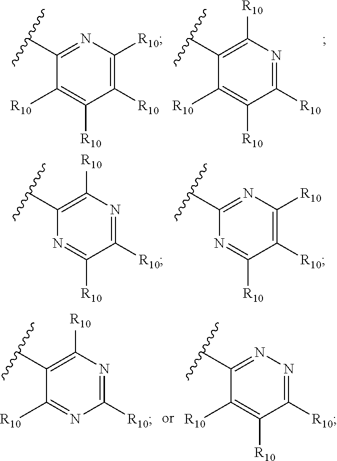

In yet a further compound of Formula (III), or pharmaceutically acceptable salts thereof, R₃ is —H, —CN, C₁-C₃ alkyl, C₁-C₃ haloalkyl; preferably R₃ is —H, —CN, or C₁-C₃ alkyl; most preferably R₃ is —H, or methyl.

In yet a further compound of Formula (III), or pharmaceutically acceptable salts thereof, R₅ is —H, halogen, C₁-C₆ alkyl, or C₁-C₆ haloalkyl; preferably R₅ is —H, halogen, C₁-C₃ alkyl or C₁-C₃ haloalkyl; more preferably R₅ is —H, halogen, methyl, or trifluoromethyl.

In yet a further compound of Formula (III), or pharmaceutically acceptable salts thereof, R₆ is —H or halogen; preferably R₆ is —H.

In yet a further compound of Formula (III), or pharmaceutically acceptable salts thereof, R₇ is —CN, C₁-C₆ alkyl, or C₁-C₆ haloalkyl; preferably R₇ is —CN, C₁-C₃ alkyl or C₁-C₃ haloalkyl; more preferably R₇ is —CN, methyl or trifluoromethyl.

In yet a further compound of Formula (III), or pharmaceutically acceptable salts thereof, R₁ is a group of the formula:

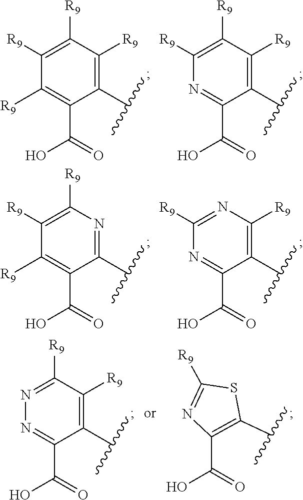

wherein each R₉ is independently —H, halogen, C₁-C₃ alkyl, C₁-C₃ haloalkyl, or C₃-C₅ cycloalkyl; preferably each R₉ is independently —H, halogen, methyl, trifluoromethyl, or cyclopropyl.

In yet a further compound of Formula (III), or pharmaceutically acceptable salts thereof, R₁ is a group of the formula:

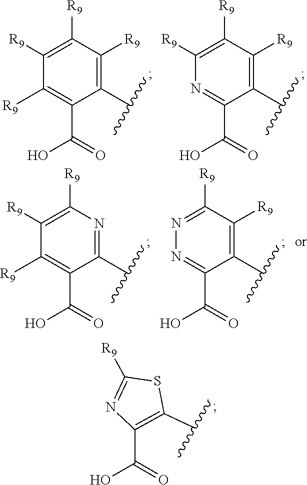

wherein each R₉ is independently —H, halogen, C₁-C₃ alkyl or C₁-C₃ haloalkyl; preferably each R₉ is independently —H, halogen, methyl, or trifluoromethyl.

In yet a further compound of Formula (III), or pharmaceutically acceptable salts thereof, R₁ is a group of the formula:

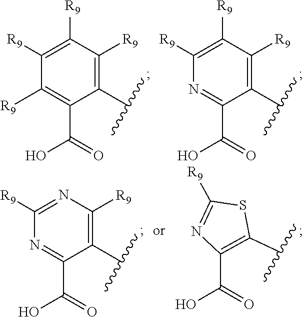

wherein each R₉ is independently —H, halogen, C₁-C₆ alkyl, C₁-C₆ haloalkyl, C₁-C₆ alkoxy, or C₃-C₅ cycloalkyl. Preferably each R₉ is independently —H, halogen, C₁-C₃ alkyl, C₁-C₃ haloalkyl, or C₃—C₅ cycloalkyl. More preferably each R₉ is independently —H, halogen, methyl, trifluoromethyl, or cyclopropyl.

In yet a further compound of Formula (III), or pharmaceutically acceptable salts thereof, R₁ is a group of the formula:

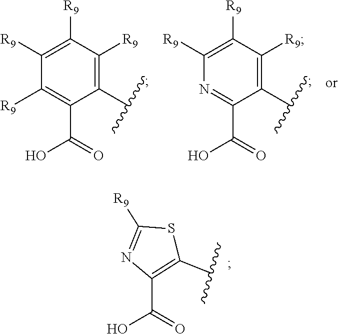

wherein each R₉ is independently —H, halogen, C₁-C₆ alkyl, C₁-C₆ haloalkyl, C₁-C₆ alkoxy, or C₃-C₅ cycloalkyl. Preferably each R₉ is independently —H, halogen, C₁-C₃ alkyl or C₁-C₃ haloalkyl. More preferably each R₉ is independently —H, halogen, methyl, or trifluoromethyl.

In yet a further compound of Formula (III), or pharmaceutically acceptable salts thereof, R₁ is a group of the formula:

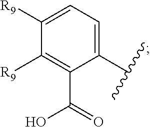

wherein each R₉ is independently —H, halogen, C₁-C₃ alkyl or C₁-C₃ haloalkyl. Preferably each R₉ is independently —H, halogen, methyl or trifluoromethyl.

In yet a further compound of Formula (III), or pharmaceutically acceptable salts thereof, R₁ is a group of the formula:

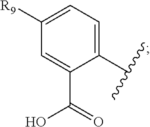

wherein R₉ is —H, halogen, or C₁-C₃ haloalkyl. Preferably R₉ is —H, or trifluoromethyl.

In yet a further compound of Formula (III), or pharmaceutically acceptable salts thereof, R₁ is a group of the formula:

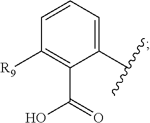

wherein R₉ is independently —H, halogen, C₁-C₃ alkyl or C₁-C₃ haloalkyl. Preferably R₉ is independently —H, halogen, methyl or trifluoromethyl.

In yet a further compound of Formula (III), or pharmaceutically acceptable salts thereof, R₁ is a group of the formula:

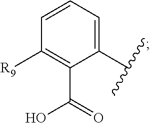

wherein R₉ is —H, halogen, or C₁-C₃ haloalkyl. Preferably R₉ is —H, or halogen.

In yet a further compound of Formula (III), or pharmaceutically acceptable salts thereof, R₁ is a group of the formula:

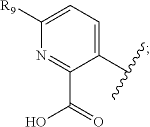

wherein R₉ is —H, halogen, C₁-C₃ alkyl, C₁-C₃ haloalkyl, or C₃-C₅ cycloalkyl. Preferably R₉ is independently —H, halogen, methyl, trifluoromethyl, or cyclopropyl.

In yet a further compound of Formula (III), or pharmaceutically acceptable salts thereof, R₁ is a group of the formula:

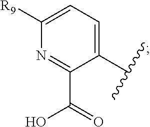

wherein R₉ is —H, halogen, or C₁-C₃ haloalkyl. Preferably R₉ is independently halogen or trifluoromethyl. More preferably R₉ is chloro or trifluoromethyl.

In yet a further compound of Formula (III), or pharmaceutically acceptable salts thereof, R₁ is a group of the formula:

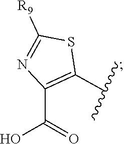

wherein R₉ is —H, halogen, C₁-C₃ alkyl or C₁-C₃ haloalkyl.

In yet a further compound of Formula (III), or pharmaceutically acceptable salts thereof, R₁ is a group of the formula:

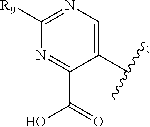

wherein R₉ is —H, halogen, C₁-C₃ alkyl, C₁-C₃ haloalkyl, or C₃-C₅ cycloalkyl. Preferably R₉ is —H, halogen, methyl, trifluoromethyl, or cyclopropyl.

In yet a further compound of Formula (III), or pharmaceutically acceptable salts thereof, R₁ is a group of the formula:

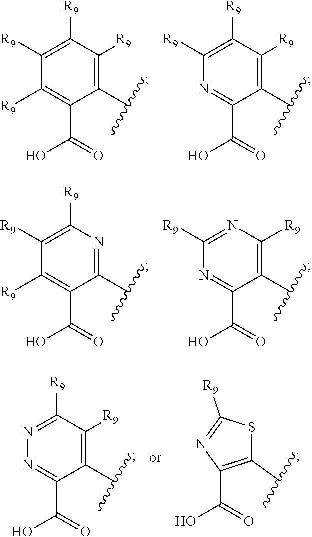

wherein each R₉ is independently —H, halogen, C₁-C₃ alkyl, C₁-C₃ haloalkyl, or C₃-C₅ cycloalkyl, and R₂ is a group of the formula:

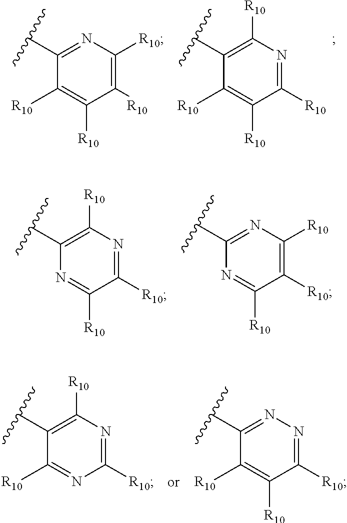

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN;
wherein each R₁₀ is independently —H, —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁—CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN; and each R₁₁ is independently —H or C₁-C₃ alkyl.

In yet a further compound of Formula (III), or pharmaceutically acceptable salts thereof, R₁ is a group of the formula:

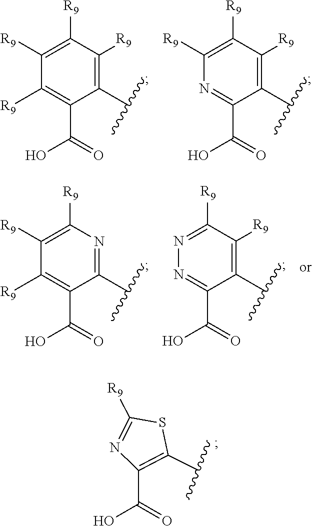

wherein each R₉ is independently —H, halogen, C₁-C₃ alkyl or C₁-C₃ haloalkyl, and R₂ is a group of the formula:

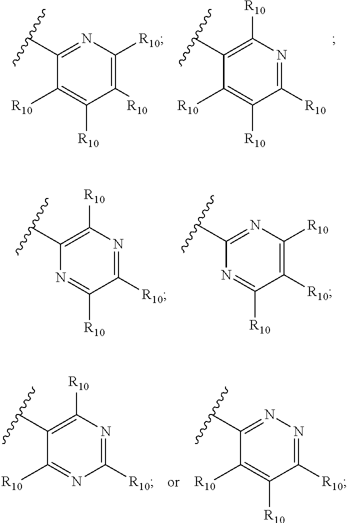

wherein each R₁₀ is independently —H, —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁—CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN; and each R₁₁ is independently —H or C₁-C₃ alkyl.

In yet a further compound of Formula (III), or pharmaceutically acceptable salts thereof, R₁ is a group of the formula:

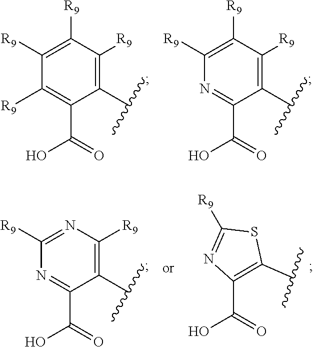

wherein each R₉ is independently —H, halogen, C₁-C₆ alkyl, C₁-C₆ haloalkyl, C₁-C₆ alkoxy, or C₃-C₅ cycloalkyl, and R₂ is a group of the formula:

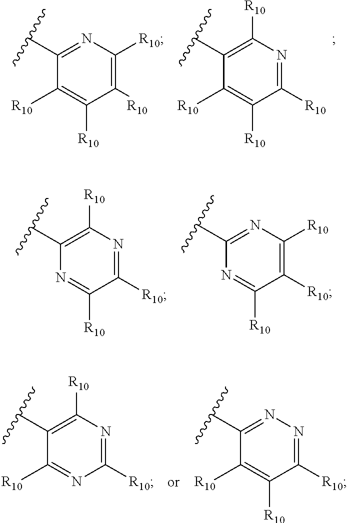

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN. Preferably each R₉ is independently —H, halogen, C₁-C₃ alkyl, C₁-C₃ haloalkyl, or C₃-C₅ cycloalkyl. Most preferably each R₉ is independently —H, halogen, methyl, trifluoromethyl, or cyclopropyl.

In yet a further compound of Formula (III), or pharmaceutically acceptable salts thereof, R₁ is a group of the formula:

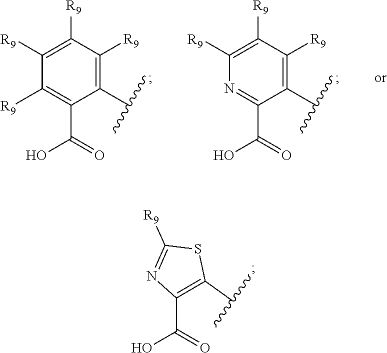

wherein each R₉ is independently —H, halogen, C₁-C₆ alkyl, C₁-C₆ haloalkyl, C₁-C₆ alkoxy, or C₃-C₅ cycloalkyl, and R₂ is a group of the formula:

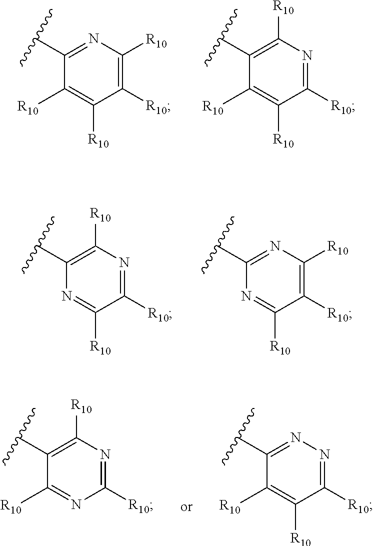

preferably each R₉ is independently —H, halogen, C₁-C₃ alkyl or C₁-C₃ haloalkyl. Most preferably each R₉ is independently —H, halogen, methyl, or trifluoromethyl.

In yet a further compound of Formula (III), or pharmaceutically acceptable salts thereof, R₁ is a group of the formula:

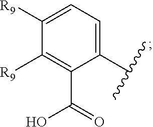

wherein each R₉ is independently —H, halogen, C₁-C₃ alkyl or C₁-C₃ haloalkyl, and R₂ is a group of the formula:

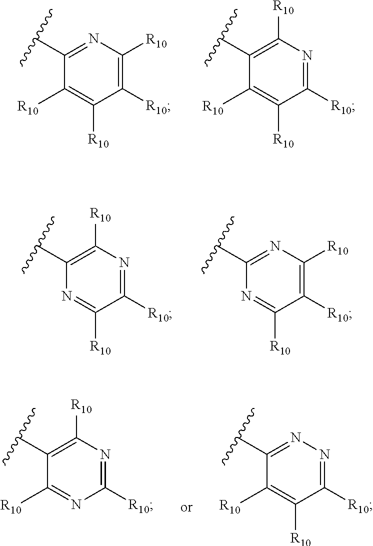

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN. Preferably each R₉ is independently —H, halogen, methyl or trifluoromethyl.

In yet a further compound of Formula (III), or pharmaceutically acceptable salts thereof, R₁ is a group of the formula:

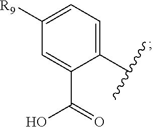

wherein R₉ is —H, halogen, or C₁-C₃ haloalkyl, and R₂ is a group of the formula:

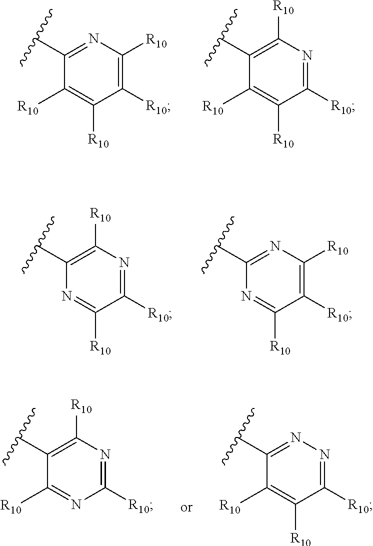

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN; preferably R₉ is —H, or trifluoromethyl.

In yet a further compound of Formula (III), or pharmaceutically acceptable salts thereof, R₁ is a group of the formula:

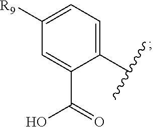

wherein R₉ is —H, halogen, or C₁-C₃ haloalkyl, and R₂ is a group of the formula:

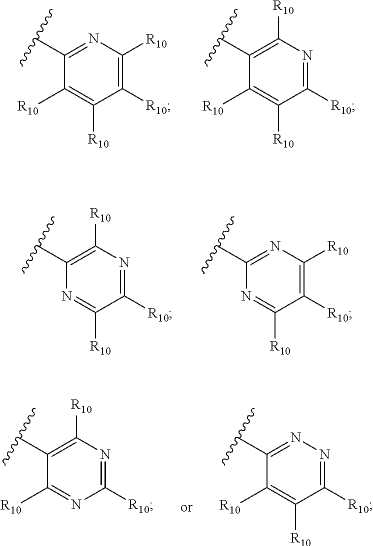

preferably R₉ is —H, or trifluoromethyl.

In yet a further compound of Formula (III), or pharmaceutically acceptable salts thereof, R₁ is a group of the formula:

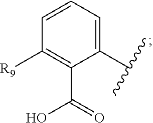

wherein R₉ is —H, halogen, or C₁-C₃ haloalkyl, and R₂ is a group of the formula:

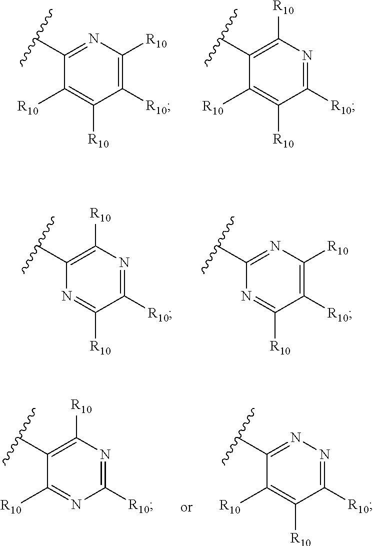

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN; preferably R₉ is —H, or halogen.

In yet a further compound of Formula (III), or pharmaceutically acceptable salts thereof, R₁ is a group of the formula:

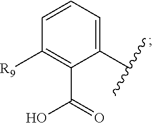

wherein R₉ is —H, halogen, or C₁-C₃ haloalkyl, and R₂ is a group of the formula:

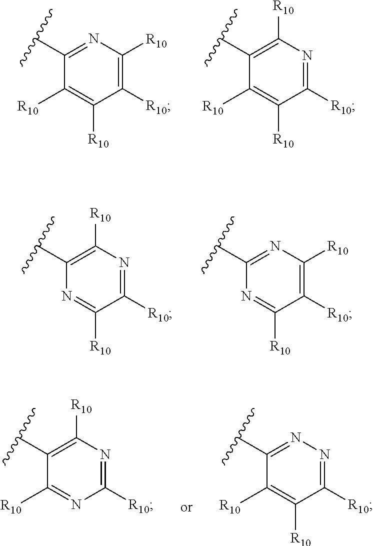

preferably R₉ is —H, or halogen.

In yet a further compound of Formula (III), or pharmaceutically acceptable salts thereof, R₁ is a group of the formula:

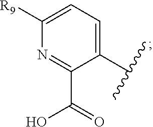

wherein R₉ is —H, halogen, C₁-C₃ alkyl, C₁-C₃ haloalkyl, or C₃-C₅ cycloalkyl, and R₂ is a group of the formula:

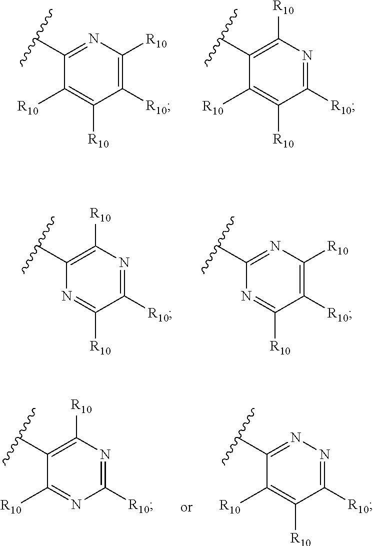

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN; preferably R₉ is —H, halogen, methyl, trifluoromethyl, or cyclopropyl.

In yet a further compound of Formula (III), or pharmaceutically acceptable salts thereof, R₁ is a group of the formula:

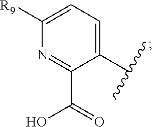

wherein R₉ is halogen, or C₁-C₃ haloalkyl, and R₂ is a group of the formula:

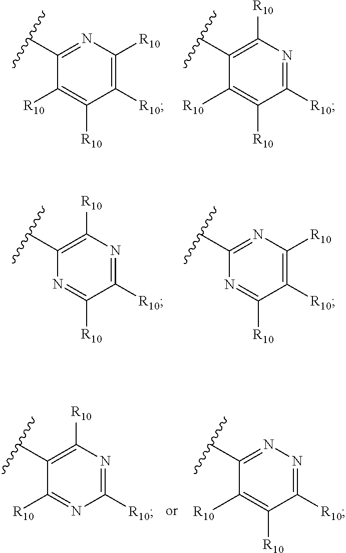

preferably R₉ is halogen or trifluoromethyl. More preferably R₉ is chloro or trifluoromethyl.

In yet a further compound of Formula (III), or pharmaceutically acceptable salts thereof, R₁ is a group of the formula:

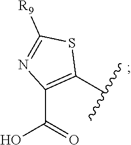

wherein R₉ is —H, halogen, C₁-C₃ alkyl or C₁-C₃ haloalkyl, and R₂ is a group of the formula:

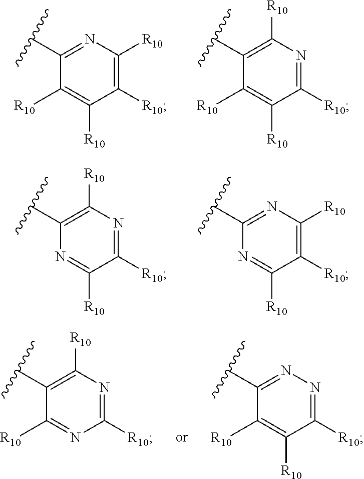

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN.

In yet a further compound of Formula (III), or pharmaceutically acceptable salts thereof, R₁ is a group of the formula:

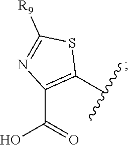

wherein R₉ is —H, halogen, C₁-C₃ alkyl or C₁-C₃ haloalkyl, and R₂ is a group of the formula:

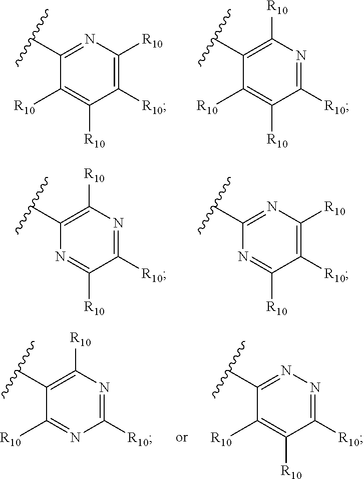

In yet a further compound of Formula (III), or pharmaceutically acceptable salts thereof, R₁ is a group of the formula:

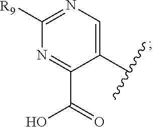

wherein R₉ is —H, halogen, C₁-C₃ alkyl, C₁-C₃ haloalkyl, or C₃-C₅ cycloalkyl, and R₂ is a group of the formula:

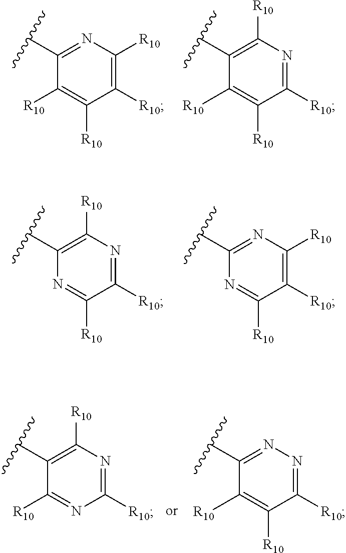

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN; preferably R₉ is —H, halogen, methyl, trifluoromethyl, or cyclopropyl.

In yet a further compound of Formula (III), or pharmaceutically acceptable salts thereof, R₃ is —H, —CN, C₁-C₃ alkyl, C₁-C₃ haloalkyl (preferably R₃ is —H, —CN, or C₁-C₃ alkyl), R₅ is —H, halogen, C₁-C₆ alkyl, or C₁-C₆ haloalkyl, R₆ is —H or halogen, R₇ is —CN, methyl or trifluoromethyl, and R₂ is a group of the formula:

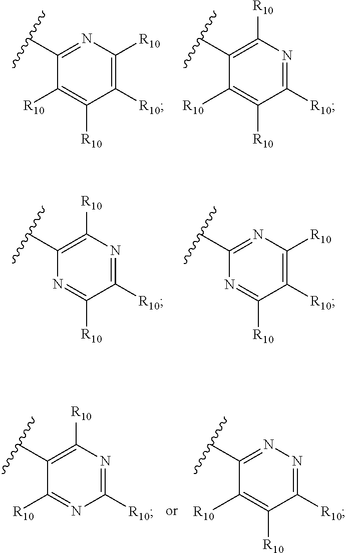

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN; more preferably R₃ is —H, or methyl, R₅ is —H, halogen, methyl, or trifluoromethyl, R₆ is —H, and R₇ is methyl.

In yet a further compound of Formula (III), or pharmaceutically acceptable salts thereof, R₃ is —H, —CN, or C₁-C₃ alkyl, R₅ is —H, halogen, C₁-C₆ alkyl, or C₁-C₆ haloalkyl, R₇ is —CN, methyl or trifluoromethyl, R₆ is —H or halogen, and R₂ is a group of the formula:

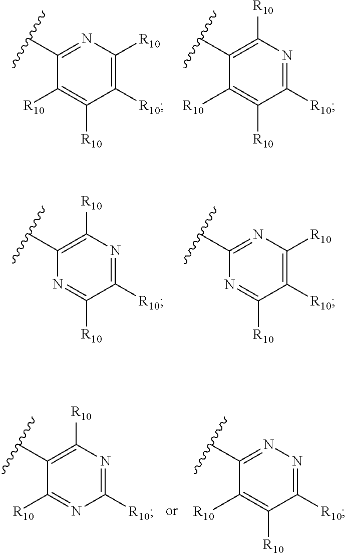

preferably R₃ is —H, or methyl, R₅ is —H, halogen, methyl, or trifluoromethyl, R₆ is —H, R₇ is methyl, and R₂ is a group of the formula:

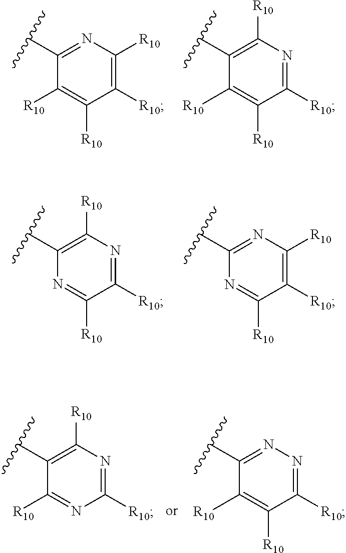

In yet a further compound of Formula (III), or pharmaceutically acceptable salts thereof, R₂ is a group of the formula:

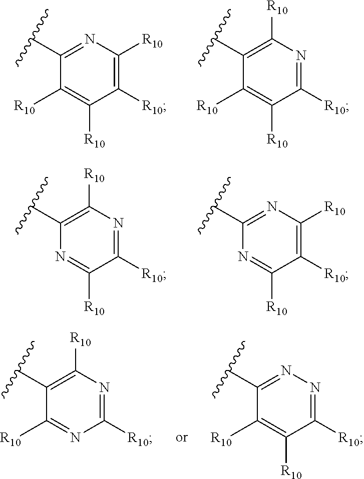

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN; R₃ is —H, —CN, C₁-C₃ alkyl, or C₁-C₃ haloalkyl, R₅ is —H, halogen, C₁-C₃ alkyl or C₁-C₃ haloalkyl, R₆ is —H or halogen, and R₁ is a group of the formula:

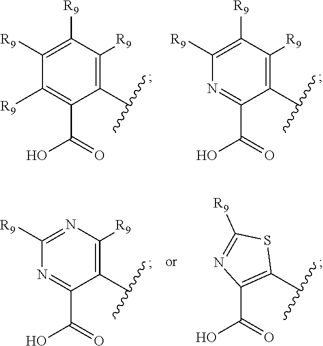

wherein each R₉ is independently —H, halogen, C₁-C₃ alkyl, C₁-C₃ haloalkyl, or C₃-C₅ cycloalkyl. Preferably R₃ is —H, methyl, or trifluoromethyl, R₅ is —H, halogen, methyl, or trifluoromethyl, R₆ is —H or halogen, and each R₉ is independently —H, halogen, methyl, trifluoromethyl, or cyclopropyl.

In yet a further compound of Formula (III), or pharmaceutically acceptable salts thereof, R₂ is a group of the formula:

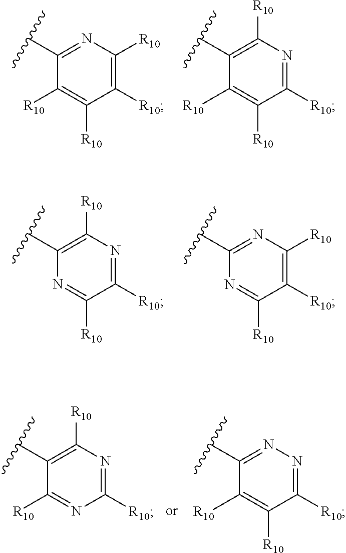

R₃ is —H, —CN, or C₁-C₃ alkyl, R₅ is —H, halogen, C₁-C₃ alkyl or C₁-C₃ haloalkyl, R₆ is —H or halogen, and R₁ is a group of the formula:

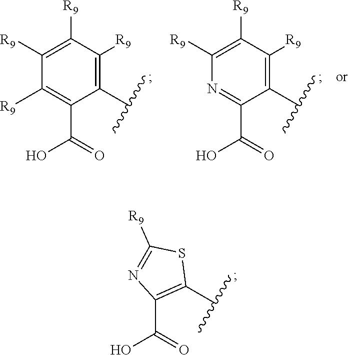

wherein each R₉ is independently —H, halogen, C₁-C₃ alkyl or C₁-C₃ haloalkyl. Preferably R₃ is —H, or methyl, R₅ is —H, halogen, methyl, or trifluoromethyl, R₆ is —H, and each R₉ is independently —H, halogen, methyl, or trifluoromethyl.

In yet a further compound of Formula (III), or pharmaceutically acceptable salts thereof, R₂ is a group of the formula:

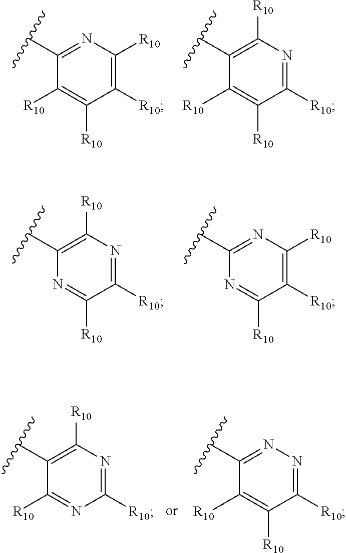

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN; R₃ is —H, —CN, C₁-C₃ alkyl, or C₁-C₃ haloalkyl, R₅ is —H, halogen, C₁-C₃ alkyl or C₁-C₃ haloalkyl, R₆ is —H or halogen, R₇ is —CN, methyl or trifluoromethyl, and R₁ is a group of the formula:

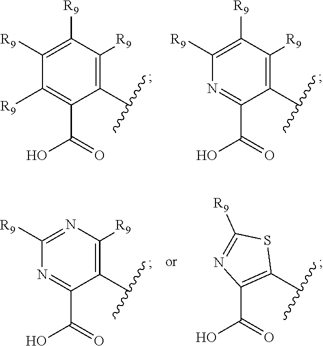

wherein each R₉ is independently —H, halogen, C₁-C₃ alkyl, C₁-C₃ haloalkyl, or C₃-C₅ cycloalkyl.

In yet a further compound of Formula (III), or pharmaceutically acceptable salts thereof, R₂ is a group of the formula:

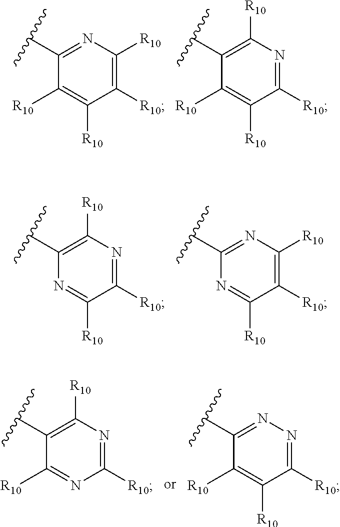

R₃ is —H, —CN, or C₁-C₃ alkyl, R₅ is —H, halogen, C₁-C₃ alkyl or C₁-C₃ haloalkyl, R₆ is —H or halogen, R₇ is —CN, methyl or trifluoromethyl, and R₁ is a group of the formula:

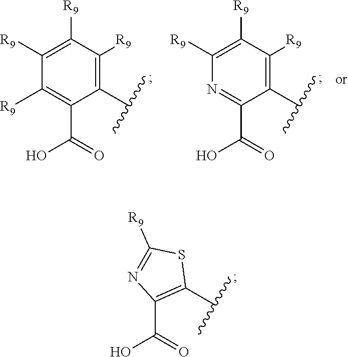

wherein each R₉ is independently —H, halogen, C₁-C₃ alkyl or C₁-C₃ haloalkyl.

In yet a further compound of Formula (III), or pharmaceutically acceptable salts thereof, R₁ is a group of the formula:

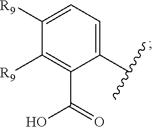

wherein each R₉ is independently —H, halogen, methyl or trifluoromethyl, R₂ is a group of the formula:

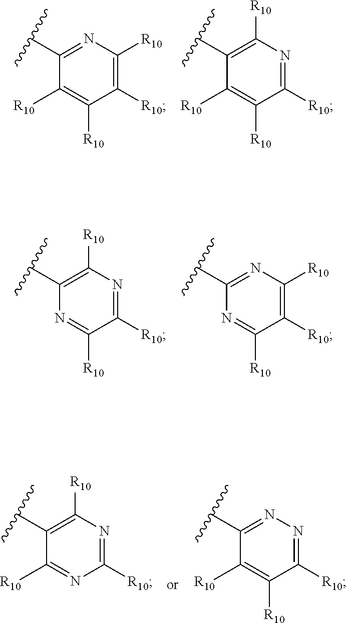

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN; R₃ is —H, methyl, or trifluoromethyl, R₅ is —H, halogen, methyl or trifluoromethyl, R₆ is —H, or halogen, and R₇ is C₁-C₃ alkyl (preferably methyl).

In yet a further compound of Formula (III), or pharmaceutically acceptable salts thereof, R₁ is a group of the formula:

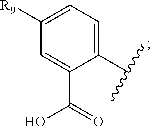

wherein R₉ is —H, halogen, or trifluoromethyl, (preferably R₉ is —H, or trifluoromethyl), R₂ is a group of the formula:

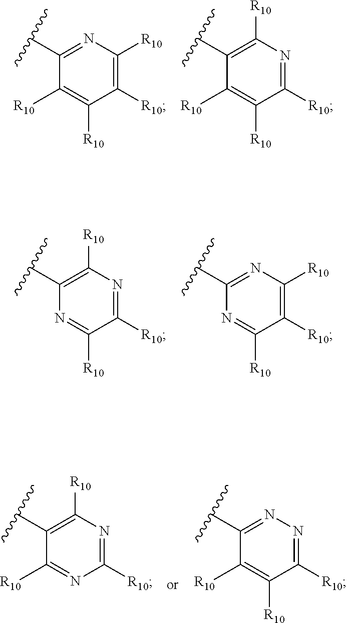

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN; R₃ is —H, or methyl, R₅ is —H, halogen, methyl, or trifluoromethyl, R₆ is —H or halogen, and R₇ is methyl.

In yet a further compound of Formula (III), or pharmaceutically acceptable salts thereof, R₁ is a group of the formula:

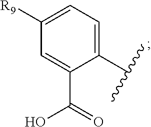

wherein R₉ is —H, halogen, or trifluoromethyl, (preferably R₉ is —H, or trifluoromethyl), R₂ is a group of the formula:

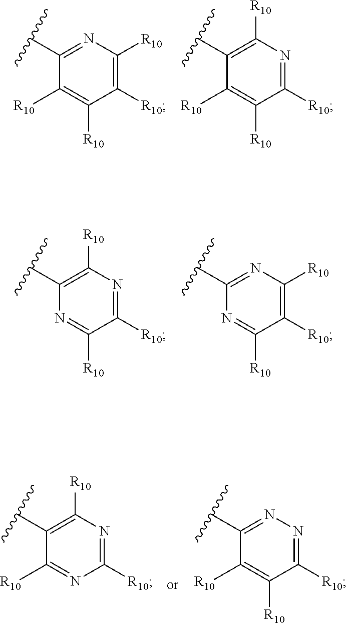

R₃ is —H, or methyl, R₅ is —H, halogen, methyl, or trifluoromethyl, R₆ is —H or halogen, and R₇ is methyl.

In yet a further compound of Formula (III), or pharmaceutically acceptable salts thereof, R₁ is a group of the formula:

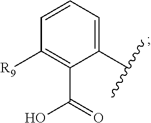

wherein R₉ is —H, halogen, or trifluoromethyl, (preferably R₉ is —H, or halogen), R₂ is a group of the formula:

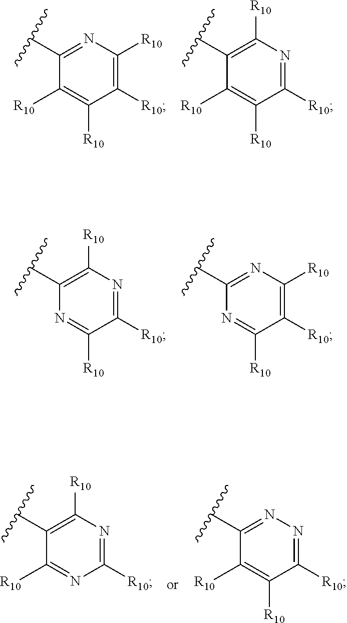

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN; R₃ is —H, or methyl, R₅ is —H, halogen, methyl, or trifluoromethyl, R₆ is —H or halogen, and R₇ is methyl.

In yet a further compound of Formula (III), or pharmaceutically acceptable salts thereof, R₁ is a group of the formula:

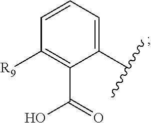

wherein R₉ is —H, halogen, or trifluoromethyl, (preferably R₉ is —H, or halogen), R₂ is a group of the formula:

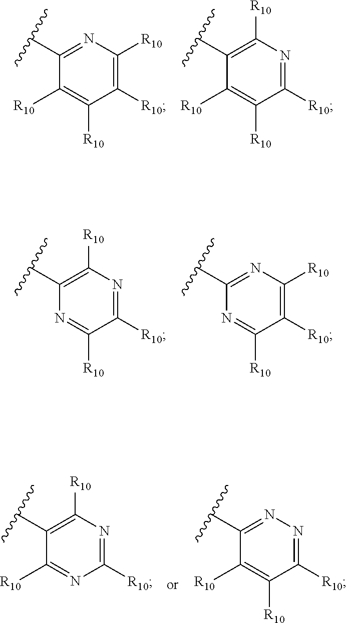

R₃ is —H, or methyl, R₅ is —H, halogen, methyl, or trifluoromethyl, R₆ is —H or halogen, and R₇ is methyl.

In yet a further compound of Formula (III), or pharmaceutically acceptable salts thereof, R₁ is a group of the formula:

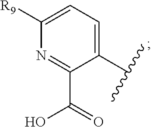

wherein R₉ is independently —H, halogen, methyl, trifluoromethyl, or cyclopropyl, R₂ is a group of the formula:

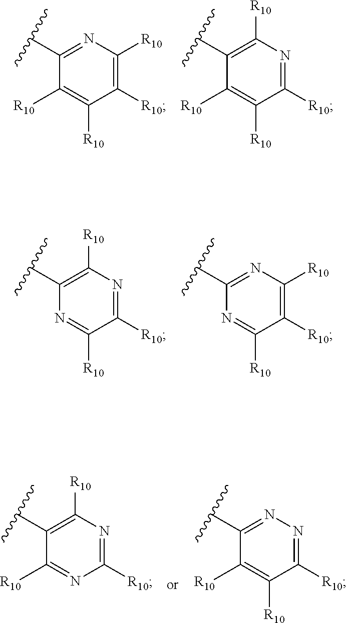

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN, R₃ is —H, methyl, or trifluoromethyl, R₅ is —H, halogen, methyl or trifluoromethyl, R₆ is —H, or halogen, and R₇ is C₁-C₃ alkyl (preferably methyl).

In yet a further compound of Formula (III), or pharmaceutically acceptable salts thereof, R₁ is a group of the formula:

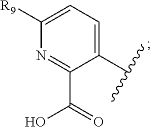

wherein R₉ is —H, halogen, or trifluoromethyl, (preferably R₉ is halogen or trifluoromethyl), R₂ is a group of the formula:

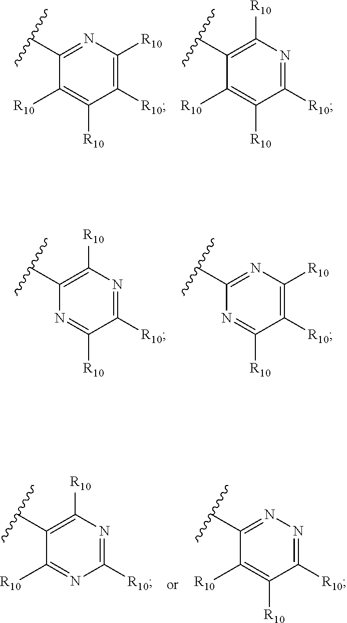

R₃ is —H, or methyl, R₅ is —H, halogen, methyl, or trifluoromethyl, R₆ is —H or halogen, and R₇ is methyl.

In yet a further compound of Formula (III), or pharmaceutically acceptable salts thereof, R₁ is a group of the formula:

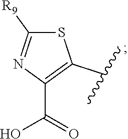

wherein R₉ is —H, halogen, C₁-C₃ alkyl or C₁-C₃ haloalkyl, R₂ is a group of the formula:

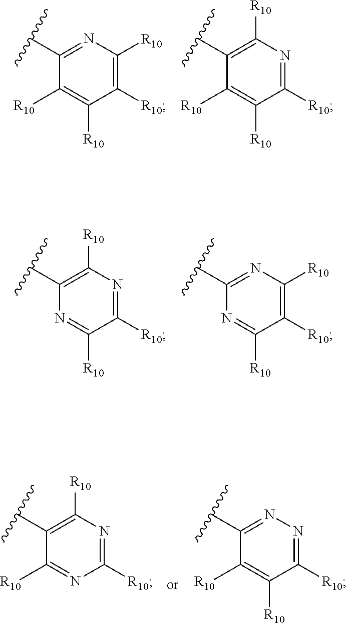

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN; R₃ is —H, or methyl, R₅ is —H, halogen, methyl, or trifluoromethyl, R₆ is —H or halogen, and R₇ is methyl.

In yet a further compound of Formula (III), or pharmaceutically acceptable salts thereof, R₁ is a group of the formula:

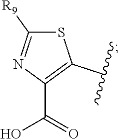

wherein R₉ is —H, halogen, C₁-C₃ alkyl or C₁-C₃ haloalkyl, R₂ is a group of the formula:

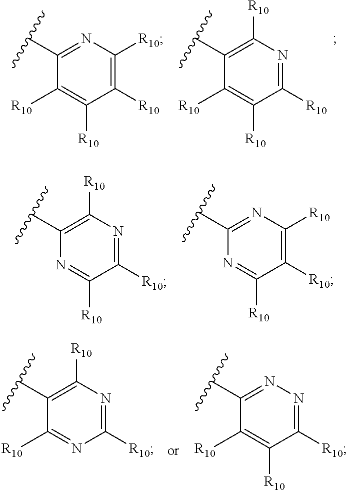

R₃ is —H, or methyl, R₅ is —H, halogen, methyl, or trifluoromethyl, R₆ is —H or halogen, and R₇ is methyl.

In yet a further compound of Formula (III), or pharmaceutically acceptable salts thereof, R₁ is a group of the formula:

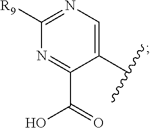

wherein R₉ is independently —H, halogen, methyl, trifluoromethyl, or cyclopropyl, R₂ is a group of the formula:

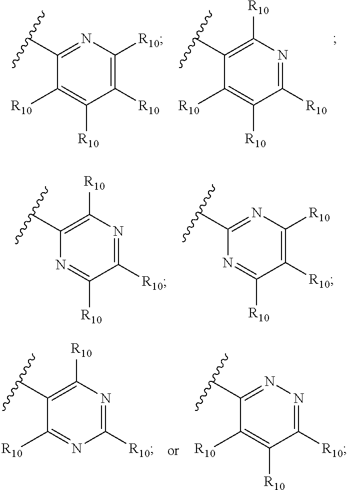

or
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN; R₃ is —H, methyl, or trifluoromethyl, R₅ is —H, halogen, methyl or trifluoromethyl, R₆ is —H, or halogen, and R₇ is C₁-C₃ alkyl (preferably methyl).

In yet a further compound of Formula (I), the compound is selected from:

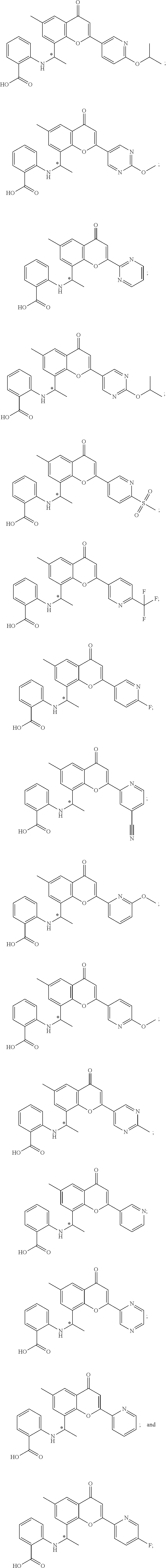

or a pharmaceutically acceptable salt of any of the foregoing,
wherein the bond at the * position is as represented,

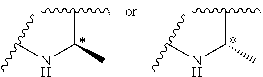

In yet a further compound of Formula (I), the compound is selected from:

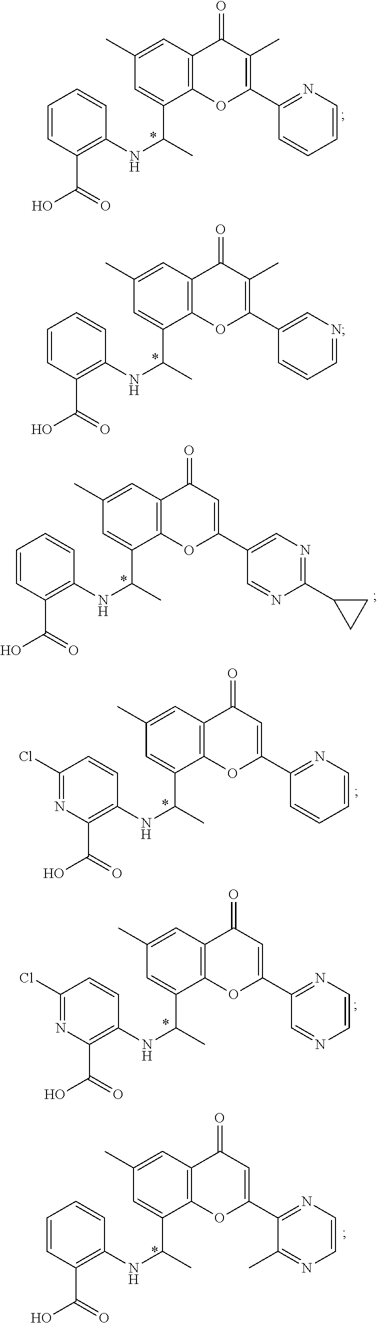 [SMILES: Cc1cc(C(C)Nc2ccccc2C(=O)O)c2oc(-c3ccccn3)c(C)c(=O)c2c1 ; Cc1cc(C(C)Nc2ccccc2C(=O)O)c2oc(-c3cccnc3)c(C)c(=O)c2c1 ; Cc1cc(C(C)Nc2ccccc2C(=O)O)c2oc(-c3cnc(C4CC4)nc3)cc(=O)c2c1 ; Cc1cc(C(C)Nc2ccc(Cl)nc2C(=O)O)c2oc(-c3ccccn3)cc(=O)c2c1 ; Cc1cc(C(C)Nc2ccc(Cl)nc2C(=O)O)c2oc(-c3cnccn3)cc(=O)c2c1 ; Cc1cc(C(C)Nc2ccccc2C(=O)O)c2oc(-c3nccnc3C)cc(=O)c2c1]

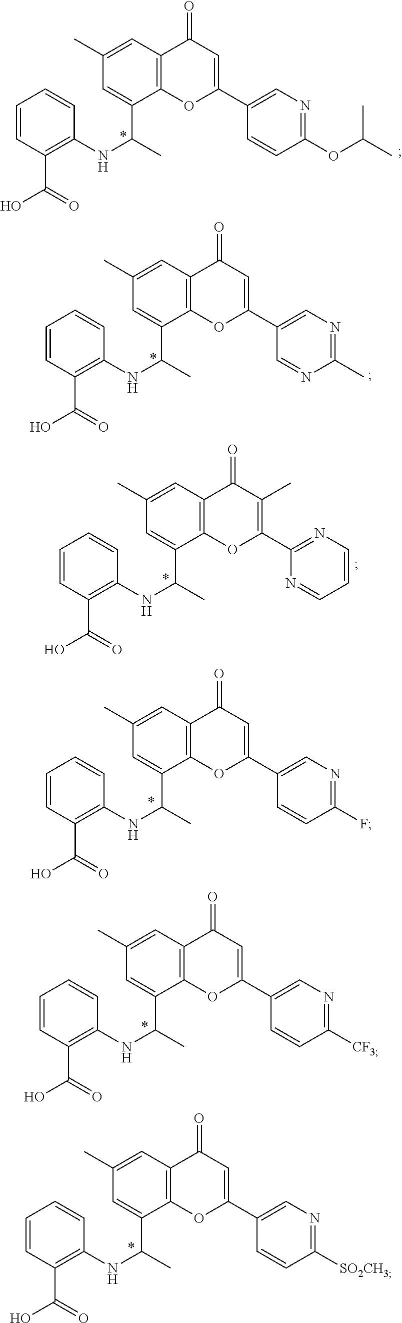 [SMILES: Cc1cc(C(C)Nc2ccccc2C(=O)O)c2oc(-c3ccc(OC(C)C)nc3)cc(=O)c2c1 ; Cc1cc(C(C)Nc2ccccc2C(=O)O)c2oc(-c3cnc(C)nc3)cc(=O)c2c1 ; Cc1cc(C(C)Nc2ccccc2C(=O)O)c2oc(-c3ncccn3)c(C)c(=O)c2c1 ; Cc1cc(C(C)Nc2ccccc2C(=O)O)c2oc(-c3ccc(F)nc3)cc(=O)c2c1 ; Cc1cc(C(C)Nc2ccccc2C(=O)O)c2oc(-c3ccc(C(F)(F)F)nc3)cc(=O)c2c1 ; Cc1cc(C(C)Nc2ccccc2C(=O)O)c2oc(-c3ccc(S(C)(=O)=O)nc3)cc(=O)c2c1]

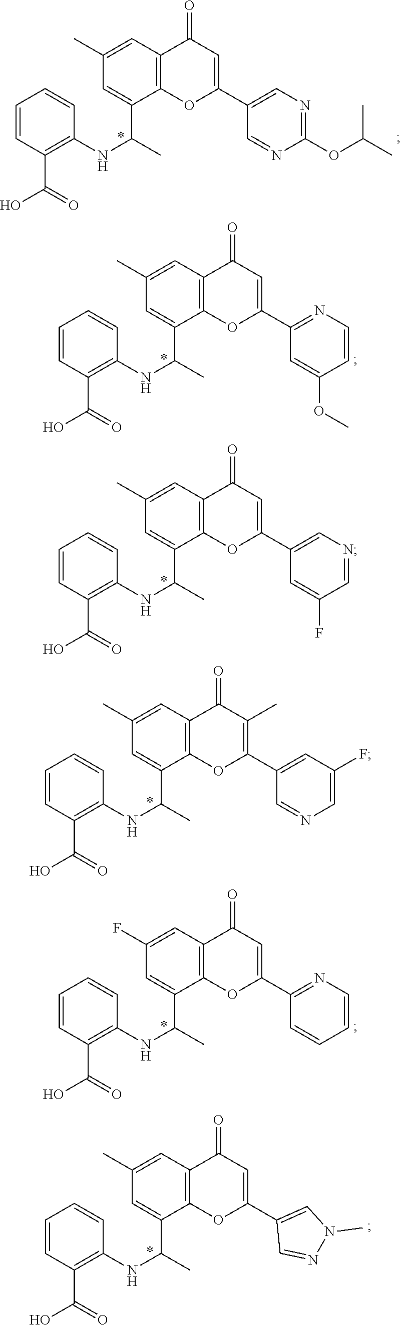 [SMILES: Cc1cc(C(C)Nc2ccccc2C(=O)O)c2oc(-c3cnc(OC(C)C)nc3)cc(=O)c2c1 ; COc1ccnc(-c2cc(=O)c3cc(C)cc(C(C)Nc4ccccc4C(=O)O)c3o2)c1 ; Cc1cc(C(C)Nc2ccccc2C(=O)O)c2oc(-c3cncc(F)c3)cc(=O)c2c1 ; Cc1cc(C(C)Nc2ccccc2C(=O)O)c2oc(-c3cncc(F)c3)c(C)c(=O)c2c1 ; CC(Nc1ccccc1C(=O)O)c1cc(F)cc2c(=O)cc(-c3ccccn3)oc12 ; Cc1cc(C(C)Nc2ccccc2C(=O)O)c2oc(-c3cnn(C)c3)cc(=O)c2c1]

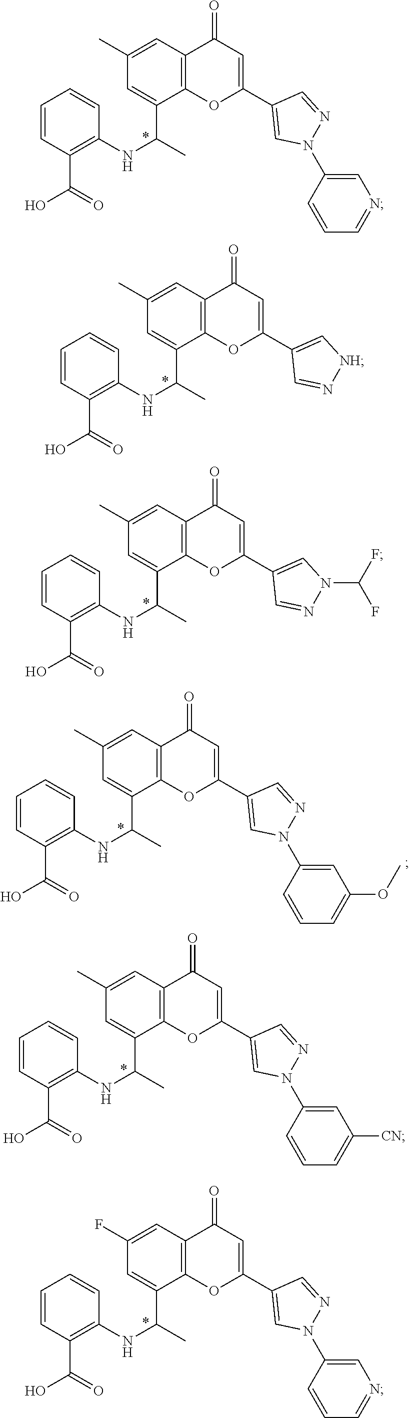 [SMILES: Cc1cc(C(C)Nc2ccccc2C(=O)O)c2oc(-c3cnn(-c4cccnc4)c3)cc(=O)c2c1 ; Cc1cc(C(C)Nc2ccccc2C(=O)O)c2oc(-c3cn[nH]c3)cc(=O)c2c1 ; Cc1cc(C(C)Nc2ccccc2C(=O)O)c2oc(-c3cnn(C(F)F)c3)cc(=O)c2c1 ; COc1cccc(-n2cc(-c3cc(=O)c4cc(C)cc(C(C)Nc5ccccc5C(=O)O)c4o3)cn2)c1 ; Cc1cc(C(C)Nc2ccccc2C(=O)O)c2oc(-c3cnn(-c4cccc(C#N)c4)c3)cc(=O)c2c1 ; CC(Nc1ccccc1C(=O)O)c1cc(F)cc2c(=O)cc(-c3cnn(-c4cccnc4)c3)oc12]

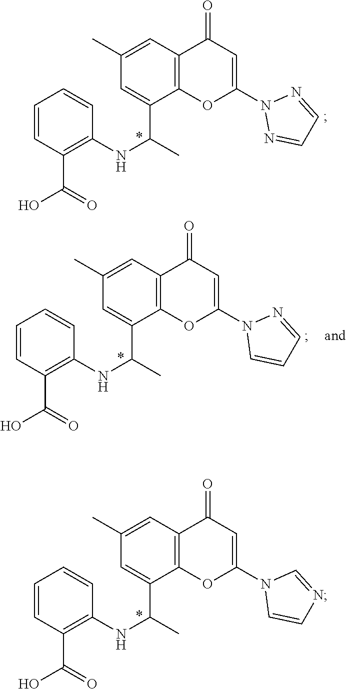 [SMILES: Cc1cc(C(C)Nc2ccccc2C(=O)O)c2oc(-n3nccn3)cc(=O)c2c1 ; Cc1cc(C(C)Nc2ccccc2C(=O)O)c2oc(-n3cccn3)cc(=O)c2c1 ; Cc1cc(C(C)Nc2ccccc2C(=O)O)c2oc(-n3ccnc3)cc(=O)c2c1]

or a pharmaceutically acceptable salt of any of the foregoing;
wherein the bond at the * position is as represented,

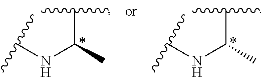

In yet a further compound of Formula (I), the compound is selected from:

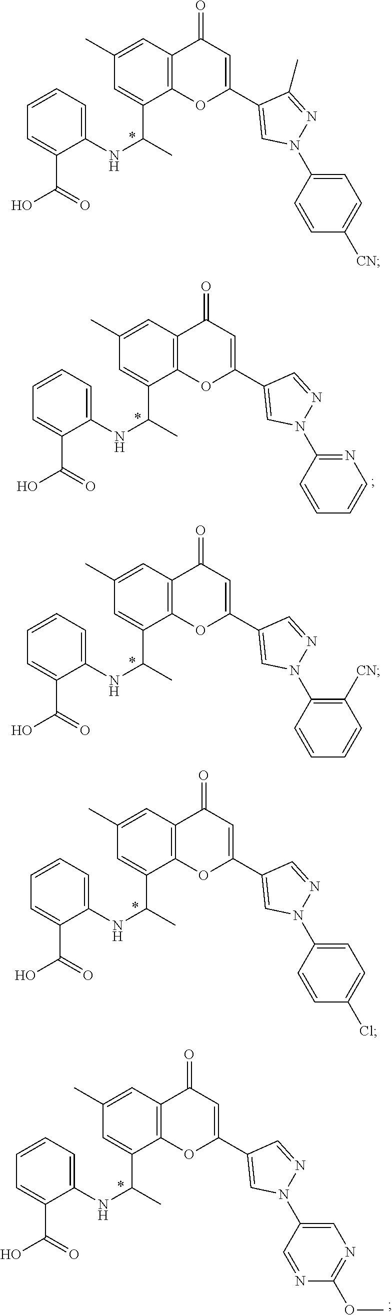 [SMILES: Cc1cc(C(C)Nc2ccccc2C(=O)O)c2oc(-c3cn(-c4ccc(C#N)cc4)nc3C)cc(=O)c2c1 ; Cc1cc(C(C)Nc2ccccc2C(=O)O)c2oc(-c3cnn(-c4ccccn4)c3)cc(=O)c2c1 ; Cc1cc(C(C)Nc2ccccc2C(=O)O)c2oc(-c3cnn(-c4ccccc4C#N)c3)cc(=O)c2c1 ; Cc1cc(C(C)Nc2ccccc2C(=O)O)c2oc(-c3cnn(-c4ccc(Cl)cc4)c3)cc(=O)c2c1 ; COc1ncc(-n2cc(-c3cc(=O)c4cc(C)cc(C(C)Nc5ccccc5C(=O)O)c4o3)cn2)cn1]

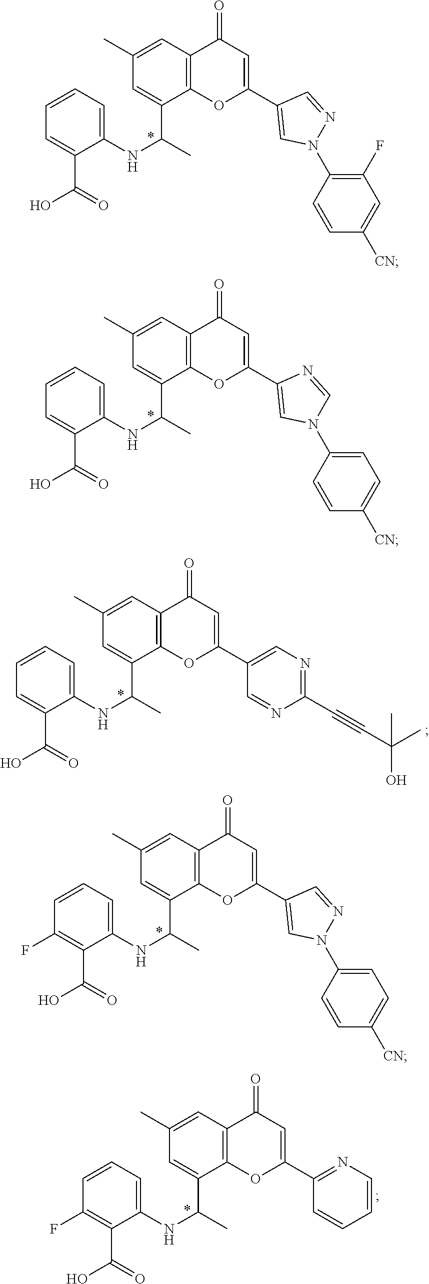 [SMILES: Cc1cc(C(C)Nc2ccccc2C(=O)O)c2oc(-c3cnn(-c4ccc(C#N)cc4F)c3)cc(=O)c2c1 ; Cc1cc(C(C)Nc2ccccc2C(=O)O)c2oc(-c3cn(-c4ccc(C#N)cc4)cn3)cc(=O)c2c1 ; Cc1cc(C(C)Nc2ccccc2C(=O)O)c2oc(-c3cnc(C#CC(C)(C)O)nc3)cc(=O)c2c1 ; Cc1cc(C(C)Nc2cccc(F)c2C(=O)O)c2oc(-c3cnn(-c4ccc(C#N)cc4)c3)cc(=O)c2c1 ; Cc1cc(C(C)Nc2cccc(F)c2C(=O)O)c2oc(-c3ccccn3)cc(=O)c2c1]

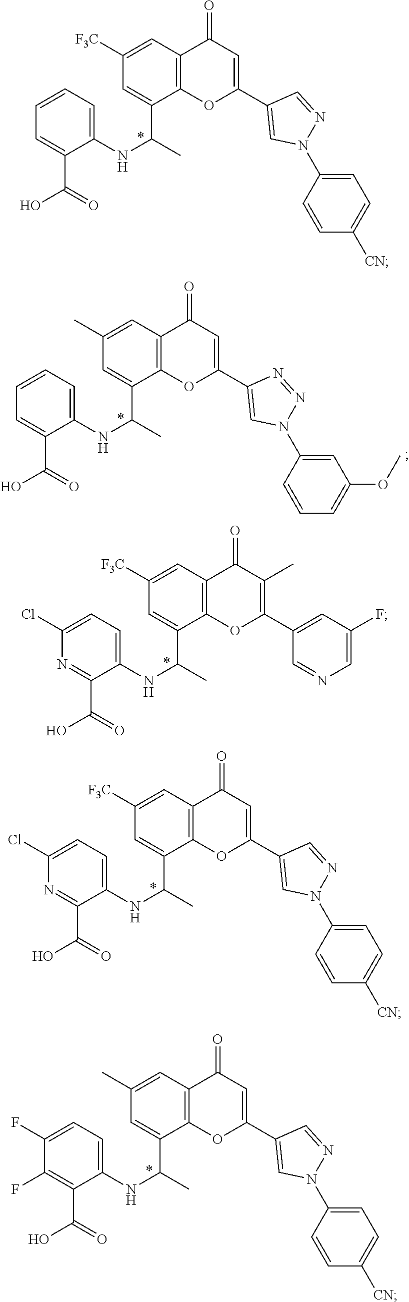 [SMILES: CC(Nc1ccccc1C(=O)O)c1cc(C(F)(F)F)cc2c(=O)cc(-c3cnn(-c4ccc(C#N)cc4)c3)oc12 ; COc1cccc(-n2cc(-c3cc(=O)c4cc(C)cc(C(C)Nc5ccccc5C(=O)O)c4o3)nn2)c1 ; Cc1c(-c2cncc(F)c2)oc2c(C(C)Nc3ccc(Cl)nc3C(=O)O)cc(C(F)(F)F)cc2c1=O ; CC(Nc1ccc(Cl)nc1C(=O)O)c1cc(C(F)(F)F)cc2c(=O)cc(-c3cnn(-c4ccc(C#N)cc4)c3)oc12 ; Cc1cc(C(C)Nc2ccc(F)c(F)c2C(=O)O)c2oc(-c3cnn(-c4ccc(C#N)cc4)c3)cc(=O)c2c1]

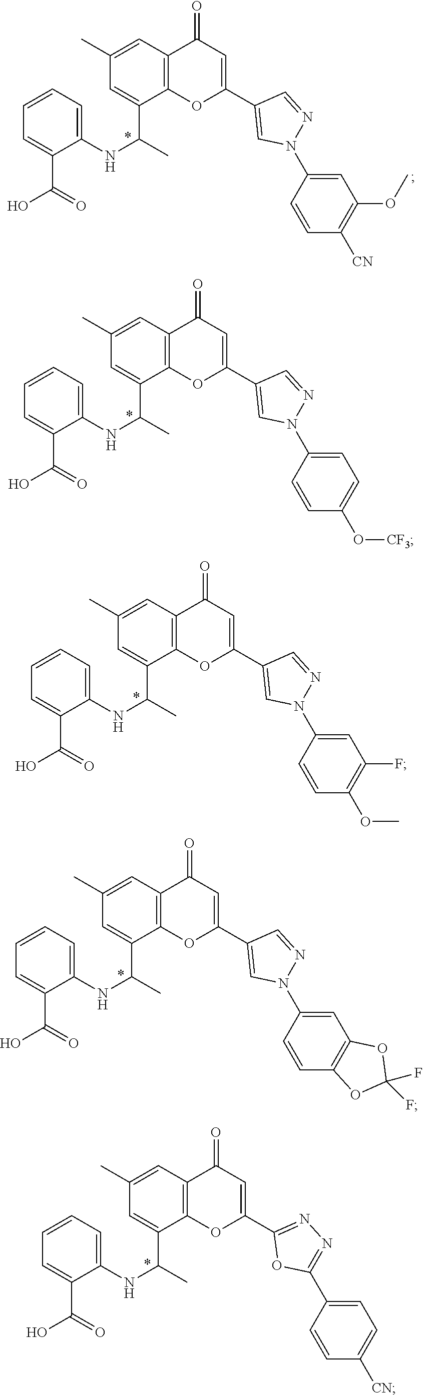 [SMILES: COc1cc(-n2cc(-c3cc(=O)c4cc(C)cc(C(C)Nc5ccccc5C(=O)O)c4o3)cn2)ccc1C#N ; Cc1cc(C(C)Nc2ccccc2C(=O)O)c2oc(-c3cnn(-c4ccc(OC(F)(F)F)cc4)c3)cc(=O)c2c1 ; COc1ccc(-n2cc(-c3cc(=O)c4cc(C)cc(C(C)Nc5ccccc5C(=O)O)c4o3)cn2)cc1F ; Cc1cc(C(C)Nc2ccccc2C(=O)O)c2oc(-c3cnn(-c4ccc5c(c4)OC(F)(F)O5)c3)cc(=O)c2c1 ; Cc1cc(C(C)Nc2ccccc2C(=O)O)c2oc(-c3nnc(-c4ccc(C#N)cc4)o3)cc(=O)c2c1]

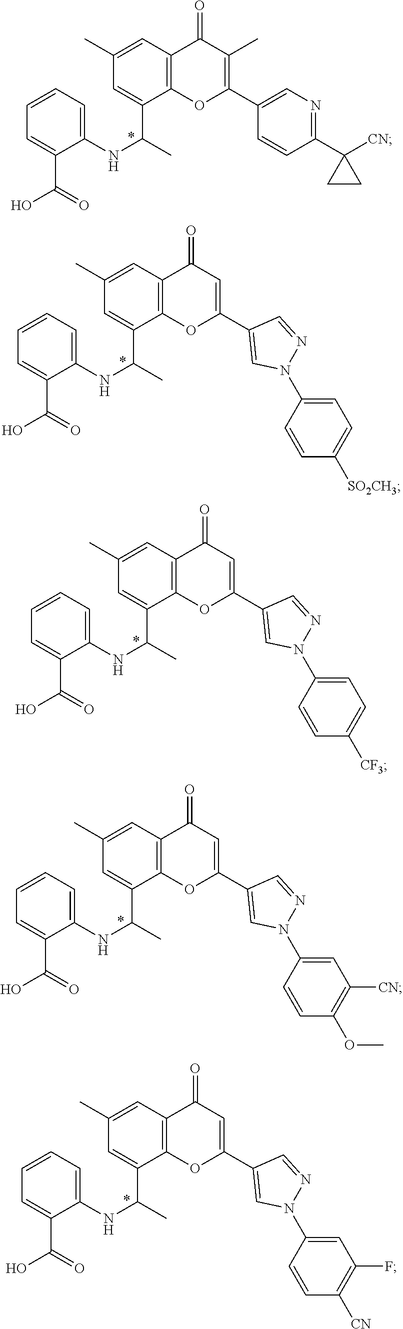 [SMILES: Cc1cc(C(C)Nc2ccccc2C(=O)O)c2oc(-c3ccc(C4(C#N)CC4)nc3)c(C)c(=O)c2c1 ; Cc1cc(C(C)Nc2ccccc2C(=O)O)c2oc(-c3cnn(-c4ccc(S(C)(=O)=O)cc4)c3)cc(=O)c2c1 ; Cc1cc(C(C)Nc2ccccc2C(=O)O)c2oc(-c3cnn(-c4ccc(C(F)(F)F)cc4)c3)cc(=O)c2c1 ; COc1ccc(-n2cc(-c3cc(=O)c4cc(C)cc(C(C)Nc5ccccc5C(=O)O)c4o3)cn2)cc1C#N ; Cc1cc(C(C)Nc2ccccc2C(=O)O)c2oc(-c3cnn(-c4ccc(C#N)c(F)c4)c3)cc(=O)c2c1]

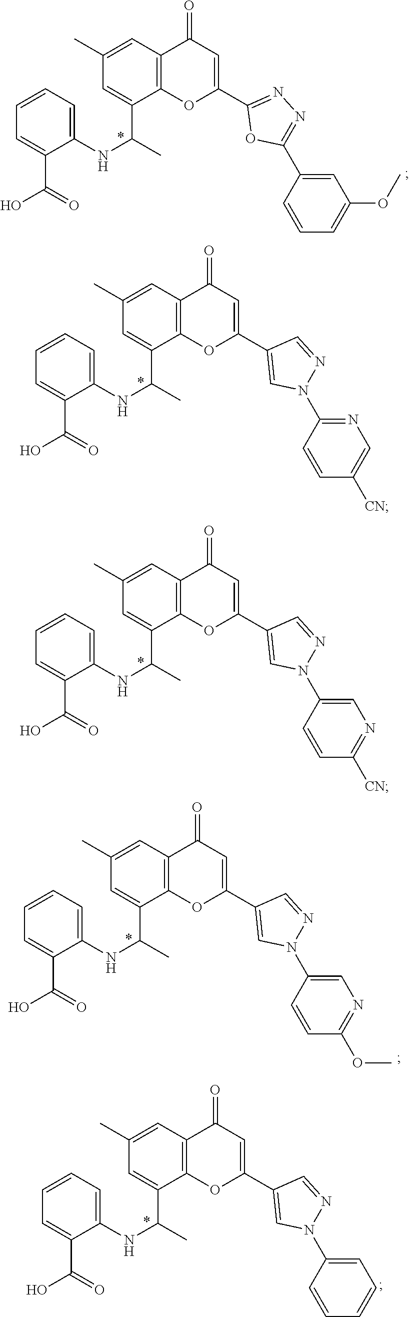 [SMILES: COc1cccc(-c2nnc(-c3cc(=O)c4cc(C)cc(C(C)Nc5ccccc5C(=O)O)c4o3)o2)c1 ; Cc1cc(C(C)Nc2ccccc2C(=O)O)c2oc(-c3cnn(-c4ccc(C#N)cn4)c3)cc(=O)c2c1 ; Cc1cc(C(C)Nc2ccccc2C(=O)O)c2oc(-c3cnn(-c4ccc(C#N)nc4)c3)cc(=O)c2c1 ; COc1ccc(-n2cc(-c3cc(=O)c4cc(C)cc(C(C)Nc5ccccc5C(=O)O)c4o3)cn2)cn1 ; Cc1cc(C(C)Nc2ccccc2C(=O)O)c2oc(-c3cnn(-c4ccccc4)c3)cc(=O)c2c1]

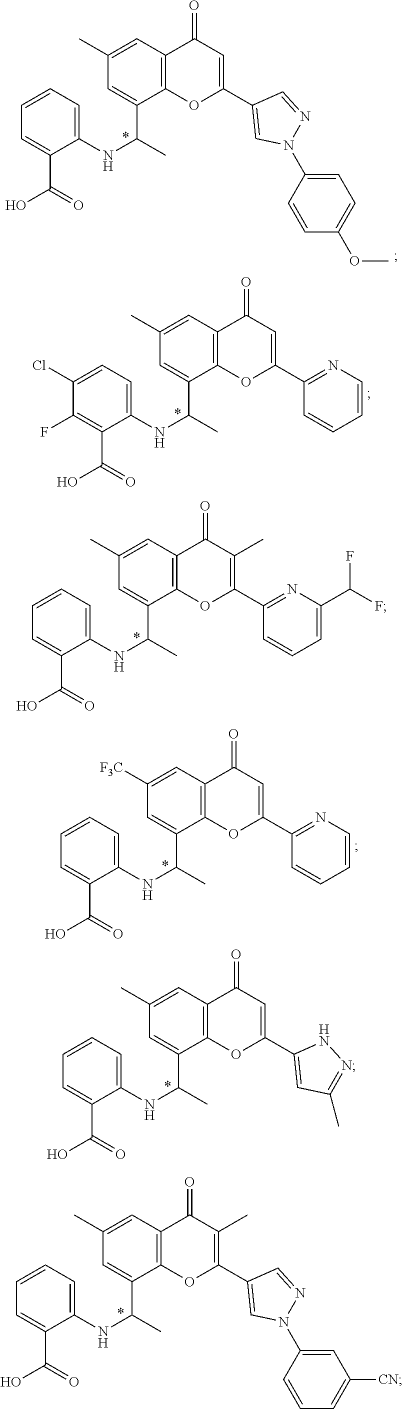 [SMILES: COc1ccc(-n2cc(-c3cc(=O)c4cc(C)cc(C(C)Nc5ccccc5C(=O)O)c4o3)cn2)cc1 ; Cc1cc(C(C)Nc2ccc(Cl)c(F)c2C(=O)O)c2oc(-c3ccccn3)cc(=O)c2c1 ; Cc1cc(C(C)Nc2ccccc2C(=O)O)c2oc(-c3cccc(C(F)F)n3)c(C)c(=O)c2c1 ; CC(Nc1ccccc1C(=O)O)c1cc(C(F)(F)F)cc2c(=O)cc(-c3ccccn3)oc12 ; Cc1cc(C(C)Nc2ccccc2C(=O)O)c2oc(-c3cc(C)n[nH]3)cc(=O)c2c1 ; Cc1cc(C(C)Nc2ccccc2C(=O)O)c2oc(-c3cnn(-c4cccc(C#N)c4)c3)c(C)c(=O)c2c1]

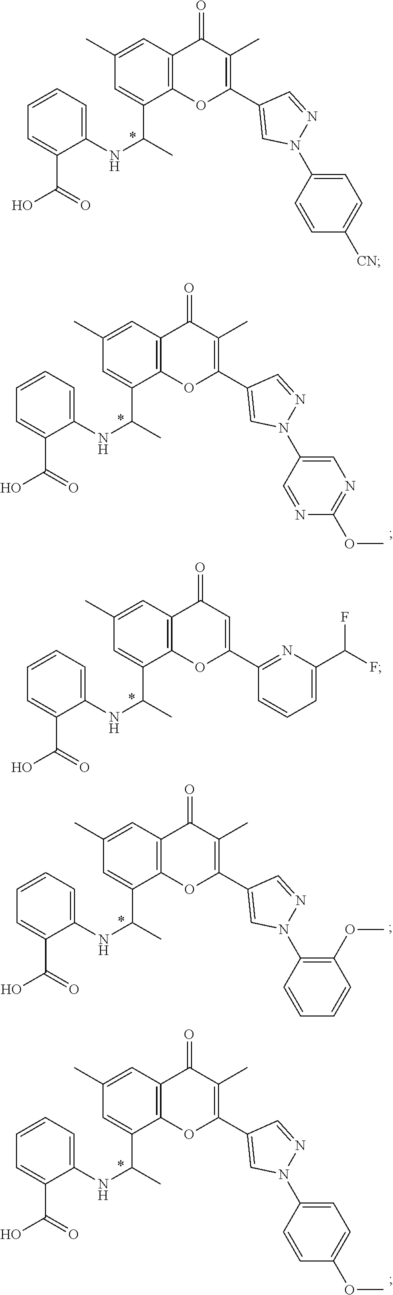 [SMILES: Cc1cc(C(C)Nc2ccccc2C(=O)O)c2oc(-c3cnn(-c4ccc(C#N)cc4)c3)c(C)c(=O)c2c1 ; COc1ncc(-n2cc(-c3oc4c(C(C)Nc5ccccc5C(=O)O)cc(C)cc4c(=O)c3C)cn2)cn1 ; Cc1cc(C(C)Nc2ccccc2C(=O)O)c2oc(-c3cccc(C(F)F)n3)cc(=O)c2c1 ; COc1ccccc1-n1cc(-c2oc3c(C(C)Nc4ccccc4C(=O)O)cc(C)cc3c(=O)c2C)cn1 ; COc1ccc(-n2cc(-c3oc4c(C(C)Nc5ccccc5C(=O)O)cc(C)cc4c(=O)c3C)cn2)cc1]

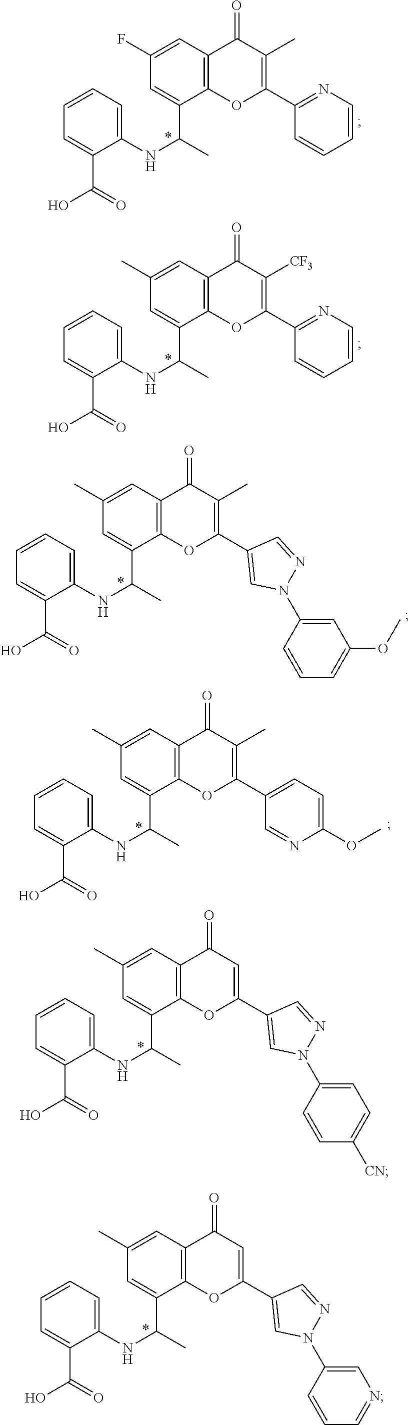 [SMILES: Cc1c(-c2ccccn2)oc2c(C(C)Nc3ccccc3C(=O)O)cc(F)cc2c1=O ; Cc1cc(C(C)Nc2ccccc2C(=O)O)c2oc(-c3ccccn3)c(C(F)(F)F)c(=O)c2c1 ; COc1cccc(-n2cc(-c3oc4c(C(C)Nc5ccccc5C(=O)O)cc(C)cc4c(=O)c3C)cn2)c1 ; COc1ccc(-c2oc3c(C(C)Nc4ccccc4C(=O)O)cc(C)cc3c(=O)c2C)cn1 ; Cc1cc(C(C)Nc2ccccc2C(=O)O)c2oc(-c3cnn(-c4ccc(C#N)cc4)c3)cc(=O)c2c1 ; Cc1cc(C(C)Nc2ccccc2C(=O)O)c2oc(-c3cnn(-c4cccnc4)c3)cc(=O)c2c1]

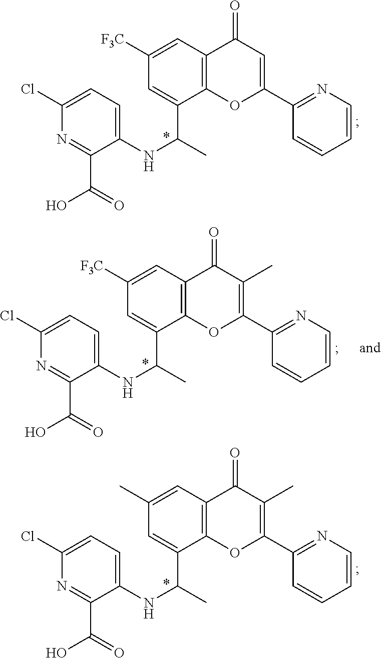 [SMILES: CC(Nc1ccc(Cl)nc1C(=O)O)c1cc(C(F)(F)F)cc2c(=O)cc(-c3ccccn3)oc12 ; Cc1c(-c2ccccn2)oc2c(C(C)Nc3ccc(Cl)nc3C(=O)O)cc(C(F)(F)F)cc2c1=O ; Cc1cc(C(C)Nc2ccc(Cl)nc2C(=O)O)c2oc(-c3ccccn3)c(C)c(=O)c2c1]

or a pharmaceutically acceptable salt of any of the foregoing;
wherein the bond at the * position is as represented,

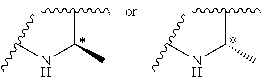

In yet a further compound of Formula (I), the compound is selected from:

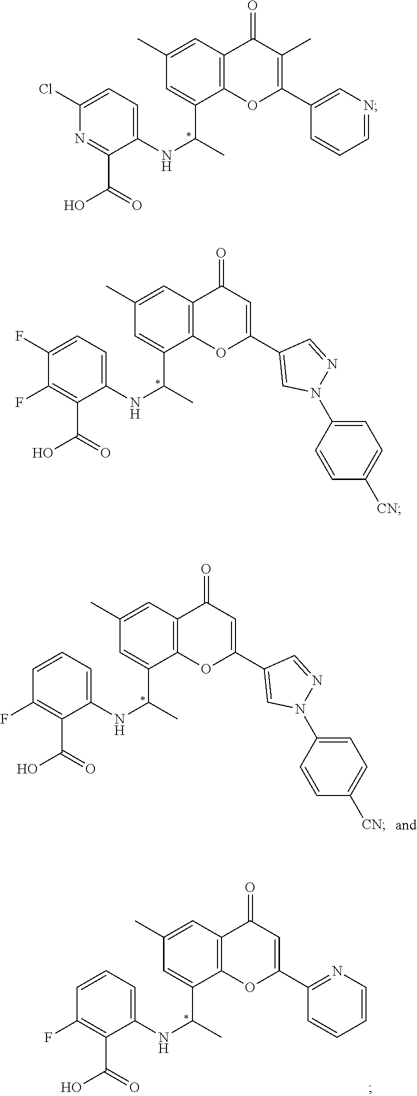 [SMILES: Cc1cc(C(C)Nc2ccc(Cl)nc2C(=O)O)c2oc(-c3cccnc3)c(C)c(=O)c2c1 ; Cc1cc(C(C)Nc2ccc(F)c(F)c2C(=O)O)c2oc(-c3cnn(-c4ccc(C#N)cc4)c3)cc(=O)c2c1 ; Cc1cc(C(C)Nc2cccc(F)c2C(=O)O)c2oc(-c3cnn(-c4ccc(C#N)cc4)c3)cc(=O)c2c1 ; Cc1cc(C(C)Nc2cccc(F)c2C(=O)O)c2oc(-c3ccccn3)cc(=O)c2c1]

or a pharmaceutically acceptable salt of any of the foregoing;
wherein the bond at the * position is as represented,

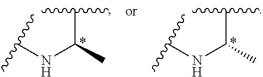

In yet a further compound of Formula (I), the compound is selected from:

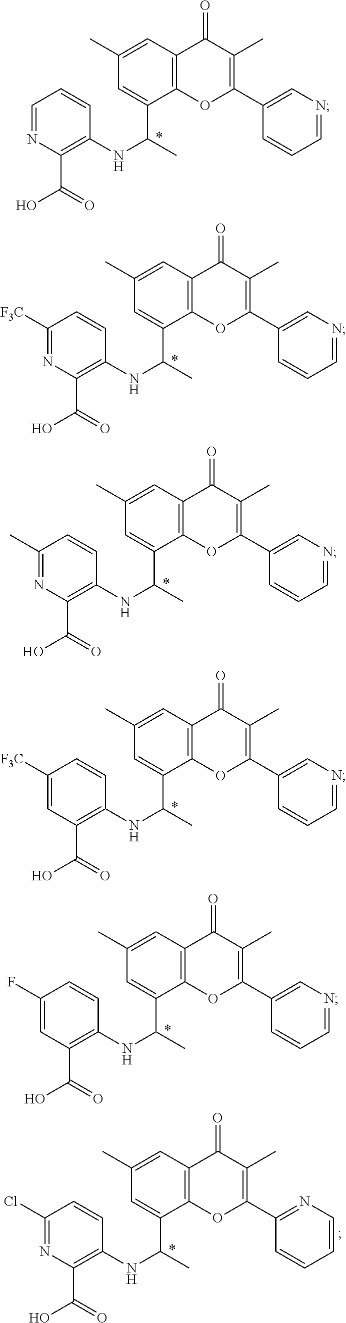 [SMILES: Cc1cc(C(C)Nc2cccnc2C(=O)O)c2oc(-c3cccnc3)c(C)c(=O)c2c1 ; Cc1cc(C(C)Nc2ccc(C(F)(F)F)nc2C(=O)O)c2oc(-c3cccnc3)c(C)c(=O)c2c1 ; Cc1cc(C(C)Nc2ccc(C)nc2C(=O)O)c2oc(-c3cccnc3)c(C)c(=O)c2c1 ; Cc1cc(C(C)Nc2ccc(C(F)(F)F)cc2C(=O)O)c2oc(-c3cccnc3)c(C)c(=O)c2c1 ; Cc1cc(C(C)Nc2ccc(F)cc2C(=O)O)c2oc(-c3cccnc3)c(C)c(=O)c2c1 ; Cc1cc(C(C)Nc2ccc(Cl)nc2C(=O)O)c2oc(-c3ccccn3)c(C)c(=O)c2c1]

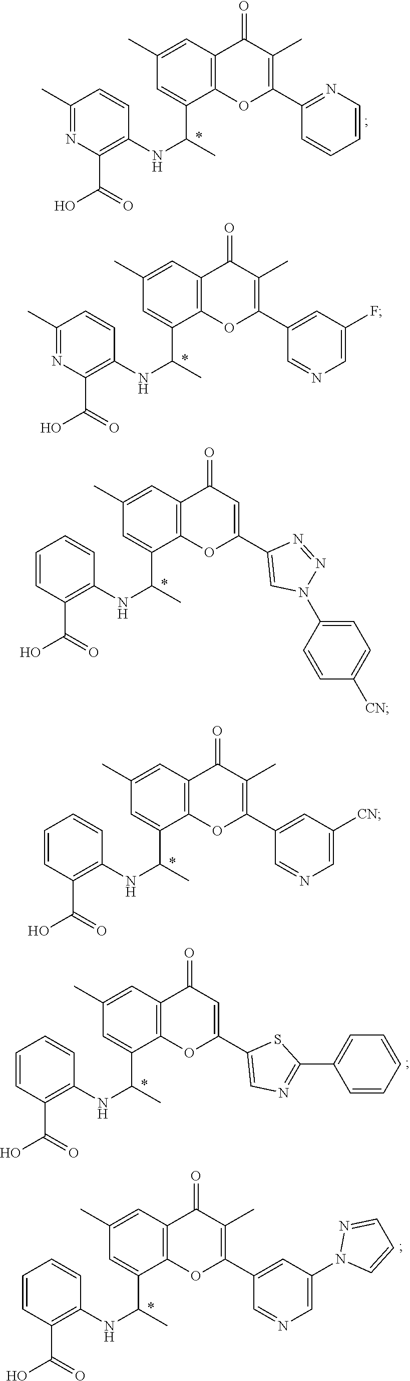 [SMILES: Cc1cc(C(C)Nc2ccc(C)nc2C(=O)O)c2oc(-c3ccccn3)c(C)c(=O)c2c1 ; Cc1cc(C(C)Nc2ccc(C)nc2C(=O)O)c2oc(-c3cncc(F)c3)c(C)c(=O)c2c1 ; Cc1cc(C(C)Nc2ccccc2C(=O)O)c2oc(-c3cn(-c4ccc(C#N)cc4)nn3)cc(=O)c2c1 ; Cc1cc(C(C)Nc2ccccc2C(=O)O)c2oc(-c3cncc(C#N)c3)c(C)c(=O)c2c1 ; Cc1cc(C(C)Nc2ccccc2C(=O)O)c2oc(-c3cnc(-c4ccccc4)s3)cc(=O)c2c1 ; Cc1cc(C(C)Nc2ccccc2C(=O)O)c2oc(-c3cncc(-n4cccn4)c3)c(C)c(=O)c2c1]

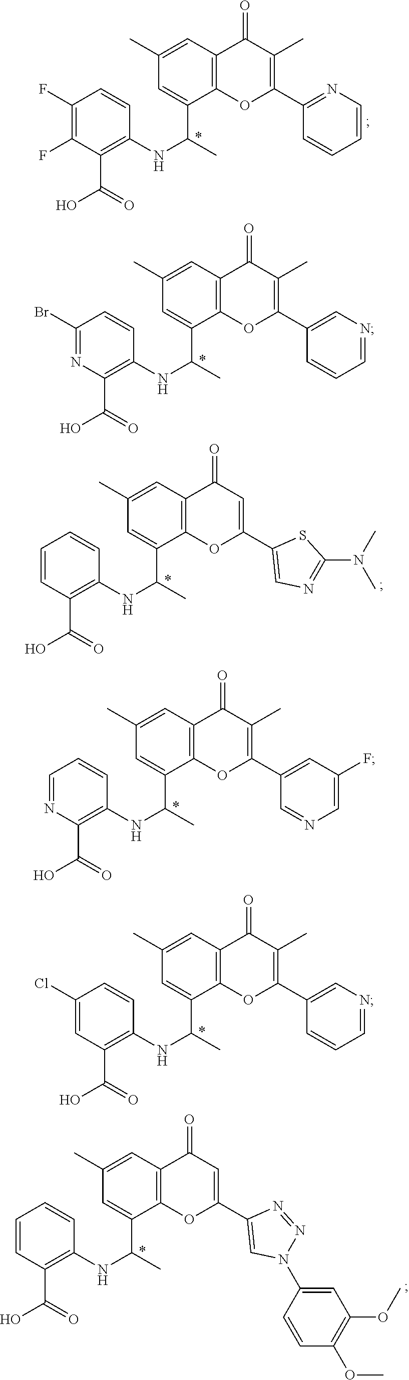 [SMILES: Cc1cc(C(C)Nc2ccc(F)c(F)c2C(=O)O)c2oc(-c3ccccn3)c(C)c(=O)c2c1 ; Cc1cc(C(C)Nc2ccc(Br)nc2C(=O)O)c2oc(-c3cccnc3)c(C)c(=O)c2c1 ; Cc1cc(C(C)Nc2ccccc2C(=O)O)c2oc(-c3cnc(N(C)C)s3)cc(=O)c2c1 ; Cc1cc(C(C)Nc2cccnc2C(=O)O)c2oc(-c3cncc(F)c3)c(C)c(=O)c2c1 ; Cc1cc(C(C)Nc2ccc(Cl)cc2C(=O)O)c2oc(-c3cccnc3)c(C)c(=O)c2c1 ; COc1ccc(-n2cc(-c3cc(=O)c4cc(C)cc(C(C)Nc5ccccc5C(=O)O)c4o3)nn2)cc1OC]

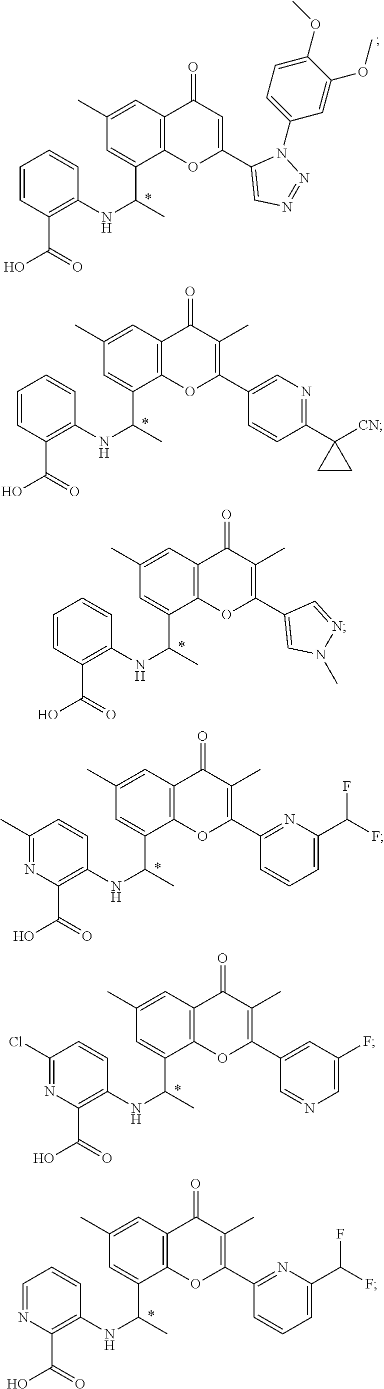

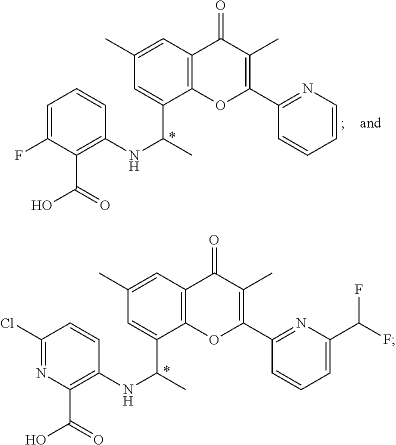 [SMILES: Cc1cc(C(C)Nc2cccc(F)c2C(=O)O)c2oc(-c3ccccn3)c(C)c(=O)c2c1 ; Cc1cc(C(C)Nc2ccc(Cl)nc2C(=O)O)c2oc(-c3cccc(C(F)F)n3)c(C)c(=O)c2c1]

or a pharmaceutically acceptable salt of any of the foregoing;
wherein the bond at the * position is as represented,

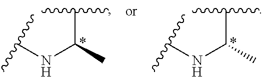

A further embodiment is a compound of Formula

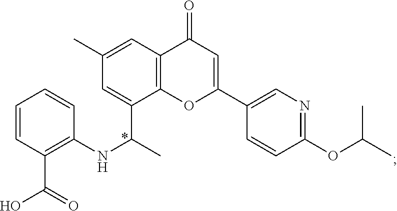 [SMILES: Cc1cc(C(C)Nc2ccccc2C(=O)O)c2oc(-c3ccc(OC(C)C)nc3)cc(=O)c2c1]

or a pharmaceutically acceptable salt thereof. In yet a further embodiment, the bond at the * position is

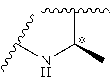 [SMILES: *N[C@@H](*)C]

In yet a further embodiment, the bond at the * position is

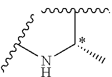 [SMILES: *N[C@H](*)C]

A further embodiment is a compound of Formula

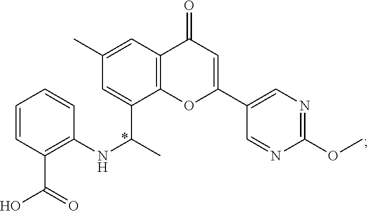 [SMILES: COc1ncc(-c2cc(=O)c3cc(C)cc(C(C)Nc4ccccc4C(=O)O)c3o2)cn1]

or a pharmaceutically acceptable salt thereof. In yet a further embodiment, the bond at the * position is

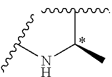 [SMILES: *N[C@@H](*)C]

In yet a further embodiment, the bond at the * position is

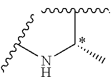 [SMILES: *N[C@H](*)C]

A further embodiment is a compound of Formula

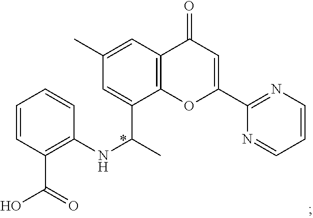 [SMILES: Cc1cc(C(C)Nc2ccccc2C(=O)O)c2oc(-c3ncccn3)cc(=O)c2c1]

or a pharmaceutically acceptable salt thereof. In yet a further embodiment, the bond at the * position is

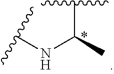 [SMILES: *N[C@@H](*)C]

In yet a further embodiment, the bond at the * position is

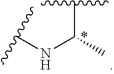 [SMILES: *N[C@H](*)C]

A further embodiment is a compound of Formula

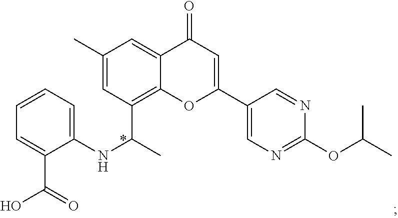 [SMILES: Cc1cc(C(C)Nc2ccccc2C(=O)O)c2oc(-c3cnc(OC(C)C)nc3)cc(=O)c2c1]

or a pharmaceutically acceptable salt thereof. In yet a further embodiment, the bond at the * position is

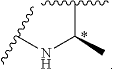 [SMILES: *N[C@@H](*)C]

In yet a further embodiment, the bond at the * position is

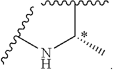 [SMILES: *N[C@H](*)C]

A further embodiment is a compound of Formula

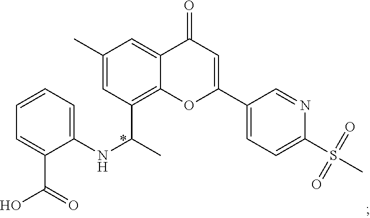 [SMILES: Cc1cc(C(C)Nc2ccccc2C(=O)O)c2oc(-c3ccc(S(C)(=O)=O)nc3)cc(=O)c2c1]

or a pharmaceutically acceptable salt thereof. In yet a further embodiment, the bond at the * position is

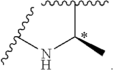 [SMILES: *N[C@@H](*)C]

In yet a further embodiment, the bond at the * position is

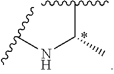 [SMILES: *N[C@H](*)C]

A further embodiment is a compound of Formula

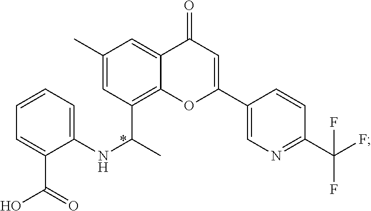 [SMILES: Cc1cc(C(C)Nc2ccccc2C(=O)O)c2oc(-c3ccc(C(F)(F)F)nc3)cc(=O)c2c1]

or a pharmaceutically acceptable salt thereof. In yet a further embodiment, the bond at the * position is

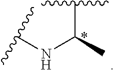 [SMILES: *N[C@@H](*)C]

In yet a further embodiment, the bond at the * position is

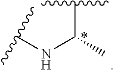 [SMILES: *N[C@H](*)C]

A further embodiment is a compound of Formula

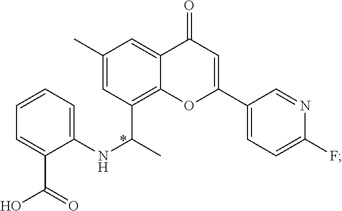 [SMILES: Cc1cc(C(C)Nc2ccccc2C(=O)O)c2oc(-c3ccc(F)nc3)cc(=O)c2c1]

or a pharmaceutically acceptable salt thereof. In yet a further embodiment, the bond at the * position is

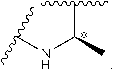 [SMILES: *N[C@@H](*)C]

In yet a further embodiment, the bond at the * position is

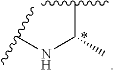 [SMILES: *N[C@H](*)C]

A further embodiment is a compound of Formula

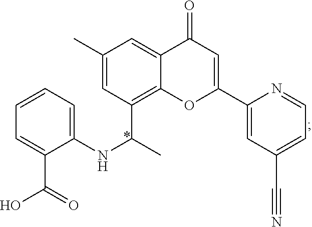 [SMILES: Cc1cc(C(C)Nc2ccccc2C(=O)O)c2oc(-c3cc(C#N)ccn3)cc(=O)c2c1]

or a pharmaceutically acceptable salt thereof. In yet a further embodiment, the bond at the * position is

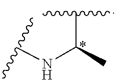 [SMILES: *N[C@@H](*)C]

In yet a further embodiment, the bond at the * position is

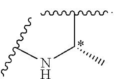 [SMILES: *N[C@H](*)C]

A further embodiment is a compound of Formula

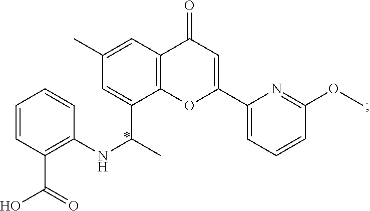 [SMILES: COc1cccc(-c2cc(=O)c3cc(C)cc(C(C)Nc4ccccc4C(=O)O)c3o2)n1]

or a pharmaceutically acceptable salt thereof. In yet a further embodiment, the bond at the * position is

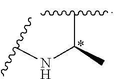 [SMILES: *N[C@@H](*)C]

In yet a further embodiment, the bond at the * position is

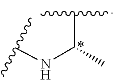 [SMILES: *N[C@H](*)C]

A further embodiment is a compound of Formula

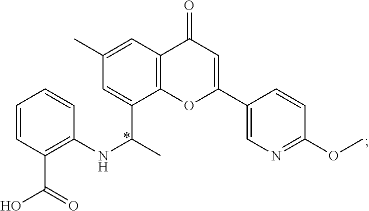 [SMILES: COc1ccc(-c2cc(=O)c3cc(C)cc(C(C)Nc4ccccc4C(=O)O)c3o2)cn1]

or a pharmaceutically acceptable salt thereof. In yet a further embodiment, the bond at the * position is

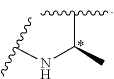 [SMILES: *N[C@@H](*)C]

In yet a further embodiment, the bond at the * position is

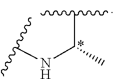 [SMILES: *N[C@H](*)C]

A further embodiment is a compound of Formula

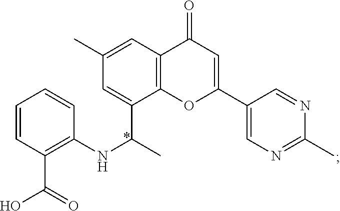 [SMILES: Cc1cc(C(C)Nc2ccccc2C(=O)O)c2oc(-c3cnc(C)nc3)cc(=O)c2c1]

or a pharmaceutically acceptable salt thereof. In yet a further embodiment, the bond at the * position is

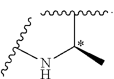 [SMILES: *N[C@@H](*)C]

In yet a further embodiment, the bond at the * position is

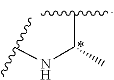 [SMILES: *N[C@H](*)C]

A further embodiment is a compound of Formula

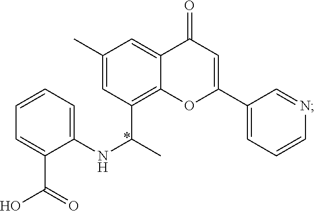 [SMILES: Cc1cc(C(C)Nc2ccccc2C(=O)O)c2oc(-c3cccnc3)cc(=O)c2c1]

or a pharmaceutically acceptable salt thereof. In yet a further embodiment, the bond at the * position is

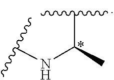 [SMILES: *N[C@@H](*)C]

In yet a further embodiment, the bond at the * position is

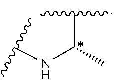 [SMILES: *N[C@H](*)C]

A further embodiment is a compound of Formula

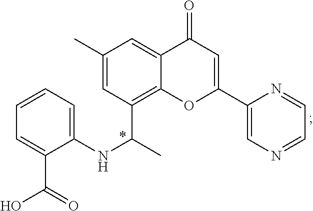 [SMILES: Cc1cc(C(C)Nc2ccccc2C(=O)O)c2oc(-c3cnccn3)cc(=O)c2c1]

or a pharmaceutically acceptable salt thereof. In yet a further embodiment, the bond at the * position is

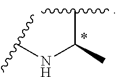 [SMILES: *N[C@@H](*)C]

In yet a further embodiment, the bond at the * position is

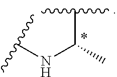 [SMILES: *N[C@H](*)C]

A further embodiment is a compound of Formula

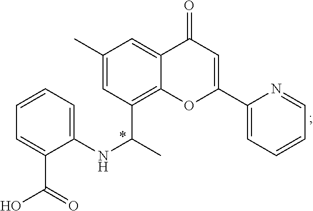 [SMILES: Cc1cc(C(C)Nc2ccccc2C(=O)O)c2oc(-c3ccccn3)cc(=O)c2c1]

or a pharmaceutically acceptable salt thereof. In yet a further embodiment, the bond at the * position is

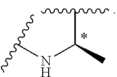 [SMILES: *N[C@@H](*)C]

In yet a further embodiment, the bond at the * position is

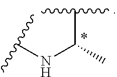 [SMILES: *N[C@H](*)C]

A further embodiment is a compound of Formula

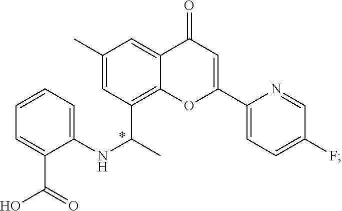 [SMILES: Cc1cc(C(C)Nc2ccccc2C(=O)O)c2oc(-c3ccc(F)cn3)cc(=O)c2c1]

or a pharmaceutically acceptable salt thereof. In yet a further embodiment, the bond at the * position is

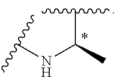 [SMILES: *N[C@@H](*)C]

In yet a further embodiment, the bond at the * position is

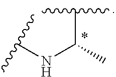 [SMILES: *N[C@H](*)C]

A further embodiment is a compound of Formula

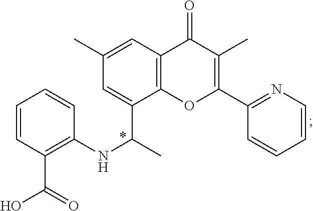 [SMILES: Cc1cc(C(C)Nc2ccccc2C(=O)O)c2oc(-c3ccccn3)c(C)c(=O)c2c1]

or a pharmaceutically acceptable salt thereof. In yet a further embodiment, the bond at the * position is

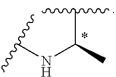 [SMILES: *N[C@@H](*)C]

In yet a further embodiment, the bond at the * position is

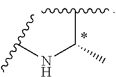 [SMILES: *N[C@H](*)C]

A further embodiment is a compound of Formula

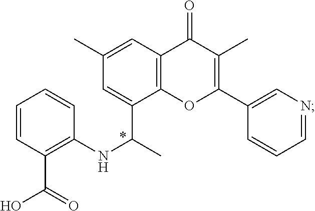 [SMILES: Cc1cc(C(C)Nc2ccccc2C(=O)O)c2oc(-c3cccnc3)c(C)c(=O)c2c1]

or a pharmaceutically acceptable salt thereof. In yet a further embodiment, the bond at the * position is

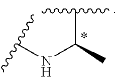 [SMILES: *N[C@@H](*)C]

In yet a further embodiment, the bond at the * position is

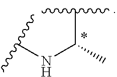 [SMILES: *N[C@H](*)C]

A further embodiment is a compound of Formula

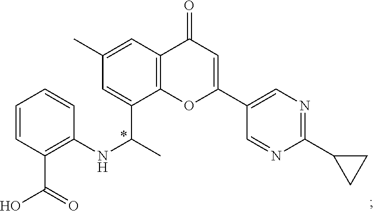 [SMILES: Cc1cc(C(C)Nc2ccccc2C(=O)O)c2oc(-c3cnc(C4CC4)nc3)cc(=O)c2c1]

or a pharmaceutically acceptable salt thereof. In yet a further embodiment, the bond at the * position is

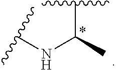 [SMILES: *N[C@@H](*)C]

In yet a further embodiment, the bond at the * position is

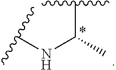 [SMILES: *N[C@H](*)C]

A further embodiment is a compound of Formula

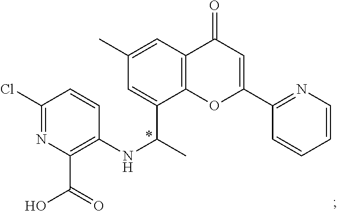 [SMILES: Cc1cc(C(C)Nc2ccc(Cl)nc2C(=O)O)c2oc(-c3ccccn3)cc(=O)c2c1]

or a pharmaceutically acceptable salt thereof. In yet a further embodiment, the bond at the * position is

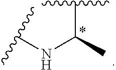 [SMILES: *N[C@@H](*)C]

In yet a further embodiment, the bond at the * position is

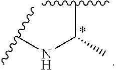 [SMILES: *N[C@H](*)C]

A further embodiment is a compound of Formula

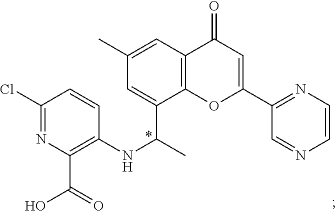 [SMILES: Cc1cc(C(C)Nc2ccc(Cl)nc2C(=O)O)c2oc(-c3cnccn3)cc(=O)c2c1]

or a pharmaceutically acceptable salt thereof. In yet a further embodiment, the bond at the * position is

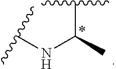 [SMILES: *N[C@@H](*)C]

In yet a further embodiment, the bond at the * position is

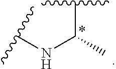 [SMILES: *N[C@H](*)C]

A further embodiment is a compound of Formula

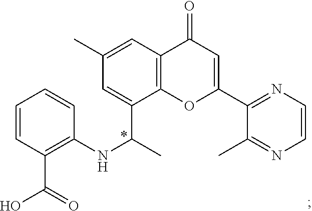 [SMILES: Cc1cc(C(C)Nc2ccccc2C(=O)O)c2oc(-c3nccnc3C)cc(=O)c2c1]

or a pharmaceutically acceptable salt thereof. In yet a further embodiment, the bond at the * position is

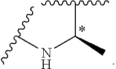 [SMILES: *N[C@@H](*)C]

In yet a further embodiment, the bond at the * position is

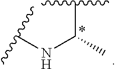 [SMILES: *N[C@H](*)C]

A further embodiment is a compound of Formula

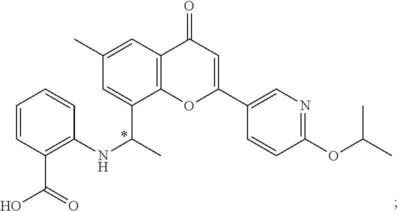 [SMILES: Cc1cc(C(C)Nc2ccccc2C(=O)O)c2oc(-c3ccc(OC(C)C)nc3)cc(=O)c2c1]

or a pharmaceutically acceptable salt thereof. In yet a further embodiment, the bond at the * position is

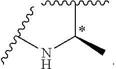 [SMILES: *N[C@@H](*)C]

In yet a further embodiment, the bond at the * position is

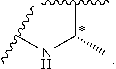 [SMILES: *N[C@H](*)C]

A further embodiment is a compound of Formula

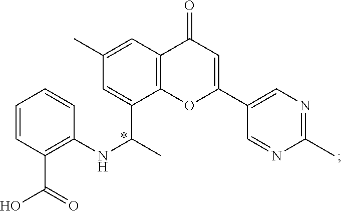 [SMILES: Cc1cc(C(C)Nc2ccccc2C(=O)O)c2oc(-c3cnc(C)nc3)cc(=O)c2c1]

or a pharmaceutically acceptable salt thereof. In yet a further embodiment, the bond at the * position is

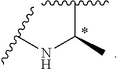 [SMILES: *N[C@@H](*)C]

In yet a further embodiment, the bond at the * position is

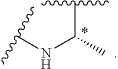 [SMILES: *N[C@H](*)C]

A further embodiment is a compound of Formula

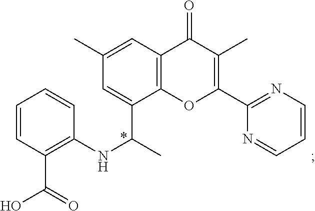 [SMILES: Cc1cc(C(C)Nc2ccccc2C(=O)O)c2oc(-c3ncccn3)c(C)c(=O)c2c1]

or a pharmaceutically acceptable salt thereof. In yet a further embodiment, the bond at the * position is

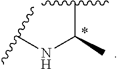 [SMILES: *N[C@@H](*)C]

In yet a further embodiment, the bond at the * position is

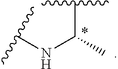 [SMILES: *N[C@H](*)C]

A further embodiment is a compound of Formula

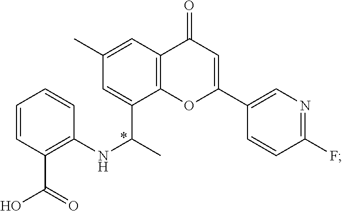 [SMILES: Cc1cc(C(C)Nc2ccccc2C(=O)O)c2oc(-c3ccc(F)nc3)cc(=O)c2c1]

or a pharmaceutically acceptable salt thereof. In yet a further embodiment, the bond at the * position is

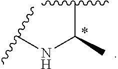 [SMILES: *N[C@@H](*)C]

In yet a further embodiment, the bond at the * position is

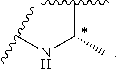 [SMILES: *N[C@H](*)C]

A further embodiment is a compound of Formula

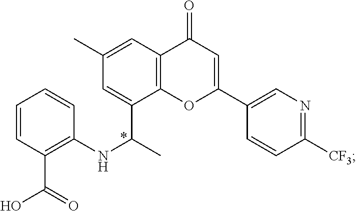 [SMILES: Cc1cc(C(C)Nc2ccccc2C(=O)O)c2oc(-c3ccc(C(F)(F)F)nc3)cc(=O)c2c1]

or a pharmaceutically acceptable salt thereof. In yet a further embodiment, the bond at the * position is

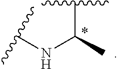 [SMILES: *N[C@@H](*)C]

In yet a further embodiment, the bond at the * position is

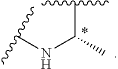 [SMILES: *N[C@H](*)C]

A further embodiment is a compound of Formula

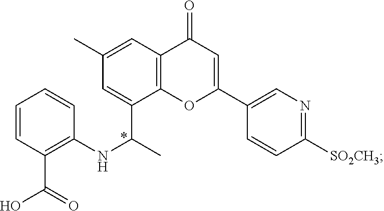 [SMILES: Cc1cc(C(C)Nc2ccccc2C(=O)O)c2oc(-c3ccc(S(C)(=O)=O)nc3)cc(=O)c2c1]

or a pharmaceutically acceptable salt thereof. In yet a further embodiment, the bond at the * position is

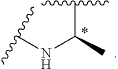 [SMILES: *N[C@@H](*)C]

In yet a further embodiment, the bond at the * position is

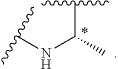 [SMILES: *N[C@H](*)C]

A further embodiment is a compound of Formula

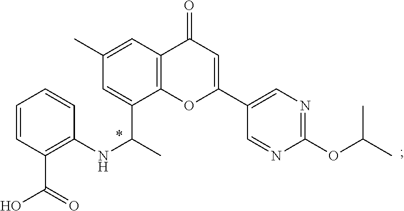 [SMILES: Cc1cc(C(C)Nc2ccccc2C(=O)O)c2oc(-c3cnc(OC(C)C)nc3)cc(=O)c2c1]

or a pharmaceutically acceptable salt thereof. In yet a further embodiment, the bond at the * position is

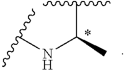 [SMILES: *N[C@@H](*)C]

In yet a further embodiment, the bond at the * position is

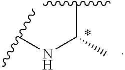 [SMILES: *N[C@H](*)C]

A further embodiment is a compound of Formula

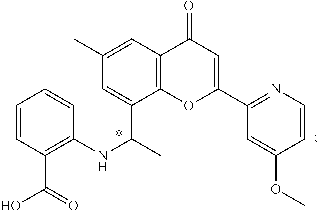 [SMILES: COc1ccnc(-c2cc(=O)c3cc(C)cc(C(C)Nc4ccccc4C(=O)O)c3o2)c1]

or a pharmaceutically acceptable salt thereof. In yet a further embodiment, the bond at the * position is

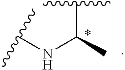 [SMILES: *N[C@@H](*)C]

In yet a further embodiment, the bond at the * position is

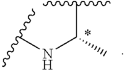 [SMILES: *N[C@H](*)C]

A further embodiment is a compound of Formula

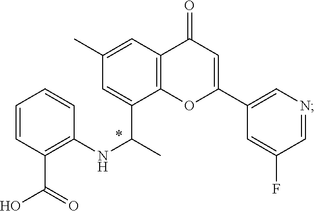 [SMILES: Cc1cc(C(C)Nc2ccccc2C(=O)O)c2oc(-c3cncc(F)c3)cc(=O)c2c1]

or a pharmaceutically acceptable salt thereof. In yet a further embodiment, the bond at the * position is

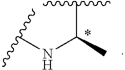 [SMILES: *N[C@@H](*)C]

In yet a further embodiment, the bond at the * position is

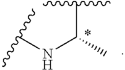 [SMILES: *N[C@H](*)C]

A further embodiment is a compound of Formula

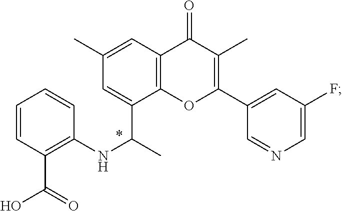 [SMILES: Cc1cc(C(C)Nc2ccccc2C(=O)O)c2oc(-c3cncc(F)c3)c(C)c(=O)c2c1]

or a pharmaceutically acceptable salt thereof. In yet a further embodiment, the bond at the * position is

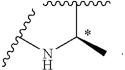 [SMILES: *N[C@@H](*)C]

In yet a further embodiment, the bond at the * position is

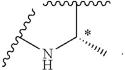 [SMILES: *N[C@H](*)C]

A further embodiment is a compound of Formula

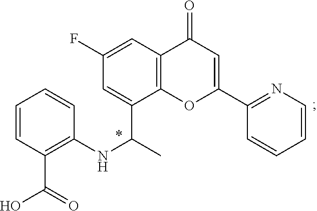 [SMILES: CC(Nc1ccccc1C(=O)O)c1cc(F)cc2c(=O)cc(-c3ccccn3)oc12]

or a pharmaceutically acceptable salt thereof. In yet a further embodiment, the bond at the * position is

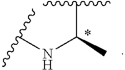 [SMILES: *N[C@@H](*)C]

In yet a further embodiment, the bond at the * position is

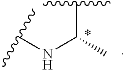 [SMILES: *N[C@H](*)C]

A further embodiment is a compound of Formula

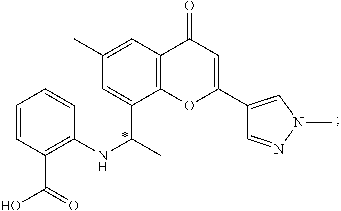 [SMILES: Cc1cc(C(C)Nc2ccccc2C(=O)O)c2oc(-c3cnn(C)c3)cc(=O)c2c1]

or a pharmaceutically acceptable salt thereof. In yet a further embodiment, the bond at the * position is

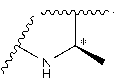 [SMILES: *N[C@@H](*)C]

In yet a further embodiment, the bond at the * position is

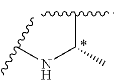 [SMILES: *N[C@H](*)C]

A further embodiment is a compound of Formula

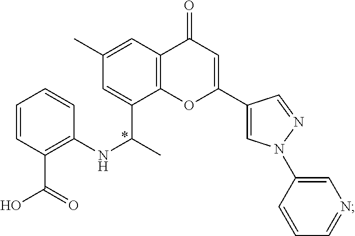 [SMILES: Cc1cc(C(C)Nc2ccccc2C(=O)O)c2oc(-c3cnn(-c4cccnc4)c3)cc(=O)c2c1]

or a pharmaceutically acceptable salt thereof. In yet a further embodiment, the bond at the * position is

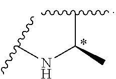 [SMILES: *N[C@@H](*)C]

In yet a further embodiment, the bond at the * position is

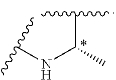 [SMILES: *N[C@H](*)C]

A further embodiment is a compound of Formula

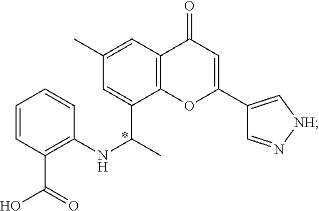 [SMILES: Cc1cc(C(C)Nc2ccccc2C(=O)O)c2oc(-c3cn[nH]c3)cc(=O)c2c1]

or a pharmaceutically acceptable salt thereof. In yet a further embodiment, the bond at the * position is

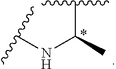 [SMILES: *N[C@@H](*)C]

In yet a further embodiment, the bond at the * position is

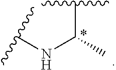 [SMILES: *N[C@H](*)C]

A further embodiment is a compound of Formula

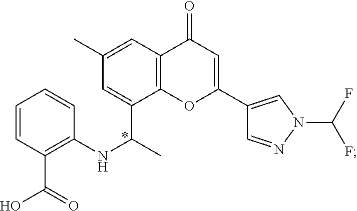 [SMILES: Cc1cc(C(C)Nc2ccccc2C(=O)O)c2oc(-c3cnn(C(F)F)c3)cc(=O)c2c1]

or a pharmaceutically acceptable salt thereof. In yet a further embodiment, the bond at the * position is

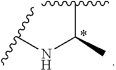 [SMILES: *N[C@@H](*)C]

In yet a further embodiment, the bond at the * position is

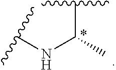 [SMILES: *N[C@H](*)C]

A further embodiment is a compound of Formula

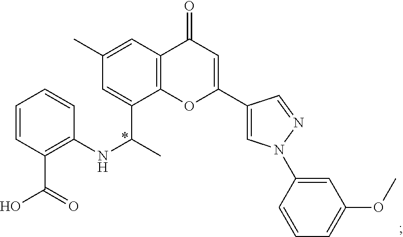 [SMILES: COc1cccc(-n2cc(-c3cc(=O)c4cc(C)cc(C(C)Nc5ccccc5C(=O)O)c4o3)cn2)c1]

or a pharmaceutically acceptable salt thereof. In yet a further embodiment, the bond at the * position is

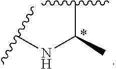 [SMILES: *N[C@@H](*)C]

In yet a further embodiment, the bond at the * position is

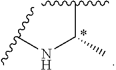 [SMILES: *N[C@H](*)C]

A further embodiment is a compound of Formula

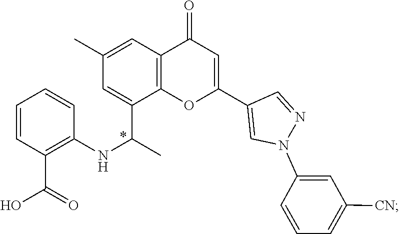 [SMILES: Cc1cc(C(C)Nc2ccccc2C(=O)O)c2oc(-c3cnn(-c4cccc(C#N)c4)c3)cc(=O)c2c1]

or a pharmaceutically acceptable salt thereof. In yet a further embodiment, the bond at the * position is

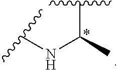 [SMILES: *N[C@@H](*)C]

In yet a further embodiment, the bond at the * position is

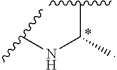 [SMILES: *N[C@H](*)C]

A further embodiment is a compound of Formula

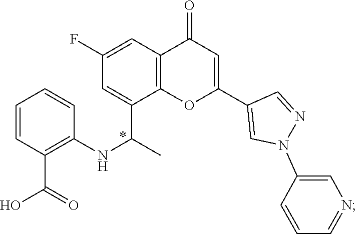 [SMILES: CC(Nc1ccccc1C(=O)O)c1cc(F)cc2c(=O)cc(-c3cnn(-c4cccnc4)c3)oc12]

or a pharmaceutically acceptable salt thereof. In yet a further embodiment, the bond at the * position is

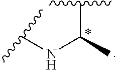 [SMILES: *N[C@@H](*)C]

In yet a further embodiment, the bond at the * position is

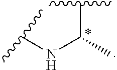 [SMILES: *N[C@H](*)C]

A further embodiment is a compound of Formula

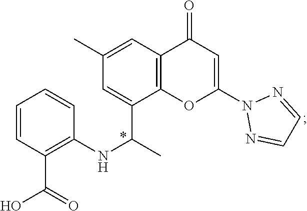 [SMILES: Cc1cc(C(C)Nc2ccccc2C(=O)O)c2oc(-n3nccn3)cc(=O)c2c1]

or a pharmaceutically acceptable salt thereof. In yet a further embodiment, the bond at the * position is

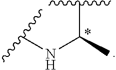 [SMILES: *N[C@@H](*)C]

In yet a further embodiment, the bond at the * position is

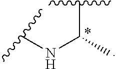 [SMILES: *N[C@H](*)C]

A further embodiment is a compound of Formula

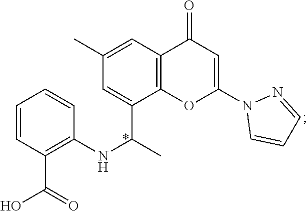 [SMILES: Cc1cc(C(C)Nc2ccccc2C(=O)O)c2oc(-n3cccn3)cc(=O)c2c1]

or a pharmaceutically acceptable salt thereof. In yet a further embodiment, the bond at the * position is

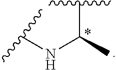 [SMILES: *N[C@@H](*)C]

In yet a further embodiment, the bond at the * position is

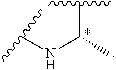 [SMILES: *N[C@H](*)C]

A further embodiment is a compound of Formula

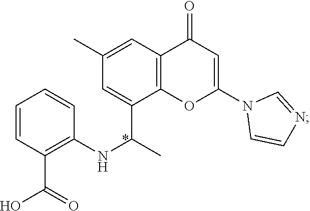 [SMILES: Cc1cc(C(C)Nc2ccccc2C(=O)O)c2oc(-n3ccnc3)cc(=O)c2c1]

or a pharmaceutically acceptable salt thereof. In yet a further embodiment, the bond at the * position is

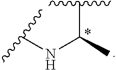 [SMILES: *N[C@@H](*)C]

In yet a further embodiment, the bond at the * position is

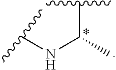 [SMILES: *N[C@H](*)C]

A further embodiment is a compound of Formula

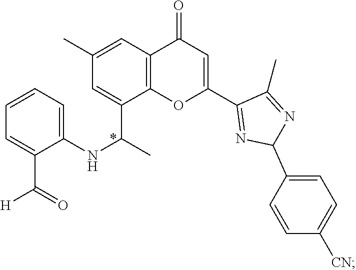 [SMILES: CC1=NC(c2ccc(C#N)cc2)N=C1c1cc(=O)c2cc(C)cc(C(C)Nc3ccccc3C=O)c2o1]

or a pharmaceutically acceptable salt thereof. In yet a further embodiment, the bond at the * position is

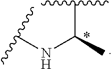 [SMILES: *N[C@@H](*)C]

In yet a further embodiment, the bond at the * position is

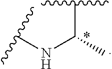 [SMILES: *N[C@H](*)C]

A further embodiment is a compound of Formula

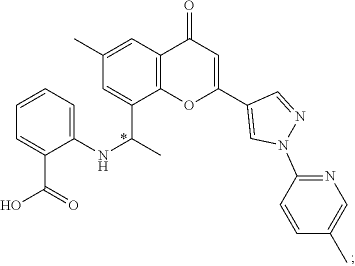 [SMILES: Cc1ccc(-n2cc(-c3cc(=O)c4cc(C)cc(C(C)Nc5ccccc5C(=O)O)c4o3)cn2)nc1]

or a pharmaceutically acceptable salt thereof. In yet a further embodiment, the bond at the * position is

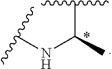 [SMILES: *N[C@@H](*)C]

In yet a further embodiment, the bond at the * position is

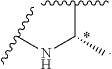 [SMILES: *N[C@H](*)C]

A further embodiment is a compound of Formula

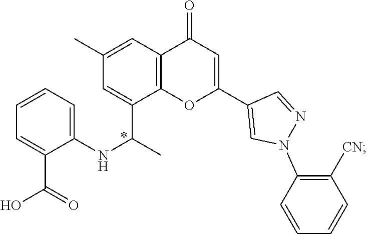 [SMILES: Cc1cc(C(C)Nc2ccccc2C(=O)O)c2oc(-c3cnn(-c4ccccc4C#N)c3)cc(=O)c2c1]

or a pharmaceutically acceptable salt thereof. In yet a further embodiment, the bond at the * position is

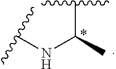 [SMILES: *N[C@@H](*)C]

In yet a further embodiment, the bond at the * position is

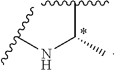 [SMILES: *N[C@H](*)C]

A further embodiment is a compound of Formula

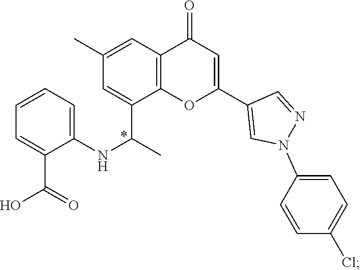 [SMILES: Cc1cc(C(C)Nc2ccccc2C(=O)O)c2oc(-c3cnn(-c4ccc(Cl)cc4)c3)cc(=O)c2c1]

or a pharmaceutically acceptable salt thereof. In yet a further embodiment, the bond at the * position is

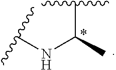 [SMILES: *N[C@@H](*)C]

In yet a further embodiment, the bond at the * position is

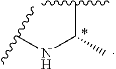 [SMILES: *N[C@H](*)C]

A further embodiment is a compound of Formula

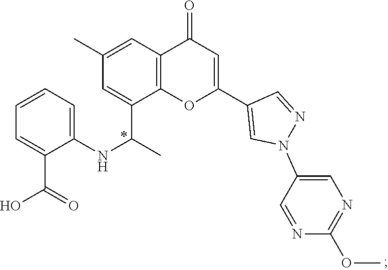 [SMILES: COc1ncc(-n2cc(-c3cc(=O)c4cc(C)cc(C(C)Nc5ccccc5C(=O)O)c4o3)cn2)cn1]

or a pharmaceutically acceptable salt thereof. In yet a further embodiment, the bond at the * position is

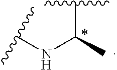 [SMILES: *N[C@@H](*)C]

In yet a further embodiment, the bond at the * position is

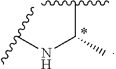 [SMILES: *N[C@H](*)C]

A further embodiment is a compound of Formula

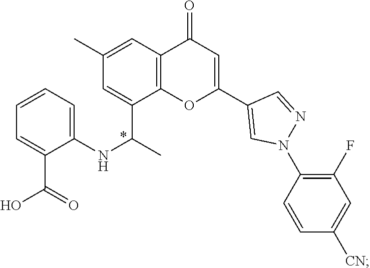 [SMILES: Cc1cc(C(C)Nc2ccccc2C(=O)O)c2oc(-c3cnn(-c4ccc(C#N)cc4F)c3)cc(=O)c2c1]

or a pharmaceutically acceptable salt thereof. In yet a further embodiment, the bond at the * position is

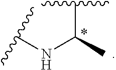 [SMILES: *N[C@@H](*)C]

In yet a further embodiment, the bond at the * position is

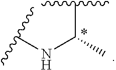 [SMILES: *N[C@H](*)C]

A further embodiment is a compound of Formula

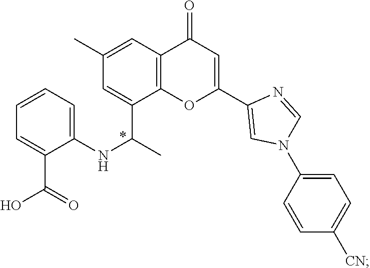 [SMILES: Cc1cc(C(C)Nc2ccccc2C(=O)O)c2oc(-c3cn(-c4ccc(C#N)cc4)cn3)cc(=O)c2c1]

or a pharmaceutically acceptable salt thereof. In yet a further embodiment, the bond at the * position is

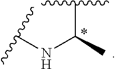 [SMILES: *N[C@@H](*)C]

In yet a further embodiment, the bond at the * position is

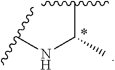 [SMILES: *N[C@H](*)C]

A further embodiment is a compound of Formula

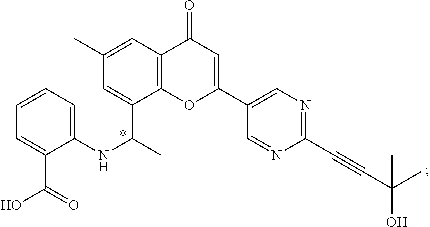 [SMILES: Cc1cc(C(C)Nc2ccccc2C(=O)O)c2oc(-c3cnc(C#CC(C)(C)O)nc3)cc(=O)c2c1]

or a pharmaceutically acceptable salt thereof. In yet a further embodiment, the bond at the * position is

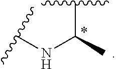 [SMILES: *N[C@@H](*)C]

In yet a further embodiment, the bond at the * position is

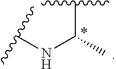 [SMILES: *N[C@H](*)C]

A further embodiment is a compound of Formula

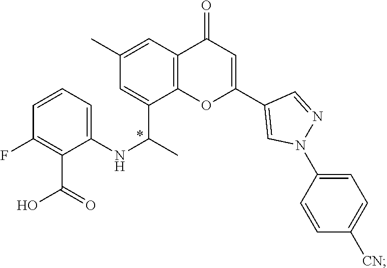 [SMILES: Cc1cc(C(C)Nc2cccc(F)c2C(=O)O)c2oc(-c3cnn(-c4ccc(C#N)cc4)c3)cc(=O)c2c1]

or a pharmaceutically acceptable salt thereof. In yet a further embodiment, the bond at the * position is

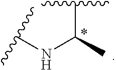 [SMILES: *N[C@@H](*)C]

In yet a further embodiment, the bond at the * position is

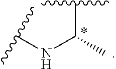 [SMILES: *N[C@H](*)C]

A further embodiment is a compound of Formula

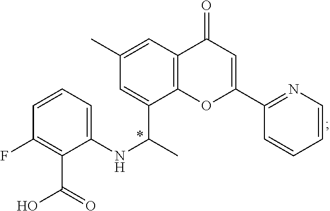 [SMILES: Cc1cc(C(C)Nc2cccc(F)c2C(=O)O)c2oc(-c3ccccn3)cc(=O)c2c1]

or a pharmaceutically acceptable salt thereof. In yet a further embodiment, the bond at the * position is

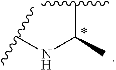 [SMILES: *N[C@@H](*)C]

In yet a further embodiment, the bond at the * position is

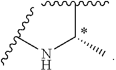 [SMILES: *N[C@H](*)C]

A further embodiment is a compound of Formula

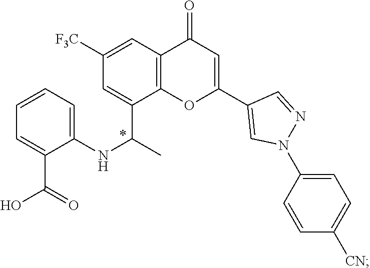 [SMILES: CC(Nc1ccccc1C(=O)O)c1cc(C(F)(F)F)cc2c(=O)cc(-c3cnn(-c4ccc(C#N)cc4)c3)oc12]

or a pharmaceutically acceptable salt thereof. In yet a further embodiment, the bond at the * position is

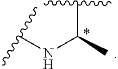 [SMILES: *N[C@@H](*)C]

In yet a further embodiment, the bond at the * position is

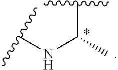 [SMILES: *N[C@H](*)C]

A further embodiment is a compound of Formula

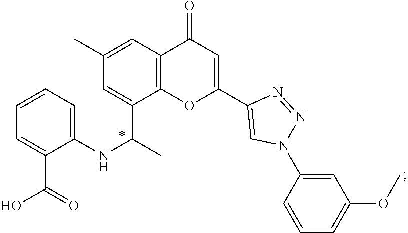 [SMILES: COc1cccc(-n2cc(-c3cc(=O)c4cc(C)cc(C(C)Nc5ccccc5C(=O)O)c4o3)nn2)c1]

or a pharmaceutically acceptable salt thereof. In yet a further embodiment, the bond at the * position is

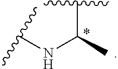 [SMILES: *N[C@@H](*)C]

In yet a further embodiment, the bond at the * position is

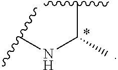 [SMILES: *N[C@H](*)C]

A further embodiment is a compound of Formula

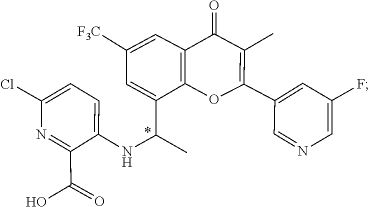 [SMILES: Cc1c(-c2cncc(F)c2)oc2c(C(C)Nc3ccc(Cl)nc3C(=O)O)cc(C(F)(F)F)cc2c1=O]

or a pharmaceutically acceptable salt thereof. In yet a further embodiment, the bond at the * position is

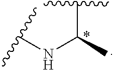 [SMILES: *N[C@@H](*)C]

In yet a further embodiment, the bond at the * position is

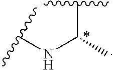 [SMILES: *N[C@H](*)C]

A further embodiment is a compound of Formula

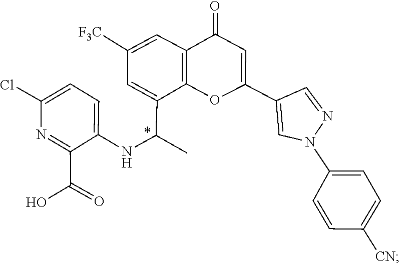 [SMILES: CC(Nc1ccc(Cl)nc1C(=O)O)c1cc(C(F)(F)F)cc2c(=O)cc(-c3cnn(-c4ccc(C#N)cc4)c3)oc12]

or a pharmaceutically acceptable salt thereof. In yet a further embodiment, the bond at the * position is

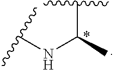 [SMILES: *N[C@@H](*)C]

In yet a further embodiment, the bond at the * position is

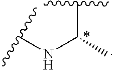 [SMILES: *N[C@H](*)C]

A further embodiment is a compound of Formula

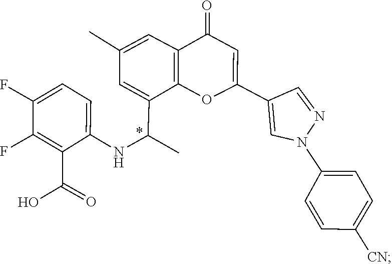 [SMILES: Cc1cc(C(C)Nc2ccc(F)c(F)c2C(=O)O)c2oc(-c3cnn(-c4ccc(C#N)cc4)c3)cc(=O)c2c1]

or a pharmaceutically acceptable salt thereof. In yet a further embodiment, the bond at the * position is

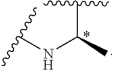 [SMILES: *N[C@@H](*)C]

In yet a further embodiment, the bond at the * position is

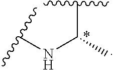 [SMILES: *N[C@H](*)C]

A further embodiment is a compound of Formula

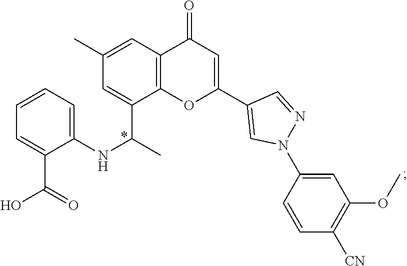 [SMILES: COc1cc(-n2cc(-c3cc(=O)c4cc(C)cc(C(C)Nc5ccccc5C(=O)O)c4o3)cn2)ccc1C#N]

or a pharmaceutically acceptable salt thereof. In yet a further embodiment, the bond at the * position is

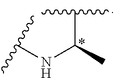 [SMILES: *N[C@@H](*)C]

In yet a further embodiment, the bond at the * position is

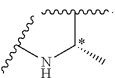 [SMILES: *N[C@H](*)C]

A further embodiment is a compound of Formula

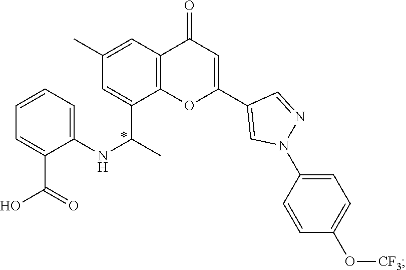 [SMILES: Cc1cc(C(C)Nc2ccccc2C(=O)O)c2oc(-c3cnn(-c4ccc(OC(F)(F)F)cc4)c3)cc(=O)c2c1]

or a pharmaceutically acceptable salt thereof. In yet a further embodiment, the bond at the * position is

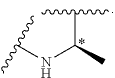 [SMILES: *N[C@@H](*)C]

In yet a further embodiment, the bond at the * position is

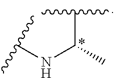 [SMILES: *N[C@H](*)C]

A further embodiment is a compound of Formula

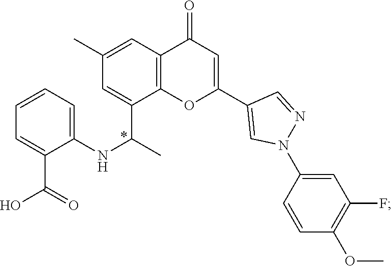 [SMILES: COc1ccc(-n2cc(-c3cc(=O)c4cc(C)cc(C(C)Nc5ccccc5C(=O)O)c4o3)cn2)cc1F]

or a pharmaceutically acceptable salt thereof. In yet a further embodiment, the bond at the * position is

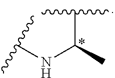 [SMILES: *N[C@@H](*)C]

In yet a further embodiment, the bond at the * position is

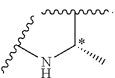 [SMILES: *N[C@H](*)C]

A further embodiment is a compound of Formula

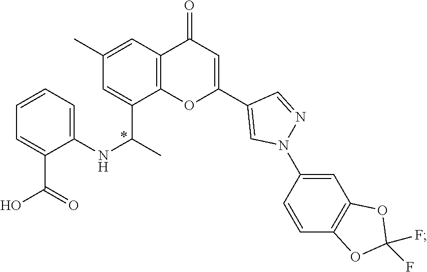 [SMILES: Cc1cc(C(C)Nc2ccccc2C(=O)O)c2oc(-c3cnn(-c4ccc5c(c4)OC(F)(F)O5)c3)cc(=O)c2c1]

or a pharmaceutically acceptable salt thereof. In yet a further embodiment, the bond at the * position is

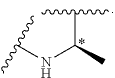 [SMILES: *N[C@@H](*)C]

In yet a further embodiment, the bond at the * position is

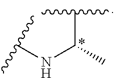 [SMILES: *N[C@H](*)C]

A further embodiment is a compound of Formula

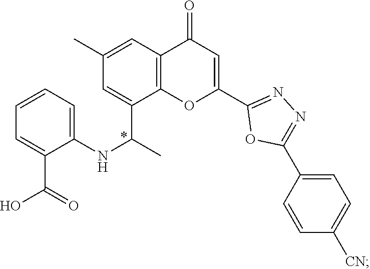 [SMILES: Cc1cc(C(C)Nc2ccccc2C(=O)O)c2oc(-c3nnc(-c4ccc(C#N)cc4)o3)cc(=O)c2c1]

or a pharmaceutically acceptable salt thereof. In yet a further embodiment, the bond at the * position is

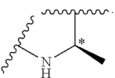 [SMILES: *N[C@@H](*)C]

In yet a further embodiment, the bond at the * position is

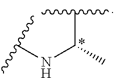 [SMILES: *N[C@H](*)C]

A further embodiment is a compound of Formula

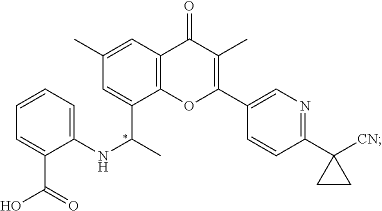 [SMILES: Cc1cc(C(C)Nc2ccccc2C(=O)O)c2oc(-c3ccc(C4(C#N)CC4)nc3)c(C)c(=O)c2c1]

or a pharmaceutically acceptable salt thereof. In yet a further embodiment, the bond at the * position is

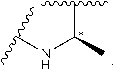 [SMILES: *N[C@@H](*)C]

In yet a further embodiment, the bond at the * position is

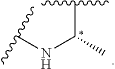 [SMILES: *N[C@H](*)C]

A further embodiment is a compound of Formula

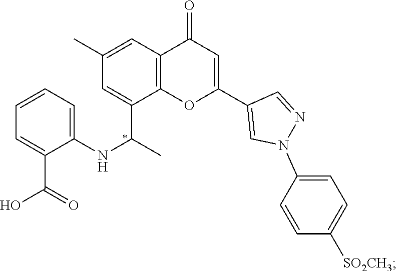 [SMILES: Cc1cc(C(C)Nc2ccccc2C(=O)O)c2oc(-c3cnn(-c4ccc(S(C)(=O)=O)cc4)c3)cc(=O)c2c1]

or a pharmaceutically acceptable salt thereof. In yet a further embodiment, the bond at the * position is

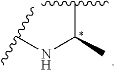 [SMILES: *N[C@@H](*)C]

In yet a further embodiment, the bond at the * position is

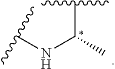 [SMILES: *N[C@H](*)C]

A further embodiment is a compound of Formula

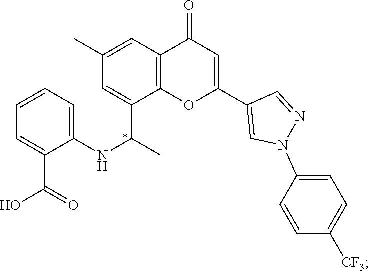 [SMILES: Cc1cc(C(C)Nc2ccccc2C(=O)O)c2oc(-c3cnn(-c4ccc(C(F)(F)F)cc4)c3)cc(=O)c2c1]

or a pharmaceutically acceptable salt thereof. In yet a further embodiment, the bond at the * position is

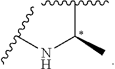 [SMILES: *N[C@@H](*)C]

In yet a further embodiment, the bond at the * position is

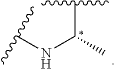 [SMILES: *N[C@H](*)C]

A further embodiment is a compound of Formula

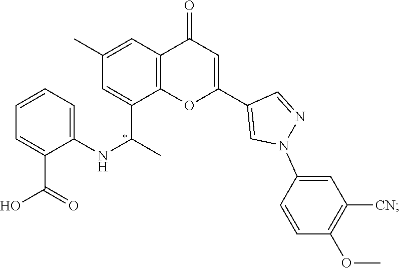 [SMILES: COc1ccc(-n2cc(-c3cc(=O)c4cc(C)cc(C(C)Nc5ccccc5C(=O)O)c4o3)cn2)cc1C#N]

or a pharmaceutically acceptable salt thereof. In yet a further embodiment, the bond at the * position is

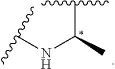 [SMILES: *N[C@@H](*)C]

In yet a further embodiment, the bond at the * position is

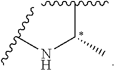 [SMILES: *N[C@H](*)C]

A further embodiment is a compound of Formula

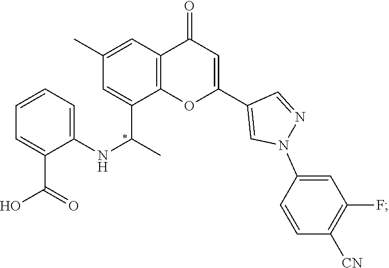 [SMILES: Cc1cc(C(C)Nc2ccccc2C(=O)O)c2oc(-c3cnn(-c4ccc(C#N)c(F)c4)c3)cc(=O)c2c1]

or a pharmaceutically acceptable salt thereof. In yet a further embodiment, the bond at the * position is

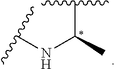 [SMILES: *N[C@@H](*)C]

In yet a further embodiment, the bond at the * position is

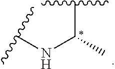 [SMILES: *N[C@H](*)C]

A further embodiment is a compound of Formula

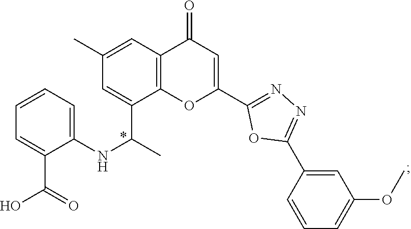 [SMILES: COc1cccc(-c2nnc(-c3cc(=O)c4cc(C)cc(C(C)Nc5ccccc5C(=O)O)c4o3)o2)c1]

or a pharmaceutically acceptable salt thereof. In yet a further embodiment, the bond at the * position is

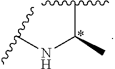 [SMILES: *N[C@@H](*)C]

In yet a further embodiment, the bond at the * position is

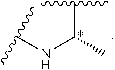 [SMILES: *N[C@H](*)C]

A further embodiment is a compound of Formula

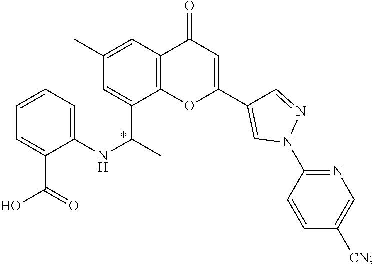 [SMILES: Cc1cc(C(C)Nc2ccccc2C(=O)O)c2oc(-c3cnn(-c4ccc(C#N)cn4)c3)cc(=O)c2c1]

or a pharmaceutically acceptable salt thereof. In yet a further embodiment, the bond at the * position is

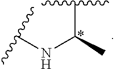 [SMILES: *N[C@@H](*)C]

In yet a further embodiment, the bond at the * position is

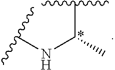 [SMILES: *N[C@H](*)C]

A further embodiment is a compound of Formula

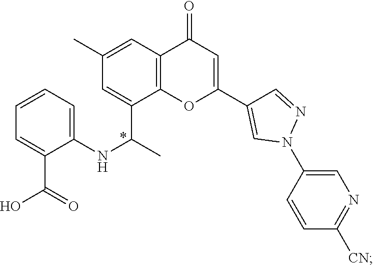 [SMILES: Cc1cc(C(C)Nc2ccccc2C(=O)O)c2oc(-c3cnn(-c4ccc(C#N)nc4)c3)cc(=O)c2c1]

or a pharmaceutically acceptable salt thereof. In yet a further embodiment, the bond at the * position is

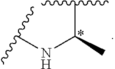 [SMILES: *N[C@@H](*)C]

In yet a further embodiment, the bond at the * position is

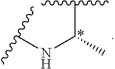 [SMILES: *N[C@H](*)C]

A further embodiment is a compound of Formula

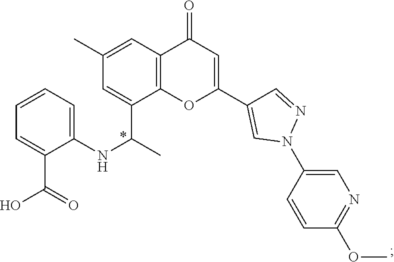 [SMILES: COc1ccc(-n2cc(-c3cc(=O)c4cc(C)cc(C(C)Nc5ccccc5C(=O)O)c4o3)cn2)cn1]

or a pharmaceutically acceptable salt thereof. In yet a further embodiment, the bond at the * position is

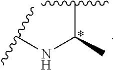 [SMILES: *N[C@@H](*)C]

In yet a further embodiment, the bond at the * position is

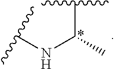 [SMILES: *N[C@H](*)C]

A further embodiment is a compound of Formula

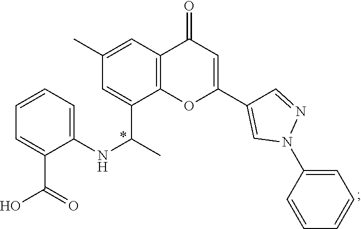 [SMILES: Cc1cc(C(C)Nc2ccccc2C(=O)O)c2oc(-c3cnn(-c4ccccc4)c3)cc(=O)c2c1]

or a pharmaceutically acceptable salt thereof. In yet a further embodiment, the bond at the * position is

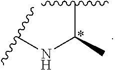 [SMILES: *N[C@@H](*)C]

In yet a further embodiment, the bond at the * position is

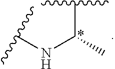 [SMILES: *N[C@H](*)C]

A further embodiment is a compound of Formula

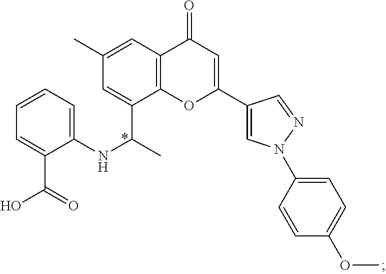 [SMILES: COc1ccc(-n2cc(-c3cc(=O)c4cc(C)cc(C(C)Nc5ccccc5C(=O)O)c4o3)cn2)cc1]

or a pharmaceutically acceptable salt thereof. In yet a further embodiment, the bond at the * position is

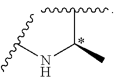 [SMILES: *N[C@@H](*)C]

In yet a further embodiment, the bond at the * position is

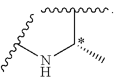 [SMILES: *N[C@H](*)C]

A further embodiment is a compound of Formula

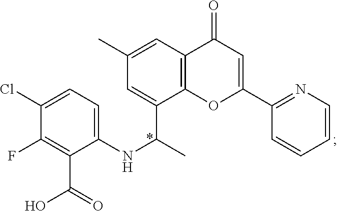 [SMILES: Cc1cc(C(C)Nc2ccc(Cl)c(F)c2C(=O)O)c2oc(-c3ccccn3)cc(=O)c2c1]

or a pharmaceutically acceptable salt thereof. In yet a further embodiment, the bond at the * position is

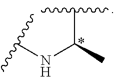 [SMILES: *N[C@@H](*)C]

In yet a further embodiment, the bond at the * position is

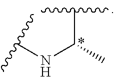 [SMILES: *N[C@H](*)C]

A further embodiment is a compound of Formula

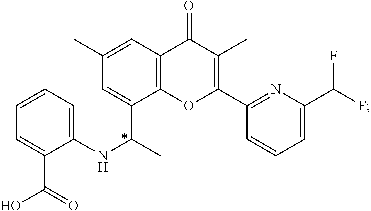 [SMILES: Cc1cc(C(C)Nc2ccccc2C(=O)O)c2oc(-c3cccc(C(F)F)n3)c(C)c(=O)c2c1]

or a pharmaceutically acceptable salt thereof. In yet a further embodiment, the bond at the * position is

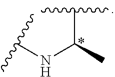 [SMILES: *N[C@@H](*)C]

In yet a further embodiment, the bond at the * position is

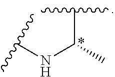 [SMILES: *N[C@H](*)C]

A further embodiment is a compound of Formula

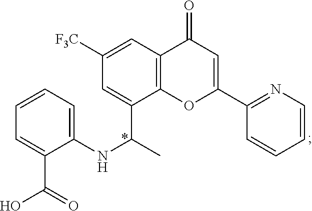 [SMILES: CC(Nc1ccccc1C(=O)O)c1cc(C(F)(F)F)cc2c(=O)cc(-c3ccccn3)oc12]

or a pharmaceutically acceptable salt thereof. In yet a further embodiment, the bond at the * position is

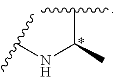 [SMILES: *N[C@@H](*)C]

In yet a further embodiment, the bond at the * position is

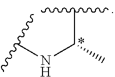 [SMILES: *N[C@H](*)C]

A further embodiment is a compound of Formula

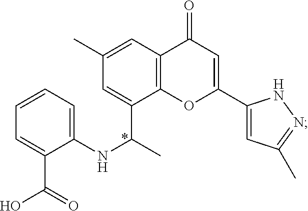 [SMILES: Cc1cc(C(C)Nc2ccccc2C(=O)O)c2oc(-c3cc(C)n[nH]3)cc(=O)c2c1]

or a pharmaceutically acceptable salt thereof. In yet a further embodiment, the bond at the * position is

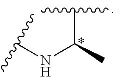 [SMILES: *N[C@@H](*)C]

In yet a further embodiment, the bond at the * position is

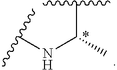 [SMILES: *N[C@H](*)C]

A further embodiment is a compound of Formula

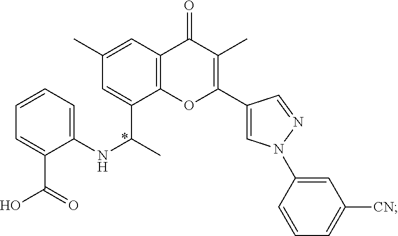 [SMILES: Cc1cc(C(C)Nc2ccccc2C(=O)O)c2oc(-c3cnn(-c4cccc(C#N)c4)c3)c(C)c(=O)c2c1]

or a pharmaceutically acceptable salt thereof. In yet a further embodiment, the bond at the * position is

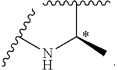 [SMILES: *N[C@@H](*)C]

In yet a further embodiment, the bond at the * position is

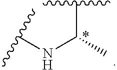 [SMILES: *N[C@H](*)C]

A further embodiment is a compound of Formula

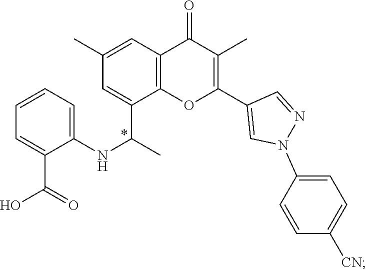 [SMILES: Cc1cc(C(C)Nc2ccccc2C(=O)O)c2oc(-c3cnn(-c4ccc(C#N)cc4)c3)c(C)c(=O)c2c1]

or a pharmaceutically acceptable salt thereof. In yet a further embodiment, the bond at the * position is

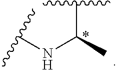 [SMILES: *N[C@@H](*)C]

In yet a further embodiment, the bond at the * position is

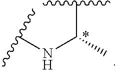 [SMILES: *N[C@H](*)C]

A further embodiment is a compound of Formula

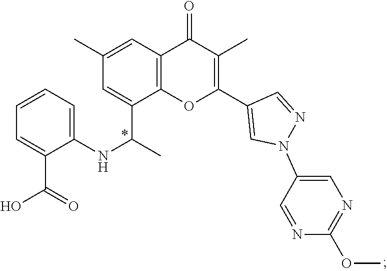 [SMILES: COc1ncc(-n2cc(-c3oc4c(C(C)Nc5ccccc5C(=O)O)cc(C)cc4c(=O)c3C)cn2)cn1]

or a pharmaceutically acceptable salt thereof. In yet a further embodiment, the bond at the *

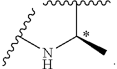 [SMILES: *N[C@@H](*)C]

In yet a further embodiment, the bond at the * position is

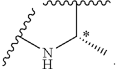 [SMILES: *N[C@H](*)C]

A further embodiment is a compound of Formula

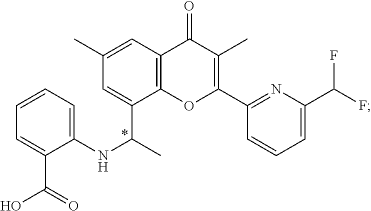 [SMILES: Cc1cc(C(C)Nc2ccccc2C(=O)O)c2oc(-c3cccc(C(F)F)n3)c(C)c(=O)c2c1]

or a pharmaceutically acceptable salt thereof. In yet a further embodiment, the bond at the * position is

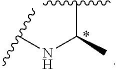 [SMILES: *N[C@@H](*)C]

In yet a further embodiment, the bond at the * position is

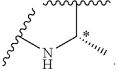 [SMILES: *N[C@H](*)C]

A further embodiment is a compound of Formula

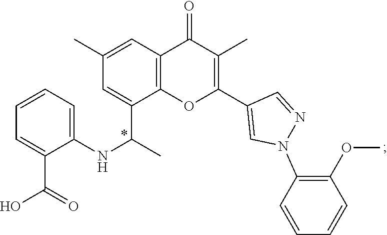 [SMILES: COc1ccccc1-n1cc(-c2oc3c(C(C)Nc4ccccc4C(=O)O)cc(C)cc3c(=O)c2C)cn1]

or a pharmaceutically acceptable salt thereof. In yet a further embodiment, the bond at the * position is

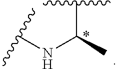 [SMILES: *N[C@@H](*)C]

In yet a further embodiment, the bond at the * position is

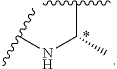 [SMILES: *N[C@H](*)C]

A further embodiment is a compound of Formula

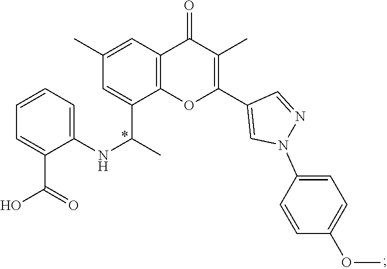 [SMILES: COc1ccc(-n2cc(-c3oc4c(C(C)Nc5ccccc5C(=O)O)cc(C)cc4c(=O)c3C)cn2)cc1]

or a pharmaceutically acceptable salt thereof. In yet a further embodiment, the bond at the * position is

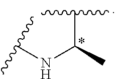 [SMILES: *N[C@@H](*)C]

In yet a further embodiment, the bond at the * position is

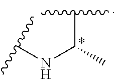 [SMILES: *N[C@H](*)C]

A further embodiment is a compound of Formula

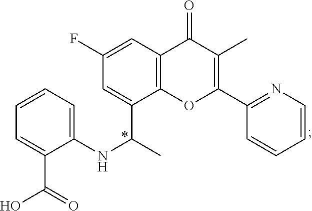 [SMILES: Cc1c(-c2ccccn2)oc2c(C(C)Nc3ccccc3C(=O)O)cc(F)cc2c1=O]

or a pharmaceutically acceptable salt thereof. In yet a further embodiment, the bond at the * position is

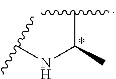 [SMILES: *N[C@@H](*)C]

In yet a further embodiment, the bond at the * position is

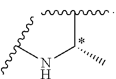 [SMILES: *N[C@H](*)C]

A further embodiment is a compound of Formula

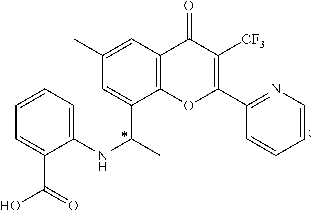 [SMILES: Cc1cc(C(C)Nc2ccccc2C(=O)O)c2oc(-c3ccccn3)c(C(F)(F)F)c(=O)c2c1]

or a pharmaceutically acceptable salt thereof. In yet a further embodiment, the bond at the * position is

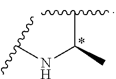 [SMILES: *N[C@@H](*)C]

In yet a further embodiment, the bond at the * position is

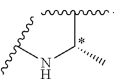 [SMILES: *N[C@H](*)C]

A further embodiment is a compound of Formula

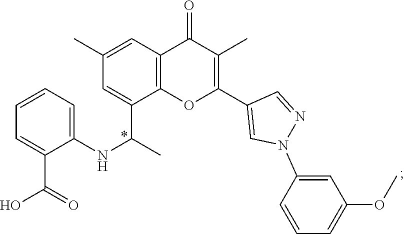 [SMILES: COc1cccc(-n2cc(-c3oc4c(C(C)Nc5ccccc5C(=O)O)cc(C)cc4c(=O)c3C)cn2)c1]

or a pharmaceutically acceptable salt thereof. In yet a further embodiment, the bond at the * position is

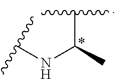 [SMILES: *N[C@@H](*)C]

In yet a further embodiment, the bond at the * position is

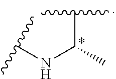 [SMILES: *N[C@H](*)C]

A further embodiment is a compound of Formula

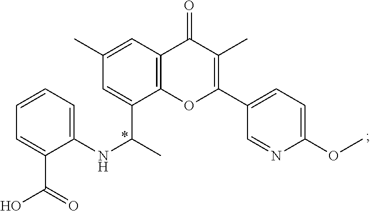 [SMILES: COc1ccc(-c2oc3c(C(C)Nc4ccccc4C(=O)O)cc(C)cc3c(=O)c2C)cn1]

or a pharmaceutically acceptable salt thereof. In yet a further embodiment, the bond at the * position is

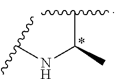 [SMILES: *N[C@@H](*)C]

In yet a further embodiment, the bond at the * position is

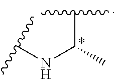 [SMILES: *N[C@H](*)C]

A further embodiment is a compound of Formula

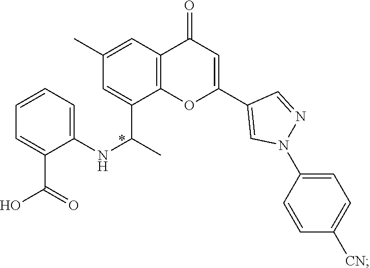 [SMILES: Cc1cc(C(C)Nc2ccccc2C(=O)O)c2oc(-c3cnn(-c4ccc(C#N)cc4)c3)cc(=O)c2c1]

or a pharmaceutically acceptable salt thereof. In yet a further embodiment, the bond at the * position is

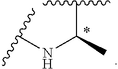 [SMILES: *N[C@@H](*)C]

In yet a further embodiment, the bond at the * position is

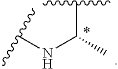 [SMILES: *N[C@H](*)C]

A further embodiment is a compound of Formula

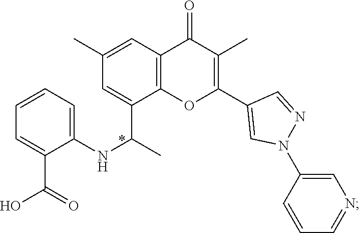 [SMILES: Cc1cc(C(C)Nc2ccccc2C(=O)O)c2oc(-c3cnn(-c4cccnc4)c3)c(C)c(=O)c2c1]

or a pharmaceutically acceptable salt thereof. In yet a further embodiment, the bond at the * position is

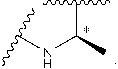 [SMILES: *N[C@@H](*)C]

In yet a further embodiment, the bond at the * position is

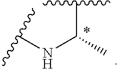 [SMILES: *N[C@H](*)C]

A further embodiment is a compound of Formula

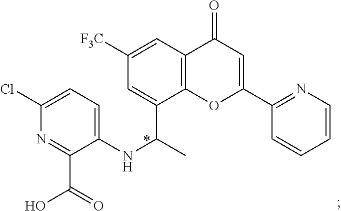 [SMILES: CC(Nc1ccc(Cl)nc1C(=O)O)c1cc(C(F)(F)F)cc2c(=O)cc(-c3ccccn3)oc12]

or a pharmaceutically acceptable salt thereof. In yet a further embodiment, the bond at the * position is

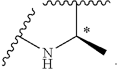 [SMILES: *N[C@@H](*)C]

In yet a further embodiment, the bond at the * position is

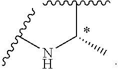 [SMILES: *N[C@H](*)C]

A further embodiment is a compound of Formula

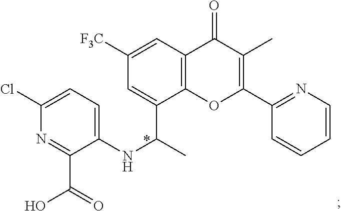 [SMILES: Cc1c(-c2ccccn2)oc2c(C(C)Nc3ccc(Cl)nc3C(=O)O)cc(C(F)(F)F)cc2c1=O]

or a pharmaceutically acceptable salt thereof. In yet a further embodiment, the bond at the * position is

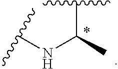 [SMILES: *N[C@@H](*)C]

In yet a further embodiment, the bond at the * position is

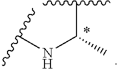 [SMILES: *N[C@H](*)C]

A further embodiment is a compound of Formula

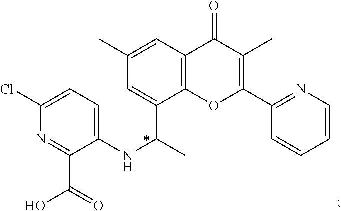 [SMILES: Cc1cc(C(C)Nc2ccc(Cl)nc2C(=O)O)c2oc(-c3ccccn3)c(C)c(=O)c2c1]

or a pharmaceutically acceptable salt thereof. In yet a further embodiment, the bond at the * position is

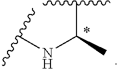 [SMILES: *N[C@@H](*)C]

In yet a further embodiment, the bond at the * position is

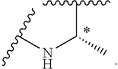 [SMILES: *N[C@H](*)C]

A further embodiment is a compound of Formula

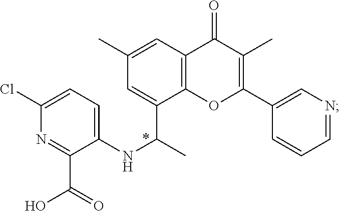 [SMILES: Cc1cc(C(C)Nc2ccc(Cl)nc2C(=O)O)c2oc(-c3cccnc3)c(C)c(=O)c2c1]

or a pharmaceutically acceptable salt thereof. In yet a further embodiment, the bond at the * position is

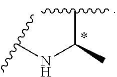 [SMILES: *N[C@@H](*)C]

In yet a further embodiment, the bond at the * position is

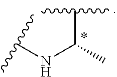 [SMILES: *N[C@H](*)C]

A further embodiment is a compound of Formula

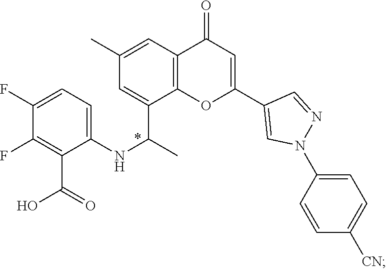 [SMILES: Cc1cc(C(C)Nc2ccc(F)c(F)c2C(=O)O)c2oc(-c3cnn(-c4ccc(C#N)cc4)c3)cc(=O)c2c1]

or a pharmaceutically acceptable salt thereof. In yet a further embodiment, the bond at the * position is

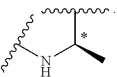 [SMILES: *N[C@@H](*)C]

In yet a further embodiment, the bond at the * position is

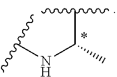 [SMILES: *N[C@H](*)C]

A further embodiment is a compound of Formula

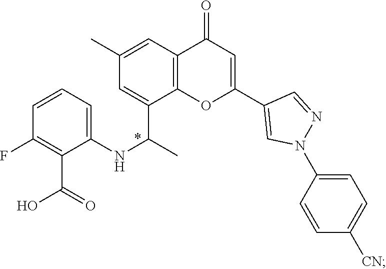 [SMILES: Cc1cc(C(C)Nc2cccc(F)c2C(=O)O)c2oc(-c3cnn(-c4ccc(C#N)cc4)c3)cc(=O)c2c1]

or a pharmaceutically acceptable salt thereof. In yet a further embodiment, the bond at the * position is

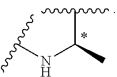 [SMILES: *N[C@@H](*)C]

In yet a further embodiment, the bond at the * position is

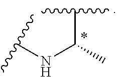 [SMILES: *N[C@H](*)C]

A further embodiment is a compound of Formula

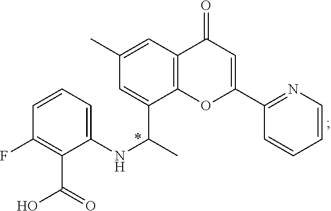 [SMILES: Cc1cc(C(C)Nc2cccc(F)c2C(=O)O)c2oc(-c3ccccn3)cc(=O)c2c1]

or a pharmaceutically acceptable salt thereof. In yet a further embodiment, the bond at the * position is

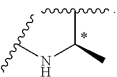 [SMILES: *N[C@@H](*)C]

In yet a further embodiment, the bond at the * position is

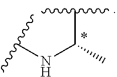 [SMILES: *N[C@H](*)C]

A further embodiment is a compound of Formula

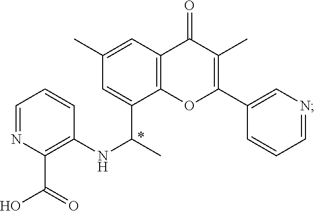 [SMILES: Cc1cc(C(C)Nc2cccnc2C(=O)O)c2oc(-c3cccnc3)c(C)c(=O)c2c1]

or a pharmaceutically acceptable salt thereof. In yet a further embodiment, the bond at the * position is

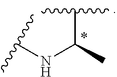 [SMILES: *N[C@@H](*)C]

In yet a further embodiment, the bond at the * position is

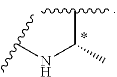 [SMILES: *N[C@H](*)C]

A further embodiment is a compound of Formula

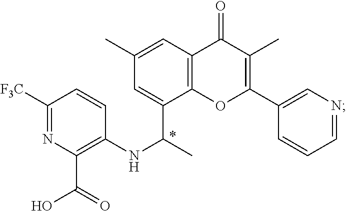 [SMILES: Cc1cc(C(C)Nc2ccc(C(F)(F)F)nc2C(=O)O)c2oc(-c3cccnc3)c(C)c(=O)c2c1]

or a pharmaceutically acceptable salt thereof. In yet a further embodiment, the bond at the * position is

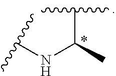 [SMILES: *N[C@@H](*)C]

In yet a further embodiment, the bond at the * position is

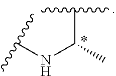 [SMILES: *N[C@H](*)C]

A further embodiment is a compound of Formula

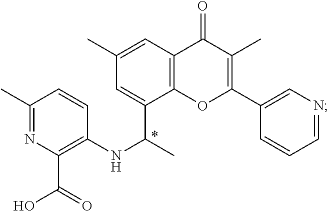 [SMILES: Cc1cc(C(C)Nc2ccc(C)nc2C(=O)O)c2oc(-c3cccnc3)c(C)c(=O)c2c1]

or a pharmaceutically acceptable salt thereof. In yet a further embodiment, the bond at the * position is

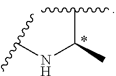 [SMILES: *N[C@@H](*)C]

In yet a further embodiment, the bond at the * position is

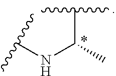 [SMILES: *N[C@H](*)C]

A further embodiment is a compound of Formula

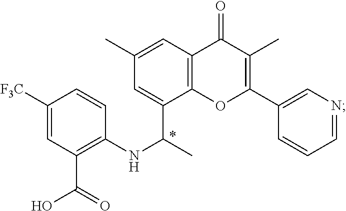 [SMILES: Cc1cc(C(C)Nc2ccc(C(F)(F)F)cc2C(=O)O)c2oc(-c3cccnc3)c(C)c(=O)c2c1]

or a pharmaceutically acceptable salt thereof. In yet a further embodiment, the bond at the * position is

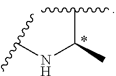 [SMILES: *N[C@@H](*)C]

In yet a further embodiment, the bond at the * position is

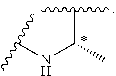 [SMILES: *N[C@H](*)C]

A further embodiment is a compound of Formula

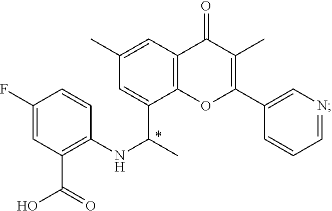 [SMILES: Cc1cc(C(C)Nc2ccc(F)cc2C(=O)O)c2oc(-c3cccnc3)c(C)c(=O)c2c1]

or a pharmaceutically acceptable salt thereof. In yet a further embodiment, the bond at the * position is

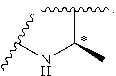 [SMILES: *N[C@@H](*)C]

In yet a further embodiment, the bond at the * position is

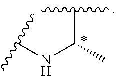 [SMILES: *N[C@H](*)C]

A further embodiment is a compound of Formula

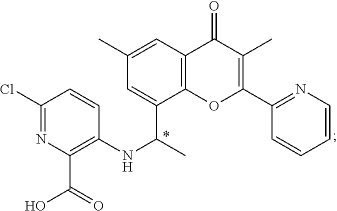 [SMILES: Cc1cc(C(C)Nc2ccc(Cl)nc2C(=O)O)c2oc(-c3ccccn3)c(C)c(=O)c2c1]

or a pharmaceutically acceptable salt thereof. In yet a further embodiment, the bond at the * position is

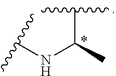 [SMILES: *N[C@@H](*)C]

In yet a further embodiment, the bond at the * position is

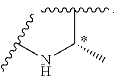 [SMILES: *N[C@H](*)C]

A further embodiment is a compound of Formula

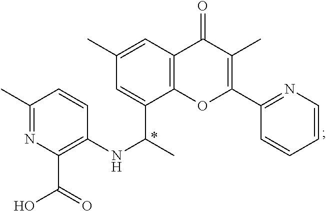 [SMILES: Cc1cc(C(C)Nc2ccc(C)nc2C(=O)O)c2oc(-c3ccccn3)c(C)c(=O)c2c1]

or a pharmaceutically acceptable salt thereof. In yet a further embodiment, the bond at the * position is

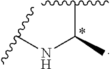 [SMILES: *N[C@@H](*)C]

In yet a further embodiment, the bond at the * position is

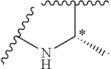 [SMILES: *N[C@H](*)C]

A further embodiment is a compound of Formula

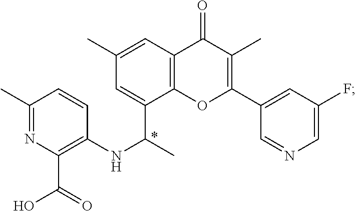 [SMILES: Cc1cc(C(C)Nc2ccc(C)nc2C(=O)O)c2oc(-c3cncc(F)c3)c(C)c(=O)c2c1]

or a pharmaceutically acceptable salt thereof. In yet a further embodiment, the bond at the * position is

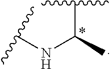 [SMILES: *N[C@@H](*)C]

In yet a further embodiment, the bond at the * position is

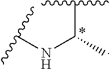 [SMILES: *N[C@H](*)C]

A further embodiment is a compound of Formula

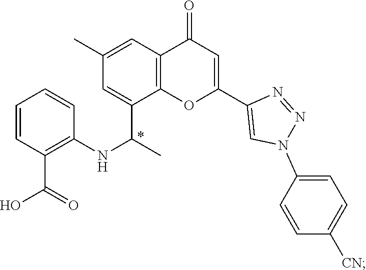 [SMILES: Cc1cc(C(C)Nc2ccccc2C(=O)O)c2oc(-c3cn(-c4ccc(C#N)cc4)nn3)cc(=O)c2c1]

or a pharmaceutically acceptable salt thereof. In yet a further embodiment, the bond at the * position is

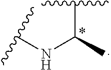 [SMILES: *N[C@@H](*)C]

In yet a further embodiment, the bond at the * position is

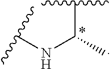 [SMILES: *N[C@H](*)C]

A further embodiment is a compound of Formula

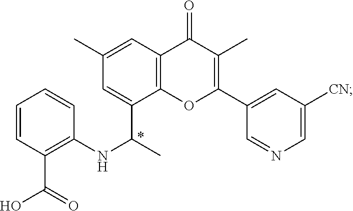 [SMILES: Cc1cc(C(C)Nc2ccccc2C(=O)O)c2oc(-c3cncc(C#N)c3)c(C)c(=O)c2c1]

or a pharmaceutically acceptable salt thereof. In yet a further embodiment, the bond at the * position is

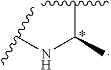 [SMILES: *N[C@@H](*)C]

In yet a further embodiment, the bond at the * position is

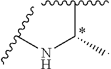 [SMILES: *N[C@H](*)C]

A further embodiment is a compound of Formula

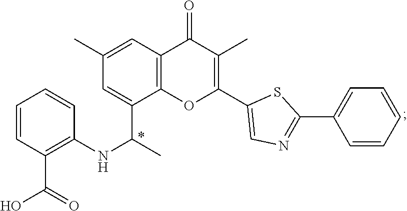 [SMILES: Cc1cc(C(C)Nc2ccccc2C(=O)O)c2oc(-c3cnc(-c4ccccc4)s3)c(C)c(=O)c2c1]

or a pharmaceutically acceptable salt thereof. In yet a further embodiment, the bond at the * position is

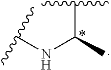 [SMILES: *N[C@@H](*)C]

In yet a further embodiment, the bond at the * position is

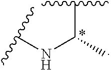 [SMILES: *N[C@H](*)C]

A further embodiment is a compound of Formula

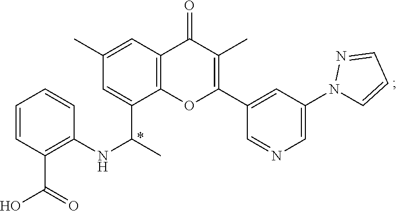 [SMILES: Cc1cc(C(C)Nc2ccccc2C(=O)O)c2oc(-c3cncc(-n4cccn4)c3)c(C)c(=O)c2c1]

or a pharmaceutically acceptable salt thereof. In yet a further embodiment, the bond at the * position is

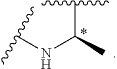 [SMILES: *N[C@@H](*)C]

In yet a further embodiment, the bond at the * position is

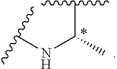 [SMILES: *N[C@H](*)C]

A further embodiment is a compound of Formula

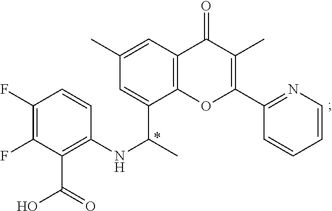 [SMILES: Cc1cc(C(C)Nc2ccc(F)c(F)c2C(=O)O)c2oc(-c3ccccn3)c(C)c(=O)c2c1]

or a pharmaceutically acceptable salt thereof. In yet a further embodiment, the bond at the * position is

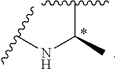 [SMILES: *N[C@@H](*)C]

In yet a further embodiment, the bond at the * position is

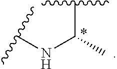 [SMILES: *N[C@H](*)C]

A further embodiment is a compound of Formula

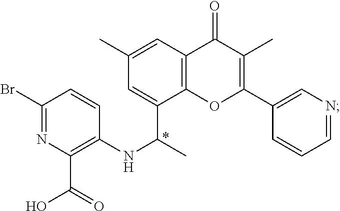 [SMILES: Cc1cc(C(C)Nc2ccc(Br)nc2C(=O)O)c2oc(-c3cccnc3)c(C)c(=O)c2c1]

or a pharmaceutically acceptable salt thereof. In yet a further embodiment, the bond at the * position is

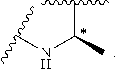 [SMILES: *N[C@@H](*)C]

In yet a further embodiment, the bond at the * position is

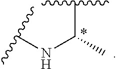 [SMILES: *N[C@H](*)C]

A further embodiment is a compound of Formula

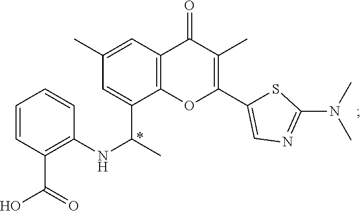 [SMILES: Cc1cc(C(C)Nc2ccccc2C(=O)O)c2oc(-c3cnc(N(C)C)s3)c(C)c(=O)c2c1]

or a pharmaceutically acceptable salt thereof. In yet a further embodiment, the bond at the * position is

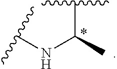 [SMILES: *N[C@@H](*)C]

In yet a further embodiment, the bond at the * position is

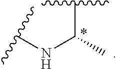 [SMILES: *N[C@H](*)C]

A further embodiment is a compound of Formula

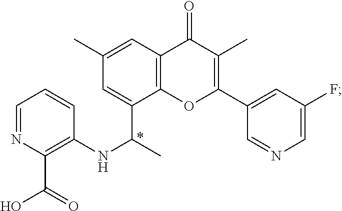 [SMILES: Cc1cc(C(C)Nc2cccnc2C(=O)O)c2oc(-c3cncc(F)c3)c(C)c(=O)c2c1]

or a pharmaceutically acceptable salt thereof. In yet a further embodiment, the bond at the * position is

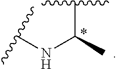 [SMILES: *N[C@@H](*)C]

In yet a further embodiment, the bond at the * position is

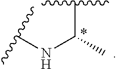 [SMILES: *N[C@H](*)C]

A further embodiment is a compound of Formula

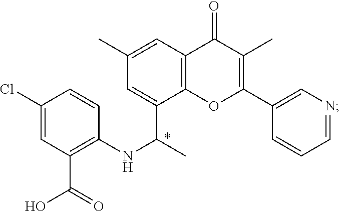 [SMILES: Cc1cc(C(C)Nc2ccc(Cl)cc2C(=O)O)c2oc(-c3cccnc3)c(C)c(=O)c2c1]

or a pharmaceutically acceptable salt thereof. In yet a further embodiment, the bond at the * position is

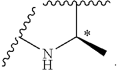 [SMILES: *N[C@@H](*)C]

In yet a further embodiment, the bond at the * position is

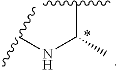 [SMILES: *N[C@H](*)C]

A further embodiment is a compound of Formula

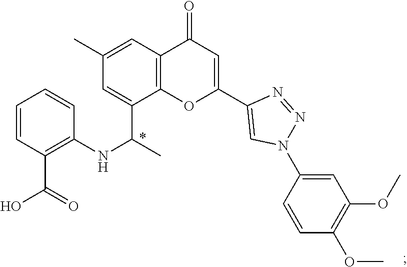 [SMILES: COc1ccc(-n2cc(-c3cc(=O)c4cc(C)cc(C(C)Nc5ccccc5C(=O)O)c4o3)nn2)cc1OC]

or a pharmaceutically acceptable salt thereof. In yet a further embodiment, the bond at the * position is

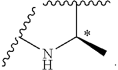 [SMILES: *N[C@@H](*)C]

In yet a further embodiment, the bond at the * position is

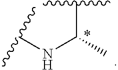 [SMILES: *N[C@H](*)C]

A further embodiment is a compound of Formula

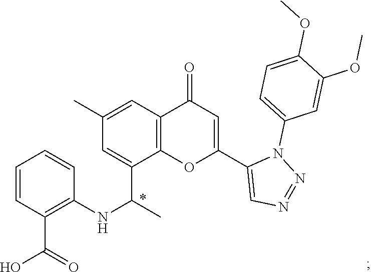 [SMILES: COc1ccc(-n2nncc2-c2cc(=O)c3cc(C)cc(C(C)Nc4ccccc4C(=O)O)c3o2)cc1OC]

or a pharmaceutically acceptable salt thereof. In yet a further embodiment, the bond at the * position is

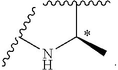 [SMILES: *N[C@@H](*)C]

In yet a further embodiment, the bond at the * position is

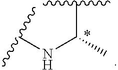 [SMILES: *N[C@H](*)C]

A further embodiment is a compound of Formula

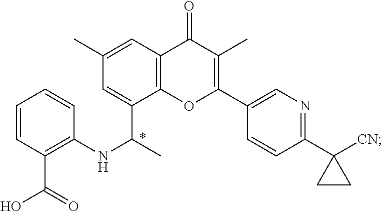 [SMILES: Cc1cc(C(C)Nc2ccccc2C(=O)O)c2oc(-c3ccc(C4(C#N)CC4)nc3)c(C)c(=O)c2c1]

or a pharmaceutically acceptable salt thereof. In yet a further embodiment, the bond at the * position is

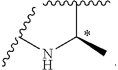 [SMILES: *N[C@@H](*)C]

In yet a further embodiment, the bond at the * position is

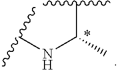 [SMILES: *N[C@H](*)C]

A further embodiment is a compound of Formula

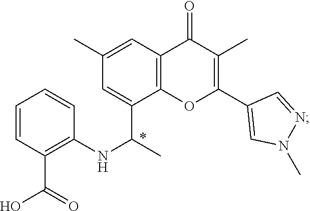 [SMILES: Cc1cc(C(C)Nc2ccccc2C(=O)O)c2oc(-c3cnn(C)c3)c(C)c(=O)c2c1]

or a pharmaceutically acceptable salt thereof. In yet a further embodiment, the bond at the * position is

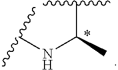 [SMILES: *N[C@@H](*)C]

In yet a further embodiment, the bond at the * position is

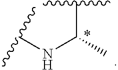 [SMILES: *N[C@H](*)C]

A further embodiment is a compound of Formula

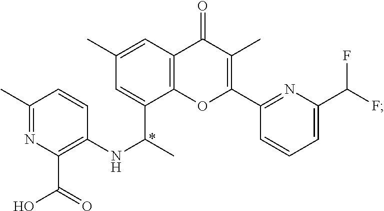 [SMILES: Cc1cc(C(C)Nc2ccc(C)nc2C(=O)O)c2oc(-c3cccc(C(F)F)n3)c(C)c(=O)c2c1]

or a pharmaceutically acceptable salt thereof. In yet a further embodiment, the bond at the * position is

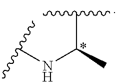 [SMILES: *N[C@@H](*)C]

In yet a further embodiment, the bond at the * position is

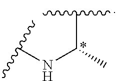 [SMILES: *N[C@H](*)C]

A further embodiment is a compound of Formula

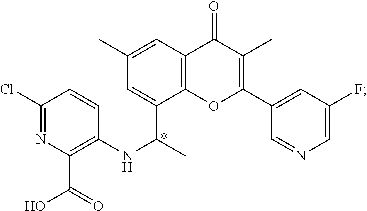 [SMILES: Cc1cc(C(C)Nc2ccc(Cl)nc2C(=O)O)c2oc(-c3cncc(F)c3)c(C)c(=O)c2c1]

or a pharmaceutically acceptable salt thereof. In yet a further embodiment, the bond at the * position is

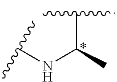 [SMILES: *N[C@@H](*)C]

In yet a further embodiment, the bond at the * position is

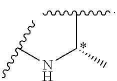 [SMILES: *N[C@H](*)C]

A further embodiment is a compound of Formula

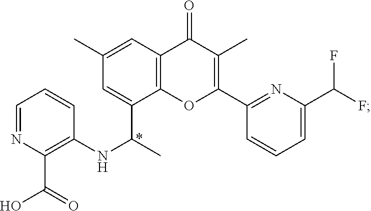 [SMILES: Cc1cc(C(C)Nc2cccnc2C(=O)O)c2oc(-c3cccc(C(F)F)n3)c(C)c(=O)c2c1]

or a pharmaceutically acceptable salt thereof. In yet a further embodiment, the bond at the * position is

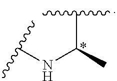 [SMILES: *N[C@@H](*)C]

In yet a further embodiment, the bond at the * position is

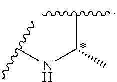 [SMILES: *N[C@H](*)C]

A further embodiment is a compound of Formula

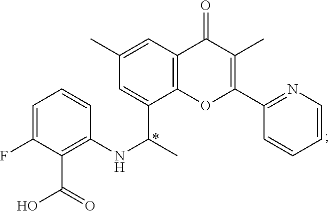 [SMILES: Cc1cc(C(C)Nc2cccc(F)c2C(=O)O)c2oc(-c3ccccn3)c(C)c(=O)c2c1]

or a pharmaceutically acceptable salt thereof. In yet a further embodiment, the bond at the * position is

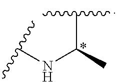 [SMILES: *N[C@@H](*)C]

In yet a further embodiment, the bond at the * position is

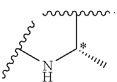 [SMILES: *N[C@H](*)C]

A further embodiment is a compound of Formula

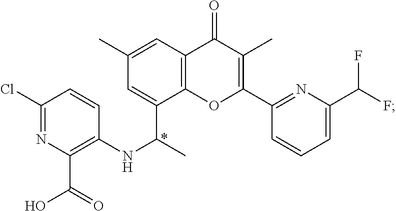 [SMILES: Cc1cc(C(C)Nc2ccc(Cl)nc2C(=O)O)c2oc(-c3cccc(C(F)F)n3)c(C)c(=O)c2c1]

or a pharmaceutically acceptable salt thereof. In yet a further embodiment, the bond at the * position is

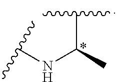 [SMILES: *N[C@@H](*)C]

In yet a further embodiment, the bond at the * position is

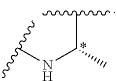 [SMILES: *N[C@H](*)C]

A pharmaceutically acceptable salt of a compound of the present invention is, for example, an acid-addition salt of a compound of the invention, which is sufficiently basic, for example, an acid-addition salt with, for example, an inorganic or organic acid, for example hydrochloric, hydrobromic, sulfuric, phosphoric, trifluoroacetic, formic, citric, methane sulfonate or maleic acid. In addition, a pharmaceutically acceptable salt of a compound of the present invention which is sufficiently acidic is an alkali metal salt, for example a sodium or potassium salt, an alkaline earth metal salt, for example a calcium or magnesium salt, an ammonium salt or a salt with an organic base which affords a pharmaceutically acceptable cation, for example a salt with methylamine, dimethylamine, diethylamine, trimethylamine, piperidine, morpholine or tris-(2-hydroxyethyl)amine. Pharmaceutically acceptable salts, and common methodology for preparing them are well known in the art (see, e.g., P. Stahl, et al. Handbook of Pharmaceutical Salts: Properties, Selection, and Use, 2^{nd} Revised Edition (Wiley-VCH, 2011); S. M. Berge, et al., “Pharmaceutical Salts,” Journal of Pharmaceutical Sciences, Vol. 66, No. 1, January 1977).

Further representative “pharmaceutically acceptable salts” include, e.g., water-soluble, and water-insoluble salts, such as the acetate, amsonate (4,4-diaminostilbene-2,2-disulfonate), benzenesulfonate, benzoate, bicarbonate, bisulfate, bitartrate, borate, bromide, butyrate, calcium, calcium edetate, camsylate, carbonate, chloride, citrate, clavulanate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate, pantothenate, phosphate/diphosphate, picrate, polygalacturonate, propionate, p-toluenesulfonate, salicylate, stearate, subacetate, succinate, sulfate, sulfosalicylate, tannate, tartrate, teoclate, tosylate, triethiodide, and valerate salts.

The compounds of the present invention can be prepared in a number of ways well known to those skilled in the art of organic synthesis. By way of example, compounds of the present invention can be synthesized using the methods described below, together with synthetic methods known in the art of synthetic organic chemistry, or variations thereon as appreciated by those skilled in the art. Preferred methods include but are not limited to those methods described below. Compounds of the present invention can be synthesized by following the steps outlined in General Schemes 1, 2 and 3. Starting materials are either commercially available or made by known procedures in the reported literature or as illustrated below.

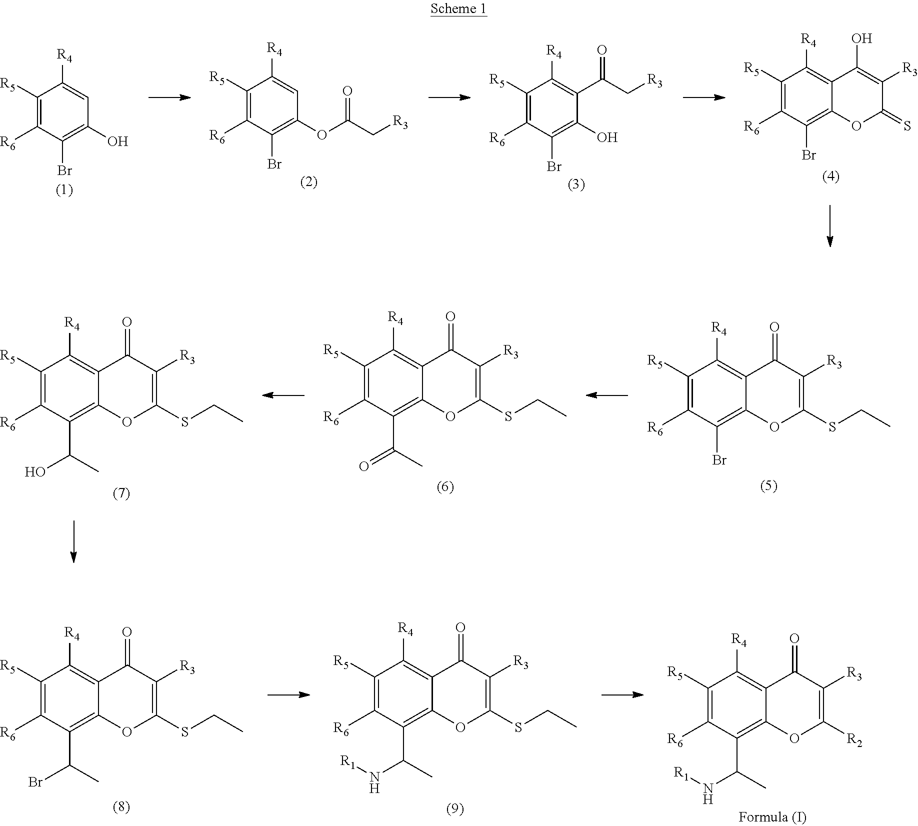

Scheme 1 depicts the preparation of compounds of Formula (I), where R is —H, R₇ is methyl, and R₈ is —H. Acylation of substituted phenol (1) may provide ester (2). Ester (2) may undergo rearrangement under Lewis acid (e.g., AlCl₃) or Brønsted acid (e.g., triflic acid) conditions to provide hydroxy aryl ketone (3). Basic deprotonation of ketone (3) in the presence of carbon disulfide gives the bicyclic chromene-2-thione (4).

Alkylation of thione (4) under basic conditions affords thiolether (5). Phenyl bromide (5) can be acylated via palladium catalysis to produce acyl chromen-4-one (6). Aryl ketone (6) can be reduced to hydroxy compound (7) with a reagent such as sodium borohydride. Use of a halogenating agent such as phosphorus tribromide can be used to convert hydroxy compound (7) to the halo compound (8).

Halo compound (8) can be used to alkylate an arylamine or heteroarylamine to give arylamine or heteroarylamine (9). Thiolether (9) can be converted to compounds of Formula (I) using transition metal catalysis to couple heteroaryl boronic acids, boronic esters, or other coupling partners, followed by hydrolysis of ester present on R₁.

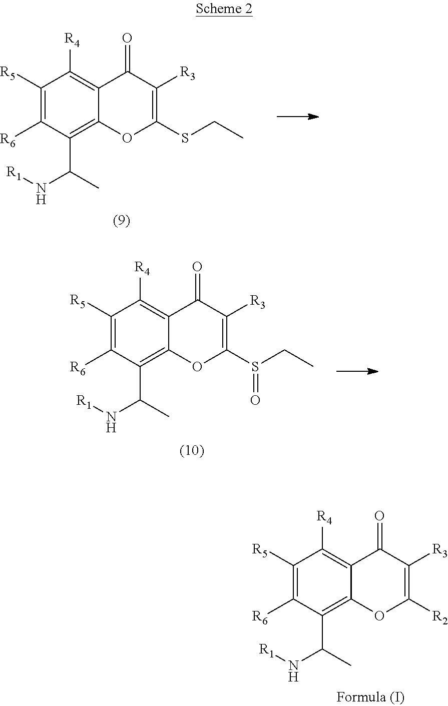

Scheme 2 depicts another preparation of compounds of Formula (I), where R is —H, R₇ is methyl, and R₈ is —H. Oxidation of thiolether (9) with an oxidant such as m-CPBA can give sulfoxide (10). Sulfoxide (10) can be converted to compounds of Formula (I) by substitution with various heteroaryl groups.

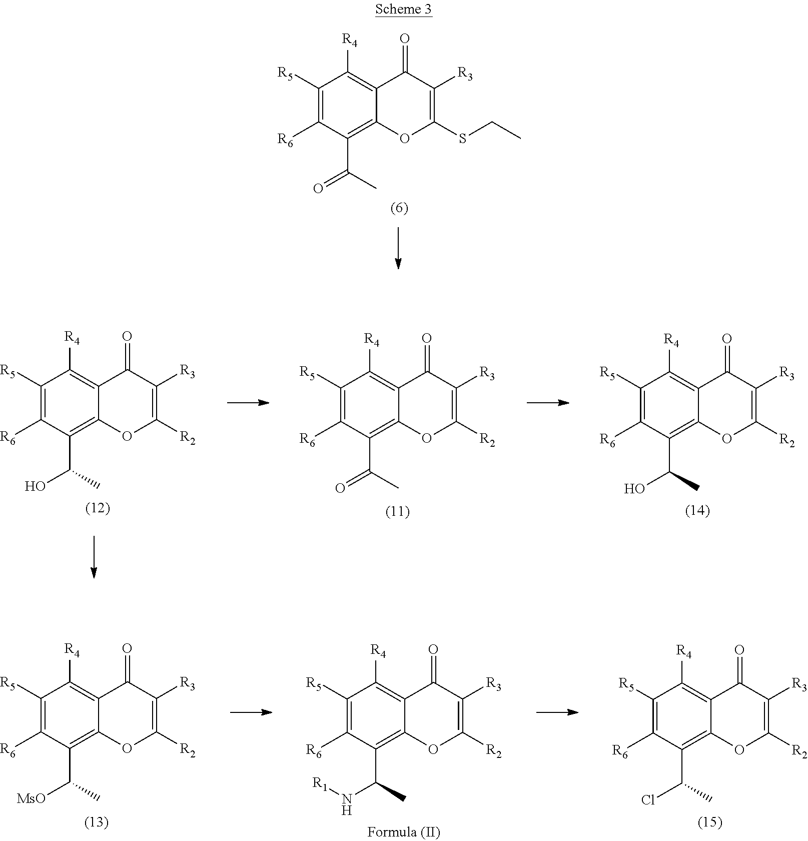

Scheme 3 depicts the preparation of compounds of Formula (II) where R is —H and R₇ is methyl. Thiolether (6) can be converted to 2-substituted chromenone (11) using transition metal catalysis to couple heteroaryl boronic acids, boronic esters, or other coupling partners. Ketone (11) can be reduced to hydroxy compound (12) with a chiral catalyst such as the Noyori catalyst. The hydroxyl compound (12) can be converted into a leaving group with methanesulfonic anhydride or methanesulfonyl chloride to give mesylate (13). Mesylate (13) can be used to alkylate an arylamine or heteroarylamine to give compounds of Formula (II) after hydrolysis of the ester present on R₁.

Alternatively, ketone (11) can be reduced to hydroxy compound (14) with a chiral catalyst such as the Noyori catalyst. The hydroxyl group can be converted to chloride (15) with a chlorinating agent such as 2,4,6-trichloro-1,3,5-triazine. Chloride (15) can then be used to alkylate an arylamine or heteroarylamine to give compounds of Formula (II) after hydrolysis of the ester present on R₁.

### Pharmaceutical Compositions

In some aspects, the present disclosure provides a pharmaceutical composition comprising a compound of Formula (I), (II), or (III) as an active ingredient. In some embodiments, the present disclosure provides a pharmaceutical composition comprising a compound of Formula (I), (II), or (III), or a pharmaceutically acceptable salt thereof, and one or more pharmaceutically acceptable carriers or excipients.

As used herein, the term “composition” is intended to encompass a product comprising the specified ingredients in the specified amounts, as well as any product which results, directly or indirectly, from combination of the specified ingredients in the specified amounts.

The compounds of Formula (I), (II), or (III) can be formulated for oral administration in forms such as tablets, capsules (each of which includes sustained release or timed release formulations), pills, powders, granules, elixirs, tinctures, suspensions, syrups and emulsions. The compounds of Formula (I), (II), or (III) can also be formulated for intravenous (bolus or in-fusion), intraperitoneal, topical, subcutaneous, intramuscular or transdermal (e.g., patch) administration, all using forms well known to those of ordinary skill in the pharmaceutical arts.

The formulation of the present disclosure may be in the form of an aqueous solution comprising an aqueous vehicle. The aqueous vehicle component may comprise water and at least one pharmaceutically acceptable excipient. Suitable acceptable excipients include those selected from the group consisting of a solubility enhancing agent, chelating agent, preservative, tonicity agent, viscosity/suspending agent, buffer, and pH modifying agent, and a mixture thereof.

According to a further aspect of the disclosure there is provided a pharmaceutical composition which comprises a compound any one of the Formulae disclosed herein, or a pharmaceutically acceptable salt, hydrate or solvate thereof, in association with a pharmaceutically acceptable diluent or carrier.

The compositions of the disclosure may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the disclosure may be obtained by conventional procedures using conventional pharmaceutical excipients, well known in the art. Thus, compositions intended for oral use may contain, for example, one or more coloring, sweetening, flavoring and/or preservative agents.

### Methods of Use

In some aspects, the present disclosure provides a method of modulating PI3K (e.g., PI3Kα) activity (e.g., in vitro or in vivo), comprising contacting a cell with a therapeutically effective amount of a compound of Formula (I), (II), or (III), or a pharmaceutically acceptable salt thereof.

In some aspects, the present disclosure provides a method of treating or preventing a disease or disorder disclosed herein in a subject in need thereof, comprising administering to the subject a therapeutically effective amount of a compound of Formula (I), (II), or (III), or a pharmaceutically acceptable salt thereof, or a pharmaceutical composition of the present disclosure.

In some aspects, the present disclosure provides a method of treating a disease or disorder disclosed herein in a subject in need thereof, comprising administering to the subject a therapeutically effective amount of a compound of Formula (I), (II), or (III), or a pharmaceutically acceptable salt thereof, or a pharmaceutical composition of the present disclosure.

In some embodiments, the disease or disorder is associated with an implicated PI3K activity. In some embodiments, the disease or disorder is a disease or disorder in which PI3K activity is implicated.

In some embodiments, the disease or disorder is a cancer.

In some embodiments, the cancer is selected from acute lymphoblastic leukemia (ALL), acute myeloid leukemia (AMIL), adrenocortical carcinoma, aids-related cancers, aids-related lymphoma, anal cancer, astrocytoma, basal cell carcinoma, bile duct cancer, bladder cancer, bone cancer, osteosarcoma, malignant fibrous histiocytoma, brain tumors, breast cancer, bronchial tumors, Burkitt lymphoma, carcinoid tumor, cancer of unknown primary, cardiac (heart) tumors, atypical teratoid/rhabdoid tumor, primary CNS lymphoma, cervical cancer, cholangiocarcinoma, chordoma, chronic lymphocytic leukemia (CLL), chronic myelogenous leukemia (CML), colorectal cancer, craniopharyngioma, cutaneous t-cell lymphoma, mycosis fungoides, Sezary syndrome, ductal carcinoma in situ (DCIS), embryonal tumors, medulloblastoma, endometrial cancer, ependymoma, esophageal cancer, esthesioneuroblastoma, Ewing sarcoma, extracranial germ cell tumor, extragonadal germ cell tumor, fallopian tube cancer, gallbladder cancer, gastric cancer, gastrointestinal carcinoid tumor, malignant gastrointestinal stromal tumors (GIST), germ cell tumors, gestational trophoblastic disease, hairy cell leukemia, head and neck cancer, hepatocellular cancer, Langerhans cell histiocytosis, Hodgkin lymphoma, islet cell tumors, pancreatic neuroendocrine tumors, Kaposi sarcoma, kidney cancer, laryngeal cancer, leukemia, liver cancer, lung cancer, lymphoma, male breast cancer, intraocular melanoma, Merkel cell carcinoma, malignant mesothelioma, metastatic cancer, metastatic squamous neck cancer, midline tract carcinoma with nut gene changes, mouth cancer, multiple endocrine neoplasia syndromes, multiple myeloma/plasma cell neoplasms, myelodysplastic syndromes, myelodysplastic neoplasms, myeloproliferative neoplasms, chronic myeloproliferative neoplasm, nasal cavity and paranasal sinus cancer, nasopharyngeal cancer, neuroblastoma, non-Hodgkin lymphoma, non-small cell lung cancer, oral cancer, lip and oral cavity cancer, oropharyngeal cancer, malignant fibrous histiocytoma of bone, ovarian cancer, pancreatic cancer, pancreatic neuroendocrine tumors (islet cell tumors), papillomatosis, paraganglioma, paranasal sinus and nasal cavity cancer, parathyroid cancer, penile cancer, pharyngeal cancer, pheochromocytoma, pituitary tumor, plasma cell neoplasm, multiple myeloma, pleuropulmonary blastoma, primary central nervous system (CNS) lymphoma, primary peritoneal cancer, prostate cancer, rectal cancer, recurrent cancer, renal cell (kidney) cancer, retinoblastoma, rhabdomyosarcoma, salivary gland cancer, sarcoma, childhood vascular tumors, skin cancer, small cell lung cancer, small intestine cancer, soft tissue sarcoma, squamous cell carcinoma of the skin, testicular cancer, oropharyngeal cancer, hypopharyngeal cancer, thymoma, thymic carcinoma, thyroid cancer, tracheobronchial tumors, transitional cell cancer of the renal pelvis and ureter, urethral cancer, uterine sarcoma, vaginal cancer, vascular tumors, vulvar cancer, and Wilms tumor.

In some embodiments, the cancer is Endometrial cancer, Breast cancer, Oesophageal squamous-cell cancer, Cervical squamous-cell carcinoma, Cervical adenocarcinoma, Colorectal adenocarcinoma, Bladder Urothelial Carcinoma, Glioblastoma, Ovarian cancer, Non-small-cell Lung cancer, Esophagogastric cancer, Nerve-sheath tumor, Head and neck squamous-cell carcinoma, Melanoma, Esophagogastric adenocarcinoma, Soft-tissue sarcoma, Prostate cancer, Fibrolamellar carcinoma, Hepatocellular carcinoma, Diffuse glioma, Colorectal cancer, Pancreatic cancer, Cholangiocarcinoma, B-cell lymphoma, Mesothelioma, Adrenocortical carcinoma, Renal non-clear-cell carcinoma, Renal clear-cell carcinoma, Germ-cell carcinoma, Thymic tumor, Pheochromocytoma, Miscellaneous neuroepithelial tumor, thyroid cancer, leukemia, or encapsulated glioma.

In some embodiments, the cancer is a breast cancer, a prostate cancer, or a brain cancer.

In some embodiments, the cancer is a breast cancer. In some embodiments, the cancer is a prostate cancer. In some embodiments, the cancer is a brain cancer.

In some embodiments, the breast cancer is metastatic breast cancer. In some embodiments, the breast cancer is ductal carcinoma in situ (DCIS). In some embodiments, the breast cancer is invasive ductal carcinoma. In some embodiments, the breast cancer is triple negative breast cancer. In some embodiments, the breast cancer is medullary carcinoma. In some embodiments, the breast cancer is tubular carcinoma. In some embodiments, the breast cancer is mucinous carcinoma. In some embodiments, the breast cancer is Paget disease of the breast or nipple. In some embodiments, the breast cancer is inflammatory breast cancer (IBC). In some embodiments, the breast cancer is hormone receptor-positive (HR+), human epidermal growth factor receptor 2-negative (HER2−) advanced or metastatic breast cancer.

In some embodiments, the prostate cancer is an adenocarcinoma. In some embodiments, the prostate cancer is a small cell carcinoma. In some embodiments, the prostate cancer is a neuroendocrine tumor. In some embodiments, the prostate cancer is a transitional cell carcinoma. In some embodiments, the prostate cancer is a sarcoma.

In some embodiments, the brain cancer is an acoustic neuroma. In some embodiments, the brain cancer is an astrocytoma. In some embodiments, the brain cancer is a brain metastasis. In some embodiments, the brain cancer is choroid plexus carcinoma. In some embodiments, the brain cancer is craniopharyngioma. In some embodiments, the brain cancer is an embryonal tumor. In some embodiments, the brain cancer is an ependymoma. In some embodiments, the brain cancer is a glioblastoma. In some embodiments, the brain cancer is a glioma. In some embodiments, the brain cancer is a medulloblastoma. In some embodiments, the brain cancer is a meningioma. In some embodiments, the brain cancer is an oligodendroglioma. In some embodiments, the brain cancer is a pediatric brain tumor. In some embodiments, the brain cancer is a pineoblastoma. In some embodiments, the brain cancer is a pituitary tumor.

In some embodiments, the disease or disorder associated with PI3K includes, but is not limited to, CLOVES syndrome (congenial lipomatous overgrowth, vascular malformations, epidermal naevi, scoliosis/skeletal and spinal syndrome), PIK3CA-related overgrowth syndrome (PROS), breast cancer, brain cancer, prostate cancer, endometrial cancer, gastric cancer, leukemia, lymphoma, sarcoma, colorectal cancer, lung cancer, ovarian cancer, skin cancer, or head and neck cancer.

In some embodiments, the diseases or disorder associated with PI3K is CLOVES syndrome (congenital lipomatous overgrowth, vascular malformations, epidermal naevi, scoliosis/skeletal and spinal syndrome).

In some embodiments, the disease or disorder associated with PI3K is PIK3CA-related overgrowth syndrome (PROS).

In some embodiments, the disease or disorder associated with PI3K is breast cancer, brain cancer, prostate cancer, endometrial cancer, gastric cancer, leukemia, lymphoma, sarcoma, colorectal cancer, lung cancer, ovarian cancer, skin cancer, or head and neck cancer.

In some embodiments, the disease or disorder associated with PI3K is a breast neoplasm, a thyroid neoplasm, an ovarian neoplasm, non-small-cell lung carcinoma, an endometrial neoplasm, or a pancreatic neoplasm. In some embodiments, the disease or disorder associated with PI3K is a breast neoplasm. In some embodiments, the disease or disorder associated with PI3K is a thyroid neoplasm. In some embodiments, the disease or disorder associated with PI3K is an ovarian neoplasm. In some embodiments, the disease or disorder associated with PI3K is non-small-cell lung carcinoma. In some embodiments, the disease or disorder associated with PI3K is an endometrial neoplasm. In some embodiments, the disease or disorder associated with PI3K is a pancreatic neoplasm.

In some embodiments, the disease or disorder associated with PI3K is breast cancer, brain cancer, prostate cancer, endometrial cancer, gastric cancer, colorectal cancer, lung cancer, ovarian cancer, skin cancer, or head and neck cancer.

In some embodiments, the disease or disorder associated with PI3K is leukemia, lymphoma, or sarcoma.

In some embodiments, the cancer is endometrial cancer, head and neck cancer, or a sarcoma.

In some embodiments, the cancer is endometrial cancer. In some embodiments the cancer is head and neck cancer. In some embodiments, the cancer is a sarcoma.

In some embodiments, the sarcoma is soft tissue sarcoma, osteosarcoma, chondrosarcoma, Ewing sarcoma, hemangioendothelioma, angiosarcoma, fibrosarcoma, myofibrosarcoma, chordoma, adamantinoma, liposarcoma, leiomyosarcoma, malignant peripheral nerve sheath tumor, rhabdomyosarcoma, synovial sarcoma, or malignant solitary fibrous tumor.

In some embodiments, the sarcoma is soft tissue sarcoma. In some embodiments the soft tissue sarcoma is liposarcoma, atypical lipomatous tumor, dermatofibrosarcoma protuberans, malignant solitary fibrous tumor, inflammatory myofibroblastic tumor, low-grade myofibroblastic sarcoma, fibrosarcoma, myxofibrosarcoma, low-grade fibromyxoid sarcoma, giant cell tumor of soft tissues, leiomyosarcoma, malignant glomus tumor, rhabdomyosarcoma, hemangioendothelioma, angiosarcoma of soft tissue, extraskeletal osteosarcoma, gastrointestinal stromal tumor, malignant gastrointestinal stromal tumor (GIST), malignant peripheral nerve sheath tumor, malignant Triton tumor, malignant granular cell tumor, malignant ossifying fibromyxoid tumor, stromal sarcoma, myoepithelial carcinoma, malignant phosphaturic mesenchymal tumor, synovial sarcoma, epithelioid sarcoma, alveolar soft part sarcoma, clear cell sarcoma of soft tissue, extraskeletal myxoid chondrosarcoma, extraskeletal Ewing sarcoma, desmoplastic small round cell tumor, extrarenal rhabdoid tumor, perivascular epithelioid cell tumor, intimal sarcoma, undifferentiated spindle cell sarcoma, undifferentiated pleomorphic sarcoma, undifferentiated round cell sarcoma, undifferentiated epithelioid sarcoma, or undifferentiated sarcoma, not otherwise specified.

In some aspects, the present disclosure provides a method of treating or preventing a cancer in a subject in need thereof, comprising administering to the subject a therapeutically effective amount of a compound of Formula (I), (II), or (III), or a pharmaceutically acceptable salt thereof, or a pharmaceutical composition of the present disclosure.

In some aspects, the present disclosure provides a method of treating a cancer in a subject in need thereof, comprising administering to the subject a therapeutically effective amount of a compound of Formula (I), (II), or (III), or a pharmaceutically acceptable salt thereof, or a pharmaceutical composition of the present disclosure.

In some aspects, the present disclosure provides a method of treating or preventing a breast cancer in a subject in need thereof, comprising administering to the subject a therapeutically effective amount of a compound of Formula (I), (II), or (III), or a pharmaceutically acceptable salt thereof, or a pharmaceutical composition of the present disclosure.

In some aspects, the present disclosure provides a method of treating a breast cancer in a subject in need thereof, comprising administering to the subject a therapeutically effective amount of a compound of Formula (I), (II), or (III), or a pharmaceutically acceptable salt thereof, or a pharmaceutical composition of the present disclosure.

In some aspects, the present disclosure provides a method of treating or preventing a prostate cancer in a subject in need thereof, comprising administering to the subject a therapeutically effective amount of a compound of Formula (I), (II), or (III), or a pharmaceutically acceptable salt thereof, or a pharmaceutical composition of the present disclosure.

In some aspects, the present disclosure provides a method of treating a prostate cancer in a subject in need thereof, comprising administering to the subject a therapeutically effective amount of a compound of Formula (I), (II), or (III), or a pharmaceutically acceptable salt thereof, or a pharmaceutical composition of the present disclosure.

In some aspects, the present disclosure provides a method of treating or preventing a brain cancer in a subject in need thereof, comprising administering to the subject a therapeutically effective amount of a compound of Formula (I), (II), or (III), or a pharmaceutically acceptable salt thereof, or a pharmaceutical composition of the present disclosure.

In some aspects, the present disclosure provides a method of treating a brain cancer in a subject in need thereof, comprising administering to the subject a therapeutically effective amount of a compound of Formula (I), (II), or (III), or a pharmaceutically acceptable salt thereof, or a pharmaceutical composition of the present disclosure.

In some aspects, the present disclosure provides a compound of Formula (I), (II), or (III), or a pharmaceutically acceptable salt thereof for use in therapy.

In some aspects, the present disclosure provides a compound of Formula (I), (II), or (III), or a pharmaceutically acceptable salt thereof for use in modulating PI3K (e.g., PI3Kα) activity (e.g., in vitro or in vivo).

In some aspects, the present disclosure provides a compound of Formula (I), (II), or (III), or a pharmaceutically acceptable salt thereof for use in treating or preventing a disease or disorder disclosed herein.

In some aspects, the present disclosure provides a compound of Formula (I), (II), or (III), or a pharmaceutically acceptable salt thereof for use in treating a disease or disorder disclosed herein.

In some aspects, the present disclosure provides a compound of Formula (I), (II), or (III), or a pharmaceutically acceptable salt thereof for use in treating or preventing a cancer.

In some aspects, the present disclosure provides a compound of Formula (I), (II), or (III), or a pharmaceutically acceptable salt thereof for use in treating a cancer.

In some aspects, the present disclosure provides a compound of Formula (I), (II), or (III), or a pharmaceutically acceptable salt thereof for use in treating or preventing a breast cancer.

In some aspects, the present disclosure provides a compound of Formula (I), (II), or (III), or a pharmaceutically acceptable salt thereof for use in treating a breast cancer.

In some aspects, the present disclosure provides a compound of Formula (I), (II), or (III), or a pharmaceutically acceptable salt thereof for use in treating or preventing a prostate cancer.

In some aspects, the present disclosure provides a compound of Formula (I), (II), or (III), or a pharmaceutically acceptable salt thereof for use in treating a prostate cancer.

In some aspects, the present disclosure provides a compound of Formula (I), (II), or (III), or a pharmaceutically acceptable salt thereof for use in treating or preventing a brain cancer.

In some aspects, the present disclosure provides a compound of Formula (I), (II), or (III), or a pharmaceutically acceptable salt thereof for use in treating a brain cancer.

In some aspects, the present disclosure provides use of a compound of Formula (I), (II), or (III), or a pharmaceutically acceptable salt thereof in the manufacture of a medicament for modulating PI3K (e.g., PI3Kα) activity (e.g., in vitro or in vivo).

In some aspects, the present disclosure provides use of a compound of Formula (I), (II), or (III), or a pharmaceutically acceptable salt thereof in the manufacture of a medicament for treating or preventing a disease or disorder disclosed herein.

In some aspects, the present disclosure provides use of a compound of Formula (I), (II), or (III), or a pharmaceutically acceptable salt thereof in the manufacture of a medicament for treating a disease or disorder disclosed herein.

In some aspects, the present disclosure provides use of a compound of Formula (I), (II), or (III), or a pharmaceutically acceptable salt thereof in the manufacture of a medicament for treating or preventing a cancer in a subject in need thereof.

In some aspects, the present disclosure provides use of a compound of Formula (I), (II), or (III), or a pharmaceutically acceptable salt thereof in the manufacture of a medicament for treating a cancer in a subject in need thereof.

In some aspects, the present disclosure provides use of a compound of Formula (I), (II), or (III), or a pharmaceutically acceptable salt thereof in the manufacture of a medicament for treating or preventing a breast cancer in a subject in need thereof.

In some aspects, the present disclosure provides use of a compound of Formula (I), (II), or (III), or a pharmaceutically acceptable salt thereof in the manufacture of a medicament for treating a breast cancer in a subject in need thereof.

In some aspects, the present disclosure provides use of a compound of Formula (I), (II), or (III), or a pharmaceutically acceptable salt thereof in the manufacture of a medicament for treating or preventing a prostate cancer in a subject in need thereof.

In some aspects, the present disclosure provides use of a compound of Formula (I), (II), or (III), or a pharmaceutically acceptable salt thereof in the manufacture of a medicament for treating a prostate cancer in a subject in need thereof.

In some aspects, the present disclosure provides use of a compound of Formula (I), (II), or (III), or a pharmaceutically acceptable salt thereof in the manufacture of a medicament for treating or preventing a brain cancer in a subject in need thereof.

In some aspects, the present disclosure provides use of a compound of Formula (I), (II), or (III), or a pharmaceutically acceptable salt thereof in the manufacture of a medicament for treating a brain cancer in a subject in need thereof.

The present disclosure provides compounds that function as modulators of PI3K activity. The present disclosure therefore provides a method of modulating PI3K activity in vitro or in vivo, said method comprising contacting a cell with a therapeutically effective amount of a compound, or a pharmaceutically acceptable salt thereof, as defined herein.

In some embodiments, PI3K modulation is inhibition of PI3K.

In some embodiments, the PI3K inhibitor is a PI3Kα inhibitor. In some embodiments, the PI3K inhibitor is a PI3Kα H1047R mutant inhibitor.

Effectiveness of compounds of the disclosure can be determined by industry-accepted assays/disease models according to standard practices of elucidating the same as described in the art and are found in the current general knowledge.

The present disclosure also provides a method of treating a disease or disorder in which PI3K activity is implicated in a patient in need of such treatment, said method comprising administering to said patient a therapeutically effective amount of a compound, or a pharmaceutically acceptable salt thereof, or a pharmaceutical composition as defined herein.

### Routes of Administration

The compounds of Formula (I), (II), or (III), or pharmaceutical compositions comprising these compounds may be administered to a subject by any convenient route of administration, whether systemically/peripherally or topically (i.e., at the site of desired action).

Routes of administration include, but are not limited to, oral (e.g. by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### EXAMPLES

Exemplary compounds of Formula (I), (II), and (III) are synthesized and tested in the examples. It is understood that compounds of Formula (I), (II), and (III) may be converted to the corresponding pharmaceutically acceptable salts of the compounds using routine techniques in the art (e.g., by saponification of an ester to the carboxylic acid salt, or by hydrolyzing an amide to form a corresponding carboxylic acid and then converting the carboxylic acid to a carboxylic acid salt).

Nuclear magnetic resonance (NMR) spectra were recorded at 400 MHz or 300 MHz as stated and at 300.3 K unless otherwise stated; the chemical shifts (δ) are reported in parts per million (ppm). Spectra were recorded using a Bruker or Varian instrument with 8, 16 or 32 scans.

LC-MS chromatograms and spectra were recorded using an Agilent 1200 or Shimadzu LC-20 AD&MS 2020 instrument using a C-18 column such as a Luna-C18 2.0×30 mm or Xbridge Shield RPC18 2.1×50 mm. Injection volumes were 0.7-8.0 μl and the flow rates were typically 0.8 or 1.2 ml/min. Detection methods were diode array (DAD) or evaporative light scattering (ELSD) as well as positive ion electrospray ionization. MS range was 100-1000 Da. Solvents were gradients of water and acetonitrile both containing a modifier (typically 0.01-0.04%) such as trifluoroacetic acid or ammonium carbonate.

### Abbreviations

AcOH/HOAc Acetic Acid
ADP Adenacohosine diphosphate
ATP Adenosine triphosphate
CDCl₃ Chloroform-d
DCM Dichloromethane
DIEA N,N-diisopropylethylamine
DMF N,N-dimethylformamide
DMSO dimethylsulfoxide
DMSO-d₆ Hexadeuterodimethylsulfoxide
eq. equivalents
EtI Ethyl iodide
EtOAc ethyl acetate
h hour(s)
HEPES 4-(2-hydroxyethyl)-1-piperazineethanesulfonic acid
¹H NMR Proton nuclear magnetic resonance spectroscopy
LC-MS Liquid chromatography-mass spectrometry
MeOH Methanol
min minute(s)
NaHMDS Sodium bis(trimethylsilyl)amide
PIP2 Phosphatidylinositol 4,5-bisphosphate
PPh₃ triphenylphosphine
ppm parts per million
rt room temperature
TFA trifluoroacetic acid
THF Tetrahydrofuran
Ti(i-PrO)₄ Titanium(IV) isopropoxide

### Intermediate 1: (2-Bromo-4-methyl-phenyl) acetate

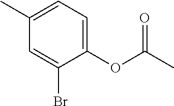

A DCM (2.4 L) mixture of 2-bromo-4-methyl-phenol (300 g, 1.6 mol) and pyridine (152 g, 1.92 mol) at 0° C. was treated with acetyl chloride and stirred at 25° C. for 16 h. The mixture was diluted with water (1500 mL), the pH adjusted to 5 with HCl (2 M aqueous), and extracted with DCM (3×500 mL). The combined organic extracts were washed with brine (2×250 mL), dried over Na₂SO₄, filtered, and concentrated to give the product as an oil (400 g, crude). ¹H NMR (400 MHz, CDCl₃) δ ppm 2.24 (s, 3H), 2.25 (s, 3H), 6.91 (d, J=8.4 Hz, 2H), 7.01-7.02 (m, 2H), 7.33 (s, 1H).

### Intermediate 2: 1-(3-Bromo-2-hydroxy-5-methyl-phenyl)ethanone

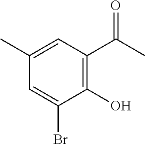

A mixture of (2-bromo-4-methyl-phenyl) acetate (50 g, 218 mmol) and AlCl₃ (102 g, 764 mmol) was degassed and purged with N₂ three times and stirred at 140° C. for 1 h. After cooling to rt, the reaction was diluted with DCM (30 mL) and dropped into 150 mL of water at 0° C. The mixture was filtered and the aqueous phase extracted with DCM (2×150 mL). The combined organic extracts were washed with brine, dried over anhydrous Na₂SO₄, filtered, and concentrated. The residue was triturated with petroleum ether (2×150 mL) to give the product as a solid (30 g, 52%). ¹H NMR (400 MHz, CDCl₃) δ ppm 2.30 (s, 3H), 2.68 (s, 3H), 7.73 (s, 1H), 7.33 (s, 1H), 12.64 (s, 1H).

### Intermediate 3: 8-Bromo-4-hydroxy-6-methyl-chromene-2-thione

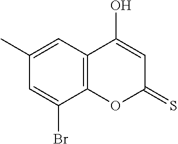

A solution of 1-(3-bromo-2-hydroxy-5-methyl-phenyl)ethanone (65 g, 284 mmol) in THE (800 mL) was treated with NaHMDS (851 mL, 1 M) at −50° C. over 30 min, allowed to warm to between −5° C. and 0° C., and stirred for 1 h. The reaction was cooled to −20° C. and treated with CS₂ (64.8 g, 851 mmol) dropwise over 1 h, allowed to warm to 25° C., and stirred for another 16 h. The reaction was quenched with H₂SO₄ (800 mL, 15%) at −50° C. over 1 h, allowed to warm to rt, and extracted with EtOAc (2×1 L). The combined organic extracts were washed with brine (1 L), dried over anhydrous Na₂SO₄, filtered, and concentrated. The residue was triturated with EtOAc (0.5 L) to give the product as a solid (210 g crude, 64%, purity ˜76%).

### Intermediate 4: 8-Bromo-2-ethylsulfanyl-6-methyl-chromen-4-one

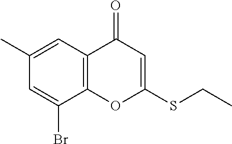 [SMILES: CCSc1cc(=O)c2cc(C)cc(Br)c2o1]

A mixture of 8-bromo-4-hydroxy-6-methyl-chromene-2-thione (20.0 g, 73.8 mmol), EtI (46 g, 295 mmol), and K₂CO₃ (12.2 g, 88.5 mmol) in acetone (200 mL) was stirred at 60° C. for 3 h. When the reaction had cooled to rt, the mixture was diluted with water (200 mL) and extracted with DCM (2×200 mL). The combined organic extracts were concentrated and purified via silica gel chromatography eluted with 20%-40% EtOAc in petroleum ether to give the product as a gum. ¹H NMR (400 MHz, CDCl₃) δ ppm 1.51 (t, J=7.2 Hz, 3H), 2.45 (s, 3H), 3.22 (q, J=7.2 Hz, 2H), 6.32 (s, 1H), 7.70 (s, 1H), 7.93 (s, 1H).

### Intermediate 5: 8-Acetyl-2-ethylsulfanyl-6-methyl-chromen-4-one

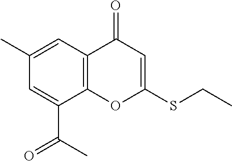 [SMILES: CCSc1cc(=O)c2cc(C)cc(C(C)=O)c2o1]

A mixture of 8-bromo-2-ethylsulfanyl-6-methyl-chromen-4-one (9.00 g, 30.0 mmol), tributyl(1-ethoxyvinyl)tin (13.3 g, 36.8 mmol) and Pd(PPh₃)₂Cl₂ (2.11 g, 3.01 mmol) in dioxane (90 mL) was stirred at 95° C. for 16 h. HCl (30 mL, 1 M) was added to the mixture and stirred at 50° C. for 0.5 h. When cooled to rt, the mixture was treated with saturated aqueous KF (100 mL) and stirred for 0.5 h, then filtered. The filter cake was washed with EtOAc (3×40 mL). The filtrate was extracted with EtOAc (2×80 mL). The combined organic extracts were concentrated and purified on a silica gel column eluted with 0-60% EtOAc in petroleum ether to give the product as a solid (5.8 g, 60%). MS ES+ m/z 263 [M+H]⁺.

### Intermediate 6: 2-Ethylsulfanyl-8-(1-hydroxyethyl)-6-methyl-chromen-4-one

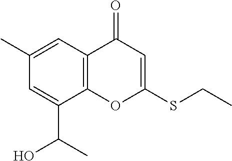 [SMILES: CCSc1cc(=O)c2cc(C)cc(C(C)O)c2o1]

A solution of 8-acetyl-2-ethylsulfanyl-6-methyl-chromen-4-one (8.30 g, 31.6 mmol) in DCM (30 mL) and MeOH (30 mL) was treated with NaBH₄ (1.32 g, 34.8 mmol) in portions at 0° C., and stirred at 15° C. for 1 h. The mixture was diluted with water (50 mL) and extracted with DCM (2×100 mL). The combined organic extracts were washed with brine (80 mL), dried over anhydrous Na₂SO₄, filtered, and concentrated. The residue was purified on a silica gel column eluted with 0-4% MeOH in DCM to give the product as a solid (6.0 g, 60%). MS ES+ m/z 265 [M+H]⁺.

### Intermediate 7: 8-(1-Bromoethyl)-2-ethylsulfanyl-6-methyl-chromen-4-one

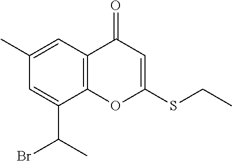 [SMILES: CCSc1cc(=O)c2cc(C)cc(C(C)Br)c2o1]

A mixture of 2-ethylsulfanyl-8-(1-hydroxyethyl)-6-methyl-chromen-4-one (5.50 g, 20.8 mmol) in DCM (50 mL) was treated dropwise with PBr₃ (16.9 g, 62.4 mmol) at 0° C., then stirred at 30° C. for 4 h. The reaction was quenched with water (20 mL) at 0° C. and the pH adjusted to 8 with saturated aqueous NaHCO₃. The mixture was extracted with DCM (2×80 mL). The combined organic extracts were washed with brine, dried over anhydrous Na₂SO₄, filtered, and concentrated to give the product as an oil (4.7 g, 61%). MS ES+ m/z 329 [M+2+H]⁺.

### Intermediate 8: tert-Butyl 2-[1-(2-ethylsulfanyl-6-methyl-4-oxo-chromen-8-yl)ethylamino]benzoate

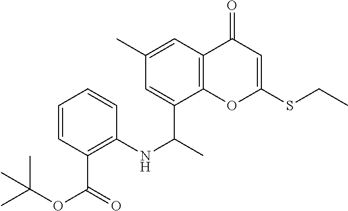 [SMILES: CCSc1cc(=O)c2cc(C)cc(C(C)Nc3ccccc3C(=O)OC(C)(C)C)c2o1]

8-(1-Bromoethyl)-2-ethylsulfanyl-6-methyl-chromen-4-one (25.0 g, 76.4 mmol), tert-butyl 2-aminobenzoate (29.5 g, 153 mmol) and DIEA (14.8 g, 20.0 mL, 115 mmol) were combined with DMF (150 mL) in a 500 mL round bottom flask and heated at 80° C. After cooling to rt, the reaction was partially concentrated to ˜100 mL, poured into 1.1 L of water, and extracted with EtOAc (2×350 mL). The combined organic layers were washed with brine (400 mL). The combined aqueous layers were re-extracted with fresh EtOAc. The organic layers were combined, dried over anhydrous Na₂SO₄, filtered, and concentrated to give a thick oil. Purified the residue via silica gel chromatography using EtOAc in DCM (0% to 10%) to provide an off-white foam. Triturated with heptanes/DCM and washed with heptanes to give the product as a white solid (27.1 g, 81%). MS ES+ m/z 440 [M+H]⁺.

### Intermediate 9: tert-Butyl 2-[1-[2-(6-isopropoxy-3-pyridyl)-6-methyl-4-oxo-chromen-8-yl]ethylamino]benzoate

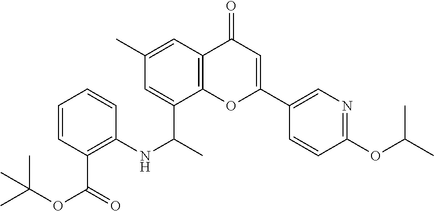 [SMILES: Cc1cc(C(C)Nc2ccccc2C(=O)OC(C)(C)C)c2oc(-c3ccc(OC(C)C)nc3)cc(=O)c2c1]

tert-Butyl 2-[1-(2-ethylsulfanyl-6-methyl-4-oxo-chromen-8-yl)ethylamino]benzoate (0.100 g, 227 μmol), (6-isopropoxypyridin-3-yl)boronic acid (82.4 mg, 455 μmol), zinc(II) acetate (83.5 mg, 455 μmol), tris(dibenzylideneacetone)dipalladium(0) (20.8 mg, 22.7 μmol), copper(I) thiophene-2-carboxylate (86.8 mg, 455 μmol), and tri(2-furyl)phosphine (26.4 mg, 114 μmol) were combined in THE (2 mL) and degassed for 5 min using argon. The reaction mixture was stirred at 75° C. overnight. Added silica gel to the reaction and concentrated. Purified via silica gel chromatography using a gradient of EtOAc (0-100%) in heptane to obtain the product (0.151 g). MS ES+ m/z 515 [M+H]⁺.

### Intermediate 10: tert-Butyl 2-[1-[2-(2-methoxypyrimidin-5-yl)-6-methyl-4-oxo-chromen-8-yl]ethylamino]benzoate

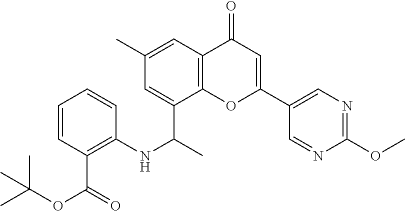 [SMILES: COc1ncc(-c2cc(=O)c3cc(C)cc(C(C)Nc4ccccc4C(=O)OC(C)(C)C)c3o2)cn1]

tert-Butyl 2-[1-(2-ethylsulfanyl-6-methyl-4-oxo-chromen-8-yl)ethylamino]benzoate (200 mg, 455 μmol), (2-methoxypyrimidin-5-yl)boronic acid (140 mg, 910 μmol), tris(dibenzylideneacetone)dipalladium(0) (42 mg, 46 μmol), cesium carbonate (445 mg, 1.365 mmol), tri(2-furyl)phosphine (10.6 mg, 46 μmol) were combined in 1,4-dioxane (5 mL) and heated at 85° C. for 12 h. The reaction mixture was filtered, concentrated, and purified via silica gel chromatography using a gradient of 10-80% ethyl acetate in heptane to give the product (90 mg, 41%). MS ES+ m/z 488 [M+H]⁺.

### Intermediate 11: Methyl 2-[1-(2-ethylsulfanyl-6-methyl-4-oxo-chromen-8-yl)ethylamino]benzoate

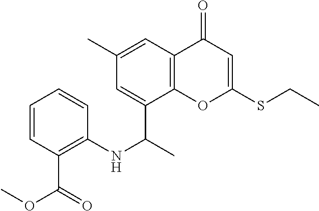 [SMILES: CCSc1cc(=O)c2cc(C)cc(C(C)Nc3ccccc3C(=O)OC)c2o1]

A mixture of 8-(1-bromoethyl)-2-ethylsulfanyl-6-methyl-chromen-4-one (4.00 g, 12.2 mmol) and methyl 2-aminobenzoate (3.70 g, 24.5 mmol) in DMF (30 mL) was stirred at 80° C. for 8 h. When cooled to rt, the mixture was diluted with water (100 mL) and extracted with EtOAc (3×80 mL). The combined organic extracts were washed with brine (3×100 mL), dried over anhydrous Na₂SO₄, filtered, and concentrated. The residue was purified by silica gel chromatography eluted with 0%-27% EtOAc in petroleum ether to give the product (4.5 g, 84%) as a solid. MS ES+ m/z 398 [M+H]⁺.

### Intermediate 12 and Intermediate 13: Methyl 2-[1-(2-ethylsulfanyl-6-methyl-4-oxo-chromen-8-yl)ethylamino]benzoate, Isomer 1 and Isomer 2

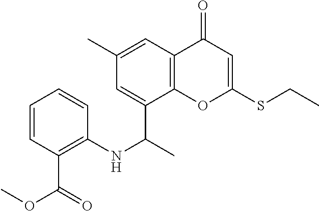 [SMILES: CCSc1cc(=O)c2cc(C)cc(C(C)Nc3ccccc3C(=O)OC)c2o1]

Methyl 2-[1-(2-ethylsulfanyl-6-methyl-4-oxo-chromen-8-yl)ethylamino]benzoate (13 g, 32.71 mmol) was separated via supercritical fluid chromatography (DAICEL CHIRALPAK AS, 250 mm×50 mm, 10 μm; 30% EtOH w/0.1% NH₄OH: 70% CO₂) to obtain the product isomers (4.3 g, 5.6 g) as white solids. MS ES+ m/z 398 [M+H]⁺, for both.

### Intermediate 14: Methyl 2-[1-(6-methyl-4-oxo-2-pyrimidin-2-yl-chromen-8-yl)ethylamino]benzoate, Isomer 2

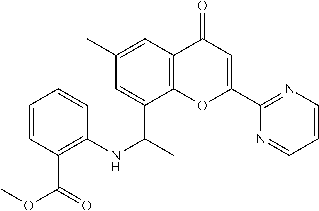 [SMILES: COC(=O)c1ccccc1NC(C)c1cc(C)cc2c(=O)cc(-c3ncccn3)oc12]

A mixture of methyl 2-[1-(2-ethylsulfanyl-6-methyl-4-oxo-chromen-8-yl)ethylamino]benzoate, Isomer 2 (300 mg, 754.74 umol), tributyl(pyrimidin-2-yl)stannane (613 mg, 1.66 mmol), Pd(PPh₃)₄ (87 mg, 75.47 umol), and CuBr (238 mg, 1.66 mmol) in THE (6 mL) was stirred at 70° C. under N₂ for 10 h to give a brown suspension. Cooled to rt, concentrated the reaction mixture, and purified via flash silica gel chromatography using a gradient of EtOAc (0-40%) in petroleum ether to give the product (150 mg, 48%) as a light yellow solid. MS ES+ m/z 416 [M+H]⁺.

### Intermediate 15 and Intermediate 16: tert-Butyl 2-[1-(2-ethylsulfanyl-6-methyl-4-oxo-chromen-8-yl)ethylamino]benzoate, Isomer 1 and Isomer 2

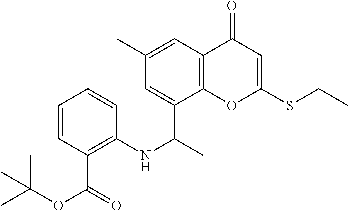 [SMILES: CCSc1cc(=O)c2cc(C)cc(C(C)Nc3ccccc3C(=O)OC(C)(C)C)c2o1]

tert-Butyl 2-[1-(2-ethylsulfanyl-6-methyl-4-oxo-chromen-8-yl)ethylamino]benzoate (22.04 g, 50.14 mmol) was separated into component isomers using a Chiralcel OJ column (8×34 cm; 20 micron) eluted with 100% MeOH with 0.2% DMEA to give isomer 1 (wet 11.3 g) and isomer 2 (wet 12.9 g). MS ES+ m/z 440 [M+H]⁺.

### Intermediate 17 and Intermediate 18: 2-[1-(2-Ethylsulfanyl-6-methyl-4-oxo-chromen-8-yl)ethylamino]benzoic acid, Isomer 1 and Isomer 2

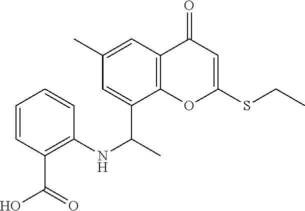 [SMILES: CCSc1cc(=O)c2cc(C)cc(C(C)Nc3ccccc3C(=O)O)c2o1]

A mixture of 8-(1-bromoethyl)-2-ethylsulfanyl-6-methyl-chromen-4-one (10 g, 31 mmol) and 2-aminobenzoic acid (8.38 g, 61.1 mmol) in DMF (70 mL) was stirred at 80° C. for 2 h. The reaction mixture was diluted with DCM (200 mL) and water (500 mL) and the pH was adjusted to ˜11 with aq. NaOH (2 M). After removal of the organic layer, the aqueous layer was washed with MTBE (200 mL×2) and the pH was adjusted to ˜3 with aq. HCl (2 M) to give a solid. After stirring for 0.5 h, the mixture was filtered and the filter cake was purified by chiral SFC (Daicel ChiralCel OJ-H; 250×30 mm; 5 μm) using a gradient of 5-50% MeOH with 0.1% aq NH3 in CO₂ to give isomer 1 (5.4 g; 45%, >99% ee) and isomer 2 (4.9 g, 41%, >99% ee). MS ES+ m/z 384 [M+H]⁺ for both.

### Intermediate 19: 2-[1-(2-Ethylsulfinyl-6-methyl-4-oxo-chromen-8-yl)ethylamino]benzoic acid, Isomer 2

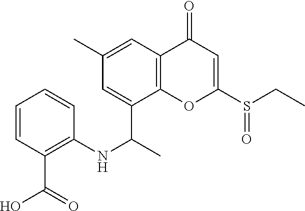 [SMILES: CCS(=O)c1cc(=O)c2cc(C)cc(C(C)Nc3ccccc3C(=O)O)c2o1]

A mixture of 2-[1-(2-ethylsulfanyl-6-methyl-4-oxo-chromen-8-yl)ethylamino]benzoic acid, Isomer 2 (850 mg, 2.22 mmol) in DCM (10 mL) was treated with m-CPBA (585 mg, 2.88 mmol, 85% purity) under N₂ at 0° C. The reaction was stirred at 25° C. for 2 h. The mixture was quenched with sat. Na₂S₂O₃ (10 mL) at 0° C. and the aqueous layer was extracted with EtOAc (2×20 mL). The combined organic layers were washed with brine (3×20 mL), dried over anhydrous Na₂SO₄, filtered, and concentrated. The residue was purified via silica gel chromatography, eluting with 0-80% EtOAc in petroleum ether to give the product (410 mg, 42%) as a solid. MS ES+ m/z 400 [M+H]⁺.

### Intermediate 20: tert-Butyl 2-[1-[6-methyl-4-oxo-2-(1H-pyrazol-4-yl)chromen-8-yl]ethylamino]benzoate, Isomer 2

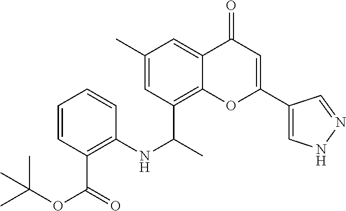 [SMILES: Cc1cc(C(C)Nc2ccccc2C(=O)OC(C)(C)C)c2oc(-c3cn[nH]c3)cc(=O)c2c1]

Combined tert-butyl 2-[1-(2-ethylsulfanyl-6-methyl-4-oxo-chromen-8-yl)ethylamino]benzoate, Isomer 2 (0.25 g, 0.57 mmol), (1H-pyrazol-4-yl)boronic acid (0.19 g, 1.71 mmol), tris(dibenzylideneacetone)dipalladium(0) (0.052 g, 0.057 mmol), copper(I) thiophene-2-carboxylate (0.22 g, 1.14 mmol), zinc(II) acetate (0.21 g, 1.14 mmol), and tri(2-furyl)phosphine (0.066 g, 0.28 mmol) in 2-methyltetrahydrofuran (12 mL) and heated at 95° C. for 24 h. Added (1H-pyrazol-4-yl)boronic acid (0.19 g, 1.71 mmol) and tris(dibenzylideneacetone)dipalladium(0) (0.052 g, 0.057 mmol) and heated at 95° C. for 24 h. The crude product mixture was quenched with water and concentrated. The residue was purified using silica column (0-15% ethyl acetate in heptane, then 5% methanol in DCM), then reversed phase chromatography (10-100% acetonitrile in water, with 0.1% TFA) to afford the product (0.17 g, 57%). MS ES+ m/z 446 [M+H]⁺.

### Intermediate 21: tert-Butyl 2-[1-[2-[1-(4-chlorophenyl)pyrazol-4-yl]-6-methyl-4-oxo-chromen-8-yl]ethylamino]benzoate, Isomer 2

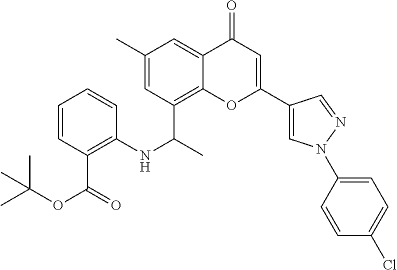 [SMILES: Cc1cc(C(C)Nc2ccccc2C(=O)OC(C)(C)C)c2oc(-c3cnn(-c4ccc(Cl)cc4)c3)cc(=O)c2c1]

Combined tert-butyl 2-[1-[6-methyl-4-oxo-2-(1H-pyrazol-4-yl)chromen-8-yl]ethylamino]benzoate, Isomer 2 (0.030 g, 0.067 mmol), 1-chloro-4-iodobenzene (0.032 g, 0.13 mmol), potassium carbonate (0.020 g, 0.14 mmol), copper(I) iodide (0.26 mg, 0.02 eq), and (1S,2S)—N1,N2-dimethylcyclohexane-1,2-diamine (1.9 mg, 0.2 eq) in 1,4-dioxane (2 mL) and heated at 110° C. for 12 h. Added 1-chloro-4-iodobenzene (0.032 g, 0.13 mmol), potassium carbonate (0.020 g, 0.14 mmol), copper(I) iodide (0.26 mg, 0.02 eq), and (1S,2S)—N1,N2-dimethylcyclohexane-1,2-diamine (1.9 mg, 0.2 eq) and heated at 110° C. for 12 h. The crude product mixture was quenched with water and concentrated. Purified using reversed phase chromatography (10-100% acetonitrile in water, with 0.1% TFA) to afford the product (0.016 g, 38%). MS ES+ m/z 556 [M+H]⁺.

### Intermediate 22: tert-Butyl 2-[1-(2-ethylsulfanyl-3-iodo-6-methyl-4-oxo-chromen-8-yl)ethylamino]benzoate, Isomer 2

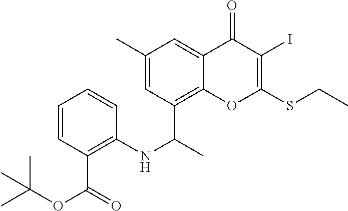 [SMILES: CCSc1oc2c(C(C)Nc3ccccc3C(=O)OC(C)(C)C)cc(C)cc2c(=O)c1I]

A dry flask equipped with a stir bar and septum was flushed with argon gas and then charged with tert-butyl 2-[1-(2-ethylsulfanyl-6-methyl-4-oxo-chromen-8-yl)ethylamino]benzoate, Isomer 2 (1.00 g, 2.27 mmol) and 2 mL of dry THF. The reaction was cooled in an ice bath. When cold, 2,2,6,6-tetramethylpiperidinylzinc chloride lithium chloride complex (1M in THF, 1.93 g, 6.82 mmol) was added dropwise via addition funnel over 30 min. After addition was complete, allowed the reaction to stir at 0° C. After 1 hr, iodine dissolved in dry THE (1M, 2.73 mL, 2.73 mmol) was added dropwise via addition funnel. After addition was complete, the reaction was stirred at 0° C. After 1 hr, the reaction was cooled to −40° C. and quenched with methanol (10 mL). Added 50 mL of an ammonium chloride/ammonia solution (aqueous 2M solution; 50 mL) and stirred the reaction at rt for 2 hr. Extracted three times with 300 mL of dichloromethane. The organics were combined, washed with aqueous sodium carbonate, collected, dried over Na₂SO₄, filtered, and concentrated. The residue was purified by reverse phase chromatography (C18) eluted with 0% to 80% acetonitrile (with 0.1% TFA) in water (with 0.1% TFA) to give the product (0.90 g, 66%). MS ES+ m/z 566 [M+H]⁺.

### Intermediate 23: tert-Butyl 2-[1-[2-ethylsulfanyl-6-methyl-4-oxo-3-(trifluoromethyl)chromen-8-yl]ethylamino]benzoate, Isomer 2

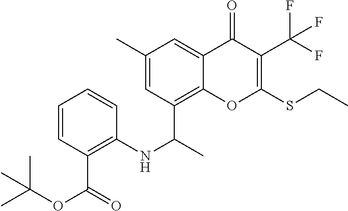 [SMILES: CCSc1oc2c(C(C)Nc3ccccc3C(=O)OC(C)(C)C)cc(C)cc2c(=O)c1C(F)(F)F]

Combined tert-butyl 2-[1-(2-ethylsulfanyl-3-iodo-6-methyl-4-oxo-chromen-8-yl)ethylamino]benzoate, Isomer 2 (0.31 g, 0.56 mmol), copper(I) iodide (0.13 g, 0.67 mmol), and methyl difluoro(fluorosulfonyl)acetate (0.53 g, 2.78 mmol) in DMF (4 mL) and stirred at 75° C. for 6 h. The reaction mixture was cooled to rt diluted with ethyl acetate (30 mL). The organics were washed with water (3×15 mL) and concentrated. The residue was purified by silica column (0-100% ethyl acetate in heptane) to give the product (0.20 g, 71%). MS ES+ m/z 508 [M+H]⁺.

### Intermediate 24: 8-Acetyl-2-[6-(difluoromethyl)-2-pyridyl]-3,6-dimethyl-chromen-4-one

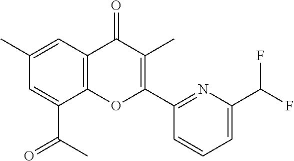 [SMILES: CC(=O)c1cc(C)cc2c(=O)c(C)c(-c3cccc(C(F)F)n3)oc12]

Intermediate 24 can be made according to the foregoing Intermediates. MS ES+ m/z 344 [M+H]⁺.

### Intermediate 25: 2-[6-(Difluoromethyl)-2-pyridyl]-8-[(1R)-1-hydroxyethyl]-3,6-dimethyl-chromen-4-one

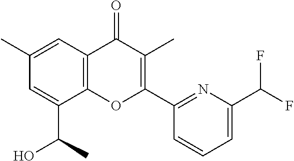 [SMILES: Cc1cc([C@@H](C)O)c2oc(-c3cccc(C(F)F)n3)c(C)c(=O)c2c1]

A solution of 8-acetyl-2-[6-(difluoromethyl)-2-pyridyl]-3,6-dimethyl-chromen-4-one (0.50 g, 1.45 mmol), formic acid (0.21 g, 4.37 mmol), and RuCl (p-cymene)[(R,R)-Ts-DPEN] (CAS 192139-92-7, 0.046 g, 0.072 mmol) in DCM (10 mL) was stirred at 0° C. 1,8-Diazabicyclo[5.4.0]undec-7-ene (0.67 g, 4.37 mmol) was added dropwise. The reaction was stirred at room temperature for 12 h and concentrated. The residue was purified by silica column (1:1 heptane:ethyl acetate) to give the product (0.41 g, 82%). MS ES+ m/z 346 [M+H]⁺.

### Intermediate 26: 8[(1S)-1-Chloroethyl]-2-[6-(difluoromethyl)-2-pyridyl]-3,6-dimethyl-chromen-4-one

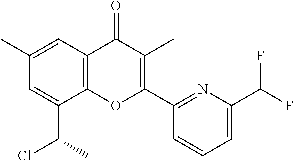 [SMILES: Cc1cc([C@H](C)Cl)c2oc(-c3cccc(C(F)F)n3)c(C)c(=O)c2c1]

A solution of trichloro[1,3,5]triazine (0.33 g, 1.78 mmol) in DMF (0.1 mL) was stirred at room temperature for 30 min. To this was added a solution of 2-[6-(difluoromethyl)-2-pyridyl]-8-[(1R)-1-hydroxyethyl]-3,6-dimethyl-chromen-4-one (0.41 g, 1.19 mmol) in DCM (10 mL). The reaction was stirred for 4 h. Diluted with saturated aqueous sodium carbonate (100 mL) and DCM (20 mL). Separated the layers. The organics were washed with brine and concentrated. The residue was purified by silica column (2:1 heptane:ethyl acetate) to give the product (0.32 g, 74%, 89% ee). MS ES+ m/z 364 [M+H]⁺.

### Example 1: 2-[1-[2-(6-Isopropoxy-3-pyridyl)-6-methyl-4-oxo-chromen-8-yl]ethylamino]benzoic acid 2,2,2-trifluoroacetic acid

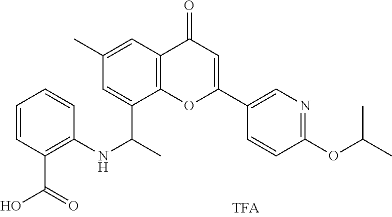 [SMILES: Cc1cc(C(C)Nc2ccccc2C(=O)O)c2oc(-c3ccc(OC(C)C)nc3)cc(=O)c2c1]

tert-Butyl 2-[1-[2-(6-isopropoxy-3-pyridyl)-6-methyl-4-oxo-chromen-8-yl]ethylamino]benzoate (0.150 g, 291 μmol) was dissolved in TFA/DCM (1.5 mL each) and stirred at 40° C. for 3 h. The reaction mixture was concentrated and directly purified using reverse phase (C-18 column, 10-100% acetonitrile[0.1% TFA] in water[0.1% TFA]) to give the product as the trifluoroacetate salt (0.052 g, 31%). MS ES+ m/z 459 [M+H]⁺.

### Example 2: 2-[1-[2-(2-Methoxypyrimidin-5-yl)-6-methyl-4-oxo-chromen-8-yl]ethylamino]benzoic acid 2,2,2-trifluoroacetic acid

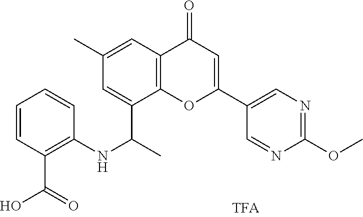 [SMILES: COc1ncc(-c2cc(=O)c3cc(C)cc(C(C)Nc4ccccc4C(=O)O)c3o2)cn1]

A solution of tert-butyl 2-[1-[2-(2-methoxypyrimidin-5-yl)-6-methyl-4-oxo-chromen-8-yl]ethylamino]benzoate (90.0 mg, 185 μmol) in DCM (2 mL) was treated with TFA (2.0 mL, 26 mmol) and heated at 40° for 3 h. The reaction mixture was concentrated and purified using a C-18 column eluting with 10-90% acetonitrile in water (0.1% TFA additive) to give the product (28 mg) as the trifluoroacetate salt. MS ES+ m/z 432 [M+H]⁺.

### Example 3: 2-[1-(6-Methyl-4-oxo-2-pyrimidin-2-yl-chromen-8-yl)ethylamino]benzoic acid, Isomer 2

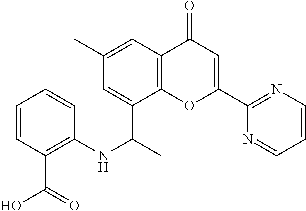 [SMILES: Cc1cc(C(C)Nc2ccccc2C(=O)O)c2oc(-c3ncccn3)cc(=O)c2c1]

A mixture of boron tribromide (180.91 mg, 722.13 umol, 69.58 uL) in DCM (0.5 mL) was added to a mixture of methyl 2-[1-(2-ethylsulfanyl-6-methyl-4-oxo-chromen-8-yl)ethylamino]benzoate, Isomer 2 (100 mg, 240.71 umol) in DCM (2 mL) at −78° C., then stirred at 20° C. for 14 h to give a yellow suspension. The reaction mixture was poured into water, extracted with DCM (2×20 mL), and the combined organic phases were concentrated to a residue. The residue was purified via preparative HPLC using 0.225% formic acid as an additive to give the product (6.65 mg; 6.9%) as a light yellow solid. MS ES+ m/z 402 [M+H]⁺.

### Example 4 and Example 5: 2-[1-[2-(2-Methoxypyrimidin-5-yl)-6-methyl-4-oxo-chromen-8-yl]ethylamino]benzoic acid, Isomer 1 and Isomer 2

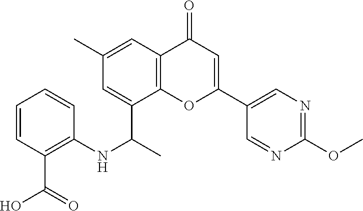 [SMILES: COc1ncc(-c2cc(=O)c3cc(C)cc(C(C)Nc4ccccc4C(=O)O)c3o2)cn1]

Dissolved 2-[1-[2-(2-methoxypyrimidin-5-yl)-6-methyl-4-oxo-chromen-8-yl]ethylyamino]benzoic acid 2,2,2-trifluoroacetic acid (22 mg) in MeOH (2.25 mL) and DCM (2.25 mL). Separated via superritical fluid chromatography (Chiralpak AD-H, 150 mm×21 mm; 30% EtOH w/0.500 DMEA: 70% CO₂) to obtain the product isomers (8.3 mg, 8.3 mg). MS ES+ m/z 432 [M+H]⁺, for both.

The following compounds in Table 1 can be made according to Schemes 1-3 or the foregoing Examples.

TABLE 1

| Example | | | MS ES+ |
|---|---|---|---|
| # | Chemical Name | Structure | m/z |
| 6 | 2-[1-[2-(2- Isopropoxypyrimidin- 5-yl)-6-methyl-4-oxo- chromen-8- yl]ethylamino]benzoic acid 2,2,2- trifluoroacetic acid | 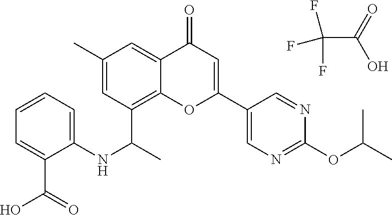 | 460 [M + H]⁺ |
| 7 | 2-[1-[6-Methyl-2-(6- methylsulfonyl-3- pyridyl)-4-oxo- chromen-8- yl]ethylamino]benzoic acid 2,2,2- trifluoroacetic acid | 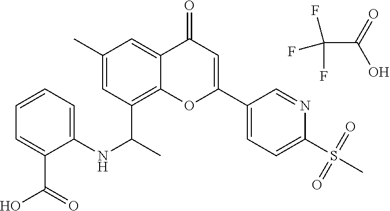 | 479 [M + H]⁺ |
| 8 | 2-[1-[6-Methyl-4-oxo- 2-[6-(trifluoromethyl)- 3-pyridyl]chromen-8- yl]ethylamino]benzoic acid 2,2,2- trifluoroacetic acid | 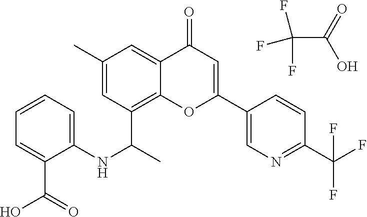 | 469 [M + H]⁺ |
| 9 | 2-[1-[2-(6-Fluoro-3- pyridyl)-6-methyl-4- oxo-chromen-8- yl]ethylamino]benzoic acid 2,2,2- trifluoroacetic acid | 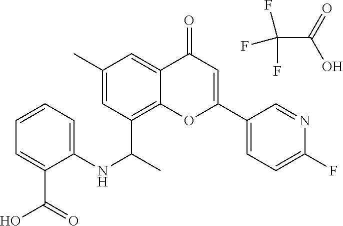 | 419 [M + H]⁺ |
| 10 | 2-[1-[2-(4-Cyano-2- pyridyl)-6-methyl-4- oxo-chromen-8- yl]ethylamino]benzoic acid 2,2,2- trifluoroacetic acid | 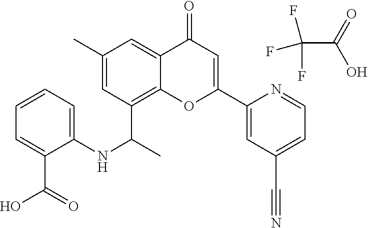 | 426 [M + H]⁺ |
| 11 | 2-[1-[2-(6-Methoxy-2- pyridyl)-6-methyl-4- oxo-chromen-8- yl]ethylamino]benzoic acid 2,2,2- trifluoroacetic acid | 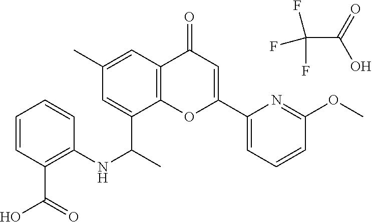 | 431 [M + H]⁺ |
| 12 | 2-[1-[2-(6-Methoxy-3- pyridyl)-6-methyl-4- oxo-chromen-8- yl]ethylamino]benzoic acid 2,2,2- trifluoroacetic acid | 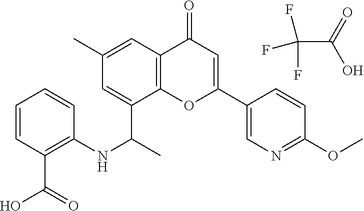 | 431 [M + H]⁺ |
| 13 | 2-[1-[6-Methyl-2-(2- methylpyrimidin-5-yl)- 4-oxo-chromen-8- yl]ethylamino]benzoic acid 2,2,2- trifluoroacetic acid | 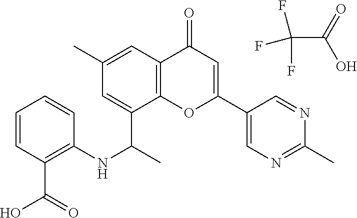 | 416 [M + H]⁺ |
| 14 | 2-[1-[6-Methyl-4-oxo- 2-(3-pyridyl)chromen- 8- yl]ethylamino]benzoic acid, Isomer 2 | 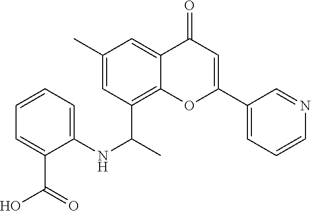 [SMILES: Cc1cc(C(C)Nc2ccccc2C(=O)O)c2oc(-c3cccnc3)cc(=O)c2c1] | 401 [M + H]⁺ |
| 15 | 2-[1-(6-Methyl-4-oxo- 2-pyrazin-2-yl- chromen-8- yl)ethylamino]benzoic acid, Isomer 1 | 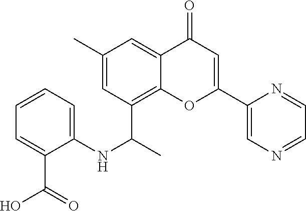 [SMILES: Cc1cc(C(C)Nc2ccccc2C(=O)O)c2oc(-c3cnccn3)cc(=O)c2c1] | 402 [M + H]⁺ |
| 16 | 2-[1-(6-Methyl-4-oxo- 2-pyrazin-2-yl- chromen-8- yl)ethylamino]benzoic acid, Isomer 2 | 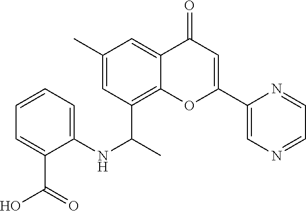 [SMILES: Cc1cc(C(C)Nc2ccccc2C(=O)O)c2oc(-c3cnccn3)cc(=O)c2c1] | 402 [M + H]⁺ |
| 17 | 2-[1-[6-Methyl-4-oxo- 2-(2-pyridyl)chromen- 8- yl]ethylamino]benzoic acid, Isomer 1 | 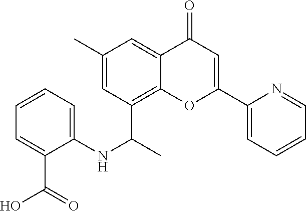 [SMILES: Cc1cc(C(C)Nc2ccccc2C(=O)O)c2oc(-c3ccccn3)cc(=O)c2c1] | 401 [M + H]⁺ |
| 18 | 2-[1-[6-Methyl-4-oxo- 2-(2-pyridyl)chromen- 8- yl]ethylamino]benzoic acid, Isomer 2 | 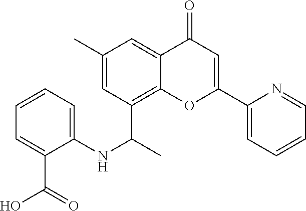 [SMILES: Cc1cc(C(C)Nc2ccccc2C(=O)O)c2oc(-c3ccccn3)cc(=O)c2c1] | 401 [M + H]⁺ |
| 19 | 2-[1-[2-(6-Methoxy-3- pyridyl)-6-methyl-4- oxo-chromen-8- yl]ethylamino]benzoic acid, Isomer 1 | 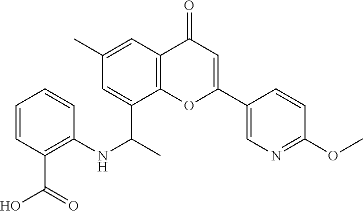 [SMILES: COc1ccc(-c2cc(=O)c3cc(C)cc(C(C)Nc4ccccc4C(=O)O)c3o2)cn1] | 431 [M + H]⁺ |
| 20 | 2-[1-[2-(6-Methoxy-3- pyridyl)-6-methyl-4- oxo-chromen-8- yl]ethylamino]benzoic acid, Isomer 2 | 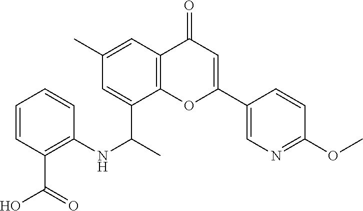 [SMILES: COc1ccc(-c2cc(=O)c3cc(C)cc(C(C)Nc4ccccc4C(=O)O)c3o2)cn1] | 431 [M + H]⁺ |
| 21 | 2-[1-[2-(6-Methoxy-2- pyridyl)-6-methyl-4- oxo-chromen-8- yl]ethylamino]benzoic acid, Isomer 1 | 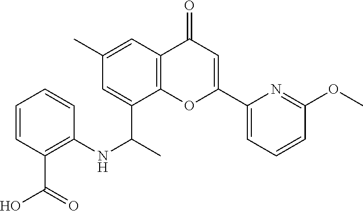 [SMILES: COc1cccc(-c2cc(=O)c3cc(C)cc(C(C)Nc4ccccc4C(=O)O)c3o2)n1] | 431 [M + H]⁺ |
| 22 | 2-[1-[2-(6-Methoxy-2- pyridyl)-6-methyl-4- oxo-chromen-8- yl]ethylamino]benzoic acid, Isomer 2 | 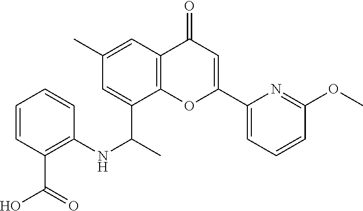 [SMILES: COc1cccc(-c2cc(=O)c3cc(C)cc(C(C)Nc4ccccc4C(=O)O)c3o2)n1] | 431 [M + H]⁺ |
| 23 | 2-[1-[2-(5-Fluoro-2- pyridyl)-6-methyl-4- oxo-chromen-8- yl]ethylamino]benzoic acid 2,2,2- trifluoroacetic acid | 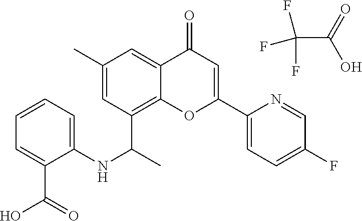 | 419 [M + H]⁺ |
| 24 | 2-[1-[3,6-Dimethyl-4- oxo-2-(2- pyridyl)chromen-8- yl]ethylamino]benzoic acid, Isomer 1 | 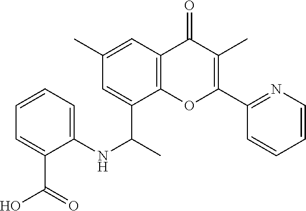 [SMILES: Cc1cc(C(C)Nc2ccccc2C(=O)O)c2oc(-c3ccccn3)c(C)c(=O)c2c1] | 415 [M + H]⁺ |
| 25 | 2-[1-[3,6-Dimethyl-4- oxo-2-(3- pyridyl)chromen-8- yl]ethylamino]benzoic acid, Isomer 1 | 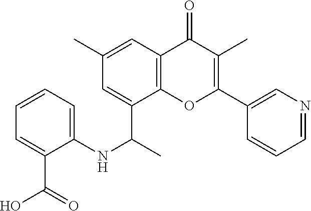 [SMILES: Cc1cc(C(C)Nc2ccccc2C(=O)O)c2oc(-c3cccnc3)c(C)c(=O)c2c1] | 415 [M + H]⁺ |
| 26 | 2-[1-[2-(2- Cyclopropylpyrimidin- 5-yl)-6-methyl-4-oxo- chromen-8- yl]ethylamino]benzoic acid, Isomer 2 | 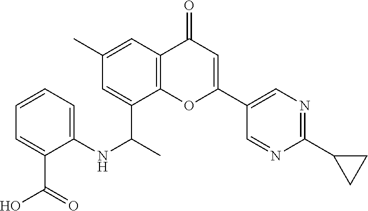 [SMILES: Cc1cc(C(C)Nc2ccccc2C(=O)O)c2oc(-c3cnc(C4CC4)nc3)cc(=O)c2c1] | 442 [M + H]⁺ |
| 27 | 6-Chloro-3-[1-[6- methyl-4-oxo-2-(2- pyridyl)chromen-8- yl]ethylamino]pyridine- 2-carboxylic acid, Isomer 1 | 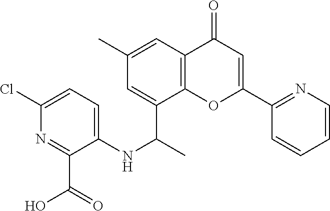 [SMILES: Cc1cc(C(C)Nc2ccc(Cl)nc2C(=O)O)c2oc(-c3ccccn3)cc(=O)c2c1] | 436 [M + H]⁺ |
| 28 | 6-Chloro-3-[1-(6- methyl-4-oxo-2- pyrazin-2-yl-chromen- 8- yl)ethylamino]pyridine- 2-carboxylic acid, Isomer 1 | 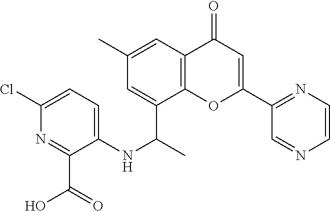 [SMILES: Cc1cc(C(C)Nc2ccc(Cl)nc2C(=O)O)c2oc(-c3cnccn3)cc(=O)c2c1] | 437 [M + H]⁺ |
| 29 | 2-[1-[6-Methyl-2-(3- methylpyrazin-2-yl)-4- oxo-chromen-8- yl]ethylamino]benzoic acid, Isomer 2 | 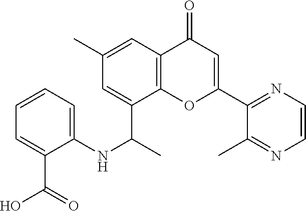 [SMILES: Cc1cc(C(C)Nc2ccccc2C(=O)O)c2oc(-c3nccnc3C)cc(=O)c2c1] | 416 [M + H]⁺ |
| 30 | 2-[1-[2-(6-Isopropoxy- 3-pyridyl)-6-methyl-4- oxo-chromen-8- yl]ethylamino]benzoic acid, Isomer 1 | 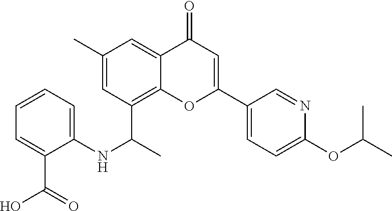 [SMILES: Cc1cc(C(C)Nc2ccccc2C(=O)O)c2oc(-c3ccc(OC(C)C)nc3)cc(=O)c2c1] | 459 [M + H]⁺ |
| 31 | 2-[1-[2-(6-Isopropoxy- 3-pyridyl)-6-methyl-4- oxo-chromen-8- yl]ethylamino]benzoic acid, Isomer 2 | 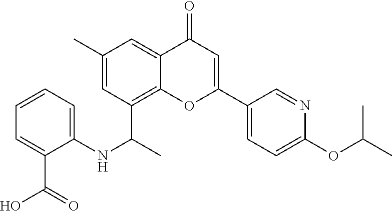 [SMILES: Cc1cc(C(C)Nc2ccccc2C(=O)O)c2oc(-c3ccc(OC(C)C)nc3)cc(=O)c2c1] | 459 [M + H]⁺ |
| 32 | 2-[1-[6-Methyl-2-(2- methylpyrimidin-5-yl)- 4-oxo-chromen-8- yl]ethylamino]benzoic acid, Isomer 1 | 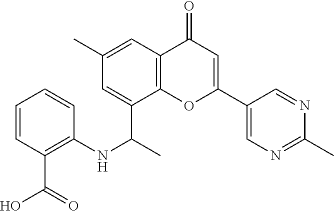 [SMILES: Cc1cc(C(C)Nc2ccccc2C(=O)O)c2oc(-c3cnc(C)nc3)cc(=O)c2c1] | 416 [M + H]⁺ |
| 33 | 2-[1-[6-Methyl-2-(2- methylpyrimidin-5-yl)- 4-oxo-chromen-8- yl]ethylamino]benzoic acid, Isomer 2 | 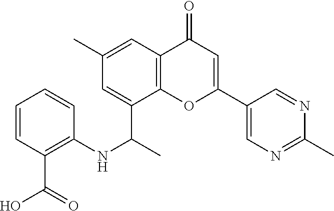 [SMILES: Cc1cc(C(C)Nc2ccccc2C(=O)O)c2oc(-c3cnc(C)nc3)cc(=O)c2c1] | 416 [M + H]⁺ |
| 34 | 2-[1-(3,6-Dimethyl-4- oxo-2-pyrimidin-2-yl- chromen-8- yl)ethylamino]benzoic acid, Isomer 1 | 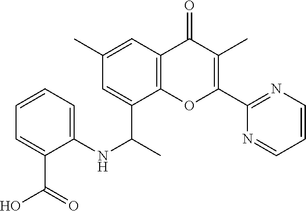 [SMILES: Cc1cc(C(C)Nc2ccccc2C(=O)O)c2oc(-c3ncccn3)c(C)c(=O)c2c1] | 416 [M + H]⁺ |
| 35 | 2-[1-[2-(6-Fluoro-3- pyridyl)-6-methyl-4- oxo-chromen-8- yl]ethylamino]benzoic acid, Isomer 1 | 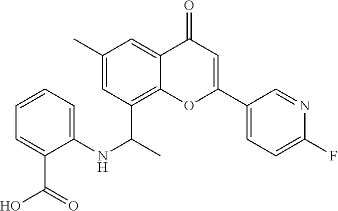 [SMILES: Cc1cc(C(C)Nc2ccccc2C(=O)O)c2oc(-c3ccc(F)nc3)cc(=O)c2c1] | 419 [M + H]⁺ |
| 36 | 2-[1-[2-(6-Fluoro-3- pyridyl)-6-methyl-4- oxo-chromen-8- yl]ethylamino]benzoic acid, Isomer 2 | 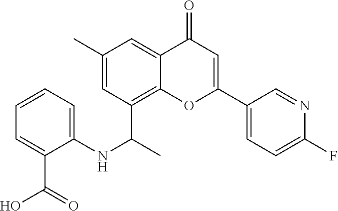 [SMILES: Cc1cc(C(C)Nc2ccccc2C(=O)O)c2oc(-c3ccc(F)nc3)cc(=O)c2c1] | 419 [M + H]⁺ |
| 37 | 2-[1-[6-Methyl-4-oxo- 2-[6-(trifluoromethyl)- 3-pyridyl]chromen-8- yl]ethylamino]benzoic acid, Isomer 1 | 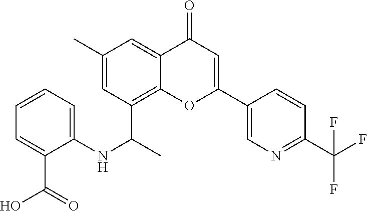 [SMILES: Cc1cc(C(C)Nc2ccccc2C(=O)O)c2oc(-c3ccc(C(F)(F)F)nc3)cc(=O)c2c1] | 469 [M + H]⁺ |
| 38 | 2-[1-[6-Methyl-4-oxo- 2-[6-(trifluoromethyl)- 3-pyridyl]chromen-8- yl]ethylamino]benzoic acid, Isomer 2 | 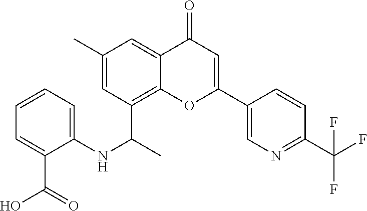 [SMILES: Cc1cc(C(C)Nc2ccccc2C(=O)O)c2oc(-c3ccc(C(F)(F)F)nc3)cc(=O)c2c1] | 469 [M + H]⁺ |
| 39 | 2-[1-[6-Methyl-2-(6- methylsulfonyl-3- pyridyl)-4-oxo- chromen-8- yl]ethylamino]benzoic acid, Isomer 1 | 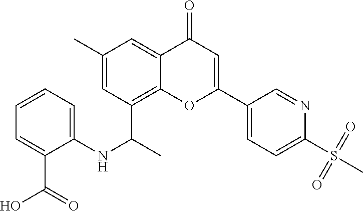 [SMILES: Cc1cc(C(C)Nc2ccccc2C(=O)O)c2oc(-c3ccc(S(C)(=O)=O)nc3)cc(=O)c2c1] | 479 [M + H]⁺ |
| 40 | 2-[1-[6-Methyl-2-(6- methylsulfonyl-3- pyridyl)-4-oxo- chromen-8- yl]ethylamino]benzoic acid, Isomer 2 | 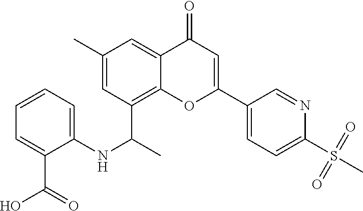 [SMILES: Cc1cc(C(C)Nc2ccccc2C(=O)O)c2oc(-c3ccc(S(C)(=O)=O)nc3)cc(=O)c2c1] | 479 [M + H]⁺ |
| 41 | 2-[1-[2-(2- Isopropoxypyrimidin- 5-yl)-6-methyl-4-oxo- chromen-8- yl]ethylamino]benzoic acid, Isomer 1 | 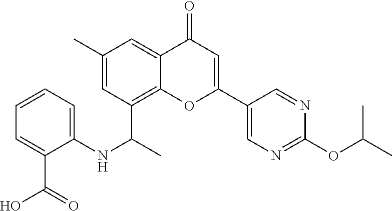 [SMILES: Cc1cc(C(C)Nc2ccccc2C(=O)O)c2oc(-c3cnc(OC(C)C)nc3)cc(=O)c2c1] | 460 [M + H]⁺ |
| 42 | 2-[1-[2-(2- Isopropoxypyrimidin- 5-yl)-6-methyl-4-oxo- chromen-8- yl]ethylamino]benzoic acid, Isomer 2 | 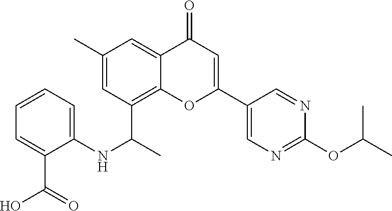 [SMILES: Cc1cc(C(C)Nc2ccccc2C(=O)O)c2oc(-c3cnc(OC(C)C)nc3)cc(=O)c2c1] | 460 [M + H]⁺ |
| 43 | 2-[1-[2-(4-Methoxy-2- pyridyl)-6-methyl-4- oxo-chromen-8- yl]ethylamino]benzoic acid, Isomer 2, 2,2,2- trifluoroacetic acid | 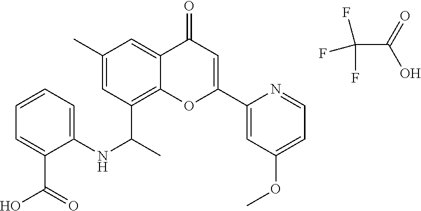 [SMILES: COc1ccnc(-c2cc(=O)c3cc(C)cc(C(C)Nc4ccccc4C(=O)O)c3o2)c1 ; O=C(O)C(F)(F)F] | 431 [M + H]⁺ |
| 44 | 2-[1-[2-(5-Fluoro-3- pyridyl)-6-methyl-4- oxo-chromen-8- yl]ethylamino]benzoic acid, Isomer 2, 2,2,2- trifluoroacetic acid | 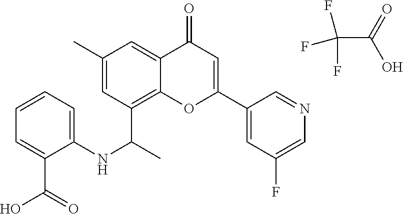 [SMILES: Cc1cc(C(C)Nc2ccccc2C(=O)O)c2oc(-c3cncc(F)c3)cc(=O)c2c1 ; O=C(O)C(F)(F)F] | 419 [M + H]⁺ |
| 45 | 2-[1-[2-(5-Fluoro-3- pyridyl)-3,6-dimethyl- 4-oxo-chromen-8- yl]ethylamino]benzoic acid, Isomer 1 | 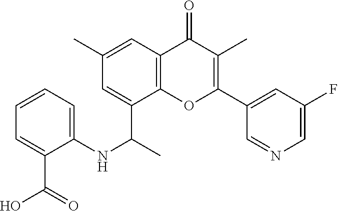 [SMILES: Cc1cc(C(C)Nc2ccccc2C(=O)O)c2oc(-c3cncc(F)c3)c(C)c(=O)c2c1] | 433 [M + H]⁺ |
| 46 | 2-[1-[6-Fluoro-4-oxo- 2-(2-pyridyl)chromen- 8- yl]ethylamino]benzoic acid, Isomer 1, 2,2,2- trifluoroacetic acid | 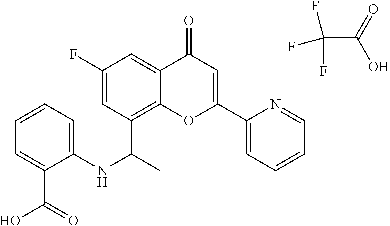 [SMILES: CC(Nc1ccccc1C(=O)O)c1cc(F)cc2c(=O)cc(-c3ccccn3)oc12 ; O=C(O)C(F)(F)F] | 405 [M + H]⁺ |
| 47 | 2-[1-[6-Methyl-2-(1- methylpyrazol-4-yl)-4- oxo-chromen-8- yl]ethylamino]benzoic acid | 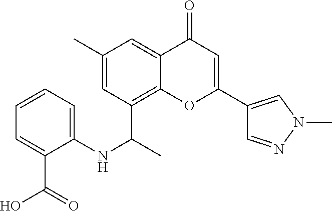 [SMILES: Cc1cc(C(C)Nc2ccccc2C(=O)O)c2oc(-c3cnn(C)c3)cc(=O)c2c1] | 404 [M + H]⁺ |
| 48 | 2-[1-[6-Methyl-4-oxo- 2-[1-(3- pyridyl)pyrazol-4- yl]chromen-8- yl]ethylamino]benzoic acid 2,2,2- trifluoroacetic acid | 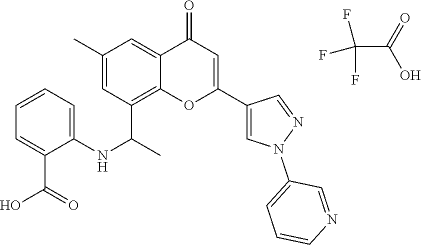 [SMILES: Cc1cc(C(C)Nc2ccccc2C(=O)O)c2oc(-c3cnn(-c4cccnc4)c3)cc(=O)c2c1 ; O=C(O)C(F)(F)F] | 467 [M + H]⁺ |
| 49 | 2-[1-[6-Methyl-4-oxo- 2-[1-(3- pyridyl)pyrazol-4- yl]chromen-8- yl]ethylamino]benzoic acid, Isomer 1 | 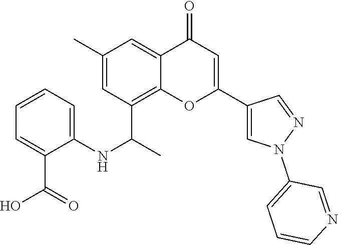 [SMILES: Cc1cc(C(C)Nc2ccccc2C(=O)O)c2oc(-c3cnn(-c4cccnc4)c3)cc(=O)c2c1] | 467 [M + H]⁺ |
| 50 | 2-[1-[6-Methyl-4-oxo- 2-[1-(3- pyridyl)pyrazol-4- yl]chromen-8- yl]ethylamino]benzoic acid, Isomer 2 | 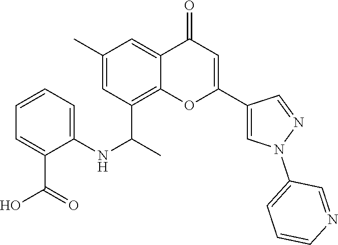 [SMILES: Cc1cc(C(C)Nc2ccccc2C(=O)O)c2oc(-c3cnn(-c4cccnc4)c3)cc(=O)c2c1] | 467 [M + H]⁺ |
| 51 | 2-[1-[6-Methyl-4-oxo- 2-(1H-pyrazol-4- yl)chromen-8- yl]ethylamino]benzoic acid, Isomer 2, 2,2,2- trifluoroacetic acid | 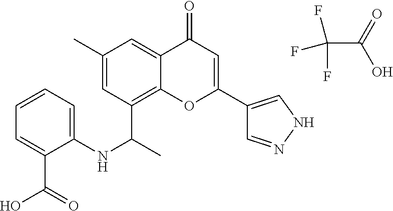 [SMILES: Cc1cc(C(C)Nc2ccccc2C(=O)O)c2oc(-c3cn[nH]c3)cc(=O)c2c1 ; O=C(O)C(F)(F)F] | 390 [M + H]⁺ |
| 52 | 2-[1-[2-[1- (Difluoromethyl)pyrazol- 4-yl]-6-methyl-4- oxo-chromen-8- yl]ethylamino]benzoic acid, Isomer 2, 2,2,2- trifluoroacetic acid | 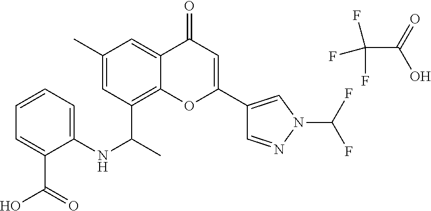 | 440 [M + H]⁺ |
| 53 | 2-[1-[2-[1-(3- Methoxyphenyl)pyrazol- 4-yl]-6-methyl-4-oxo- chromen-8- yl]ethylamino]benzoic acid, Isomer 2, 2,2,2- trifluoroacetic acid | 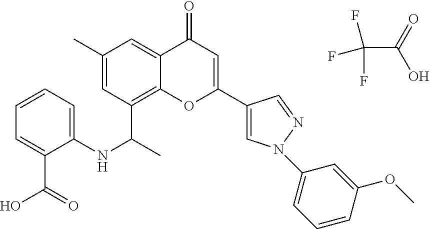 [SMILES: COc1cccc(-n2cc(-c3cc(=O)c4cc(C)cc(C(C)Nc5ccccc5C(=O)O)c4o3)cn2)c1 ; O=C(O)C(F)(F)F] | 496 [M + H]⁺ |
| 54 | 2-[1-[2-[1-(3- Cyanophenyl)pyrazol- 4-yl]-6-methyl-4-oxo- chromen-8- yl]ethylamino]benzoic acid, Isomer 2, 2,2,2- trifluoroacetic acid | 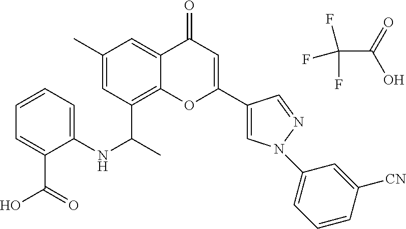 | 491 [M + H]⁺ |
| 55 | 2-[1-[6-Fluoro-4-oxo- 2-[1-(3- pyridyl)pyrazol-4- yl]chromen-8- yl]ethylamino]benzoic acid, Isomer 1, 2,2,2- trifluoroacetic acid | 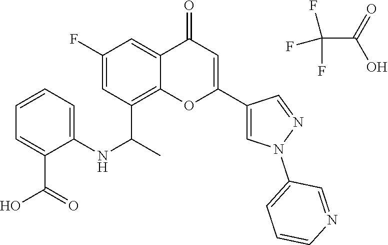 [SMILES: CC(Nc1ccccc1C(=O)O)c1cc(F)cc2c(=O)cc(-c3cnn(-c4cccnc4)c3)oc12 ; O=C(O)C(F)(F)F] | 471 [M + H]⁺ |
| 56 | Methyl 2-[1-[6-methyl- 4-oxo-2-(6-oxo-1H- pyrazin-3-yl)chromen- 8- yl]ethylamino]benzoate, Isomer 2 | 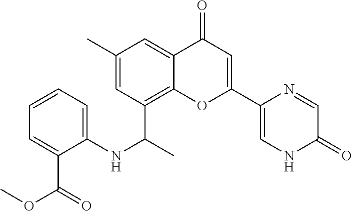 [SMILES: COC(=O)c1ccccc1NC(C)c1cc(C)cc2c(=O)cc(-c3c[nH]c(=O)cn3)oc12] | 432 [M + H]⁺ |
| 60 | 6-Chloro-3-[1-[3,6- dimethyl-4-oxo-2-(2- pyridyl)chromen-8- yl]ethylamino]pyridine- 2-carboxylic acid, Isomer 1 | 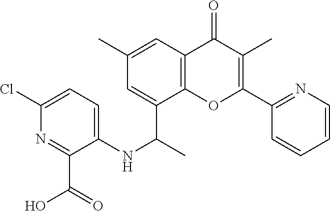 [SMILES: Cc1cc(C(C)Nc2ccc(Cl)nc2C(=O)O)c2oc(-c3ccccn3)c(C)c(=O)c2c1] | 450 [M + H]⁺ |
| 61 | 6-Chloro-3-[1-[3- methyl-4-oxo-2-(2- pyridyl)-6- (trifluoromethyl) chromen-8- yl]ethylamino]pyridine- 2-carboxylic acid, Isomer 1 | 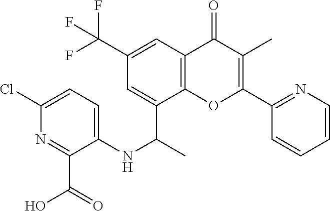 [SMILES: Cc1c(-c2ccccn2)oc2c(C(C)Nc3ccc(Cl)nc3C(=O)O)cc(C(F)(F)F)cc2c1=O] | 504 [M + H]⁺ |
| 62 | 6-Chloro-3-[1-[4-oxo- 2-(2-pyridyl)-6- (trifluoromethyl) chromen-8- yl]ethylamino]pyridine- 2-carboxylic acid, Isomer 1 | 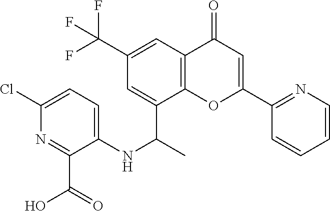 [SMILES: CC(Nc1ccc(Cl)nc1C(=O)O)c1cc(C(F)(F)F)cc2c(=O)cc(-c3ccccn3)oc12] | 490 [M + H]⁺ |
| 63 | 2-[1-[3,6-Dimethyl-4- oxo-2-[1-(3- pyridyl)pyrazol-4- yl]chromen-8- yl]ethylamino]benzoic acid, Isomer 1, 2,2,2- trifluoroacetic acid | 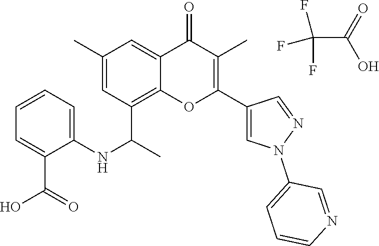 | 481 [M + H]⁺ |
| 64 | 2-[1-2-[1-(4- Cyanophenyl)pyrazol- 4-yl]-6-methyl-4-oxo- chromen-8- yl]ethylamino]benzoic acid, Isomer 2, 2,2,2- trifluoroacetic acid | 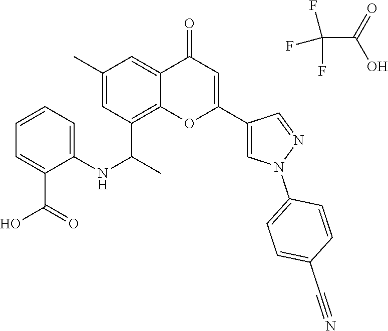 [SMILES: Cc1cc(C(C)Nc2ccccc2C(=O)O)c2oc(-c3cnn(-c4ccc(C#N)cc4)c3)cc(=O)c2c1 ; O=C(O)C(F)(F)F] | 491 [M + H]⁺ |
| 65 | 2-[1-[2-(6-Methoxy-3- pyridyl)-3,6-dimethyl- 4-oxo-chromen-8- yl]ethylamino]benzoic acid, Isomer 1, 2,2,2- trifluoroacetic acid | 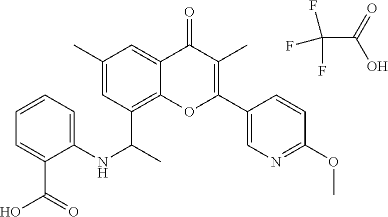 | 445 [M + H]⁺ |
| 66 | 2-[1-2-[1-(3- Methoxyphenyl)pyrazol- 4-yl]-3,6-dimethyl-4- oxo-chromen-8- yl]ethylamino]benzoic acid, Isomer 1, 2,2,2- trifluoroacetic acid | 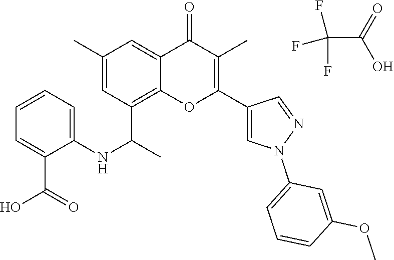 [SMILES: COc1cccc(-n2cc(-c3oc4c(C(C)Nc5ccccc5C(=O)O)cc(C)cc4c(=O)c3C)cn2)c1 ; O=C(O)C(F)(F)F] | 510 [M + H]⁺ |
| 67 | 2-[1-[6-Methyl-4-oxo- 2-(2-pyridyl)-3- (trifluoromethyl) chromen-8- yl]ethylamino]benzoic acid, Isomer 1, 2,2,2- trifluoroacetic acid | 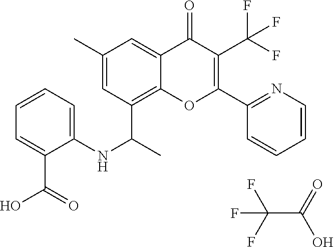 | 469 [M + H]⁺ |
| 68 | 2-[1-[6-Fluoro-3- methyl-4-oxo-2-(2- pyridyl)chromen-8- yl]ethylamino]benzoic acid, Isomer 1, 2,2,2- trifluoroacetic acid | 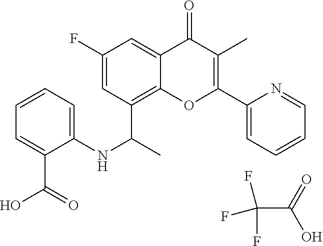 | 419 [M + H]⁺ |
| 69 | 2-[1-[2-[1-(4- Methoxyphenyl)pyrazol- 4-yl]-3,6-dimethyl-4- oxo-chromen-8- yl]ethylamino]benzoic acid, Isomer 1, 2,2,2- trifluoroacetic acid | 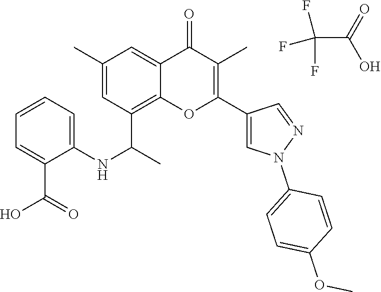 | 510 [M + H]⁺ |
| 70 | 2-[1-2-[1-(2- Methoxyphenyl)pyrazol- 4-yl]-3,6-dimethyl-4- oxo-chromen-8- yl]ethylamino]benzoic acid, Isomer 1, 2,2,2- trifluoroacetic acid | 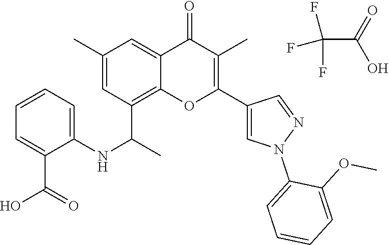 | 510 [M + H]⁺ |
| 71 | 2-[1-[2-[6- (Difluoromethyl)-2- pyridyl]-6-methyl-4- oxo-chromen-8- yl]ethylamino]benzoic acid, Isomer 2 | 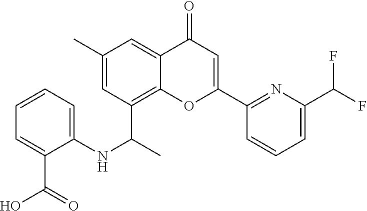 [SMILES: Cc1cc(C(C)Nc2ccccc2C(=O)O)c2oc(-c3cccc(C(F)F)n3)cc(=O)c2c1] | 451 [M + H]⁺ |
| 72 | 2-[1-[2-[1-(2- Methoxypyrimidin-5- yl)pyrazol-4-yl]-3, 6- dimethyl-4-oxo- chromen-8- yl]ethylamino]benzoic acid, Isomer 1, 2,2,2- trifluoroacetic acid | 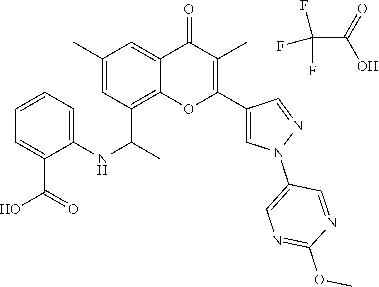 | 512 [M + H]⁺ |
| 73 | 2-[1-[2-[1-(4- Cyanophenyl)pyrazol- 4-yl]-3,6-dimethyl-4- oxo-chromen-8- yl]ethylamino]benzoic acid, Isomer 1, 2,2,2- trifluoroacetic acid | 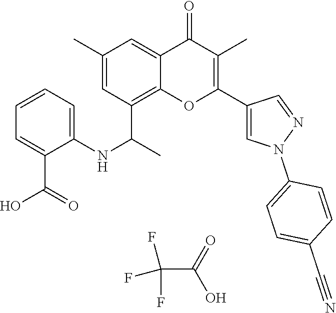 [SMILES: Cc1cc(C(C)Nc2ccccc2C(=O)O)c2oc(-c3cnn(-c4ccc(C#N)cc4)c3)c(C)c(=O)c2c1 ; O=C(O)C(F)(F)F] | 505 [M + H]⁺ |
| 74 | 2-[1-[2-[1-(3- Cyanophenyl)pyrazol- 4-yl]-3,6-dimethyl-4- oxo-chromen-8- yl]ethylamino]benzoic acid, Isomer 1, 2,2,2- trifluoroacetic acid | 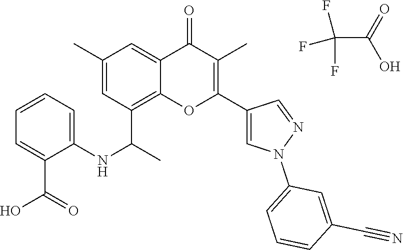 [SMILES: Cc1cc(C(C)Nc2ccccc2C(=O)O)c2oc(-c3cnn(-c4cccc(C#N)c4)c3)c(C)c(=O)c2c1 ; O=C(O)C(F)(F)F] | 505 [M + H]⁺ |
| 75 | 2-[1-[6-Methyl-2-(3- methyl-1H-pyrazol-5- yl)-4-oxo-chromen-8- yl]ethylamino]benzoic acid, Isomer 2, 2,2,2- trifluoroacetic acid | 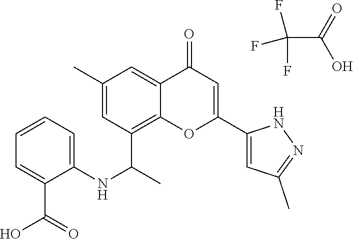 [SMILES: Cc1cc(C(C)Nc2ccccc2C(=O)O)c2oc(-c3cc(C)n[nH]3)cc(=O)c2c1 ; O=C(O)C(F)(F)F] | 404 [M + H]⁺ |
| 76 | 2-[1-[4-Oxo-2-(2- pyridyl)-6- (trifluoromethyl) chromen-8- yl]ethylamino]benzoic acid, Isomer 1 | 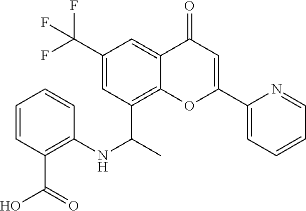 [SMILES: CC(Nc1ccccc1C(=O)O)c1cc(C(F)(F)F)cc2c(=O)cc(-c3ccccn3)oc12] | 455 [M + H]⁺ |
| 77 | 2-[1-[2-[6- (Difluoromethyl)-2- pyridyl]-3,6-dimethyl- 4-oxo-chromen-8- yl]ethylamino]benzoic acid, Isomer 1, 2,2,2- trifluoroacetic acid | 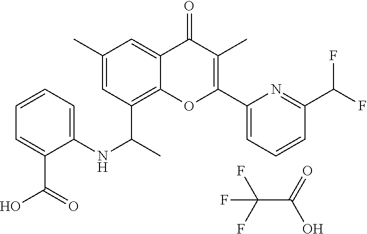 | 465 [M + H]⁺ |
| 78 | 3-Chloro-2-fluoro-6-[1- [6-methyl-4-oxo-2-(2- pyridyl)chromen-8- yl]ethylamino]benzoic acid, Isomer 1 | 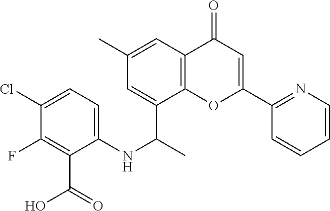 [SMILES: Cc1cc(C(C)Nc2ccc(Cl)c(F)c2C(=O)O)c2oc(-c3ccccn3)cc(=O)c2c1] | 453 [M + H]⁺ |
| 79 | 2-[1-[2-[1-(4- Methoxyphenyl)pyrazol- 4-yl]-6-methyl-4-oxo- chromen-8- yl]ethylamino]benzoic acid, Isomer 2, 2,2,2- trifluoroacetic acid | 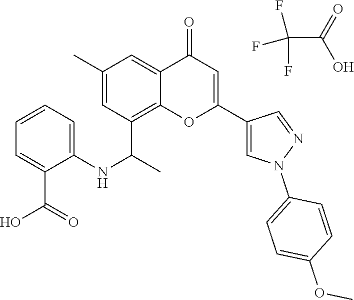 | 496 [M + H]⁺ |
| 80 | 2-[1-[6-Methyl-4-oxo- 2-(1-phenylpyrazol-4- yl)chromen-8- yl]ethylamino]benzoic acid, Isomer 2, 2,2,2- trifluoroacetic acid | 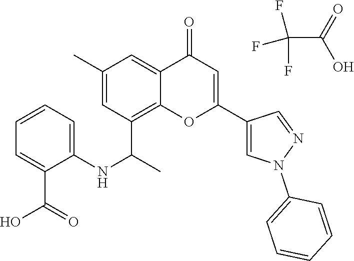 | 466 [M + H]⁺ |
| 81 | 2-[1-[2-[1-(6-Methoxy- 3-pyridyl)pyrazol-4- yl]-6-methyl-4-oxo- chromen-8- yl]ethylamino]benzoic acid, Isomer 2, 2,2,2- trifluoroacetic acid | 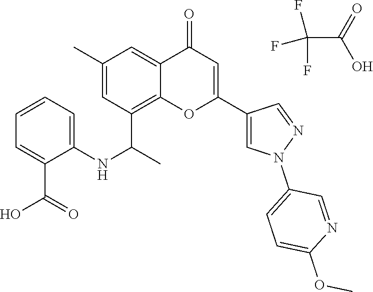 | 497 [M + H]⁺ |
| 82 | 2-[1-[2-[1-(6-Cyano-3- pyridyl)pyrazol-4-yl]- 6-methyl-4-oxo- chromen-8- yl]ethylamino]benzoic acid, Isomer 2, 2,2,2- trifluoroacetic acid | 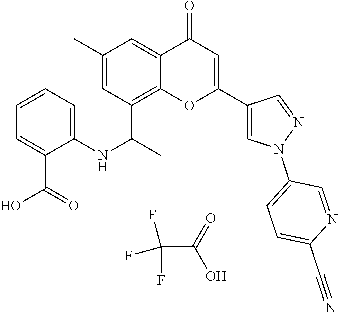 [SMILES: Cc1cc(C(C)Nc2ccccc2C(=O)O)c2oc(-c3cnn(-c4ccc(C#N)nc4)c3)cc(=O)c2c1 ; O=C(O)C(F)(F)F] | 492 [M + H]⁺ |
| 83 | 2-[1-[2-[1-(5-Cyano-2- pyridyl)pyrazol-4-yl]- 6-methyl-4-oxo- chromen-8- yl]ethylamino]benzoic acid, Isomer 2, 2,2,2- trifluoroacetic acid | 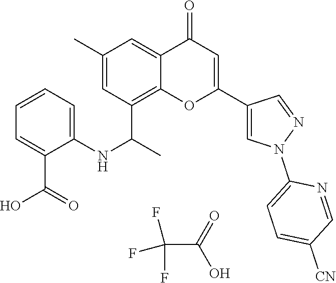 [SMILES: Cc1cc(C(C)Nc2ccccc2C(=O)O)c2oc(-c3cnn(-c4ccc(C#N)cn4)c3)cc(=O)c2c1 ; O=C(O)C(F)(F)F] | 492 [M + H]⁺ |
| 84 | 2-[1-[2-[5-(3- Methoxyphenyl)-1,3,4- oxadiazol-2-yl]-6- methyl-4-oxo- chromen-8- yl]ethylamino]benzoic acid, Isomer 2 | 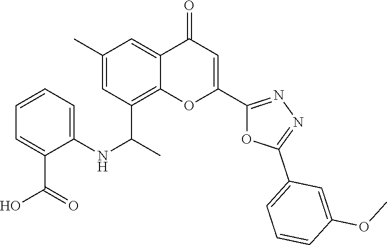 [SMILES: COc1cccc(-c2nnc(-c3cc(=O)c4cc(C)cc(C(C)Nc5ccccc5C(=O)O)c4o3)o2)c1] | 498 [M + H]⁺ |
| 85 | 2-[1-[2-[1-(4-Cyano-3- fluoro-phenyl)pyrazol- 4-yl]-6-methyl-4-oxo- chromen-8- yl]ethylamino]benzoic acid, Isomer 2, 2,2,2- trifluoroacetic acid | 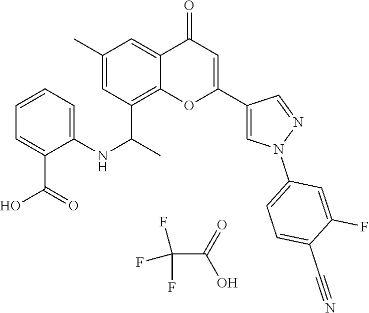 [SMILES: Cc1cc(C(C)Nc2ccccc2C(=O)O)c2oc(-c3cnn(-c4ccc(C#N)c(F)c4)c3)cc(=O)c2c1 ; O=C(O)C(F)(F)F] | 509 [M + H]⁺ |
| 86 | 2-[1-[2-[1-(3-Cyano-4- methoxy- phenyl)pyrazol-4-yl]-6- methyl-4-oxo- chromen-8- yl]ethylamino]benzoic acid, Isomer 2, 2,2,2- trifluoroacetic acid | 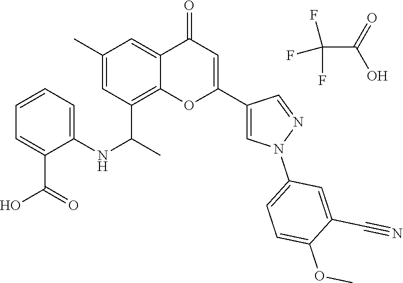 | 521 [M + H]⁺ |
| 87 | 2-[1-[6-Methyl-4-oxo- 2-[1-[4- (trifluoromethyl)phenyl] pyrazol-4-yl]chromen- 8- yl]ethylamino]benzoic acid, Isomer 2, 2,2,2- trifluoroacetic acid | 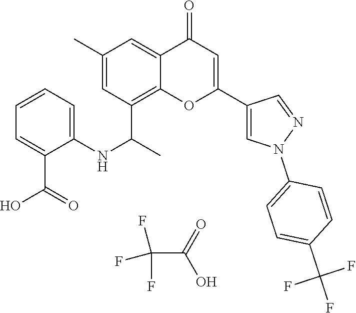 [SMILES: Cc1cc(C(C)Nc2ccccc2C(=O)O)c2oc(-c3cnn(-c4ccc(C(F)(F)F)cc4)c3)cc(=O)c2c1 ; O=C(O)C(F)(F)F] | 534 [M + H]⁺ |
| 88 | 2-[1-[6-Methyl-2-[1-(4- methylsulfonylphenyl) pyrazol-4-yl]-4-oxo- chromen-8- yl]ethylamino]benzoic acid, Isomer 2, 2,2,2- trifluoroacetic acid | 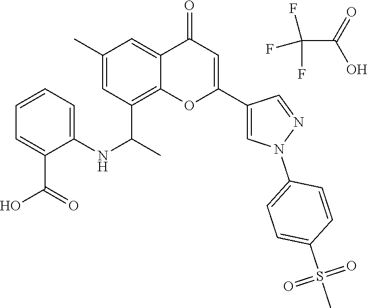 | 544 [M + H]⁺ |
| 89 | 2-[1-[2-[6-(1- Cyanocyclopropyl)-3- pyridyl]-3,6-dimethyl- 4-oxo-chromen-8- yl]ethylamino]benzoic acid, Isomer 1, 2,2,2- trifluoroacetic acid | 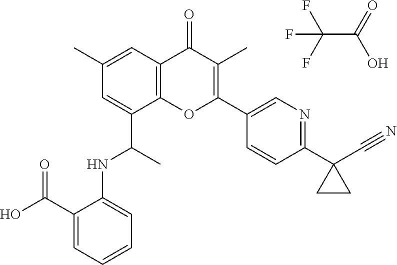 | 480 [M + H]⁺ |
| 90 | 2-[1-[2-[5-(4- Cyanophenyl)-1,3,4- oxadiazol-2-yl]-6- methyl-4-oxo- chromen-8- yl]ethylamino]benzoic acid, Isomer 2 | 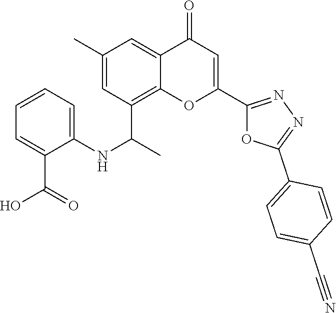 [SMILES: Cc1cc(C(C)Nc2ccccc2C(=O)O)c2oc(-c3nnc(-c4ccc(C#N)cc4)o3)cc(=O)c2c1] | 493 [M + H]⁺ |
| 91 | 2-[1-[2-[1-(2,2- Difluoro-1,3- benzodioxol-5- yl)pyrazol-4-yl]-6- methyl-4-oxo- chromen-8- yl]ethylamino]benzoic acid, Isomer 2, 2,2,2- trifluoroacetic acid | 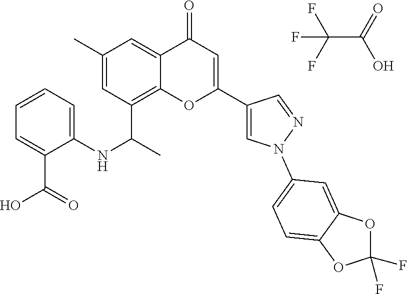 | 546 [M + H]⁺ |
| 92 | 2-[1-[2-[1-(3-Fluoro-4- methoxy- phenyl)pyrazol-4-yl]-6- methyl-4-oxo- chromen-8- yl]ethylamino]benzoic acid, Isomer 2, 2,2,2- trifluoroacetic acid | 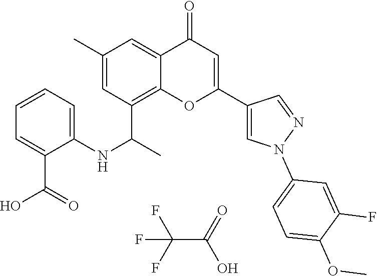 [SMILES: COc1ccc(-n2cc(-c3cc(=O)c4cc(C)cc(C(C)Nc5ccccc5C(=O)O)c4o3)cn2)cc1F ; O=C(O)C(F)(F)F] | 514 [M + H]⁺ |
| 93 | 2-[1-[6-Methyl-4-oxo- 2-[1-[4- (trifluoromethoxy) phenyl]pyrazol-4- yl]chromen-8- yl]ethylamino]benzoic acid, Isomer 2, 2,2,2- trifluoroacetic acid | 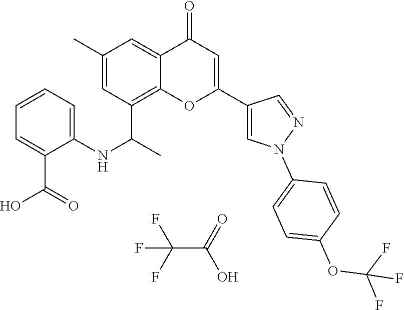 [SMILES: Cc1cc(C(C)Nc2ccccc2C(=O)O)c2oc(-c3cnn(-c4ccc(OC(F)(F)F)cc4)c3)cc(=O)c2c1 ; O=C(O)C(F)(F)F] | 550 [M + H]⁺ |
| 94 | 2-[1-[2-[1-(4-Cyano-3- methoxy- phenyl)pyrazol-4-yl]-6- methyl-4-oxo- chromen-8- yl]ethylamino]benzoic acid, Isomer 2, 2,2,2- trifluoroacetic acid | 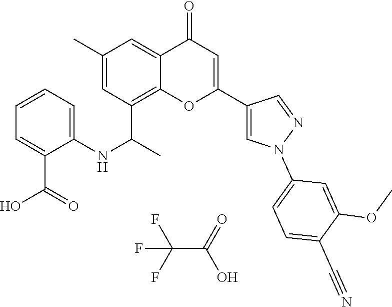 [SMILES: COc1cc(-n2cc(-c3cc(=O)c4cc(C)cc(C(C)Nc5ccccc5C(=O)O)c4o3)cn2)ccc1C#N ; O=C(O)C(F)(F)F] | 521 [M + H]⁺ |
| 95 | 2,3-Difluoro-6-[1-[2- [1-(4- cyanophenyl)pyrazol- 4-yl]-6-methyl-4-oxo- chromen-8- yl]ethylamino]benzoic acid, Isomer 1, 2,2,2- trifluoroacetic acid | 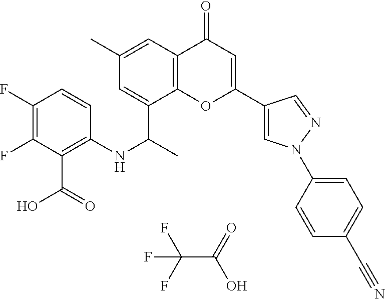 [SMILES: Cc1cc(C(C)Nc2ccc(F)c(F)c2C(=O)O)c2oc(-c3cnn(-c4ccc(C#N)cc4)c3)cc(=O)c2c1 ; O=C(O)C(F)(F)F] | 527 [M + H]⁺ |
| 96 | 6-Chloro-3-[1-[2-[1-(4- cyanophenyl)pyrazol- 4-yl]-4-oxo-6- (trifluoromethyl) chromen-8- yl]ethylamino]pyridine- 2-carboxylic acid, Isomer 1 | 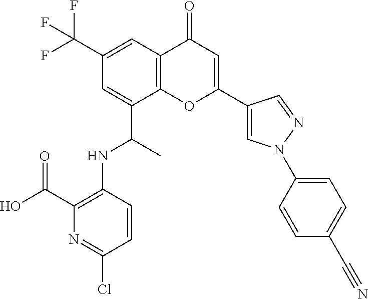 [SMILES: CC(Nc1ccc(Cl)nc1C(=O)O)c1cc(C(F)(F)F)cc2c(=O)cc(-c3cnn(-c4ccc(C#N)cc4)c3)oc12] | 580 [M + H]⁺ |
| 97 | 6-Chloro-3-[1-[2-(5- fluoro-3-pyridyl)-3- methyl-4-oxo-6- (trifluoromethyl) chromen-8- yl]ethylamino]pyridine- 2-carboxylic acid, Isomer 1 | 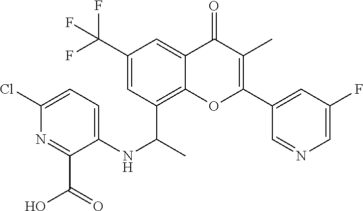 [SMILES: Cc1c(-c2cncc(F)c2)oc2c(C(C)Nc3ccc(Cl)nc3C(=O)O)cc(C(F)(F)F)cc2c1=O] | 522 [M + H]⁺ |
| 98 | 2-[1-2-[1-(3- Methoxyphenyl)triazol- 4-yl]-6-methyl-4-oxo- chromen-8- yl]ethylamino]benzoic acid, Isomer 2 | 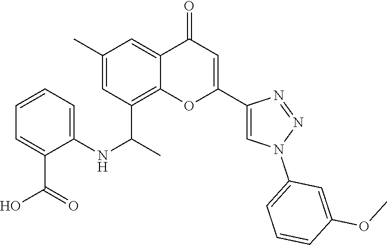 [SMILES: COc1cccc(-n2cc(-c3cc(=O)c4cc(C)cc(C(C)Nc5ccccc5C(=O)O)c4o3)nn2)c1] | 497 [M + H]⁺ |
| 99 | 2-[1-2-[1-(4- Cyanophenyl)pyrazol- 4-yl]-4-oxo-6- (trifluoromethyl) chromen-8- yl]ethylamino]benzoic acid, Isomer 1 | 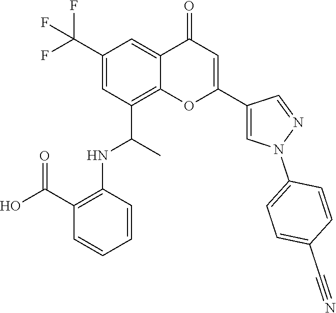 [SMILES: CC(Nc1ccccc1C(=O)O)c1cc(C(F)(F)F)cc2c(=O)cc(-c3cnn(-c4ccc(C#N)cc4)c3)oc12] | 545 [M + H]⁺ |
| 100 | 2-Fluoro-6-[1-[6- methyl-4-oxo-2-(2- pyridyl)chromen-8- yl]ethylamino]benzoic acid 2,2,2- trifluoroacetic acid | 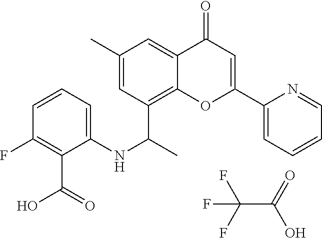 | 419 [M + H]⁺ |
| 101 | 2-[1-[2-[1-(4- Cyanophenyl)pyrazol- 4-yl]-6-methyl-4-oxo- chromen-8- yl]ethylamino]-6- fluoro-benzoic acid 2,2,2-trifluoroacetic acid | 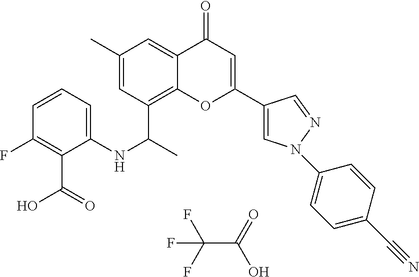 [SMILES: Cc1cc(C(C)Nc2cccc(F)c2C(=O)O)c2oc(-c3cnn(-c4ccc(C#N)cc4)c3)cc(=O)c2c1 ; O=C(O)C(F)(F)F] | 509 [M + H]⁺ |
| 102 | 2-[1-[2-[2-(3-Hydroxy- 3-methyl-but-1- ynyl)pyrimidin-5-yl]-6- methyl-4-oxo- chromen-8- yl]ethylamino]benzoic acid, Isomer 2, 2,2,2- trifluoroacetic acid | 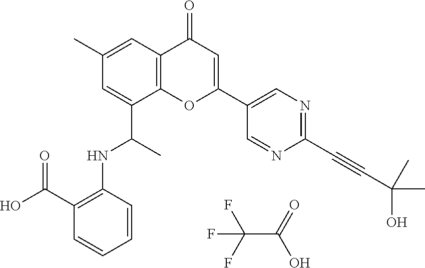 [SMILES: Cc1cc(C(C)Nc2ccccc2C(=O)O)c2oc(-c3cnc(C#CC(C)(C)O)nc3)cc(=O)c2c1 ; O=C(O)C(F)(F)F] | 484 [M + H]⁺ |
| 103 | 2-[1-[2-[1-(4- Cyanophenyl)imidazol- 4-yl]-6-methyl-4-oxo- chromen-8- yl]ethylamino]benzoic acid, Isomer 2, 2,2,2- trifluoroacetic acid | 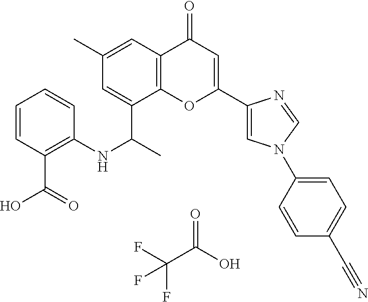 [SMILES: Cc1cc(C(C)Nc2ccccc2C(=O)O)c2oc(-c3cn(-c4ccc(C#N)cc4)cn3)cc(=O)c2c1 ; O=C(O)C(F)(F)F] | 491 [M + H]⁺ |
| 104 | 2-[1-[2-[1-(4-Cyano-2- fluoro-phenyl)pyrazol- 4-yl]-6-methyl-4-oxo- chromen-8- yl]ethylamino]benzoic acid, Isomer 2, 2,2,2- trifluoroacetic acid | 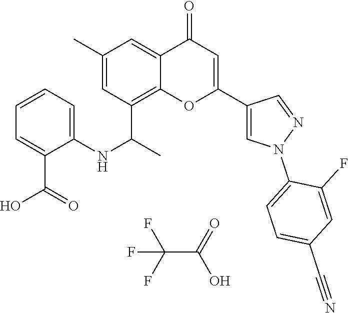 [SMILES: Cc1cc(C(C)Nc2ccccc2C(=O)O)c2oc(-c3cnn(-c4ccc(C#N)cc4F)c3)cc(=O)c2c1 ; O=C(O)C(F)(F)F] | 509 [M + H]⁺ |
| 105 | 2-[1-[2-[1-(2- Methoxypyrimidin-5- yl)pyrazol-4-yl]-6- methyl-4-oxo- chromen-8- yl]ethylamino]benzoic acid, Isomer 2, 2,2,2- trifluoroacetic acid | 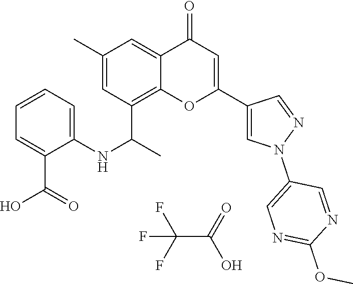 | 498 [M + H]⁺ |
| 106 | 2-[1-2-[1-(4- Chiorophenyl)pyrazol- 4-yl]-6-methyl-4-oxo- chromen-8- yl]ethylamino]benzoic acid, Isomer 2, 2,2,2- trifluoroacetic acid | 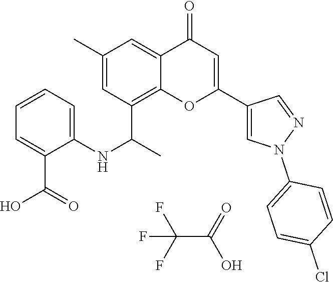 | 500 [M + H]⁺ |
| 107 | 2-[[(1R)-1-[2-[1-(2- Cyanophenyl)pyrazol- 4-yl]-6-methyl-4-oxo- chromen-8- yl]ethylamino]benzoic acid, Isomer 2, 2,2,2- trifluoroacetic acid | 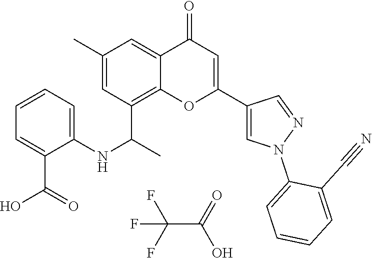 | 491 [M + H]⁺ |
| 108 | 2-[1-[6-Methyl-4-oxo- 2-[1-(2- pyridyl)pyrazol-4- yl]chromen-8- yl]ethylamino]benzoic acid, Isomer 2, 2,2,2- trifluoroacetic acid | 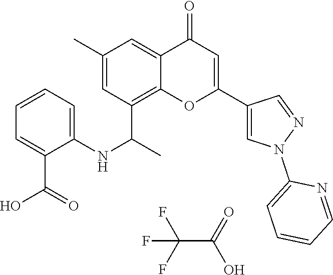 | 467 [M + H]⁺ |
| 109 | 2-[1-[2-[1-(4- Cyanophenyl)-3- methyl-pyrazol-4-yl]-6- methyl-4-oxo- chromen-8- yl]ethylamino]benzoic acid, Isomer 2, 2,2,2- trifluoroacetic acid | 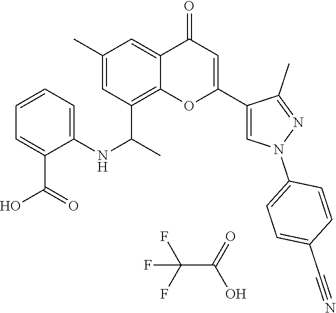 | 505 [M + H]⁺ |
| 110 | 2-Fluoro-6-[1-[6- methyl-4-oxo-2-(2- pyridyl)chromen-8- yl]ethylamino]benzoic acid, Isomer 1 | 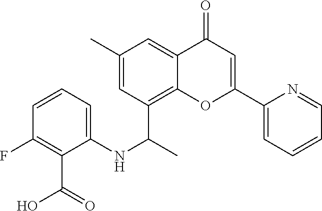 [SMILES: Cc1cc(C(C)Nc2cccc(F)c2C(=O)O)c2oc(-c3ccccn3)cc(=O)c2c1] | 419 [M + H]⁺ |
| 111 | 2-Fluoro-6-[1-[6- methyl-4-oxo-2-(2- pyridyl)chromen-8- yl]ethylamino]benzoic acid, Isomer 2 | 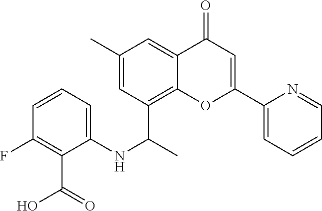 [SMILES: Cc1cc(C(C)Nc2cccc(F)c2C(=O)O)c2oc(-c3ccccn3)cc(=O)c2c1] | 419 [M + H]⁺ |
| 112 | 2-[1-[2-[1-(4- Cyanophenyl)pyrazol- 4-yl]-6-methyl-4-oxo- chromen-8- yl]ethylamino]-6- fluoro-benzoic acid, Isomer 1 | 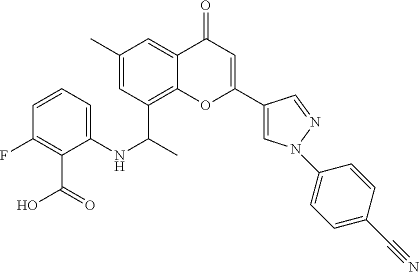 [SMILES: Cc1cc(C(C)Nc2cccc(F)c2C(=O)O)c2oc(-c3cnn(-c4ccc(C#N)cc4)c3)cc(=O)c2c1] | 509 [M + H]⁺ |
| 113 | 2-[1-2-[1-(4- Cyanophenyl)pyrazol- 4-yl]-6-methyl-4-oxo- chromen-8- yl]ethylamino]-6- fluoro-benzoic acid, Isomer 2 | 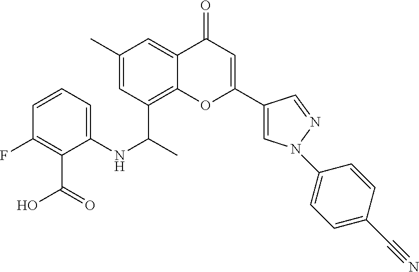 [SMILES: Cc1cc(C(C)Nc2cccc(F)c2C(=O)O)c2oc(-c3cnn(-c4ccc(C#N)cc4)c3)cc(=O)c2c1] | 509 [M + H]⁺ |
| 114 | 6-[1-[2-[1-(4- Cyanophenyl)pyrazol- 4-yl]-6-methyl-4-oxo- chromen-8- yl]ethylamino]-2,3- difluoro-benzoic acid, Isomer 1 | 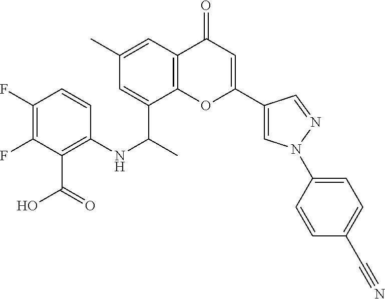 [SMILES: Cc1cc(C(C)Nc2ccc(F)c(F)c2C(=O)O)c2oc(-c3cnn(-c4ccc(C#N)cc4)c3)cc(=O)c2c1] | 527 [M + H]⁺ |
| 115 | 6-Chloro-3-[1-[3,6- dimethyl-4-oxo-2-(3- pyridyl)chromen-8- yl]ethylamino]pyridine- 2-carboxylic acid, Isomer 1 | 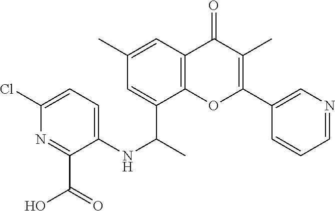 [SMILES: Cc1cc(C(C)Nc2ccc(Cl)nc2C(=O)O)c2oc(-c3cccnc3)c(C)c(=O)c2c1] | 450 [M + H]⁺ |
| 116 | 3-[1-[3,6-Dimethyl-4- oxo-2-(3- pyridyl)chromen-8- yl]ethylamino]pyridine- 2-carboxylic acid, Isomer 1 | 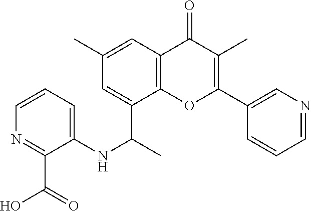 [SMILES: Cc1cc(C(C)Nc2cccnc2C(=O)O)c2oc(-c3cccnc3)c(C)c(=O)c2c1] | 416 [M + H]⁺ |
| 117 | 3-[1-[3,6-Dimethyl-4- oxo-2-(3- pyridyl)chromen-8- yl]ethylamino]-6- (trifluoromethyl) pyridine-2- carboxylic acid, Isomer 1 | 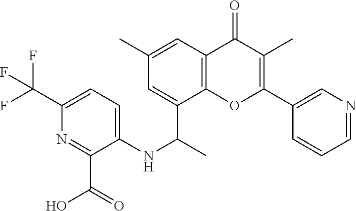 [SMILES: Cc1cc(C(C)Nc2ccc(C(F)(F)F)nc2C(=O)O)c2oc(-c3cccnc3)c(C)c(=O)c2c1] | 484 [M + H]⁺ |
| 118 | 3-[1-[3,6-Dimethyl-4- oxo-2-(3- pyridyl)chromen-8- yl]ethylamino]-6- methyl-pyridine-2- carboxylic acid, Isomer 1 | 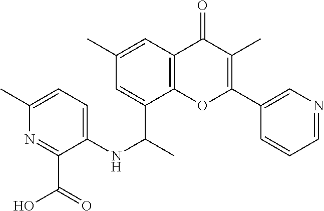 [SMILES: Cc1cc(C(C)Nc2ccc(C)nc2C(=O)O)c2oc(-c3cccnc3)c(C)c(=O)c2c1] | 430 [M + H]⁺ |
| 119 | 2-[1-[3,6-Dimethyl-4- oxo-2-(3- pyridyl)chromen-8- yl]ethylamino]-5- (trifluoromethyl) benzoic acid, Isomer 1 | 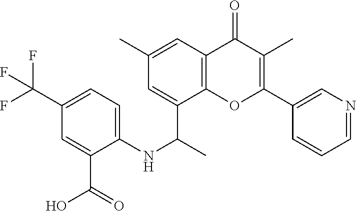 [SMILES: Cc1cc(C(C)Nc2ccc(C(F)(F)F)cc2C(=O)O)c2oc(-c3cccnc3)c(C)c(=O)c2c1] | 483 [M + H]⁺ |
| 120 | 2-[1-[3,6-Dimethyl-4- oxo-2-(3- pyridyl)chromen-8- yl]ethylamino]-5- fluoro-benzoic acid, Isomer 1 | 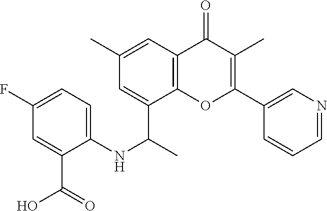 [SMILES: Cc1cc(C(C)Nc2ccc(F)cc2C(=O)O)c2oc(-c3cccnc3)c(C)c(=O)c2c1] | 433 [M + H]⁺ |
| 121 | 6-Chloro-3-[1-[3,6- dimethyl-4-oxo-2-(2- pyridyl)chromen-8- yl]ethylamino]pyridine- 2-carboxylic acid, Isomer 1, 2,2,2- trifluoroacetic acid | 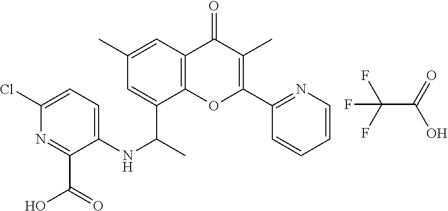 | 450 [M + H]⁺ |
| 122 | 3-[1-[3,6-Dimethyl-4- oxo-2-(2- pyridyl)chromen-8- yl]ethylamino]-6- methyl-pyridine-2- carboxylic acid, Isomer 1 | 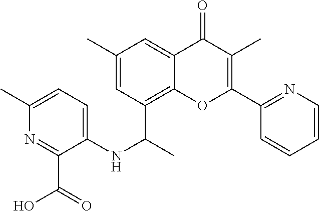 [SMILES: Cc1cc(C(C)Nc2ccc(C)nc2C(=O)O)c2oc(-c3ccccn3)c(C)c(=O)c2c1] | 430 [M + H]⁺ |
| 123 | 3-[1-[2-(5-Fluoro-3- pyridyl)-3,6-dimethyl- 4-oxo-chromen-8- yl]ethylamino]-6- methyl-pyridine-2- carboxylic acid, Isomer 1 | 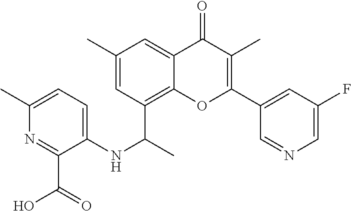 [SMILES: Cc1cc(C(C)Nc2ccc(C)nc2C(=O)O)c2oc(-c3cncc(F)c3)c(C)c(=O)c2c1] | 448 [M + H]⁺ |
| 124 | 2-[1-[2-[1-(4- Cyanophenyl)triazol-4- yl]-6-methyl-4-oxo- chromen-8- yl]ethylamino]benzoic acid, Isomer 2, 2,2,2- trifluoroacetic acid | 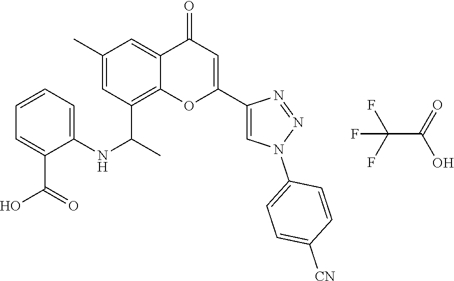 [SMILES: Cc1cc(C(C)Nc2ccccc2C(=O)O)c2oc(-c3cn(-c4ccc(C#N)cc4)nn3)cc(=O)c2c1 ; O=C(O)C(F)(F)F] | 492 [M + H]⁺ |
| 125 | 2-[1-[2-(5-Cyano-3- pyridyl)-3,6-dimethyl- 4-oxo-chromen-8- yl]ethylamino]benzoic acid, Isomer 1, 2,2,2- trifluoroacetic acid | 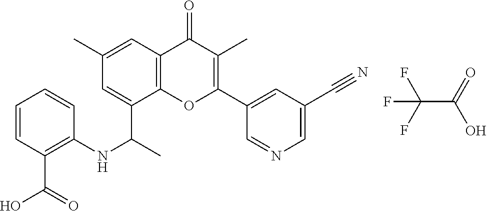 | 440 [M + H]⁺ |
| 126 | 2-[1-[3,6-Dimethyl-4- oxo-2-(2- phenylthiazol-5- yl)chromen-8- yl]ethylamino]benzoic acid, Isomer 1, 2,2,2- trifluoroacetic acid | 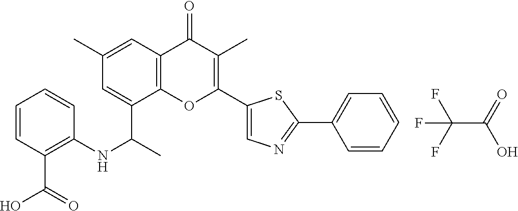 | 497 [M + H]⁺ |
| 127 | 2-[1-[3,6-Dimethyl-4- oxo-2-(5-pyrazol-1-yl- 3-pyridyl)chromen-8- yl]ethylamino]benzoic acid, Isomer 1, 2,2,2- trifluoroacetic acid | 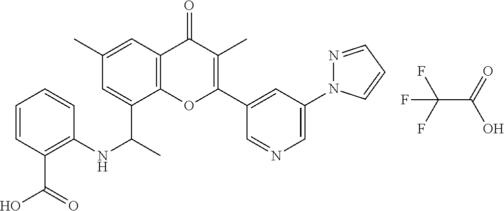 | 481 [M + H]⁺ |
| 128 | 6-[1-[3,6-Dimethyl-4- oxo-2-(2- pyridyl)chromen-8- yl]ethylamino]-2,3- difluoro-benzoic acid, Isomer 1 | 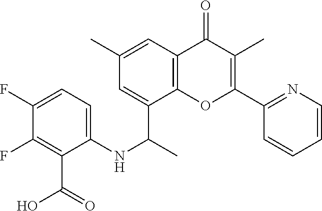 [SMILES: Cc1cc(C(C)Nc2ccc(F)c(F)c2C(=O)O)c2oc(-c3ccccn3)c(C)c(=O)c2c1] | 451 [M + H]⁺ |
| 129 | 6-Bromo-3-[1-[3,6- dimethyl-4-oxo-2-(3- pyridyl)chromen-8- yl]ethylamino]pyridine- 2-carboxylic acid, Isomer 1 | 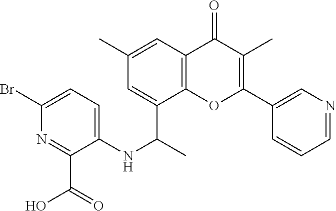 [SMILES: Cc1cc(C(C)Nc2ccc(Br)nc2C(=O)O)c2oc(-c3cccnc3)c(C)c(=O)c2c1] | 496 [M + H]⁺ |
| 130 | 2-[1-[2-[2- (Dimethylamino)thiazol- 5-yl]-3,6-dimethyl-4- oxo-chromen-8- yl]ethylamino]benzoic acid, Isomer 1 | 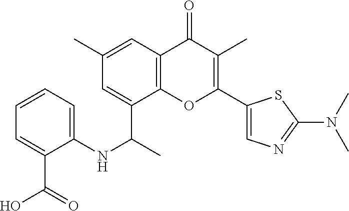 [SMILES: Cc1cc(C(C)Nc2ccccc2C(=O)O)c2oc(-c3cnc(N(C)C)s3)c(C)c(=O)c2c1] | 464 [M + H]⁺ |
| 131 | 3-[1-[2-(5-Fluoro-3- pyridyl)-3,6-dimethyl- 4-oxo-chromen-8- yl]ethylamino]pyridine- 2-carboxylic acid, Isomer 1 | 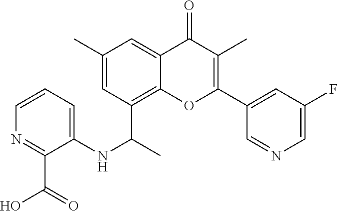 [SMILES: Cc1cc(C(C)Nc2cccnc2C(=O)O)c2oc(-c3cncc(F)c3)c(C)c(=O)c2c1] | 434 [M + H]⁺ |
| 132 | 5-Chloro-2-[1-[3,6- dimethyl-4-oxo-2-(3- pyridyl)chromen-8- yl]ethylamino]benzoic acid, Isomer 1 | 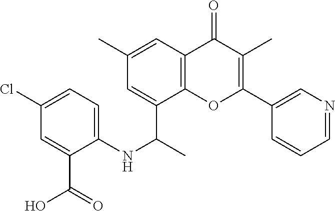 [SMILES: Cc1cc(C(C)Nc2ccc(Cl)cc2C(=O)O)c2oc(-c3cccnc3)c(C)c(=O)c2c1] | 449 [M + H]⁺ |
| 133 | 2-[1-[2-[1-(3,4- Dimethoxyphenyl) triazol-4-yl]-6-methyl-4- oxo-chromen-8- yl]ethylamino]benzoic acid, Isomer 2, 2,2,2- trifluoroacetic acid | 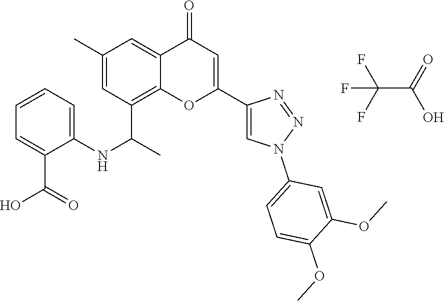 [SMILES: COc1ccc(-n2cc(-c3cc(=O)c4cc(C)cc(C(C)Nc5ccccc5C(=O)O)c4o3)nn2)cc1OC ; O=C(O)C(F)(F)F] | 527 [M + H]⁺ |
| 134 | 2-[1-[2-[3-(3,4- Dimethoxyphenyl) triazol-4-yl]-6-methyl-4- oxo-chromen-8- yl]ethylamino]benzoic acid, Isomer 2, 2,2,2- trifluoroacetic acid | 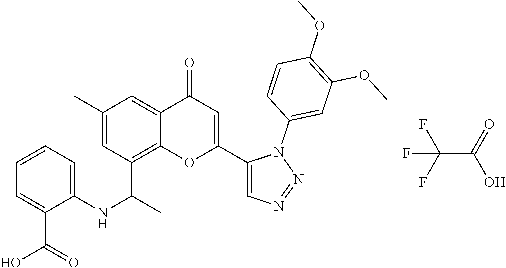 | 527 [M + H]⁺ |
| 135 | 2-[1-[2-[6-(1- Cyanocyclopropyl)-3- pyridyl]-3,6-dimethyl- 4-oxo-chromen-8- yl]ethylamino]benzoic acid, Isomer 1 | 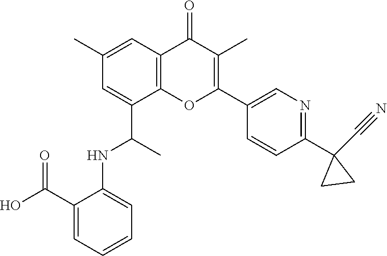 [SMILES: Cc1cc(C(C)Nc2ccccc2C(=O)O)c2oc(-c3ccc(C4(C#N)CC4)nc3)c(C)c(=O)c2c1] | 480 [M + H]⁺ |
| 136 | 2-[1-[3,6-Dimethyl-2- (1-methylpyrazol-4-yl)- 4-oxo-chromen-8- yl]ethylamino]benzoic acid, Isomer 1 | 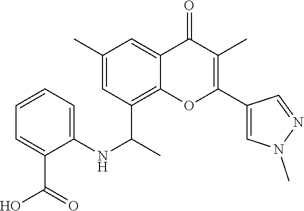 [SMILES: Cc1cc(C(C)Nc2ccccc2C(=O)O)c2oc(-c3cnn(C)c3)c(C)c(=O)c2c1] | 418 [M + H]⁺ |
| 137 | 3-[1-[2-[6- (Difluoromethyl)-2- pyridyl]-3,6-dimethyl- 4-oxo-chromen-8- yl]ethylamino]-6- methyl-pyridine-2- carboxylic acid, Isomer 1 | 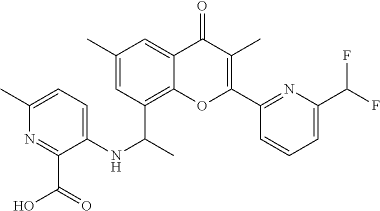 [SMILES: Cc1cc(C(C)Nc2ccc(C)nc2C(=O)O)c2oc(-c3cccc(C(F)F)n3)c(C)c(=O)c2c1] | 480 [M + H]⁺ |
| 138 | 6-Chloro-3-[1-[2-(5- fluoro-3-pyridyl)-3,6- dimethyl-4-oxo- chromen-8- yl]ethylamino]pyridine- 2-carboxylic acid, Isomer 1 | 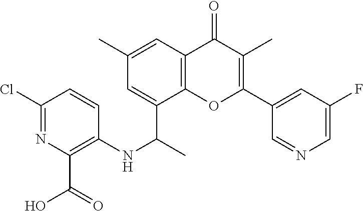 [SMILES: Cc1cc(C(C)Nc2ccc(Cl)nc2C(=O)O)c2oc(-c3cncc(F)c3)c(C)c(=O)c2c1] | 468 [M + H]⁺ |
| 139 | 3-[1-[2-[6- (Difluoromethyl)-2- pyridyl]-3,6-dimethyl- 4-oxo-chromen-8- yl]ethylamino]pyridine- 2-carboxylic acid; 2,2,2- trifluoroacetic acid, Isomer 1 | 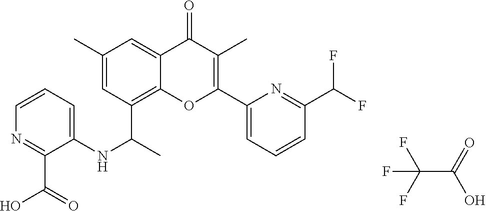 [SMILES: Cc1cc(C(C)Nc2cccnc2C(=O)O)c2oc(-c3cccc(C(F)F)n3)c(C)c(=O)c2c1 ; O=C(O)C(F)(F)F] | 466 [M + H]⁺ |
| 140 | 2-[1-[3,6-Dimethyl-4- oxo-2-(2- pyridyl)chromen-8- yl]ethylamino]-6- fluoro-benzoic acid, Isomer 1 | 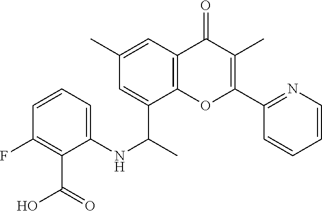 [SMILES: Cc1cc(C(C)Nc2cccc(F)c2C(=O)O)c2oc(-c3ccccn3)c(C)c(=O)c2c1] | 433 [M + H]⁺ |
| 141 | 6-Chloro-3-[1-[2-[6- (difluoromethyl)-2- pyridyl]-3,6-dimethyl- 4-oxo-chromen-8- yl]ethylamino]pyridine- 2-carboxylic acid, Isomer 1 | 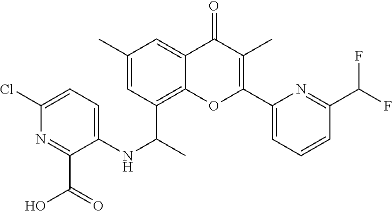 [SMILES: Cc1cc(C(C)Nc2ccc(Cl)nc2C(=O)O)c2oc(-c3cccc(C(F)F)n3)c(C)c(=O)c2c1] | 500 [M + H]⁺ |

### Example 57: 2-[1-[6-Methyl-4-oxo-2-(triazol-2-yl)chromen-8-yl]ethylamino]benzoic acid, Isomer 2, 2,2,2-trifluoroacetic acid

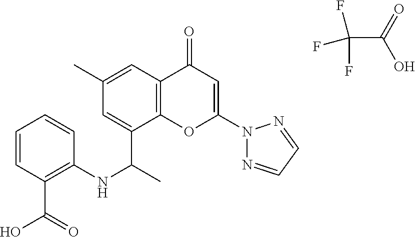 [SMILES: Cc1cc(C(C)Nc2ccccc2C(=O)O)c2oc(-n3nccn3)cc(=O)c2c1 ; O=C(O)C(F)(F)F]

1H-1,2,3-triazole (54.5 mg, 0.79 mmol) was dissolved in acetonitrile (2 mL), cooled to 0° C., and treated with sodium hydride (31.5 mg, 60 wt % in oil, 0.79 mmol). After stirring at 0° C. for 15 min, the suspension was treated with 2-[l-(2-ethylsulfinyl-6-methyl-4-oxo-chromen-8-yl)ethylamino]benzoic acid, Isomer 2 (150 mg, 0.38 mmol). The yellow suspension was stirred at room temperature. After 30 min, the suspension was treated with 1.5 mL of DMF to solubilize the suspension. After 5 min, the reaction was concentrated, dissolved in water (1 mL) and acetonitrile (2.3 mL), and purified using reverse phase (C-18 column, 10-100% acetonitrile[0.1% TFA] in water[0.1% TFA]) to give the product as the trifluoroacetate salt (30 mg, 15%). MS ES+m/z 391 [M+H]⁺.

The following compounds in Table 2 can be made according to Schemes 1-3 or the foregoing Examples.

TABLE 2

| Example | | | MS ES+ |
|---|---|---|---|
| # | Chemical Name | Structure | m/z |
| 58 | 2-[1-(6-Methyl-4-oxo- 2-pyrazol-1-yl- chromen-8- yl)ethylamino]benzoic acid, Isomer 2, 2,2,2- trifluoroacetic acid | 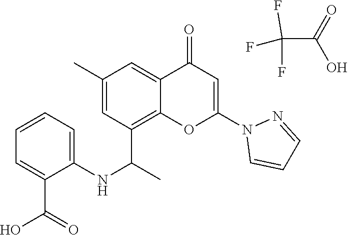 | 390 [M + H]⁺ |
| 59 | 2-[1-(2-Imidazol-1-yl- 6-methyl-4-oxo- chromen-8- yl)ethylamino]benzoic acid, Isomer 2, 2,2,2- trifluoroacetic acid | 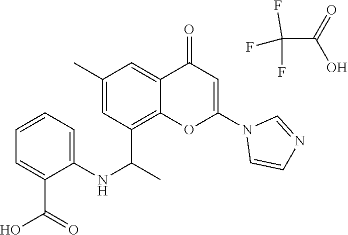 | 390 [M + H]⁺ |

### Example 142: 2-[[(1R)-1-[2-[6-(Difluoromethyl)-2-pyridyl]-3,6-dimethyl-4-oxo-chromen-8-yl]ethyl]amino]benzoic acid

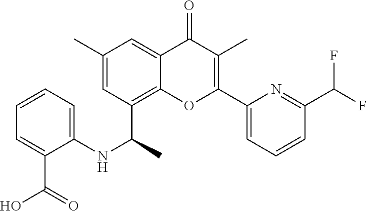 [SMILES: Cc1cc([C@@H](C)Nc2ccccc2C(=O)O)c2oc(-c3cccc(C(F)F)n3)c(C)c(=O)c2c1]

A solution of 8-[(1S)-1-chloroethyl]-2-[6-(difluoromethyl)-2-pyridyl]-3,6-dimethyl-chromen-4-one (0.20 g, 0.55 mmol) and 2-aminobenzoic acid (0.23 g, 1.65 mmol) in isopropanol (4 mL) was stirred at room temperature. Triethylamine (0.22 g, 2.20 mmol) was added. The reaction was stirred at reflux for 2 h, cooled to room temperature, and concentrated. Diluted with DCM (20 mL) and 0.1M aqueous hydrochloric acid (10 mL). Separated the layers. The organics were washed with brine and concentrated. The residue was purified by silica column (20:1 DCM:MeOH) to give the product (0.15 g, 60%). MS ES+ m/z 465 [M+H]⁺.

TABLE 3

| Ex # | NMR Line Listing |
|---|---|
| 1 | ¹H NMR (400 MHz, CHLOROFORM-d) δ ppm 1.42 (br d, J = 5.4 Hz, 6H), 1.78 (br d, |
| | J = 6.0 Hz, 3H), 2.42 (s, 3H), 5.32 (br s, 1H), 5.38-5.47 (m, 1H), 6.41 (br d, J = 8.6 Hz, |
| | 1H), 6.66 (br t, J = 7.2 Hz, 1H), 6.83 (s, 1H), 6.86 (br d, J = 8.6 Hz, 1H), 7.23-7.27 (m, |
| | 1H), 7.56 (br s, 1H), 7.96 (br s, 1H), 8.06 (br d, J = 8.4 Hz, 2H), 8.24 (br s, 1H), 8.84 (br |
| | s, 1H) |
| 2 | ¹H NMR (400 MHz, DMSO-d6) δ ppm 1.53-1.66 (m, 3H), 2.24-2.35 (m, 3H), 3.97 |
| | (br s, 3H), 5.31 (br s, 1H), 6.44-6.53 (m, 2H), 7.07-7.21 (m, 2H), 7.42-7.53 (m, |
| | 1H), 7.65-7.71 (m, 1H), 7.71-7.79 (m, 1H), 8.28-8.45 (m, 1H), 9.25 (s, 1H), 9.18- |
| | 9.35 (m, 1H), 12.54-12.93 (m, 1H) |
| 3 | ¹H NMR (400 MHz, DMSO-d6) δ ppm 1.70 (d, J = 6.4 Hz, 3H), 2.37 (s, 3H), 5.34-5.35 |
| | (m, 1H), 6.50-6.56 (m, 2H), 7.16-7.18 (m, 1H), 7.34 (s, 1H), 7.61 (d, J = 2.0 Hz, 1H), |
| | 7.71 (t, J = 4.8 Hz, 1H), 7.76-7.80 (m, 2H), 8.48 (s, 1H), 9.09 (d, J = 4.8 Hz, 2H) |
| 6 | ¹H NMR (400 MHz, DMSO-d6) δ ppm 1.35-1.40 (m, 6H), 1.66 (s, 3H), 2.36 (s, 3H), |
| | 5.29-5.41 (m, 2H), 6.55 (d, J = 7.9 Hz, 2H), 7.17 (s, 1H), 7.22 (s, 1H), 7.54 (s, 1H), |
| | 7.75 (s, 1H), 7.81 (d, J = 7.9 Hz, 1H), 8.43 (s, 1H), 9.30 (s, 2H), 12.79 (s, 1H) |
| 7 | ¹H NMR (400 MHz, CHLOROFORM-d) δ ppm 1.72-1.73 (m, 3H), 2.41 (s, 3H), 3.30 |
| | (s, 3H), 5.14 (d, J = 4.8 Hz, 1H), 6.37 (d, J = 7.9 Hz, 1H), 6.59 (t, J = 7.4 Hz, 1H), 6.96 (s, |
| | 1H), 7.16-7.20 (m, 1H), 7.60 (s, 1H), 7.91 (s, 1H), 7.96 (d, J = 7.5 Hz, 1H), 8.24 (d, |
| | J = 7.9 Hz, 1H), 8.50 (d, J = 7.7 Hz, 1H), 9.26 (s, 1H) |
| 8 | ¹H NMR (400 MHz, CHLOROFORM-d) δ ppm 1.75 (s, 3H), 2.42 (s, 3H), 3.38 (s, |
| | 1H), 5.23 (s, 1H), 6.33-6.41 (m, 1H), 6.61 (s, 1H), 7.00 (s, 1H), 7.34-7.39 (m, 1H), |
| | 7.63 (s, 1H), 7.91 (s, 2H), 8.00 (s, 1H), 8.48 (s, 1H), 9.32 (s, 1H) |
| 9 | ¹H NMR (400 MHz, DMSO-d6) δ ppm 1.61-1.72 (m, 3H), 2.37 (s, 3H), 5.36 (s, 1H), |
| | 6.55 (d, J = 8.07 Hz, 2H), 7.22 (s, 2H), 7.44 (d, J = 8.44 Hz, 1H), 7.56 (s, 1H), 7.74-7.84 |
| | (m, 2H), 8.43 (s, 1H), 8.71 (t, J = 7.82 Hz, 1H), 9.03 (s, 1H), 12.78 (s, 1H) |
| 10 | ¹H NMR (400 MHz, DMSO-d6) δ ppm 1.55-1.66 (m, 3H), 2.25-2.36 (m, 3H), 5.48- |
| | 5.56 (m, 1H), 6.48 (br d, J = 7.46 Hz, 2H), 7.15 (br s, 1H), 7.22 (br s, 1H), 7.51 (br s, |
| | 1H), 7.71 (br s, 1H), 7.75 (br d, J = 7.09 Hz, 1H), 8.07 (br s, 1H), 8.35 (br s, 1H), 8.60 |
| | (br s, 1H), 8.98 (br s, 1H), 12.70 (br s, 1H) |
| 11 | ¹H NMR (400 MHz, DMSO-d6) δ ppm 1.63-1.78 (m, 3H), 2.30-2.43 (m, 3H), 3.99 |
| | (s, 3H), 5.29-5.45 (m, 1H), 6.45-6.66 (m, 2H), 7.04-7.11 (m, 1H), 7.20-7.29 (m, |
| | 2H), 7.54-7.64 (m, 1H), 7.73-7.78 (m, 1H), 7.78-7.84 (m, 1H), 7.84-7.91 (m, 1H), |
| | 7.91-8.00 (m, 1H), 8.38-8.61 (m, 1H), 12.57-12.97 (m, 1H) |
| 12 | ¹H NMR (400 MHz, DMSO-d6) δ ppm 1.67 (br d, J = 5.75 Hz, 3 H), 2.28-2.41 (m, 3 |
| | H), 3.97 (br s, 3 H), 5.25-5.46 (m, 1 H), 6.46-6.67 (m, 2 H), 6.95-7.06 (m, 1 H), |
| | 7.06-7.13 (m, 1 H), 7.20-7.34 (m, 1 H), 7.47-7.61 (m, 1 H), 7.70-7.80 (m, 1 H), |
| | 7.80-7.89 (m, 1 H), 8.35-8.51 (m, 2 H), 8.92-9.05 (m, 1 H), 12.59-12.92 (m, 1 H) |
| 13 | ¹H NMR (400 MHz, DMSO-d6) δ ppm 1.60 (br d, J = 5.75 Hz, 3H), 2.29 (br s, 3H), |
| | 2.67 (br s, 3H), 5.14-5.35 (m, 1H), 6.49 (br d, J = 7.3 Hz, 2H), 7.12-7.22 (m, 2H), |
| | 7.47-7.52 (m, 1H), 7.67-7.71 (m, 1H), 7.72-7.78 (m, 1H), 8.24-8.49 (m, 1H), 9.23- |
| | 9.42 (m, 2H), 12.52-12.94 (m, 1H) |
| 14 | ¹H NMR (400 MHz, DMSO-d6) δ ppm 1.67 (d, J = 6.4 Hz, 3H), 2.36 (s, 3H), 5.34-5.35 |
| | (m, 1H), 6.50-6.56 (m, 2H), 7.18-7.19 (m, 1H), 7.21 (s, 1H), 7.56 (d, J = 2.0 Hz, 1H), |
| | 7.62 (dd, J = 7.6, 4.8 Hz, 1H), 7.76 (s, 1H), 7.81 (d, J = 8.0 Hz, 1H), 8.50-8.52 (m, 1H), |
| | 8.55 (s, 1H), 8.78 (d, J = 4.8 Hz, 1H), 9.32 (d, J = 2.0 Hz, 1H) |
| 15 | ¹H NMR (400 MHz, DMSO-d6) δ ppm 1.70 (d, J = 6.4 Hz, 3H), 2.37 (s, 3H), 5.40-5.44 |
| | (m, 1H), 6.53-6.58 (m, 2H), 7.19-7.25 (m, 2H), 7.59 (s, 1H), 7.78-7.82 (m, 2H), 8.48 |
| | (d, J = 5.6 Hz, 1H), 8.89-8.91 (m, 2H), 9.45 (s, 1H), 12.76 (s, 1H) |
| 16 | ¹H NMR (400 MHz, DMSO-d6) δ ppm 1.70 (d, J = 6.4 Hz, 3H), 2.37 (s, 3H), 5.41-5.44 |
| | (m, 1H), 6.53-6.58 (m, 2H), 7.20-7.25 (m, 2H), 7.59 (d, J = 2.0 Hz, 1H), 7.78-7.82 (m, |
| | 2H), 8.47 (d, J = 6.0 Hz, 1H), 8.89-8.91 (m, 2H), 9.45 (s, 1H), 12.75 (s, 1H) |
| 17 | ¹H NMR (400 MHz, DMSO-d6) δ ppm 1.69 (d, J = 6.8 Hz, 3H), 2.37 (s, 3H), 5.37-5.39 |
| | (s, 1H), 6.53-6.57 (m, 2H), 7.21-7.23 (m, 1H), 7.24 (s, 1H), 7.58 (d, J = 2.0 Hz, 1H), |
| | 7.64 (dd, J = 6.8, 4.4 Hz, 1H), 7.77 (s, 1H), 7.81 (d, J = 8.0 Hz, 1H), 8.05-8.08 (m, 1H), |
| | 8.26 (d, J = 7.6 Hz, 1H), 8.50 (s, 1H), 8.80 (d, J = 4.0 Hz, 1H), 12.79 (br s, 1H) |
| 18 | ¹H NMR (400 MHz, DMSO-d6) δ ppm 1.69 (d, J = 6.8 Hz, 3H), 2.37 (s, 3H), 5.37-5.39 |
| | (m, 1H), 6.53-6.57 (m, 2H), 7.21-7.24 (m, 1H), 7.24 (s, 1H), 7.58 (d, J = 2.0 Hz, 1H), |
| | 7.63-7.65 (m, 1H), 7.77 (s, 1H), 7.80-7.82 (m, 1H), 8.05-8.08 (m, 1H), 8.26 (d, J = 8.0 |
| | Hz, 1H), 8.51 (s, 1H), 8.80 (d, J = 4.0 Hz, 1H), 13.09 (br s, 1H) |
| 21 | ¹H NMR (400 MHz, DMSO-d6) δ ppm 1.63-1.78 (m, 3H), 2.30-2.43 (m, 3H), 3.99 |
| | (s, 3H), 5.29-5.45 (m, 1H), 6.45-6.66 (m, 2H), 7.04-7.11 (m, 1H), 7.20-7.29 (m, |
| | 2H), 7.54-7.64 (m, 1H), 7.73-7.78 (m, 1H), 7.78-7.84 (m, 1H), 7.84-7.91 (m, 1H), |
| | 7.91-8.00 (m, 1H), 8.38-8.61 (m, 1H), 12.57-12.97 (m, 1H) |
| 22 | ¹H NMR (400 MHz, DMSO-d6) δ ppm 1.63-1.78 (m, 3H), 2.30-2.43 (m, 3H), 3.99 |
| | (s, 3H), 5.29-5.45 (m, 1H), 6.45-6.66 (m, 2H), 7.04-7.11 (m, 1H), 7.20-7.29 (m, |
| | 2H), 7.54-7.64 (m, 1H), 7.73-7.78 (m, 1H), 7.78-7.84 (m, 1H), 7.84-7.91 (m, 1H), |
| | 7.91-8.00 (m, 1H), 8.38-8.61 (m, 1H), 12.57-12.97 (m, 1H) |
| 23 | ¹H NMR (400 MHz, DMSO-d6) δ ppm 1.69 (d, J = 6.72 Hz, 3H), 2.36-2.39 (m, 3H), |
| | 5.36-5.44 (m, 1H), 6.52-6.59 (m, 2H), 7.19 (s, 1H), 7.23 (t, J = 7.27 Hz, 1H), 7.58 (d, |
| | J = 1.96 Hz, 1H), 7.76-7.79 (m, 1H), 7.81-7.83 (m, 1H), 7.97-8.02 (m, 1H), 8.35- |
| | 8.38 (m, 1H), 8.45 (br d, J = 6.24 Hz, 1H), 8.84 (d, J = 2.81 Hz, 1H), 12.77 (br s, 1H) |
| 24 | ¹H NMR (400 MHz, DMSO-d6) δ ppm 1.62 (d, J = 6.8 Hz, 3H), 2.25 (s, 3H), 2.37 (s, |
| | 3H), 5.18-5.21 (m, 1H), 6.48-6.56 (m, 2H), 7.20 (t, J = 7.2 Hz, 1H), 7.55 (d, J = 2.0 Hz, |
| | 1H), 7.58-7.60 (m, 1H), 7.79-7.81 (m, 2H), 8.05 (d, J = 4.0 Hz, 2H), 8.36 (d, J = 6.0 Hz, |
| | 1H), 8.81 (d, J = 4.4 Hz, 1H), 12.78 (s, 1H) |
| 25 | ¹H NMR (400 MHz, DMSO-d6) δ ppm 1.59 (d, J = 6.4 Hz, 3H), 2.09 (s, 3H), 2.37 (s, |
| | 3H), 5.12-5.15 (m, 1H), 6.47 (d, J = 8.4 Hz, 1H), 6.55 (t, J = 7.6 Hz, 1H), 7.20 (t, J = 7.2 |
| | Hz, 1H), 7.53-7.54 (m, 1H), 7.64-7.66 (m, 1H), 7.79-7.81 (m, 2H), 8.24 (d, J = 8.2 Hz, |
| | 1H), 8.35 (d, J = 6.0 Hz, 1H), 8.78 (s, 1H), 9.03 (s, 1H), 12.71 (s, 1H) |
| 26 | ¹H NMR (400 MHz, DMSO-d6) δ ppm 1.12-1.19 (m, 4H), 1.65 (d, J = 5.6 Hz, 3H), |
| | 2.30-2.33 (m, 1H), 2.35 (s, 3H), 5.34-5.35 (m, 1H), 6.51-6.55 (m, 2H), 7.17-7.20 (m, |
| | 2H), 7.55 (s, 1H), 7.74 (s, 1H), 7.81 (d, J = 6.4 Hz, 1H), 8.52 (s, 1H), 9.28-9.30 (m, 2H) |
| 27 | ¹H NMR (400 MHz, CHLOROFORM-d) δ ppm 1.83 (d, J = 6.4 Hz, 3H), 2.41 (s, 3H), |
| | 5.23-5.29 (m, 1H), 6.86 (d, J = 9.2 Hz, 1H), 7.17 (d, J = 8.8 Hz, 1H), 7.47-7.49 (s, 3H), |
| | 7.91-7.93 (m, 1H), 7.95 (s, 1H), 7.98-8.04 (m, 1H), 8.34 (d, J = 5.6 Hz, 1H), 8.79 (d, |
| | J = 4.0 Hz, 1H) |
| 28 | ¹H NMR (400 MHz, DMSO-d6) δ ppm 1.68 (d, J = 6.8 Hz, 3H), 2.36 (s, 3H), 5.39-5.40 |
| | (m, 1H), 6.98 (d, J = 8.8 Hz, 2H), 7.13 (d, J = 8.4 Hz, 1H), 7.24 (s, 1H), 7.57 (s, 1H), 7.77 |
| | (s, 1H), 8.88-8.89 (m, 1H), 8.90-8.91 (m, 1H), 9.23 (s, 1H), 9.47 (s, 1H) |
| 29 | ¹H NMR (400 MHz, DMSO-d6) δ ppm 1.60 (d, J = 6.4 Hz, 3H), 2.37 (s, 3H), 2.86 (s, |
| | 3H), 5.19-5.22 (m, 1H), 6.43 (d, J = 8.4 Hz, 1H), 6.53 (t, J = 7.6 Hz, 1H), 6.93 (s, 1H), |
| | 7.15-7.20 (m, 1H), 7.59 (d, J = 2.0 Hz, 1H), 7.78-7.80 (m, 2H), 8.41 (s, 1H), 8.70 (d, |
| | J = 2.0 Hz, 1H), 8.75 (d, J = 2.4 Hz, 1H) |
| 34 | ¹H NMR (400 MHz, DMSO-d6) δ ppm 1.65 (d, J = 6.8 Hz, 3H), 2.22 (s, 3H), 2.38 (s, |
| | 3H), 5.15-5.18 (m, 1H), 6.49-6.53 (m, 2H), 7.16-7.20 (m, 1H), 7.59 (d, J = 2.0 Hz, 1H), |
| | 7.70 (t, J = 4.8 Hz, 1H), 7.75-7.78 (m, 2H), 8.38 (d, J = 6.0 Hz, 1H), 9.10 (d, J = 4.8 Hz, |
| | 2H) |
| 43 | ¹H NMR (400 MHz, DMSO-d6) δ ppm 1.64 (d, J = 6.72 Hz, 3H), 2.37 (s, 3H), 4.10 (s, |
| | 3H), 5.19-5.27 (m, 1H), 6.47 (d, J = 8.19 Hz, 1H), 6.53-6.58 (m, 1H), 6.95 (s, 1H), |
| | 7.20-7.25 (m, 1H), 7.52-7.58 (m, 2H), 7.76-7.84 (m, 2H), 8.37-8.43 (m, 1H), 8.77 |
| | (br d, J = 6.24 Hz, 1H), 9.03 (s, 1H), 12.82 (br s, 1H) |
| 44 | ¹H NMR (400 MHz, DMSO-d6) δ ppm 1.67 (d, J = 6.60 Hz, 3H), 2.36 (s, 3H), 5.35- |
| | 5.44 (m, 1H), 6.50-6.59 (m, 2H), 7.17-7.25 (m, 1H), 7.29 (s, 1H), 7.57 (d, J = 2.08 |
| | Hz, 1H), 7.76 (d, J = 1.34 Hz, 1H), 7.81 (dd, J = 8 13, 1.65 Hz, 1H), 8.41 (br d, J = 6.48 |
| | Hz, 1H), 8.43-8.48 (m, 1H), 8.82 (d, J = 2.69 Hz, 1H), 9.18-9.23 (m, 1H), 12.75 (br s, |
| | 1H) |
| 45 | ¹H NMR (400 MHz, DMSO-d6) δ ppm 1.57 (d, J = 6.4 Hz, 3H), 2.08 (s, 3H), 2.36 (s, |
| | 3H), 5.13-5.14 (m, 1H), 6.44 (d, J = 8.0 Hz, 1H), 6.50 (t, J = 7.6 Hz, 1H), 7.14 (t, J = 8.0 |
| | Hz, 1H), 7.54 (d, J = 2.4 Hz, 1H), 7.77-7.79 (m, 2H), 8.21-8.25 (m, 1H), 8.59 (s, 1H), |
| | 8.81 (d, J = 2.8 Hz, 1H), 8.90 (s, 1H) |
| 46 | ¹H NMR (400 MHz, DMSO-d6) δ ppm 1.72 (d, J = 6.6 Hz, 3H), 5.43 (br t, J = 6.1 Hz, |
| | 1H), 6.53 (d, J = 8.4 Hz, 1H), 6.58 (t, J = 7.5 Hz, 1H), 7.22 (t, J = 7.8 Hz, 1H), 7.28 (s, |
| | 1H), 7.56 (dd, J = 8.9, 3.0 Hz, 1H), 7.63-7.68 (m, 2H), 7.83 (d, J = 7.8 Hz, 1H), 8.09 (t, |
| | J = 7.8 Hz, 1H), 8.30 (d, J = 7.9 Hz, 1H), 8.39-8.51 (m, 1H), 8.82 (d, J = 4.6 Hz, 1H), |
| | 12.85 (br s, 1H) |
| 47 | ¹H NMR (400 MHz, DMSO-d6) δ ppm 1.64 (br d, J = 6.5 Hz, 3H), 2.35 (s, 3H), 3.94 (s, |
| | 3H), 5.33 (br s, 1H), 6.50-6.54 (m, 1H), 6.55-6.59 (m, 1H), 6.72-6.77 (m, 1H), 7.24 (t, |
| | J = 7.8 Hz, 1H), 7.52 (s, 1H), 7.71 (s, 1H), 7.82 (d, J = 7.9 Hz, 1H), 8.21 (s, 1H), 8.43 (br |
| | d, J = 5.1 Hz, 1H), 8.53-8.59 (m, 1H), 12.77 (br s, 1H) |
| 48 | ¹H NMR (400 MHz, DMSO-d6) δ ppm 1.66 (br d, J = 5 14 Hz, 3H), 2.36 (br s, 3H), |
| | 5.45-5.53 (m, 1H), 6.55 (br d, J = 0.98 Hz, 2H), 6.95 (br s, 1H), 7.20-7.27 (m, 1H), |
| | 7.54 (br s, 1H), 7.61-7.68 (m, 1H), 7.71-7.76 (m, 1H), 7.79-7.85 (m, 1H), 8.35 (br |
| | s, 1H), 8.41-8.47 (m, 1H), 8.63 (br d, J = 0.98 Hz, 2H), 9.22-9.27 (m, 1H), 9.36-9.41 |
| | (m, 1H), 12.84 (br s, 1H) |
| 51 | ¹H NMR (400 MHz, DMSO-d6) δ ppm 1.57 (d, J = 6.60 Hz, 3H), 2.28 (s, 3H), 5.23- |
| | 5.31 (m, 1H), 6.43-6.51 (m, 2H), 6.73 (s, 1H), 7.13-7.19 (m, 1H), 7.42-7.46 (m, |
| | 1H), 7.63 (s, 1H), 7.74 (d, J = 7.95 Hz, 1H), 8.33-8.38 (m, 2H), 12.70 (br s, 1H) |
| 52 | ¹H NMR (400 MHz, CHLOROFORM-d) δ ppm 1.69 (d, J = 6.7 Hz, 3H), 2.34 (s, 3H), |
| | 5.12 (q, J = 6.7 Hz, 1H), 6.37 (d, J = 8.6 Hz, 1H), 6.57 (t, J = 7.6 Hz, 1H), 6.63 (s, 1H), |
| | 7.20-7.23 (m, 1H), 7.47 (d, J = 1.8 Hz, 1H), 7.86 (s, 1H), 7.94 (dd, J = 8.0, 1.3 Hz, 1H), |
| | 8.02 (s, 1H), 8.24 (s, 1H) |
| 53 | ¹H NMR (400 MHz, DMSO-d6) δ ppm 1.59 (d, J = 6.60 Hz, 3H), 2.29 (s, 3H), 3.79 (s, |
| | 3H), 5.34-5.43 (m, 1H), 6.45-6.53 (m, 2H), 6.85 (s, 1H), 6.91-6.93 (m, 1H), 7.12- |
| | 7.21 (m, 1H), 7.38-7.49 (m, 4H), 7.66 (s, 1H), 7.74 (d, J = 7.47 Hz, 1H), 8.36 (br d, |
| | J = 5.26 Hz, 1H), 8.45 (s, 1H), 9.21 (s, 1H), 12.69 (br s, 1H) |
| 54 | ¹H NMR (400 MHz, DMSO-d6) δ ppm 1.59 (br d, J = 6.48 Hz, 3H), 2.29 (s, 3H), 5.35- |
| | 5.43 (m, 1H), 6.43-6.51 (m, 2H), 6.87 (s, 1H), 7.17 (t, J = 7.08 Hz, 1H), 7.47 (s, 1H), |
| | 7.66 (s, 1H), 7.69-7.78 (m, 2H), 7.83 (br d, J = 7.58 Hz, 1H), 8.25 (br d, J = 8.31 Hz, |
| | 1H), 8.37 (br s, 2H), 8.55 (s, 1H), 9.30 (s, 1H), 12.74 (br s, 1H) |
| 55 | ¹H NMR (400 MHz, DMSO-d6) δ ppm 1.69 (d, J = 6.48 Hz, 3H), 5.49-5.59 (m, 1H), |
| | 6.50-6.61 (m, 2H), 7.02 (s, 1H), 7.22-7.28 (m, 1H), 7.50-7.54 (m, 1H), 7.55-7.68 |
| | (m, 2H), 7.84 (d, J = 7.82 Hz, 1H), 8.35-8.47 (m, 2H), 8.62-8.70 (m, 2H), 9.25 (d, |
| | J = 2.45 Hz, 1H), 9.42 (s, 1H), 12.86 (br s, 1H) |
| 56 | ¹H NMR (400 MHz, DMSO-d6) δ ppm 1.64 (d, J = 6.8 Hz, 3H), 2.33 (s, 3H), 3.84 (s, |
| | 3H), 5.38-5.45 (m, 1H), 6.54-6.60 (m, 2H), 6.84 (s, 1H), 7.24-7.26 (m, 1H), 7.51 (d, |
| | J = 2.0 Hz, 1H), 7.72 (s, 1H), 7.82 (d, J = 8.0, 1H), 8.15 (s, 1H), 8.22 (d, J = 6.4 Hz, 1H), |
| | 8.30 (s, 1H) |
| 57 | ¹H NMR (400 MHz, DMSO-d6) δ ppm 1.69 (d, J = 6.11 Hz, 3H), 2.37 (s, 3H), 5.19- |
| | 5.29 (m, 1H), 6.49-6.58 (m, 2H), 6.83 (s, 1H), 7.15-7.24 (m, 1H), 7.56-7.61 (m, |
| | 1H), 7.74-7.78 (m, 1H), 7.80 (d, J = 7.70 Hz, 1H), 8.41-8.47 (m, 3H), 12.76 (br s, 1H) |
| 58 | ¹H NMR (400 MHz, CHLOROFORM-d) δ ppm 1.79 (br d, J = 6.7 Hz, 3H), 2.44 (s, |
| | 3H), 5.20 (q, J = 6.4 Hz, 1H), 6.43 (d, J = 8.6 Hz, 1H), 6.62 (s, 1H), 6.68 (t, J = 7.5 Hz, |
| | 1H), 6.99 (s, 1H), 7.56 (s, 1H), 7.89 (s, 1H), 7.98 (s, 1H), 8.05 (br d, J = 7.9 Hz, 1H), |
| | 8.22 (s, 2H) |
| 59 | ¹H NMR (400 MHz, DMSO-d6) δ ppm 1.64 (br d, J = 6.4 Hz, 3H), 2.36 (s, 3H), 5.34 (br |
| | s, 1H), 6.51-6.60 (m, 2H), 6.94 (s, 1H), 7.34 (s, 1H), 7.22 (br t, J = 7.6 Hz, 1H), 7.55 (s, |
| | 1H), 7.75 (s, 1H), 7.82 (br d, J = 7.9 Hz, 1H), 8.10 (s, 1H), 8.40 (br s, 1H), 8.78 (s, 1H), |
| | 12.45-13.08 (m, 1H) |
| 60 | ¹H NMR (400 MHz, DMSO-d6) δ ppm 1.63 (d, J = 6.4 Hz, 3H), 2.23 (s, 3H), 2.37 (s, |
| | 3H), 5.19-5.23 (m, 1H), 7.08 (d, J = 8.8 Hz, 1H), 7.27 (d, J = 8.8 Hz, 1H), 7.55 (s, 1H), |
| | 7.57-7.60 (m, 1H), 7.79 (s, 1H), 8.04-8.05 (m, 2H), 8.36 (d, J = 5.6 Hz, 1H), 8.79 (d, |
| | J = 4.4 Hz, 1H) |
| 61 | ¹H NMR (500.11 MHz, DMSO-d6) δ ppm 1.68 (d, J = 6.6 Hz, 3H), 2.31 (s, 3H), 5.30- |
| | 5.23 (m, 1H), 7.08-7.09 (m, 1H), 7.25-7.27 (m, 2H), 7.62-7.64 (m, 1H), 7.97 (s, 1H), |
| | 8.08-8.14 (m, 2H), 8.27 (s, 1H), 8.82-8.86 (m, 1H) |
| 62 | ¹H NMR (400.21 MHz, DMSO-d6) δ ppm 1.78 (d, J = 6.6 Hz, 3H), 5.48-5.54 (m, 1H), |
| | 7.19 (d, J = 9.0 Hz, 1H), 7.32 (d, J = 9.0 Hz, 1H), 7.36 (s, 1H), 7.69 (ddd, J = 7.6, 4.7, 1.0 |
| | Hz, 1H), 8.02 (d, J = 2.2 Hz, 1H), 8.12 (td, J = 7.8, 1.8 Hz, 1H), 8.25 (d, J = 1.7 Hz, 1H), |
| | 8.33 (d, J = 7.8 Hz, 1H), 8.44 (d, J = 6.4 Hz, 1H), 8.83-8.85 (m, 1H) |
| 63 | ¹H NMR (400 MHz, DMSO-d6) δ ppm 1.66 (d, J = 6.60 Hz, 3H), 2.30 (s, 3H), 2.36 (s, |
| | 3H), 5.37-5.45 (m, 1H), 6.51-6.57 (m, 2H), 7.19-7.26 (m, 1H), 7.53 (s, 1H), 7.65 |
| | (dd, J = 8.25, 4.71 Hz, 1H), 7.75-7.84 (m, 2H), 8.42 (br d, J = 6.97 Hz, 2H), 8.51 (s, |
| | 1H), 8.64 (d, J = 4.77 Hz, 1H), 9.20 (s, 1H), 9.25-9.29 (m, 1H), 12.80 (br s, 1H) |
| 64 | ¹H NMR (400 MHz, DMSO-d6) δ ppm 1.59 (br d, J = 6.11 Hz, 3H), 2.29 (s, 3H), 5.36- |
| | 5.47 (m, 1H), 6.43-6.53 (m, 2H), 6.89 (s, 1H), 7.17 (t, J = 6.98 Hz, 1H), 7.47 (s, 1H), |
| | 7.66 (s, 1H), 7.75 (br d, J = 7.70 Hz, 1H), 8.01 (br d, J = 7.21 Hz, 2H), 8.12 (br d, J = 7.34 |
| | Hz, 2H), 8.37 (br d, J = 5.38 Hz, 1H), 8.58 (s, 1H), 9.34 (s, 1H), 12.74 (br s, 1H) |
| 65 | ¹H NMR (400 MHz, DMSO-d6) δ ppm 1.59 (br d, J = 6.48 Hz, 3H), 2.10 (s, 3H), 2.36 |
| | (s, 3H), 3.96 (s, 3H), 5.10-5.18 (m, 1H), 6.47 (br d, J = 8.44 Hz, 1H), 6.55 (t, J = 7.52 |
| | Hz, 1H), 7.04 (d, J = 8.68 Hz, 1H), 7.21 (br t, J = 7.70 Hz, 1H), 7.52 (s, 1H), 7.74-7.84 |
| | (m, 2H), 8.15 (d, J = 8.87 Hz, 1H), 8.36 (br s, 1H), 8.67 (s, 1H), 12.81 (br s, 1H) |
| 66 | ¹H NMR (400 MHz, DMSO-d6) δ ppm 1.58 (br d, J = 6.60 Hz, 3H), 2.22 (s, 3H), 2.29 |
| | (s, 3H), 3.79 (s, 3H), 5.27-5.35 (m, 1H), 6.44-6.52 (m, 2H), 6.93 (br d, J = 8.31 Hz, |
| | 1H), 7.16 (br t, J = 7.89 Hz, 1H), 7.39-7.53 (m, 4H), 7.69 (s, 1H), 7.74 (br d, J = 8.07 |
| | Hz, 1H), 8.30-8.41 (m, 2H), 9.01 (s, 1H), 12.72 (br s, 1H) |
| 67 | ¹H NMR (400 MHz, DMSO-d6) δ ppm 1.56 (d, J = 6.6 Hz, 3H), 2.40 (s, 3H), 5.11-5.26 |
| | (m, 1H), 6.44 (d, J = 8.6 Hz, 1H), 6.55 (t, J = 7.5 Hz, 1H), 7.20 (t, J = 7.7 Hz, 1H), 7.63 (s, |
| | 1H), 7.65-7.70 (m, 1H), 7.80 (d, J = 7.9 Hz, 1H), 7.84 (s, 1H), 8.00 (d, J = 7.7 Hz, 1H), |
| | 8.11 (t, J = 7.8 Hz, 1H), 8.32 (br s, 1H), 8.78 (d, J = 4.3 Hz, 1H), 12.78 (br s, 1H) |
| 68 | ¹H NMR (400 MHz, METHANOL-d4) δ ppm 1.67 (d, J = 6.7 Hz, 3H), 2.25 (s, 3H), |
| | 5.26 (q, J = 6.6 Hz, 1H), 6.45 (d, J = 8.4 Hz, 1H), 6.58 (t, J = 7.6 Hz, 1H), 7.18 (t, J = 7.8 |
| | Hz, 1H), 7.49 (dd, J = 8.9, 3.0 Hz, 1H), 7.61 (dd, J = 7.0, 5.5 Hz, 1H), 7.70 (dd, J = 8.0, 3.0 |
| | Hz, 1H), 7.92 (d, J = 7.9 Hz, 1H), 7.99 (d, J = 7.3 Hz, 1H), 8.06-8.11 (m, 1H), 8.81 (d, |
| | J = 4.6 Hz, 1H) |
| 69 | ¹H NMR (400 MHz, DMSO-d6) δ ppm 1.58 (br d, J = 6.48 Hz, 3H), 2.22 (s, 3H), 2.29 |
| | (s, 3H), 3.76 (s, 3H), 5.27-5.36 (m, 1H), 6.42-6.52 (m, 2H), 7.05 (d, J = 8.93 Hz, 2H), |
| | 7.16 (br t, J = 7.70 Hz, 1H), 7.45 (s, 1H), 7.69 (s, 1H), 7.75 (br d, J = 8.19 Hz, 1H), 7.82 |
| | (d, J = 8.93 Hz, 2H), 8.28-8.37 (m, 2H), 8.91 (s, 1H), 12.72 (br s, 1H) |
| 70 | ¹H NMR (400 MHz, DMSO-d6) δ ppm 1.60 (br d, J = 6.48 Hz, 3H), 2.19 (s, 3H), 2.29 |
| | (s, 3H), 3.80 (s, 3H), 5.23-5.31 (m, 1H), 6.42-6.50 (m, 2H), 7.08 (t, J = 7.64 Hz, 1H), |
| | 7.15 (t, J = 7.76 Hz, 1H), 7.24 (d, J = 8.31 Hz, 1H), 7.37-7.46 (m, 2H), 7.62 (d, J = 7.82 |
| | Hz, 1H), 7.68 (s, 1H), 7.73 (d, J = 8.07 Hz, 1H), 8.30 (s, 1H), 8.37 (br d, J = 5.75 Hz, |
| | 1H), 8.78 (s, 1H), 12.73 (br s, 1H) |
| 71 | ¹H NMR (500 MHz, DMSO-d6) δ ppm 1.70 (d, J = 6.7 Hz, 3H), 2.37 (s, 3H), 5.37-5.42 |
| | (m, 1H), 6.54-6.57 (m, 2H), 7.05 (d, J = 54.7 Hz, 1H), 7.20-7.23 (m, 1H), 7.26 (s, 1H), |
| | 7.59 (d, J = 1.8 Hz, 1H), 7.78 (d, J = 1.1 Hz, 1H), 7.81 (dd, J = 1.5, 8.1 Hz, 1H), 7.94 (d, |
| | J = 7.7 Hz, 1H), 8.28 (t, J = 7.9 Hz, 1H), 8.42 (d, J = 7.9 Hz, 1H), 8.45-8.50 (m, 1H), |
| | 12.89-12.90 (m, 1H) |
| 72 | ¹H NMR (400 MHz, DMSO-d6) δ ppm 1.58 (br d, J = 6.48 Hz, 3H), 2.22 (s, 3H), 2.29 |
| | (s, 3H), 3.93 (s, 3H), 5.28-5.36 (m, 1H), 6.41-6.50 (m, 2H), 7.15 (br t, J = 7.82 Hz, |
| | 1H), 7.45 (s, 1H), 7.69 (s, 1H), 7.74 (d, J = 7.95 Hz, 1H), 8.35 (br d, J = 5.01 Hz, 1H), |
| | 8.43 (s, 1H), 9.05 (s, 1H), 9.14 (s, 2H), 12.74 (br s, 1H) |
| 73 | ¹H NMR (400 MHz, DMSO-d6) δ ppm 1.58 (br d, J = 6.36 Hz, 3H), 2.23 (s, 3H), 2.29 |
| | (s, 3H), 5.28-5.36 (m, 1H), 6.42-6.52 (m, 2H), 7.16 (br t, J = 7.83 Hz, 1H), 7.46 (s, |
| | 1H), 7.69 (s, 1H), 7.75 (br d, J = 8.19 Hz, 1H), 8.02 (m, J = 8.31 Hz, 2H), 8.17 (m, J = 8.44 |
| | Hz, 2H), 8.34 (br d, J = 5.26 Hz, 1H), 8.45 (s, 1H), 9.15 (s, 1H), 12.73 (br s, 1H) |
| 74 | ¹H NMR (400 MHz, DMSO-d6) δ ppm 1.59 (br d, J = 6.11 Hz, 3H), 2.23 (s, 3H), 2.29 |
| | (br s, 3H), 5.27-5.36 (m, 1H), 6.43-6.52 (m, 2H), 7.16 (br t, J = 7.27 Hz, 1H), 7.45 (br |
| | s, 1H), 7.67-7.77 (m, 3H), 7.83 (br d, J = 7.46 Hz, 1H), 8.29 (br d, J = 7.95 Hz, 1H), |
| | 8.34 (br s, 1H), 8.43 (s, 1H), 8.47 (br s, 1H), 9.12 (s, 1H), 12.73 (br s, 1H) |
| 75 | ¹H NMR (400 MHz, DMSO-d6) δ ppm 1.65 (d, J = 6.36 Hz, 3H), 2.31 (s, 3H), 2.35 (s, |
| | 3H), 5.25-5.35 (m, 1H), 6.46-6.52 (m, 1H), 6.52-6.58 (m, 1H), 6.77 (s, 1H), 6.79 (s, |
| | 1H), 7.17-7.27 (m, 1H), 7.51-7.54 (m, 1H), 7.71-7.74 (m, 1H), 7.79-7.84 (m, 1H), |
| | 8.42 (br d, J = 5.62 Hz, 1H), 12.79 (br s, 1H), 13.29 (br s, 1 H) |
| 76 | ¹H NMR (500.11 MHz, DMSO-d6) δ ppm 1.75 (d, J = 6.7 Hz, 3H), 5.45-5.48 (m, 1H), |
| | 6.52 (d, J = 8.5 Hz, 1H), 6.57 (t, J = 7.5 Hz, 1H), 7.17-7.20 (m, 1H), 7.36 (s, 1H), 7.67- |
| | 7.69 (m, 1H), 7.84 (dd, J = 1.5, 7.9 Hz, 1H), 7.98 (d, J = 2.1 Hz, 1H), 8.12 (td, J = 7.8, |
| | 1.7 Hz, 1H), 8.24 (d, J = 1.5 Hz, 1H), 8.33 (d, J = 8.0 Hz, 1H), 8.79-8.80 (m, 1H), 8.83- |
| | 8.84 (m, 1H) |
| 77 | ¹H NMR (400 MHz, METHANOL-d4) δ ppm 1.69 (d, J = 6.6 Hz, 3H), 2.33 (s, 3H), |
| | 2.41 (s, 3H), 5.24 (q, J = 6.6 Hz, 1H), 6.49 (d, J = 8.6 Hz, 1H), 6.55 (t, J = 7.5 Hz, 1H), |
| | 6.82 (t, J = 55.2 Hz, 1H), 7.17 (t, J = 7.8 Hz, 1H), 7.61 (s, 1H), 7.82-7.92 (m, 3H), 8.10- |
| | 8.23 (m, 2H) |
| 78 | ¹H NMR (400 MHz, DMSO-d6) δ ppm 1.67 (d, J = 6.8 Hz, 3H), 2.38 (s, 3H), 5.32-5.35 |
| | (s, 1H), 6.35 (d, J = 9.2 Hz, 1H), 7.24-7.29 (m, 2H), 7.56 (d, J = 2.0 Hz, 1H), 7.64 (dd, |
| | J = 6.8, 4.8 Hz, 1H), 7.77 (s, 1H), 8.07-8.09 (m, 1H), 8.20 (s, 1H), 8.26 (d, J = 8.0 Hz, |
| | 1H), 8.80 (d, J = 4.0 Hz, 1H) |
| 79 | ¹H NMR (400 MHz, DMSO-d6) δ ppm 1.59 (d, J = 6.60 Hz, 3H), 2.26-2.32 (m, 3H), |
| | 3.76 (s, 3H), 5.33-5.43 (m, 1H), 6.44-6.54 (m, 2H), 6.83 (s, 1H), 7.05 (d, J = 9.05 Hz, |
| | 2H), 7.17 (s, 1H), 7.47 (d, J = 1.96 Hz, 1H), 7.65 (d, J = 1.22 Hz, 1H), 7.71-7.81 (m, |
| | 3H), 8.41 (s, 2H), 9.09 (s, 1H), 12.70 (br s, 1H) |
| 80 | ¹H NMR (400 MHz, DMSO-d6) δ ppm 1.59 (d, J = 6.60 Hz, 3H), 2.29 (s, 3H), 5.36- |
| | 5.45 (m, 1H), 6.42-6.56 (m, 2H), 6.86 (s, 1H), 7.18 (t, J = 7.27 Hz, 1H), 7.29-7.41 (m, |
| | 1H), 7.45-7.54 (m, 3H), 7.66 (s, 1H), 7.75 (dd, J = 7.89, 1.28 Hz, 1H), 7.88 (d, J = 7.83 |
| | Hz, 2H), 8.36 (br s, 1H), 8.47 (s, 1H), 9.20 (s, 1H), 12.70 (br s, 1H) |
| 81 | ¹H NMR (400 MHz, DMSO-d6) δ ppm 1.66 (d, J = 6.60 Hz, 3H), 2.36 (s, 3H), 3.90- |
| | 3.95 (m, 3H), 5.40-5.51 (m, 1H), 6.48-6.61 (m, 2H), 6.90 (s, 1H), 7.05 (d, J = 8.93 |
| | Hz, 1H), 7.19-7.29 (m, 1H), 7.54 (d, J = 2.08 Hz, 1H), 7.73 (d, J = 1.47 Hz, 1H), 7.80- |
| | 7.85 (m, 1H), 8.22-8.27 (m, 1H), 8.38-8.50 (m, 1H), 8.55 (s, 1H), 8.75 (d, J = 2.69 |
| | Hz, 1H), 9.22 (s, 1H), 12.79 (br s, 1H) |
| 82 | ¹H NMR (400 MHz, DMSO-d6) δ ppm 1.66 (d, J = 6.60 Hz, 3H), 2.36 (s, 3H), 5.43- |
| | 5.54 (m, 1H), 6.50-6.59 (m, 2H), 6.97 (s, 1H), 7.20-7.27 (m, 1H), 7.55 (d, J = 1.83 |
| | Hz, 1H), 7.73 (s, 1H), 7.80-7.85 (m, 1H), 8.29 (d, J = 8.56 Hz, 1H), 8.44 (br d, J = 5.87 |
| | Hz, 1H), 8.57-8.62 (m, 1H), 8.72 (s, 1H), 9.42 (d, J = 2.45 Hz, 1H), 9.48 (s, 1H), 12.78 |
| | (br s, 1H) |
| 83 | ¹H NMR (400 MHz, DMSO-d6) δ ppm 1.60 (d, J = 6.72 Hz, 3H), 2.29 (s, 3H), 5.31- |
| | 5.40 (m, 1H), 6.49 (t, J = 7.52 Hz, 1H), 6.53 (d, J = 8.44 Hz, 1H), 7.00 (s, 1H), 7.16- |
| | 7.22 (m, 1H), 7.49 (d, J = 1.96 Hz, 1H), 7.63-7.70 (m, 1H), 7.72-7.77 (m, 1H), 8.09 |
| | (d, J = 8.56 Hz, 1H), 8.36 (br d, J = 6.36 Hz, 1H), 8.44-8.50 (m, 1H), 8.62 (s, 1H), 8.98 |
| | (d, J = 1.59 Hz, 1H), 9.39 (s, 1H), 12.71 (br s, 1H) |
| 84 | ¹H NMR (500 MHz, DMSO-d6) δ ppm 1.74 (d, J = 6.7 Hz, 3H), 2.40 (s, 3H), 3.89 (s, |
| | 3H), 5.27-5.32 (m, 1H), 6.55-6.58 (m, 2H), 7.22-7.25 (m, 1H), 7.28-7.31 (m, 1H), 7.35 |
| | (s, 1H), 7.60 (t, J = 8.0 Hz, 1H), 7.66 (d, J = 1.9 Hz, 1H), 7.71 (m, 1H), 7.78-7.82 (m, |
| | 3H), 8.52-8.56 (m, 1H), 12.87-12.89 (m, 1H) |
| 85 | ¹H NMR (400 MHz, DMSO-d6) δ ppm 1.59 (d, J = 6.60 Hz, 3H), 2.29 (s, 3H), 5.36- |
| | 5.45 (m, 1H), 6.42-6.53 (m, 2H), 6.89 (s, 1H), 7.13-7.19 (m, 1H), 7.43-7.50 (m, |
| | 1H), 7.65-7.67 (m, 1H), 7.73-7.77 (m, 1H), 7.96-8.02 (m, 1H), 8.06-8.15 (m, 2H), |
| | 8.36 (br d, J = 6.11 Hz, 1H), 8.61 (s, 1H), 9.34 (s, 1H), 12.70 (br s, 1H) |
| 86 | ¹H NMR (400 MHz, DMSO-d6) δ ppm 1.59 (d, J = 6.60 Hz, 3H), 2.29 (s, 3H), 3.92 (s, |
| | 3H), 5.33-5.42 (m, 1H), 6.42-6.53 (m, 2H), 6.82 (s, 1H), 7.17 (t, J = 7.87 Hz, 1H), |
| | 7.39 (d, J = 9.29 Hz, 1H), 7.47 (d, J = 2.08 Hz, 1H), 7.66 (d, J = 1.34 Hz, 1H), 7.73-7.77 |
| | (m, 1H), 8.14-8.18 (m, 1H), 8.23 (d, J = 2.81 Hz, 1H), 8.36 (br d, J = 6.11 Hz, 1H), 8.47 |
| | (s, 1H), 9.17 (s, 1H), 12.72 (br s, 1H) |
| 87 | ¹H NMR (400 MHz, DMSO-d6) δ ppm 1.60 (d, J = 6.60 Hz, 3H), 2.29 (s, 3H), 5.36- |
| | 5.45 (m, 1H), 6.45-6.53 (m, 2H), 6.88 (s, 1H), 7.18 (t, J = 7.83 Hz, 1H), 7.48 (d, J = 2.08 |
| | Hz, 1H), 7.66 (d, J = 1.34 Hz, 1H), 7.73-7.78 (m, 1H), 7.89 (d, J = 8.68 Hz, 2H), 8.12 |
| | (d, J = 8.56 Hz, 2H), 8.37 (br d, J = 5.99 Hz, 1H), 8.54 (s, 1H), 9.34 (s, 1H), 12.70 (br s, |
| | 1H) |
| 88 | ¹H NMR (400 MHz, DMSO-d6) δ ppm 1.67 (d, J = 6.60 Hz, 3H), 2.37 (s, 3H), 3.29 (s, |
| | 3H), 5.41-5.54 (m, 1H), 6.48-6.64 (m, 2H), 6.96 (s, 1H), 7.20-7.30 (m, 1H), 7.56 |
| | (d, J = 2.08 Hz, 1H), 7.74 (d, J = 1.34 Hz, 1H), 7.80-7.85 (m, 1H), 8.10-8.16 (m, 2H), |
| | 8.23 (s, 1H), 8.24-8.26 (m, 1H), 8.44 (br d, J = 5.62 Hz, 1H), 8.64 (s, 1H), 9.43 (s, 1H), |
| | 12.78 (br s, 1H) |
| 89 | ¹H NMR (400 MHz, DMSO-d6) δ ppm 1.58 (d, J = 6.72 Hz, 3H), 1.83 (m, 2H), 1.92 (m, |
| | 2H), 2.09 (s, 3H), 2.37 (s, 3H), 5.14 (m, 1H), 6.48 (d, J = 8.4 Hz, 1H), 6.55 (t, J = 7.0, 1.8 |
| | Hz, 1H), 7.2 (t, J = 8.47, 1.7 Hz, 1H), 7.54 (d, J = 2.2 Hz, 1H), 7.78 (m, 3H), 8.28 (dd, |
| | J = 8.31, 2.32, 1H), 8.35 (d, J = 2.0 Hz, 1H), 8.96 (d, J = 1.8 Hz, 1H), 12.76 (br s, 1H) |
| 90 | ¹H NMR (500 MHz, DMSO-d6) δ ppm 1.73 (d, J = 6.7 Hz, 3H), 2.40 (s, 3H), 5.27-5.32 |
| | (m, 1H), 6.54-6.58 (m, 2H), 7.22-7.25 (m, 1H), 7.37 (s, 1H), 7.66 (d, J = 1.8 Hz, 1H), |
| | 7.80-7.83 (m, 2H), 8.16-8.18 (m, 2H), 8.39 (d, J = 8.3 Hz, 2H), 8.45-8.48 (m, 1H), |
| | 12.77-12.79 (m, 1H) |
| 91 | ¹H NMR (400 MHz, DMSO-d6) δ ppm 1.59 (d, J = 6.60 Hz, 3H), 2.29 (s, 3H), 5.35- |
| | 5.44 (m, 1H), 6.43-6.52 (m, 2H), 6.84 (s, 1H), 7.17 (t, J = 7.38 Hz, 1H), 7.47 (d, J = 2.08 |
| | Hz, 1H), 7.55 (d, J = 8.80 Hz, 1H), 7.65 (d, J = 1.34 Hz, 1H), 7.70-7.79 (m, 2H), 7.99 (d, |
| | J = 2.20 Hz, 1H), 8.30-8.43 (m, 1H), 8.49 (s, 1H), 9.17 (s, 1H), 12.71 (br s, 1H) |
| 92 | ¹H NMR (400 MHz, DMSO-d6) δ ppm 1.59 (d, J = 6.60 Hz, 3H), 2.29 (s, 3H), 3.84 (s, |
| | 3H), 5.35-5.44 (m, 1H), 6.41-6.56 (m, 2H), 6.83 (s, 1H), 7.17 (t, J = 7.80 Hz, 1H), |
| | 7.29 (t, J = 9.11 Hz, 1H), 7.47 (d, J = 1.96 Hz, 1H), 7.61-7.71 (m, 2H), 7.71-7.83 (m, |
| | 2H), 8.36 (br d, J = 6.24 Hz, 1H), 8.44 (s, 1H), 9.13 (s, 1H), 12.69 (br s, 1H) |
| 93 | ¹H NMR (400 MHz, DMSO-d6) δ ppm 1.59 (d, J = 6.60 Hz, 3H), 2.29 (s, 3H), 5.33- |
| | 5.46 (m, 1H), 6.41-6.56 (m, 2H), 6.86 (s, 1H), 7.14-7.21 (m, 1H), 7.47 (d, J = 1.96 |
| | Hz, 1H), 7.53 (d, J = 8.56 Hz, 2H), 7.66 (d, J = 1.34 Hz, 1H), 7.73-7.78 (m, 1H), 7.90- |
| | 8.10 (m, 2H), 8.29-8.45 (m, 1H), 8.50 (s, 1H), 9.23 (s, 1H), 12.71 (br s, 1H) |
| 94 | ¹H NMR (400 MHz, DMSO-d6) δ ppm 1.60 (d, J = 6.72 Hz, 3H), 2.29 (s, 3H), 3.98 (s, |
| | 3H), 5.31-5.41 (m, 1H), 6.43-6.55 (m, 2H), 6.86 (s, 1H), 7.13-7.22 (m, 1H), 7.49 |
| | (d, J = 2.08 Hz, 1H), 7.58-7.70 (m, 3H), 7.72-7.77 (m, 1H), 7.88 (d, J = 8.44 Hz, 1H), |
| | 8.37 (br d, J = 5.50 Hz, 1H), 8.54 (s, 1H), 9.36 (s, 1H), 12.69 (br s, 1H) |
| 95 | ¹H NMR (400 MHz, DMSO-d6) δ ppm 1.62 (d, J = 6.60 Hz, 3H), 2.38 (s, 3H), 5.71 (br |
| | t, J = 5.38 Hz, 1H), 6.43-6.52 (m, 1H), 6.90 (s, 1H), 7.15 (ddd, J = 13.27, 8.80, 4.95 Hz, |
| | 1H), 7.36 (br d, J = 7.34 Hz, 1H), 7.55 (d, J = 2.08 Hz, 1H), 7.67-7.74 (m, 1H), 8.06- |
| | 8.11 (m, 2H), 8.15-8.19 (m, 2H), 8.55 (s, 1H), 9.35 (s, 1H), 13.68 (br s, 1H) |
| 96 | ¹H NMR (400 MHz, ACETONITRILE-d3) δ ppm 1.75 (d, J = 6.6 Hz, 3H), 5.39-5.47 |
| | (m, 1H), 6.74 (s, 1H), 7.05 (br d, J = 8.8 Hz, 1H), 7.22 (d, J = 8.8 Hz, 1H), 7.84-7.95 (m, |
| | 3H), 8.03 (d, J = 8.7 Hz, 2H), 8.25 (d, J = 1.6 Hz, 1H), 8.30-8.36 (m, 2H), 8.86 (s, 1H) |
| 97 | ¹H NMR (500 MHz, DMSO-d6) δ ppm 1.66 (d, J = 6.7 Hz, 3H), 2.12 (s, 3H), 5.28-5.33 |
| | (m, 1H), 7.16 (d, J = 9.1 Hz, 1H), 7.30 (d, J = 8.9 Hz, 1H), 8.23-8.26 (m, 1H), 8.02 (d, |
| | J = 2.0 Hz, 1H), 8.28-8.31 (m, 2H), 8.85 (d, J = 2.6 Hz, 1H), 8.92 (s, 1H), 13.30-13.35 |
| | (m, 1H) |
| 98 | ¹H NMR (500 MHz, DMSO-d6) δ ppm 1.66 (d, J = 6.7 Hz, 3H), 2.37 (s, 3H), 3.89 (s, |
| | 3H), 5.47-5.52 (m, 1H), 6.51-6.56 (m, 2H), 7.02 (s, 1H), 7.17 (dt, J = 7.5, 2.0 Hz, 1H), |
| | 7.20-7.24 (m, 1H), 7.56-7.62 (m, 4H), 7.77 (s, 1H), 7.82 (dd, J = 1.4, 7.9 Hz, 1H), 8.46- |
| | 8.49 (m, 1H), 9.62 (s, 1H), 12.86-12.88 (m, 1H) |
| 99 | ¹H NMR (400 MHz, ACETONITRILE-d3) δ ppm 1.12 (d, J = 6.1 Hz, 3H), 5.51 (s, 1H), |
| | 6.56 (d, J = 9.1 Hz, 1H), 6.63 (t, J = 7.5 Hz, 1H), 6.81 (s, 1H), 7.24 (s, 1H), 7.89-7.99 |
| | (m, 4H), 8.09 (d, J = 7.9 Hz, 2H), 8.30 (s, 1H), 8.40 (s, 1H), 8.43 (d, J = 6.3 Hz, 1H), 8.93 |
| | (s, 1H) |
| 100 | ¹H NMR (400 MHz, METHANOL-d4) δ ppm 1.75 (d, J = 6.7 Hz, 3H), 2.42 (s, 3H), |
| | 5.39 (q, J = 6.7 Hz, 1H), 6.28-6.34 (m, 2H), 7.13 (td, J = 8.4, 6.0 Hz, 1H), 7.41 (s, 1H), |
| | 7.58 (ddd, J = 7.6, 4.7, 1.1 Hz, 1H), 7.64 (d, J = 2.2 Hz, 1H), 7.89 (d, J = 1.3 Hz, 1H), 8.04 |
| | (td, J = 7.8, 1.8 Hz, 1H), 8.23 (d, J = 7.9 Hz, 1H), 8.76 (d, J = 4.9 Hz, 1H) |
| 101 | ¹H NMR (400 MHz, METHANOL-d4) δ ppm 1.80 (d, J = 6.6 Hz, 3H), 2.49 (s, 3H), |
| | 5.51 (q, J = 6.2 Hz, 1H), 6.39-6.50 (m, 2H), 6.93-7.00 (m, 1H), 7.27 (td, J = 8.3, 6.2 |
| | Hz, 1H), 7.62-7.71 (m, 1H), 7.89 (d, J = l.2 Hz, 1H), 8.08 (d, J = 7.7 Hz, 2H), 8.26 (d, |
| | J = 8.5 Hz, 2H), 8.56-8.63 (m, 1H), 9.35 (s, 1H) |
| 102 | ¹H NMR (400 MHz, DMSO-d6) δ ppm 1.52 (s, 6H), 1.67 (d, J = 6.72 Hz, 3H), 2.37 (s, |
| | 3H), 3.43 (br s, 1H), 5.29-5.40 (m, 1H), 6.52-6.58 (rm, 2H), 7.18-7.25 (m, 1H), |
| | 7.32 (s, 1H), 7.58 (d, J = 2.08 Hz, 1H), 7.76 (d, J = 1.22 Hz, 1H), 7.79-7.83 (m, 1H), |
| | 8.43 (br d, J = 5.75 Hz, 1H), 9.46 (s, 2H), 12.79 (br s, 1H) |
| 103 | ¹H NMR (400 MHz, DMSO-d6) δ ppm 1.65 (d, J = 6.60 Hz, 3H), 2.36 (s, 3H), 5.47- |
| | 5.55 (m, 1H), 6.51-6.57 (m, 2H), 6.82 (s, 1H), 7.21-7.26 (m, 1H), 7.54 (d, J = 2.32 |
| | Hz, 1H), 7.73-7.75 (m, 1H), 7.82 (dd, J = 7.95, 1.71 Hz, 1H), 8.06-8.13 (m, 4H), 8.42 |
| | (br d, J = 6.24 Hz, 1H), 8.74 (dd, J = 7.40, 1.41 Hz, 2H), 12.74 (br s, 1H) |
| 104 | ¹H NMR (400 MHz, DMSO-d6) δ ppm 1.58 (d, J = 6.60 Hz, 3H), 2.29 (s, 3H), 5.27- |
| | 5.36 (m, 1H), 6.45-6.50 (m, 2H), 6.93 (s, 1H), 7.13-7.19 (m, 1H), 7.48 (d, J = 2.20 |
| | Hz, 1H), 7.50-7.60 (m, 1H), 7.65-7.67 (m, 1H), 7.88 (s, 1H), 8.01-8.06 (m, 1H), |
| | 8.17-8.22 (m, 1H), 8.36 (br d, J = 6.36 Hz, 1H), 8.60 (s, 1H), 9.06 (d, J = 2.08 Hz, 1H), |
| | 12.67 (br s, 1 H) |
| 105 | ¹H NMR (400 MHz, DMSO-d6) δ ppm 1.58 (d, J = 6.60 Hz, 3H), 2.29 (s, 3H), 3.93 (s, |
| | 3H), 5.33-5.42 (m, 1H), 6.40-6.52 (m, 3H), 6.85 (s, 1H), 7.12-7.20 (m, 1H), 7.47 |
| | (d, J = 1.83 Hz, 1H), 7.65 (s, 1H), 7.72-7.76 (m, 1H), 8.37 (br d, J = 6.24 Hz, 1H), 8.55 |
| | (s, 1H), 9.10 (s, 1H), 9.20 (s, 1H), 12.71 (br s, 1H) |
| 106 | ¹H NMR (400 MHz, DMSO-d6) δ ppm 1.59 (br d, J = 6.48 Hz, 3H), 2.29 (s, 3H), 5.35- |
| | 5.44 (m, 1H), 6.41-6.56 (m, 2H), 6.85 (s, 1H), 7.18 (t, J = 7.04 Hz, 1H), 7.47 (s, 1H), |
| | 7.58 (br d, J = 8.93 Hz, 2H), 7.66 (s, 1H), 7.72-7.77 (m, 1H), 7.91 (br d, J = 8.93 Hz, |
| | 2H), 8.36 (br d, J = 6.11 Hz, 1H), 8.48 (s, 1H), 9.22 (s, 1H), 12.69 (br s, 1H) |
| 107 | ¹H NMR (400 MHz, DMSO-d6) δ ppm 1.58 (d, J = 6.60 Hz, 3H), 2.29 (s, 3H), 5.31- |
| | 5.43 (m, 1H), 6.41-6.51 (m, 2H), 6.89 (s, 1H), 7.12-7.19 (m, 1H), 7.46 (d, J = 2.08 |
| | Hz, 1H), 7.58-7.68 (m, 2H), 7.71-7-76 (m, 1H), 7.81-7.93 (m, 2H), 8.02 (d, J = 7.58 |
| | Hz, 1H), 8.35 (br d, J = 5.26 Hz, 1H), 8.59 (s, 1H), 9.17 (s, 1H), 12.69 (br s, 1H) |
| 108 | ¹H NMR (400 MHz, l,4-DIOXANE-d8) δ ppm 1.75 (br d, J = 6.60 Hz, 3H), 2.38 (s, |
| | 3H), 5.26-5.40 (m, 1H), 6.46-6.62 (m, 2H), 6.81 (s, 1H), 7.17-7.25 (m, 1H), 7.29- |
| | 7.39 (m, 1H), 7.50 (d, J = 1.71 Hz, 1H), 7.83-7.97 (m, 3H), 8.10 (br d, J = 8.07 Hz, 1H), |
| | 8.36 (br s, 1H), 8.45-8.54 (m, 2H), 9.24 (s, 1H) |
| 109 | ¹H NMR (400 MHz, DMSO-d6) δ ppm 1.57 (d, J = 6.60 Hz, 3H), 2.28 (s, 3H), 2.59 (s, |
| | 3H), 5.26-5.35 (m, 1H), 6.39 (d, J = 8.44 Hz, 1H), 6.48 (t, J = 7.52 Hz, 1H), 6.69 (s, |
| | 1H), 7.15 (t, J = 7.28 Hz, 1H), 7.45 (d, J = 2.08 Hz, 1H), 7.68 (d, J = 1.47 Hz, 1H), 7.73- |
| | 7.79 (m, 1H), 7.96-8.01 (m, 2H), 8.04-8.09 (m, 2H), 8.33 (br d, J = 5.87 Hz, 1H), |
| | 9.29 (s, 1H), 12.71 (br s, 1H) |
| 110 | ¹H NMR (400 MHz, METHANOL-d4) δ ppm 1.75 (d, J = 6.7 Hz, 3H), 2.42 (s, 3H), |
| | 5.39 (q, J = 6.7 Hz, 1H), 6.28-6.34 (m, 2H), 7.13 (td, J = 8.4, 6.0 Hz, 1H), 7.41 (s, 1H), |
| | 7.58 (ddd, J = 7.6, 4.7, 1.1 Hz, 1H), 7.64 (d, J = 2.2 Hz, 1H), 7.89 (d, J = 1.3 Hz, 1H), 8.04 |
| | (td, J = 7.8, 1.8 Hz, 1H), 8.23 (d, J = 7.9 Hz, 1H), 8.76 (d, J = 4.9 Hz, 1H) |
| 111 | ¹H NMR (400 MHz, METHANOL-d4) δ ppm 1.75 (d, J = 6.7 Hz, 3H), 2.42 (s, 3H), |
| | 5.39 (q, J = 6.7 Hz, 1H), 6.28-6.34 (m, 2H), 7.13 (td, J = 8.4, 6.0 Hz, 1H), 7.41 (s, 1H), |
| | 7.58 (ddd, J = 7.6, 4.7, 1.1 Hz, 1H), 7.64 (d, J = 2.2 Hz, 1H), 7.89 (d, J = 1.3 Hz, 1H), 8.04 |
| | (td, J = 7.8, 1.8 Hz, 1H), 8.23 (d, J = 7.9 Hz, 1H), 8.76 (d, J = 4.9 Hz, 1H) |
| 112 | ¹H NMR (400 MHz, METHANOL-d4) δ ppm 1.80 (d, J = 6.6 Hz, 3 H), 2.49 (s, 3H), |
| | 5.51 (q, J = 6.2 Hz, 1H), 6.39-6.50 (m, 2H), 6.93-7.00 (m, 1H), 7.27 (td, J = 8.3, 6.2 |
| | Hz, 1H), 7.62-7.71 (m, 1H), 7.89 (d, J = 1.2 Hz, 1H), 8.08 (d, J = 7.7 Hz, 2H), 8.26 (d, |
| | J = 8.5 Hz, 2H), 8.56-8.63 (m, 1H), 9.35 (s, 1H) |
| 113 | ¹H NMR (400 MHz, METHANOL-d4) δ ppm 1.80 (d, J = 6.6 Hz, 3H), 2.49 (s, 3H), |
| | 5.51 (q, J = 6.2 Hz, 1H), 6.39-6.50 (m, 2H), 6.93-7.00 (m, 1H), 7.27 (td, J = 8.3, 6.2 |
| | Hz, 1H), 7.62-7.71 (m, 1H), 7.89 (d, J = 1.2 Hz, 1H), 8.08 (d, J = 7.7 Hz, 2H), 8.26 (d, |
| | J = 8.5 Hz, 2H), 8.56-8.63 (m, 1H), 9.35 (s, 1H) |
| 115 | ¹H NMR (400 MHz, DMSO-d6) δ ppm 1.58 (d, J = 6.4 Hz, 3H), 2.08 (s, 3H), 2.37 (s, |
| | 3H), 5.10-5.13 (m, 1H), 6.97 (d, J = 8.8 Hz, 1H), 7.19 (d, J = 8.8 Hz, 1H), 7.52 (d, J = 2.0 |
| | Hz, 1H), 7.63 (dd, J = 8.0, 5.2 Hz, 1H), 7.79 (s, 1H), 8.23-8.25 (m, 1H), 8.72 (s, 1H), |
| | 8.77 (d, J = 4.8 Hz, 1H), 9.01 (s, 1H) |
| 116 | ¹H NMR (400 MHz, DMSO-d6) δ ppm 1.55 (d, J = 4.4 Hz, 3H), 2.07 (s, 3H), 2.26-2.32 |
| | (m, 3H), 5.06-5.08 (m, 1H), 6.88-6.93 (m, 1H), 7.06-7.08 (m, 1H), 7.39-7.47 (m, 2H), |
| | 7.58-7.59 (m, 1H), 7.75 (s, 1H), 8.21 (d, J = 7.2 Hz, 1H), 8.73 (s, 1H), 9.00 (br s, 1H), |
| | 9.62-9.66 (m, 1H) |
| 117 | ¹H NMR (400 MHz, DMSO-d6) δ ppm 1.59 (d, J = 6.8 Hz, 3H), 2.08 (s, 3H), 2.36 (s, |
| | 3H), 5.12-5.17 (m, 1H), 6.95 (d, J = 8.8 Hz, 1H), 7.36 (d, J = 4.8 Hz, 1H), 7.55 (d, J = 3.6 |
| | Hz, 1H), 7.56-7.64 (m, 1H), 7.79 (s, 1H), 8.24-8.26 (m, 1H), 8.75-8.77 (m, 1H), 9.01 |
| | (s, 1H), 9.36 (s, 1H) |
| 118 | ¹H NMR (400 MHz, DMSO-d6) δ ppm 1.59 (d, J = 6.4 Hz, 3H), 2.08 (s, 3H), 2.35 (s, |
| | 3H), 2.37 (s, 3H), 5.14-5.17 (m, 1H), 7.02 (d, J = 8.4 Hz, 1H), 7.16 (d, J = 8.4 Hz, 1H), |
| | 7.53 (s, 1H), 7.63-7.66 (m, 1H), 7.79 (s, 1H), 8.23-8.27 (m, 1H), 8.51 (s, 1H), 8.77- |
| | 8.79 (m, 1H), 9.02 (s, 1H) |
| 119 | ¹H NMR (400 MHz, DMSO-d6) δ ppm 1.62 (d, J = 6.8 Hz, 3H), 2.08 (s, 3H), 2.38 (s, |
| | 3H), 5.19-5.26 (m, 1H), 6.67 (d, J = 9.2 Hz, 1H), 7.49-7.57 (m, 1H), 7.57 (d, J = 2.0 Hz, |
| | 1H), 7.61-7.65 (m, 1H), 7.81 (s, 1H), 8.04 (d, J = 2.0 Hz, 1H), 8.22-8.24 (m, 1H), 8.72- |
| | 8.78 (m, 2 H), 9.02 (d, J = 2.0 Hz, 1H) |
| 120 | ¹H NMR (400 MHz, DMSO-d6) δ ppm 1.57 (d, J = 6.8 Hz, 3H), 2.09 (s, 3H), 2.40 (s, |
| | 3H), 5.02-5.10 (m, 1H), 6.32-6.49 (m, 1H), 6.95-7.12 (m, 1H), 7.33-7.47 (m, 2H), |
| | 7.50-7.54 (m, 1H), 7.79 (s, 1H), 8.24 (d, J = 8.0 Hz, 1H), 8.79 (s, 1H), 9.03 (s, 1H), |
| | 13.12 (br s, 1H) |
| 121 | ¹H NMR (400 MHz, DMSO-d6) δ ppm 1.65 (d, J = 6.6 Hz, 3H), 2.25 (s, 3H), 2.39 (s, |
| | 3H), 5.23 (quin, J = 6.6 Hz, 1H), 7.11 (d, J = 9.0 Hz, 1H), 7.30 (d, J = 8.9 Hz, 1H), 7.56 (d, |
| | J = 2.1 Hz, 1H), 7.60 (q, J = 4.5 Hz, 1H), 7.81 (s, 1H), 8.06 (d, J = 4.3 Hz, 2H), 8.32 (d, |
| | J = 6.6 Hz, 1H), 8.81 (dt, J = 4.7, 1.2 Hz, 1H), 12.68-13.52 (m, 1H) |
| 122 | ¹H NMR (500.11 MHz, DMSO-d6) d ppm 1.64 (d, J = 6.7 Hz, 3H), 2.25 (s, 3H), 2.36 |
| | (d, J = 6.8 Hz, 6H), 5.19-5.25 (m, 1H), 7.08 (d, J = 8.8 Hz, 1H), 7.20 (d, J = 8.7 Hz, 1H), |
| | 7.54 (d, J = 1.7 Hz, 1H), 7.58-7.61 (m, 1H), 7.79 (s, 1H), 8.04-8.07 (m, 2H), 8.45 (d, J = |
| | 6.6 Hz, 1H), 8.81 (d, J = 4.8 Hz, 1H) |
| 123 | ¹H NMR (500.11 MHz, DMSO-d6) d ppm 1.60 (d, J = 6.7 Hz, 3H), 2.08 (s, 3H), 2.37 |
| | (d, J = 5.8 Hz, 6H), 5.17-5.22 (m, 1H), 7.08-7.11 (m, 1H), 7.19-7.21 (m, 1H), 7.55 (d, |
| | J = 1.8 Hz, 1H), 7.80 (s, 1H), 8.21-8.24 (m, 1H), 8.40-8.41 (m, 1H), 8.82 (d, J = 2.7 Hz, |
| | 1H), 8.89 (s, 1H) |
| 124 | ¹H NMR (400 MHz, DMSO-d6) δ ppm 1.65 (d, J = 6.6 Hz, 3H), 2.36 (s, 3H), 5.48 (br t, |
| | J = 6.3 Hz, 1H), 6.50 (d, J = 8.4 Hz, 1H), 6.54 (t, J = 7.5 Hz, 1H), 7.02 (s, 1H), 7.21 (t, |
| | J = 7.4 Hz, 1H), 7.56 (d, J = 2.2 Hz, 1H), 7.76 (d, J = 1.3 Hz, 1H), 7.82 (dd, J = 7.9, 1.7 Hz, |
| | 1H), 8.16-8.21 (m, 2H), 8.27-8.31 (m, 2H), 8.44 (br d, J = 6.1 Hz, 1H), 9.73 (s, 1H), |
| | 12.76 (br s, 1H) |
| 125 | ¹H NMR (400 MHz, DMSO-d6) δ ppm 1.52 (d, J = 6.0 Hz, 3H), 2.00 (m, 3H), 2.31 (s, |
| | 3H), 5.12 (m, 1H), 6.45 (m, 2H), 7.12 (m, 1H), 7.48 (d, J = 2.08 Hz, 1H), 7.71 (m, 2H), |
| | 8.25 (m, 1H), 8.71 (m, 1H), 9.15 (d, J = 1.96 Hz, 1H), 9.21 (d, J = 2.08 Hz, 1H), 12.65 |
| | (br s, 1H) |
| 126 | ¹H NMR (400 MHz, DMSO-d6) δ ppm 1.62 (d, J = 6.0 Hz, 3H), 2.27 (s, 3H), 2.30 (s, |
| | 3H), 5.19 (m, 1H), 6.47 (m, 2H), 7.17 (m, 1H), 7.5 (m, 4H), 7.7 (m, 1H), 7.75 (dd, J = |
| | 7.95, 1.59 Hz, 1H), 8.0 (m, 2H), 8.33 (d, J = 6.11 Hz, 1H), 8.54 (s, 1H), 12.68 (br s, 1H) |
| 127 | ¹H NMR (400 MHz, DMSO-d6) δ ppm 1.61 (d, J = 6.72 Hz, 3H), 2.13 (m, 3H), 2.39 |
| | (s, 3H), 5.18 (m, 1H), 6.51 (m, 2H), 6.67 (m, 1H), 7.18 (m, 1H), 7.56 (d, J = 2.08 Hz, |
| | 1H), 7.78 (dd, J = 7.59, 1.59 Hz, 1H), 7.81 (m, 1H), 7.89 (d, J = 1.71 Hz, 1H), 8.35 (m, |
| | 1H), 8.65 (m, 1H), 8.7 (d, J = 2.57 Hz, 1H), 8.95 (d, J = 1.83 Hz, 1H), 9.32 (d, J = 2.45 |
| | Hz, 1H), 12.71 (br s, 1H) |
| 128 | ¹H NMR (400 MHz, CDCl₃) δ ppm 1.60 (d, J = 6.8 Hz, 3H), 2.33 (s, 3H), 2.39 (s, 3H), |
| | 5.07-5.09 (m, 1H), 5.87 (d, J = 7.6 Hz, 1H), 6.84-6.91 (m, 1H), 7.42 (s, 1H), 7.55-7.58 |
| | (m, 1H), 7.80 (d, J = 7.6 Hz, 1H), 7.96-8.01 (m, 1H), 8.93 (d, J = 4.4 Hz, 1H) |
| 129 | ¹H NMR (400 MHz, DMSO-d6) δ ppm 1.59 (d, J = 7.2 Hz, 3H), 2.07 (s, 3H), 2.37 (s, |
| | 3H), 5.14-5.17 (m, 1H), 6.96 (d, J = 8.2 Hz, 1H), 7.35 (d, J = 8.8 Hz, 1H), 7.53 (s, 1H), |
| | 7.61-7.65 (m, 1H), 7.79 (s, 1H), 8.21-8.23 (m, 1H), 8.24 (s, 1H), 8.76-8.78 (m, 1H), |
| | 9.01 (s, 1H) |
| 130 | ¹H NMR (400 MHz, DMSO-d6) δ ppm 1.63 (d, J = 4.8 Hz, 3H), 2.23 (s, 3H), 2.34 (s, |
| | 3H), 3.15-3.16 (s, 6H), 5.14-5.16 (m, 1H), 6.43 (d, J = 8.0 Hz, 1H), 6.54 (t, J = 6.8 Hz, |
| | 1H), 7.18-7.20 (m, 1H), 7.48 (s, 1H), 7.72 (s, 1H), 7.82 (d, J = 7.6 Hz, 1H), 7.94 (d, |
| | J = 2.0 Hz, 1H), 8.52 (s, 1H) |
| 131 | ¹H NMR (500.11 MHz, DMSO-d6) d ppm 1.53-1.56 (m, 3H), 2.09 (s, 3H), 2.28 (s, |
| | 3H), 5.14-5.08 (m, 1H), 6.92 (d, J = 8.1 Hz, 1H), 7.07-7.11 (m, 1H), 7.39-7.48 (m, 2H), |
| | 7.77 (d, J = 1.1 Hz, 1H), 8.25-8.28 (m, 1H), 8.80 (d, J = 2.7 Hz, 1H), 8.91 (s, 1H), 9.65 |
| | (d, J = 7.2 Hz, 1H) |
| 132 | ¹H NMR (400 MHz, ACETONE-d6) δ ppm 1.66 (d, J = 6.8 Hz, 3H), 2.13 (s, 3H), 2.39 |
| | (s, 3H), 5.24-5.27 (m, 1H), 6.59 (d, J = 8.8 Hz, 1H), 7.16 (dd, J = 8.8, 2.4 Hz, 1H), 7.58- |
| | 7.59 (m, 1H), 7.60-7.62 (m, 1H), 7.85-7.86 (m, 2H), 8.24 (d, J = 7.6 Hz, 1H), 8.43-8.45 |
| | (m, 1 H), 8.77 (s, 1H), 9.03 (s, 1H) |
| 133 | ¹H NMR (400 MHz, METHANOL-d4): δ ppm 1.75 (d, J = 6.6 Hz, 3H), 2.42 (s, 3H), |
| | 3.92 (s, 3H), 3.95 (s, 3H), 5.41-5.49 (m, 1H), 6.51-6.60 (m, 2H), 7.13-7.22 (m, 3H), |
| | 7.48-7.53 (m, 1H), 7.57 (d, J = 2.4 Hz, 1H), 7.65 (d, J = 1.9 Hz, 1H), 7.86-7.92 (m, |
| | 2H), 9.14 (s, 1H) |
| 134 | ¹H NMR (400 MHz, METHANOL-d4) δ ppm 1.30 (d, J = 6.6 Hz, 3H), 2.32 (s, 3H), |
| | 3.55 (s, 3H), 3.79 (s, 3H), 3.96-4.02 (m, 1H), 5.89 (d, J = 8.5 Hz, 1H), 6.56 (t, J = 7.6 |
| | Hz, 1H), 6.87 (s, 1H), 7.01 (d, J = 8.6 Hz, 1H), 7.13 (t, J = 7.7 Hz, 1H), 7.21 (dd, J = 8.4, |
| | 2.4 Hz, 1H), 7.26 (d, J = 2.4 Hz, 1H), 7.44 (d, J = 2.0 Hz, 1H), 7.79 (s, 1H), 7.90 (d, J = 8.0 |
| | Hz, 1H), 8.50 (s, 1H) |
| 135 | ¹H NMR (400 MHz, DMSO-d6) δ ppm 1.45-1.56 (m, 3H), 1.72-1.80 (m, 2H), 1.82- |
| | 1.90 (m, 2H), 1.99-2.04 (m, 3H), 2.28-2.32 (m, 3H), 5.01-5.12 (m, 1H), 6.36-6.42 |
| | (m, 1H), 6.44-6.50 (m, 1H), 7.05-7.20 (m, 1H), 7.38-7.49 (m, 1H), 7.61-7.81 (m, |
| | 3H), 8.16-8.26 (m, 1H), 8.26-8.38 (m, 1H), 8.85-8.94 (m, 1H), 12.58-12.80 (m, 1H) |
| 136 | ¹H NMR (500.11 MHz, DMSO-d6) d ppm 1.64 (d, J = 6.7 Hz, 3H), 2.20 (s, 3H), 2.35 |
| | (s, 3H), 3.98 (s, 3H), 5.30 (quintet, J = 6.2 Hz, 1H), 6.49 (d, J = 8.5 Hz, 1H), 6.55 (t, J = |
| | 7.6 Hz, 1H), 7.21-7.24 (m, 1H), 7.49 (d, J = 2.0 Hz, 1H), 7.73 (d, J = 1.2 Hz, 1H), 7.82 |
| | (dd, J = 1.5, 8.0 Hz, 1H), 8.10 (s, 1H), 8.42 (d, J = 5.8 Hz, 1H), 8.49 (s, 1H), 12.85- |
| | 12.86 (m, 1H) |
| 137 | ¹H NMR (400 MHz, DMSO-d6) δ ppm 1.64-1.65 (m, 3H), 2.28 (s, 3H), 2.35 (s, 3H), |
| | 2.37 (s, 3H), 5.20-5.26 (m, 1H), 6.96-7.24 (m, 3H), 7.50-7.60 (m, 1H) 7.76-7.85 (m, |
| | 1H) 7.86-7.95 (m, 1H) 8.18-8.34 (m, 2H) 8.40-8.56 (m, 1H) |
| 138 | ¹H NMR (400 MHz, DMSO-d6) δ ppm 1.55 (d, J = 6.6 Hz, 3H), 2.09 (s, 3H), 2.36 (s, |
| | 3H), 5.09 (br s, 1H), 6.85 (d, J = 8.8 Hz, 1H), 7.05 (d, J = 8.8 Hz, 1H), 7.52 (d, J = 1.9 Hz, |
| | 1H), 7.78 (s, 1H), 8.27 (dt, J = 9.6, 2.1 Hz, 1H), 8.82 (d, J = 2.8 Hz, 1H), 8.91 (s, 1H), |
| | 9.39 (br s, 1H) |
| 139 | ¹H NMR (400 MHz, METHANOL-d4) δ ppm 1.75 (d, J = 6.6 Hz, 3H), 2.31 (s, 3H), |
| | 2.42 (s, 3H), 5.32 (q, J = 6.6 Hz, 1H), 6.83 (t, J = 55.1 Hz, 1 H), 7.37-7.56 (m, 2H), 7.65 |
| | (d, J = 1.9 Hz, 1H), 7.77 (d, J = 4.8 Hz, 1H), 7.86 (d, J = 7.6 Hz, 1H), 7.92 (s, 1H), 8.14- |
| | 8.27 (m, 2H) |
| 140 | ¹H NMR (400 MHz, DMSO-d6) δ ppm 1.61 (d, J = 6.6 Hz, 3H), 2.26 (s, 3H), 2.39 (s, |
| | 3H), 5.18 (quin, J = 6.5 Hz, 1H), 6.31 (d, J = 8.6 Hz, 1H), 6.36 (dd, J = 11.8, 7.9 Hz, 1H), |
| | 7.17 (td, J = 8.3, 6.3 Hz, 1H), 7.56 (d, J = 2.1 Hz, 1H), 7.59-7.62 (m, 1H), 7.80 (dd, J = 2.1, |
| | 0.9 Hz, 1H), 8.02-8.12 (m, 3H), 8.82 (dt, J = 4.7, 1.4 Hz, 1H), 13.15 (br s, 1H) |
| 141 | ¹H NMR (400 MHz, DMSO-d6) δ ppm 1.66 (d, J = 6.6 Hz, 3H), 2.28 (s, 3H), 2.38 (s, |
| | 3H), 5.25 (quin, J = 6.4 Hz, 1H), 7.08-7.13 (m, 2H), 7.29 (d, J = 9.0 Hz, 1H), 7.57 (d, |
| | J = 2.0 Hz, 1H), 7.81 (s, 1H), 7.90 (dd, J = 7.2, 1.3 Hz, 1H), 8.22-8.30 (m, 2H), 8.36 (br |
| | d, J = 6.4 Hz, 1H), 13.02 (br s, 1H) |
| 142 | ¹H NMR (400 MHz, CHLOROFORM-d) δ ppm 1.73 (d, J = 6.7 Hz, 3H), 2.42 (s, 3H), |
| | 2.43 (s, 3H), 5.20 (d, J = 6.3 Hz, 1H), 6.43 (d, J = 8.5 Hz, 1H), 6.64 (ddd, J = 8.1, 7.1, |
| | 1.0 Hz, 1H), 6.73 (t, J = 55.3 Hz, 1H), 7.25 (ddd, J = 8.7, 7.5, 2.2 Hz, 2H), 7.54 (d, J = |
| | 2.2 Hz, 1H), 7.80 (dd, J = 7.8, 1.1 Hz, 1H), 7.96-8.00 (m, 2H), 8.02 (dd, J = 8.1, 1.7 |
| | Hz, 1H), 8.08 (t, J = 7.9 Hz, 1H), 8.17 (s, 1H) |

PI3K-Alpha kinase (PIK3CA) activity, wild-type and H1047R mutant and determining IC50 values for inhibitors

Recombinant, catalytically active human full length PIK3KA Wild-type and H1047R mutant were purchased as 1:1 complex of N-terminal 6×his tagged p110α (catalytic) and untagged p85α (regulatory subunit) from EMD Millipore Sigma (cat. no. 14-602M and 14-792M, respectively). PIP2diC8 (Avanti Polar Lipids Inc., cat. no. 850185) or Soy PI (Avanti Polar Lipids Inc., cat. No. 840044P) was used as lipid substrate. PIP2diC8 or PI lyophilized powder was dissolved in milliQ water to a concentration of 1 mM just before use. 10 mM stock compounds in DMSO were serially diluted 1:3 to generate a 10-point curve and plated using an acoustic liquid handler system (Echo 550 series instrument, Labcyte). A 10× intermediate compound plate (200 uM starting compound concentration and 10% DMSO) was prepared before starting the reaction. A typical reaction mixture (50 uL) comprised 40 mM HEPES buffer, pH 7.4, 25 mM MgCl₂, 0.01% v/v triton-X-100, 1% v/v DMSO, 20 mM NaCl, 1-5 nM WT or H1047R PI3K protein, 20 uM ATP, and 50 uM PIP2diC8 or Soy PI. 1% DMSO buffer alone without test compound was employed as MAX control (full activity in the absence of any inhibitor), and no enzyme control was used to determine the level of background Adenosine 5′-diphosphate (ADP) (MIN control). First, Wild-type (WT) and H1047R mutant protein in kinase buffer with all components except ATP were incubated with or without compound at 27° C. for 1 h. After the pre-incubation, the reaction was initiated by the addition of 20 μL of 50 uM ATP (20 uM final concentration). The reaction was allowed to proceed until about 10% conversion of ATP (2 uM ADP) at 27° C. After that time, 5 μL of reaction was mixed with 5 μL of ADP-Kinase Glo Reagent (ADP-Glo Kinase assay kit, Promega cat. no. V9102) supplemented with MgCl₂ 10 mM to stop the reaction and deplete the remaining ATP for 40 min at room temperature. Then, 10 μL of Kinase Detection Reagent (ADP-Glo Kinase assay kit, Promega cat. no. V9102) was added to simultaneously convert ADP to ATP and allow the newly synthesized ATP to be measured using a luciferase/luciferin reaction. After 30 min at room temperature the light generated was measured using a luminometer (EnVision plate reader, Perkin Elmer). Process data through Genedata-Screener tool. Relative IC₅₀ values are determined using luminescence units by calculating percent inhibition with respect to on-plate “MIN” and “MAX” controls. Data was analyzed using a 4-parameter nonlinear logistic equation (four-parameter logistic concentration-response curve):

$$Y = bot + [ ( top - bot ) / 1 + ( x / IC 50 ) slope ]$$

where Y=% inhibition, X=concentration yielding y % inhibition, Bottom=minimum value of y attained by curve, Top=maximum value of y attained by curve and Slope=steepness of curve at IC50.

$$\% Inh = [ ( median Max - x / median Max - median Min ) ] \cdot 100$$

IC50: concentration of compound that reduces a given response (ligand binding, enzyme response) by 50%. IC50 relative: concentration giving half the compound's maximum response.

For IC₅₀ values shown in Table A, “A” means IC₅₀<0.5 μM; “B” means IC₅₀ ranging between 0.5 μM and 1.0 μM; “C” means IC₅₀ ranging between 1 μM and 5 μM; “D” means IC₅₀ ranging between 5 μM and 10 μM; “E” means IC₅₀>10 μM.

TABLE A
PI3K-α (PIK3CA) Biochemical IC₅₀ of
PI3K wild-type (WT) and H1047R mutant,
using Soy PI lipid substrate

| | IC₅₀ | IC₅₀ |
|---|---|---|
| Example # | H1047R | WT |
| 1 | B | E |
| 2 | A | D |
| 3 | A | E |
| 4¹ | A | A |
| 5¹ | E | E |
| 6 | A | E |
| 7 | A | E |
| 8 | A | D |
| 9 | A | D |
| 10 | B | C |
| 11 | A | B |
| 12 | A | C |
| 13 | B | E |
| 14 | A | C |
| 15¹ | A | C |
| 16¹ | E | E |
| 17 | A | C |
| 18¹ | E | E |
| 19 | D | E |
| 20 | A | C |
| 21 | D | E |
| 22 | A | C |
| 23 | A | D |
| 24 | A | C |
| 25 | A | B |
| 26 | A | D |
| 27 | A | C |
| 28 | A | C |
| 29 | A | D |
| 30¹ | D | E |
| 31¹ | A | C |
| 32¹ | A | D |
| 33¹ | E | E |
| 34 | A | C |
| 35 | E | E |
| 36 | A | D |
| 37 | E | E |
| 38 | A | E |
| 39 | E | E |
| 40 | A | E |
| 41 | E | E |
| 42 | A | D |
| 43 | B | D |
| 44 | A | C |
| 45 | A | B |
| 46 | A | D |
| 47¹ | B | C |
| 48 | A | D |
| 49 | E | E |
| 50 | A | C |
| 51 | A | C |
| 52 | A | C |
| 53 | A | C |
| 54 | A | C |
| 55 | A | D |
| 56 | C | E |
| 57 | A | E |
| 58 | A | D |
| 59¹ | A | C |
| 60 | A | B |
| 61 | A | B |
| 62 | A | D |
| 63 | A | C |
| 64 | A | C |
| 65 | A | C |
| 66 | A | B |
| 67 | A | C |
| 68 | A | D |
| 69 | A | B |
| 70 | C | C |
| 71 | A | D |
| 72 | A | C |
| 73 | A | B |
| 74 | A | B |
| 75 | A | D |
| 76 | A | D |
| 77 | A | B |
| 78 | A | D |
| 79 | A | C |
| 80 | A | D |
| 81 | B | D |
| 82 | A | C |
| 83 | A | E |
| 84 | A | C |
| 85 | A | C |
| 86 | A | C |
| 87 | A | D |
| 88 | A | C |
| 89 | A | C |
| 90 | A | E |
| 91 | B | E |
| 92 | A | D |
| 93 | B | E |
| 94 | A | C |
| 95 | C | E |
| 96 | A | C |
| 97 | A | C |
| 98 | A | C |
| 99 | B | C |
| 100 | A | E |
| 101 | A | B |
| 102 | B | D |
| 103 | A | D |
| 104 | B | E |
| 105 | B | E |
| 106 | A | C |
| 107 | A | E |
| 108 | B | C |
| 109 | B | D |
| 110 | A | C |
| 111 | C | C |
| 112 | D | E |
| 113 | A | C |
| 114 | C | D |
| 115 | A | B |
| 116 | A | D |
| 117 | A | C |
| 118 | A | C |
| 119 | A | C |
| 120 | A | C |
| 121 | A | C |
| 122 | A | D |
| 123 | A | C |
| 124 | A | C |
| 125 | A | B |
| 126 | A | A |
| 127 | A | C |
| 128 | A | B |
| 129 | A | A |
| 130 | A | B |
| 131 | C | E |
| 132 | A | B |

¹PIP2diC8 lipid substrate
*For Example 12, IC₅₀ WT/IC₅₀ H1047R = 16.9

PI3K-Alpha kinase (PIK3CA) activity in vitro cell based assay and determining IC50 values for inhibitors

The MDA-MB-453 (ATCC-HTB-131) cell line was obtained from the American Type Culture Collection (Manassas, VA). Cells were maintained in Dulbecco's Modified Eagle Media (DMEM, Gibco 11965-092) supplemented with 10% Fetal Bovine Serum, heat inactivated (FBS HI, Gibco 10082-147), 1× non-essential amino acids (NEAA, Gibco 11140-050), and 1 mM sodium pyruvate (Gibco 11360-070). Cultures were maintained in a humidified incubator at 37° C. under 5% CO₂/95% air.

For compound testing in 0% FBS, MDA-MB-453 cells were seeded at a density of 1.5×10⁴ cells per well in white 384-well plates in 20 μl of Minimum Essential Media (MEM) assay media with 1×NEAA, 1 mM sodium pyruvate, and 1 μg/mL human insulin (Sigma I9278). Compounds dissolved in 10 mM stock solutions in DMSO were serially diluted 1:3 in DMSO to generate a 10-point dilution series and plated using an acoustic liquid handler system (Echo 550 Series Liquid Handler, Labcyte). A 5× intermediate compound dilution plate in MEM with 1×NEAA and 1 mM sodium pyruvate (150 μM starting compound concentration in 1.5% DMSO) was then prepared. Five μl of the intermediate serially diluted compounds were added to the cell plate to final concentrations ranging from 30 mM to 0.0015 mM in 0.3% DMSO. 0.3% DMSO alone was used to establish the maximum (MAX) signal and GDC-0032 at a final concentration of 1 μM was used as a reference compound for the minimum (MIN) signal. After 3 hours treatment, the medium was removed, and the cells lysed in 10 μL of 1× SureFire Lysis buffer with shaking for 10 minutes at room temperature. The Acceptor Mix (Reaction Buffer 1+Reaction Buffer 2+Activation Buffer+SureFire Ultra Acceptor Beads) was prepared by diluting Activation buffer 25-fold in combined Reaction Buffer 1 and Reaction Buffer 2. The Acceptor beads were diluted 50-fold in the combined Reaction Buffers. Five μL of Acceptor Mix was added to each well, the plate was sealed and covered with foil and incubated for 1 hour at room temperature. The Donor Mix (dilution buffer+SureFire Ultra Donor Beads) was prepared by diluting Donor Beads 50-fold in dilution buffer. Five μL of the Donor Mix was added to each well and the plate sealed and covered with foil and incubated for 1 hour at room temperature in the dark. The plates were read on a Neo2 plate reader instrument from Biotek using standard AlphaLisa settings. Compounds were tested in duplicate and the average % inhibition at each compound concentration was used to generate a single dose response curve. The data were processed using the Genedata-Screener tool. Relative IC₅₀ values were determined using luminescence units by calculating percent inhibition with respect to the in-plate “MIN” (GDC-0032 reference control) and “MAX” (DMSO) controls. The data was analyzed using a 4-parameter nonlinear logistic equation (four-parameter logistic concentration-response curve):

$$Y = bottom + [ ( top - bottom ) / 1 + ( X / IC 50 ) slope ]$$

where Y=% inhibition, X=concentration of inhibitor, bottom=minimum value of y attained by curve-fit, top=maximum value of y attained by curve-fit and slope=steepness of curve at the IC₅₀.

$$\% Inhibition = [ \text{} ( signal at X - median Min ) / ( median Max - median Min ) ] \times 100$$

IC₅₀: concentration of compound that reduces a given response (ligand binding, enzyme response) by 50%. Relative IC₅₀: concentration giving half the compound's maximum response.

For IC₅₀ values shown in Table B, “A” means IC₅₀<50 nM; “B” means IC₅₀ ranging between 50 nM and 100 nM; “C” means IC₅₀ ranging between 100 nM and 500 nM; “D” means IC₅₀>500 nM.

TABLE B
PI3K-α (PIK3CA) in vitro cell
based assay IC₅₀

| Example # | IC₅₀ |
|---|---|
| 5 | D |
| 19 | D |
| 21 | D |
| 22 | B |
| 24 | A |
| 25 | A |
| 29 | C |
| 30 | D |
| 33 | D |
| 35 | D |
| 36 | A |
| 37 | D |
| 39 | D |
| 41 | D |
| 42 | B |
| 44 | A |
| 45 | A |
| 46 | A |
| 49 | D |
| 52 | B |
| 55 | C |
| 60 | A |
| 61 | A |
| 65 | A |
| 70 | A |
| 72 | B |
| 73 | B |
| 74 | A |
| 75 | B |
| 76 | B |
| 79 | B |
| 80 | A |
| 81 | B |
| 82 | B |
| 83 | B |
| 84 | C |
| 85 | C |
| 86 | C |
| 89 | A |
| 97 | A |
| 110 | C |
| 111 | D |
| 112 | D |
| 113 | C |
| 114 | D |
| 115 | A |
| 116 | B |
| 117 | A |
| 118 | B |
| 119 | B |
| 120 | A |
| 121 | A |
| 122 | B |
| 123 | A |
| 124 | B |
| 125 | A |
| 126 | B |
| 127 | A |
| 128 | B |
| 129 | A |
| 130 | A |
| 131 | B |
| 132 | A |
| 133 | C |
| 134 | D |
| 136 | A |
| 138 | A |
| 139 | C |
| 140 | B |
| 141 | A |

1. A compound of the Formula: 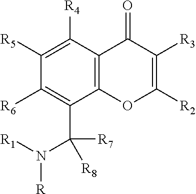
or pharmaceutically acceptable salt thereof, wherein:
R is —H or C₁-C₃ alkyl;
R₁ is a group of the formula: 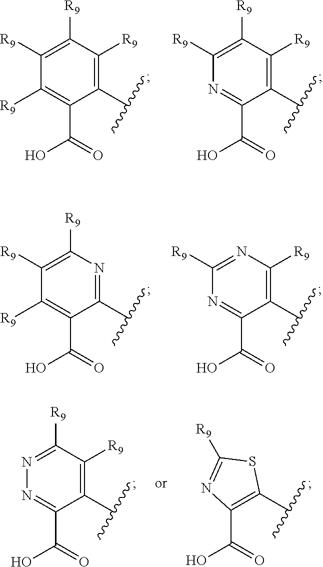
R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyridine, pyrimidine, pyridazine, pyrazine, pyrazole, isoxazole, isothiazole, imidazole, oxazole, or thiazole; wherein the optionally substituted C₁-C₆ alkyl, C₂-C₆ alkenyl, or C₂-C₆ alkynyl is each optionally substituted with a —CN, —OH, oxetanyl, or C₁-C₃ alkoxy; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN;
R₃ is —H, halogen, —CN, C₁-C₆ alkyl, C₁-C₆ haloalkyl, C₃-C₅ cycloalkyl, a heterocycle of 3 to 5 ring atoms containing 1, 2, or 3 ring heteroatoms independently selected from N, O, or S, or a heteroaryl of 5 ring atoms containing 1, 2, or 3 ring heteroatoms independently selected from N, O, or S;
each of R₄, R₅ and R₆ is independently —H, halogen, C₁-C₆ alkyl or C₁-C₆ haloalkyl;
R₇ is —CN, C₁-C₆ alkyl or C₁-C₆ haloalkyl;
R₈ is —H or C₁-C₆ alkyl;
each R₉ is independently —H, halogen, C₁-C₆ alkyl, C₁-C₆ haloalkyl, C₁-C₆ alkoxy, or C₃-C₅ cycloalkyl;
each R₁₁ is independently —H or C₁-C₃ alkyl.

2. (canceled)

3. The compound of claim 1, or pharmaceutically acceptable salt thereof, having the Formula:

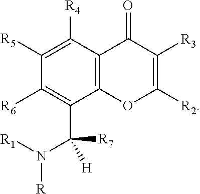

4. The compound of claim 1, or pharmaceutically acceptable salt thereof, wherein R is —H.

5. (canceled)

6. The compound of claim 1, or pharmaceutically acceptable salt thereof, wherein R₁ is a group of the formula:

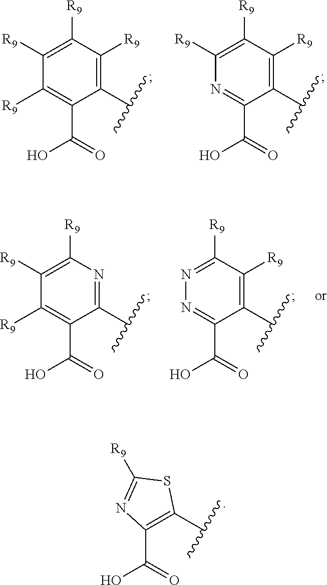

7. (canceled)

8. The compound of claim 1, or pharmaceutically acceptable salt thereof, wherein R₁ is a group of the formula

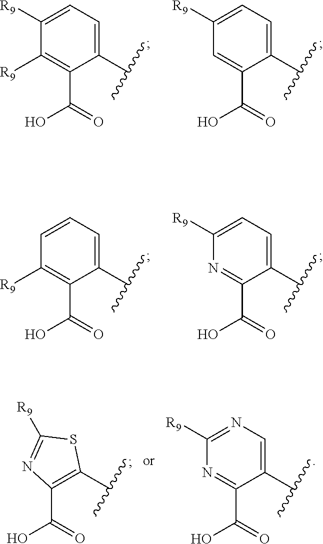

9. (canceled)

10. The compound of claim 1, or pharmaceutically acceptable salt thereof, wherein each R₉ is independently —H, halogen, C₁-C₃ alkyl, C₁-C₃ haloalkyl, C₁-C₃ alkoxy, or C₃-C₅ cycloalkyl.

11. The compound of claim 1, or pharmaceutically acceptable salt thereof, wherein each R₉ is independently —H, halogen, C₁-C₃ alkyl, C₁-C₃ haloalkyl, or C₃-C₅ cycloalkyl.

12. The compound of claim 1, or pharmaceutically acceptable salt thereof, wherein each R₉ is independently —H, halogen, C₁-C₃ alkyl or C₁-C₃ haloalkyl.

13. (canceled)

14. The compound of claim 1, or pharmaceutically acceptable salt thereof, wherein R₁ is a group of the formula

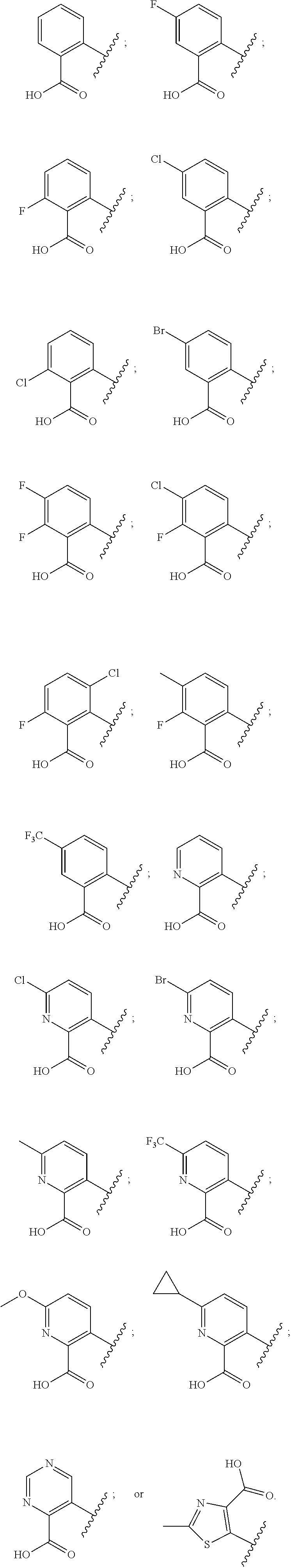

15. The compound of claim 1, or pharmaceutically acceptable salt thereof, wherein R₁ is a group of the formula

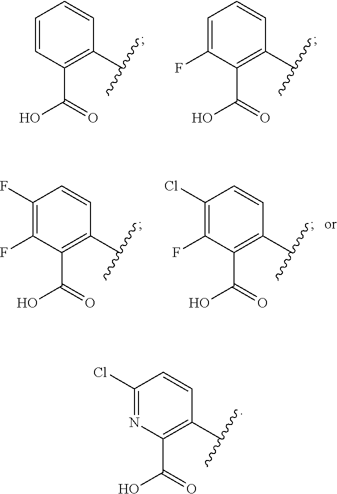 [SMILES: *c1ccccc1C(=O)O ; *c1cccc(F)c1C(=O)O ; *c1ccc(F)c(F)c1C(=O)O ; *c1ccc(Cl)c(F)c1C(=O)O ; *c1ccc(Cl)nc1C(=O)O]

16. (canceled)

17. The compound of claim 1, or pharmaceutically acceptable salt thereof, wherein R₃ is —H, —CN, C₁-C₆ alkyl, or C₁-C₆ haloalkyl.

18.-20. (canceled)

21. The compound of claim 1, or pharmaceutically acceptable salt thereof, wherein R₃ is —H or methyl.

22. The compound of claim 1, or pharmaceutically acceptable salt thereof, wherein R₄ is —H or halogen.

23. The compound of claim 1, or pharmaceutically acceptable salt thereof, wherein R₄ is —H.

24. The compound of claim 1, or pharmaceutically acceptable salt thereof, wherein R₅ is —H, halogen, C₁-C₃ alkyl or C₁-C₃ haloalkyl.

25. The compound of claim 1, or pharmaceutically acceptable salt thereof, wherein R₆ is —H or halogen.

26. (canceled)

27. (canceled)

28. The compound of claim 1, or pharmaceutically acceptable salt thereof, wherein R₇ is methyl.

29. The compound of claim 1, or pharmaceutically acceptable salt thereof, wherein R₈ is —H.

30.-41. (canceled)

42. The compound of claim 1, or pharmaceutically acceptable salt thereof, wherein R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —NR₁₁R₁₁, —NR₁₁CO₂R₁₁, an optionally substituted C₁-C₆ alkyl, an optionally substituted C₃-C₅ cycloalkyl, an optionally substituted heterocycle selected from pyrrolidine, pyrrolidinone, piperidine or morpholine, an optionally substituted phenyl, or an optionally substituted heteroaryl selected from pyridine, pyrazole, isoxazole, isothiazole, imidazole, oxazole, or thiazole; wherein the optionally substituted C₁-C₆ alkyl is optionally substituted with a —CN, —OH, or C₁-C₃ alkoxy; the optionally substituted C₃-C₅ cycloalkyl, phenyl, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN.

43. The compound of claim 1, or pharmaceutically acceptable salt thereof, wherein R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from C₁-C₆ haloalkyl, an optionally substituted C₁-C₆ alkyl, an optionally substituted phenyl, an optionally substituted 1,3-benzodioxole, or an optionally substituted heteroaryl selected from pyridine, pyrimidine, pyridazine, pyrazine, pyrazole, isoxazole, isothiazole, imidazole, oxazole, or thiazole; wherein the optionally substituted C₁-C₆ alkyl, is optionally substituted with a —CN, —OH, oxetanyl, or C₁-C₃ alkoxy; and the optionally substituted phenyl, 1,3-benzodioxole, or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN.

44. The compound of claim 1, or pharmaceutically acceptable salt thereof, wherein R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from C₁-C₆ haloalkyl, C₁-C₆ alkyl, an optionally substituted phenyl, an optionally substituted 1,3-benzodioxole, or an optionally substituted heteroaryl selected from pyridine or pyrimidine; wherein the optionally substituted phenyl, 1,3-benzodioxole, or heteroaryl is each optionally substituted with one to three substituents each independently selected from halogen, C₁-C₃ haloalkyl, C₁-C₃ alkoxy, C₁-C₃ haloalkoxy, —SO₂R₁₁, or —CN.

45. The compound of claim 1, or pharmaceutically acceptable salt thereof, wherein R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from:

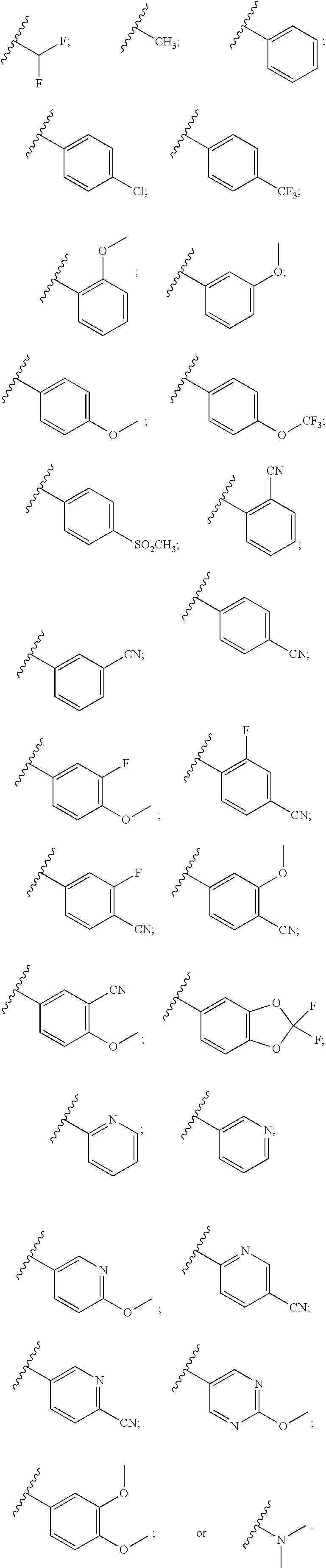

46. The compound of claim 1, or pharmaceutically acceptable salt thereof, wherein R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from:

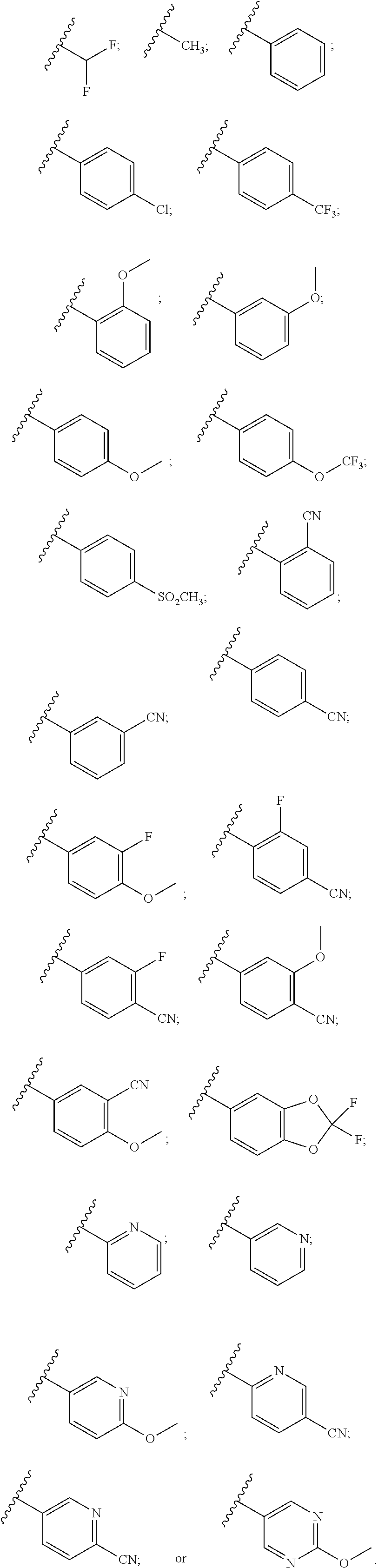

47. The compound of claim 1, or pharmaceutically acceptable salt thereof, wherein R₂ is a group of the formula:

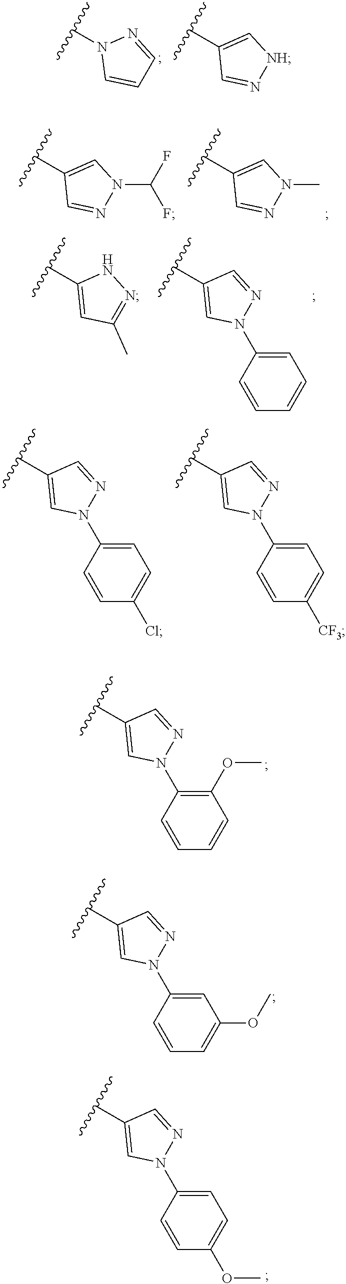

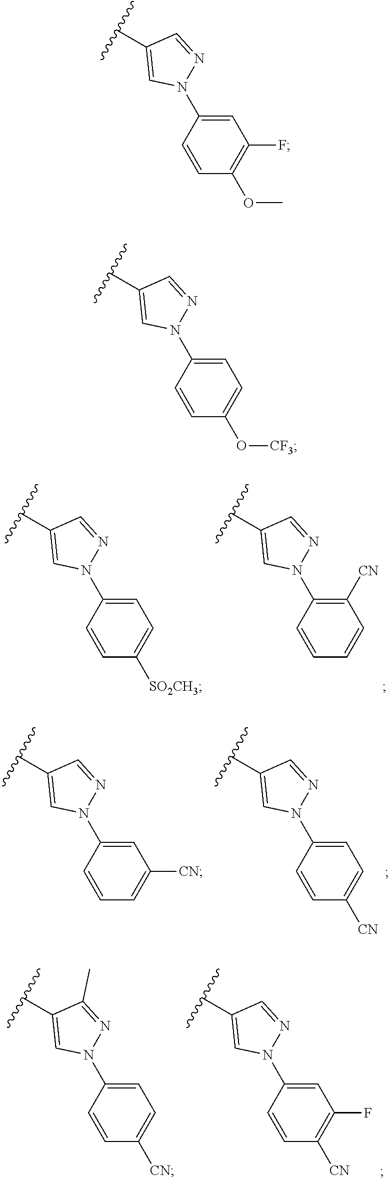

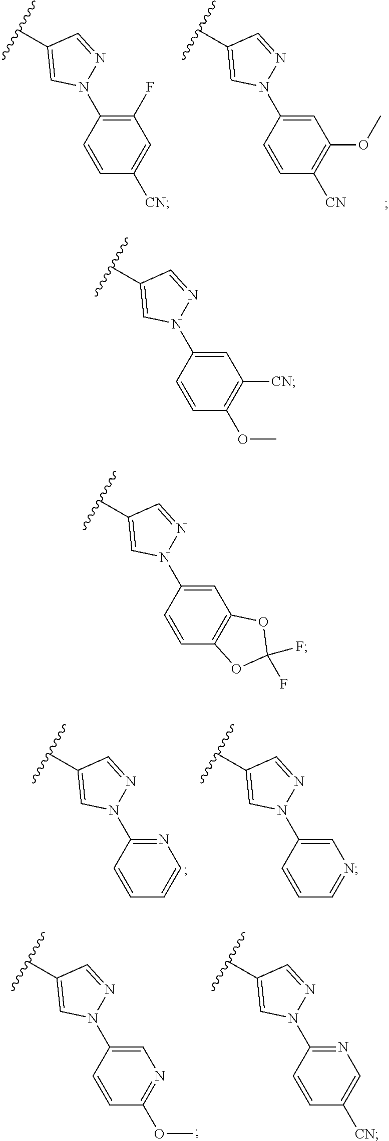

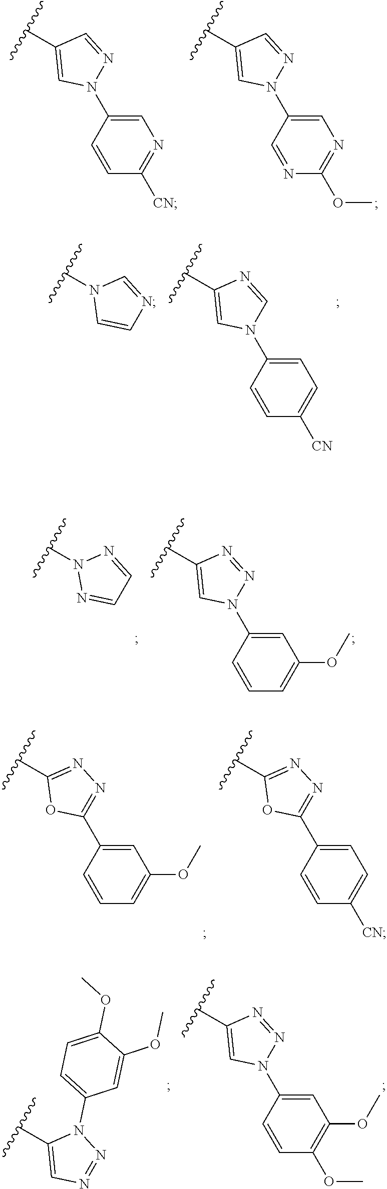

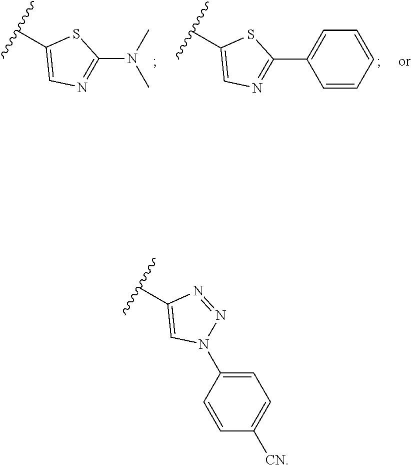

48. The compound of claim 1, or pharmaceutically acceptable salt thereof, wherein R₂ is a group of the formula:

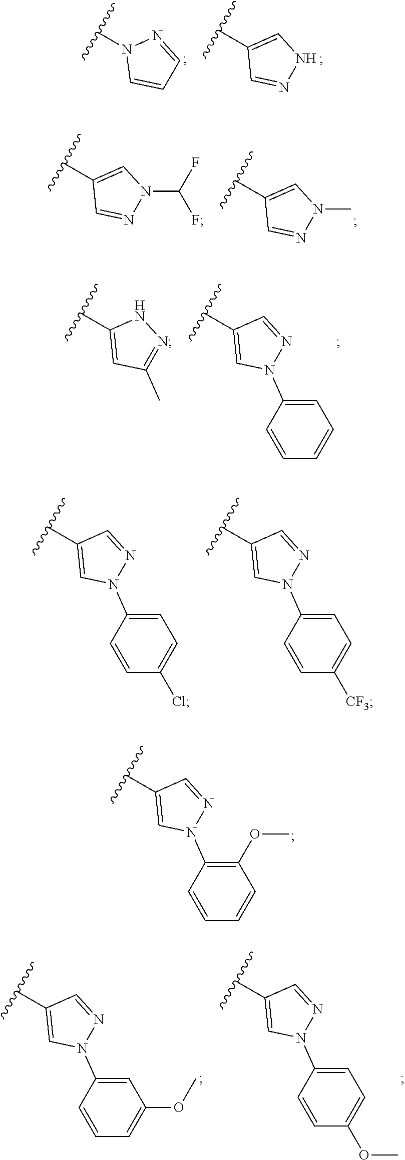

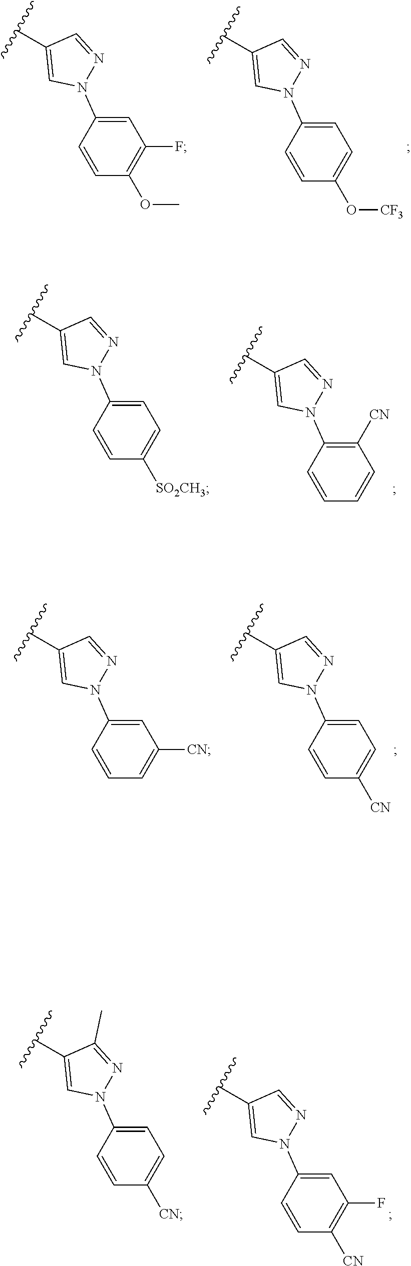

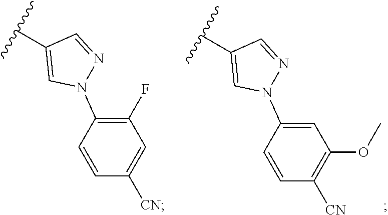 [SMILES: *c1cnn(-c2ccc(C#N)cc2F)c1 ; *c1cnn(-c2ccc(C#N)c(OC)c2)c1]

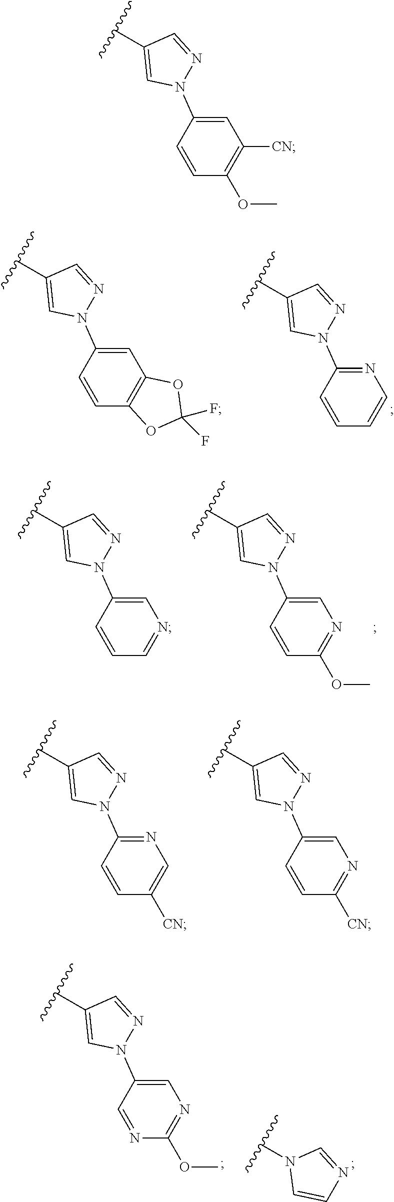

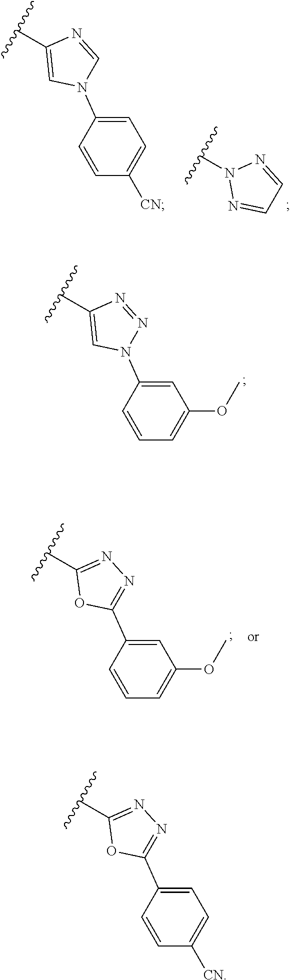 [SMILES: *c1cn(-c2ccc(C#N)cc2)cn1 ; *n1nccn1 ; *c1cn(-c2cccc(OC)c2)nn1 ; *c1nnc(-c2cccc(OC)c2)o1 ; *c1nnc(-c2ccc(C#N)cc2)o1]

49. (canceled)

50. The compound of claim 1, selected from: 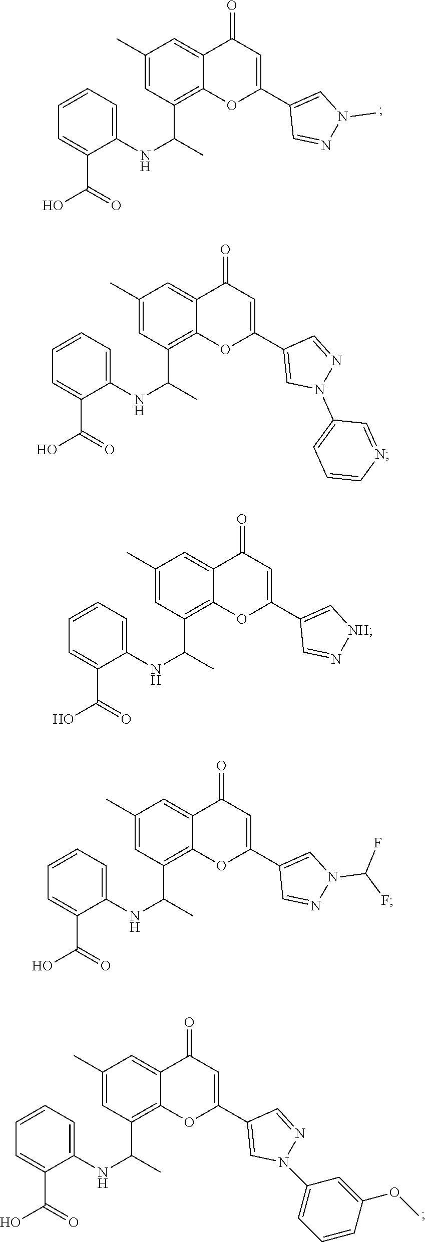 [SMILES: Cc1cc(C(C)Nc2ccccc2C(=O)O)c2oc(-c3cnn(C)c3)cc(=O)c2c1 ; Cc1cc(C(C)Nc2ccccc2C(=O)O)c2oc(-c3cnn(-c4cccnc4)c3)cc(=O)c2c1 ; Cc1cc(C(C)Nc2ccccc2C(=O)O)c2oc(-c3cn[nH]c3)cc(=O)c2c1 ; Cc1cc(C(C)Nc2ccccc2C(=O)O)c2oc(-c3cnn(C(F)F)c3)cc(=O)c2c1 ; COc1cccc(-n2cc(-c3cc(=O)c4cc(C)cc(C(C)Nc5ccccc5C(=O)O)c4o3)cn2)c1] 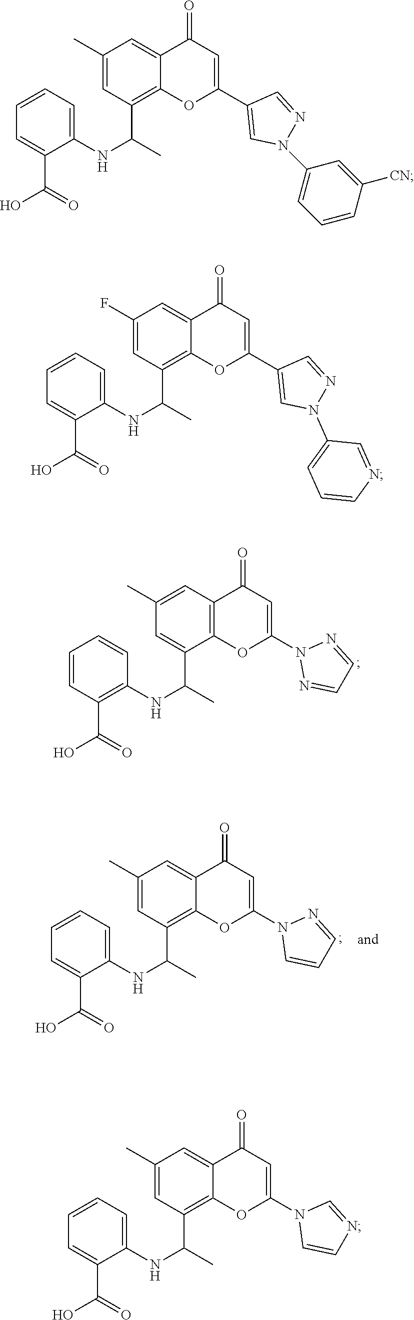 [SMILES: Cc1cc(C(C)Nc2ccccc2C(=O)O)c2oc(-c3cnn(-c4cccc(C#N)c4)c3)cc(=O)c2c1 ; CC(Nc1ccccc1C(=O)O)c1cc(F)cc2c(=O)cc(-c3cnn(-c4cccnc4)c3)oc12 ; Cc1cc(C(C)Nc2ccccc2C(=O)O)c2oc(-n3nccn3)cc(=O)c2c1 ; Cc1cc(C(C)Nc2ccccc2C(=O)O)c2oc(-n3cccn3)cc(=O)c2c1 ; Cc1cc(C(C)Nc2ccccc2C(=O)O)c2oc(-n3ccnc3)cc(=O)c2c1]
or a pharmaceutically acceptable salt thereof.

51. The compound of claim 1, selected from: 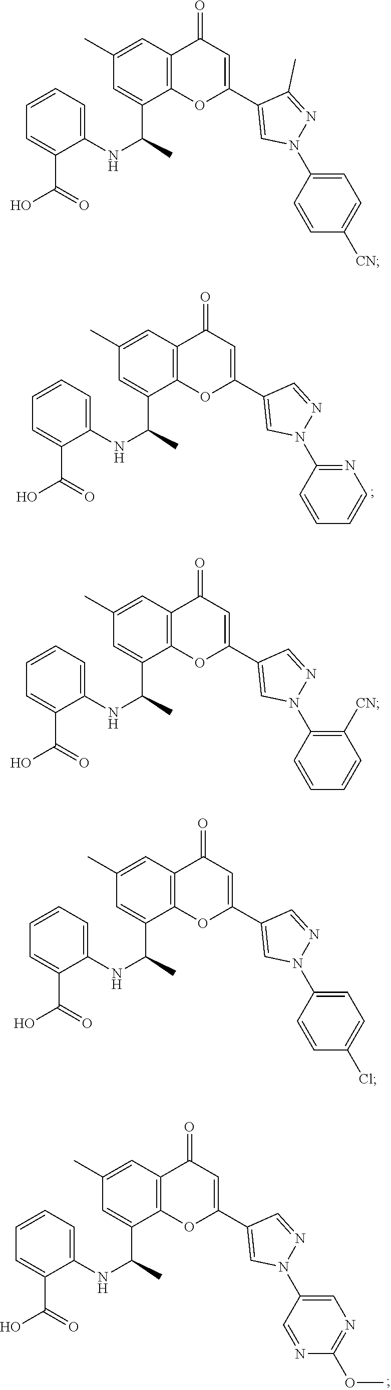 [SMILES: Cc1cc([C@@H](C)Nc2ccccc2C(=O)O)c2oc(-c3cn(-c4ccc(C#N)cc4)nc3C)cc(=O)c2c1 ; Cc1cc([C@@H](C)Nc2ccccc2C(=O)O)c2oc(-c3cnn(-c4ccccn4)c3)cc(=O)c2c1 ; Cc1cc([C@@H](C)Nc2ccccc2C(=O)O)c2oc(-c3cnn(-c4ccccc4C#N)c3)cc(=O)c2c1 ; Cc1cc([C@@H](C)Nc2ccccc2C(=O)O)c2oc(-c3cnn(-c4ccc(Cl)cc4)c3)cc(=O)c2c1 ; COc1ncc(-n2cc(-c3cc(=O)c4cc(C)cc([C@@H](C)Nc5ccccc5C(=O)O)c4o3)cn2)cn1] 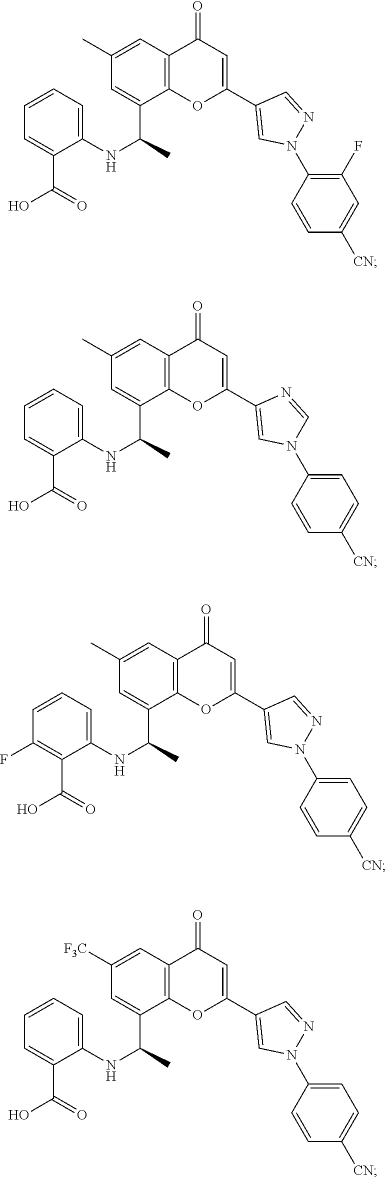 [SMILES: Cc1cc([C@@H](C)Nc2ccccc2C(=O)O)c2oc(-c3cnn(-c4ccc(C#N)cc4F)c3)cc(=O)c2c1 ; Cc1cc([C@@H](C)Nc2ccccc2C(=O)O)c2oc(-c3cn(-c4ccc(C#N)cc4)cn3)cc(=O)c2c1 ; Cc1cc([C@@H](C)Nc2cccc(F)c2C(=O)O)c2oc(-c3cnn(-c4ccc(C#N)cc4)c3)cc(=O)c2c1 ; C[C@@H](Nc1ccccc1C(=O)O)c1cc(C(F)(F)F)cc2c(=O)cc(-c3cnn(-c4ccc(C#N)cc4)c3)oc12] 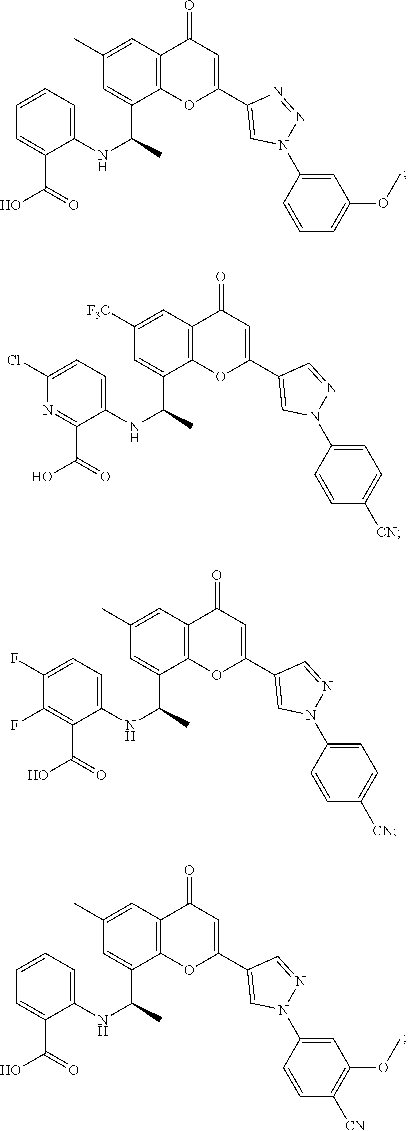 [SMILES: COc1cccc(-n2cc(-c3cc(=O)c4cc(C)cc([C@@H](C)Nc5ccccc5C(=O)O)c4o3)nn2)c1 ; C[C@@H](Nc1ccc(Cl)nc1C(=O)O)c1cc(C(F)(F)F)cc2c(=O)cc(-c3cnn(-c4ccc(C#N)cc4)c3)oc12 ; Cc1cc([C@@H](C)Nc2ccc(F)c(F)c2C(=O)O)c2oc(-c3cnn(-c4ccc(C#N)cc4)c3)cc(=O)c2c1 ; COc1cc(-n2cc(-c3cc(=O)c4cc(C)cc([C@@H](C)Nc5ccccc5C(=O)O)c4o3)cn2)ccc1C#N] 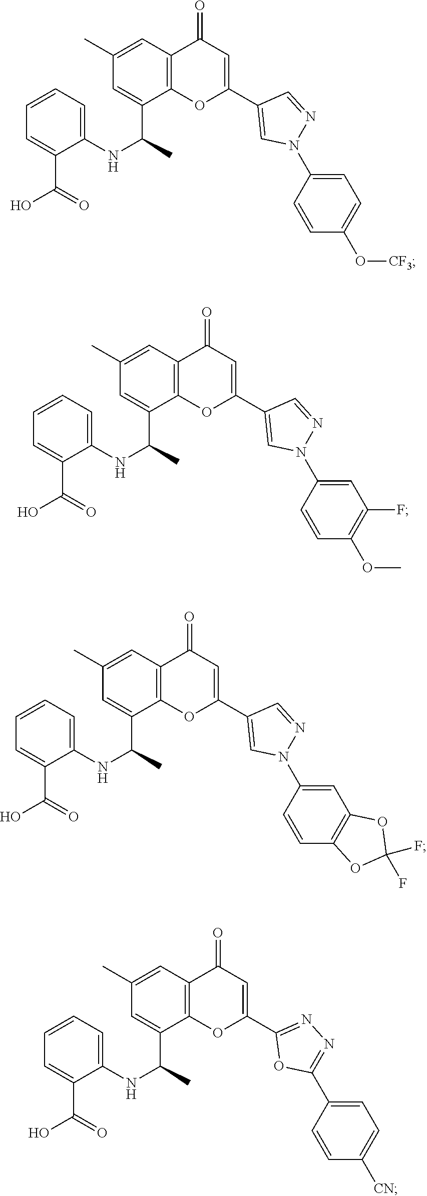 [SMILES: Cc1cc([C@@H](C)Nc2ccccc2C(=O)O)c2oc(-c3cnn(-c4ccc(OC(F)(F)F)cc4)c3)cc(=O)c2c1 ; COc1ccc(-n2cc(-c3cc(=O)c4cc(C)cc([C@@H](C)Nc5ccccc5C(=O)O)c4o3)cn2)cc1F ; Cc1cc([C@@H](C)Nc2ccccc2C(=O)O)c2oc(-c3cnn(-c4ccc5c(c4)OC(F)(F)O5)c3)cc(=O)c2c1 ; Cc1cc([C@@H](C)Nc2ccccc2C(=O)O)c2oc(-c3nnc(-c4ccc(C#N)cc4)o3)cc(=O)c2c1] 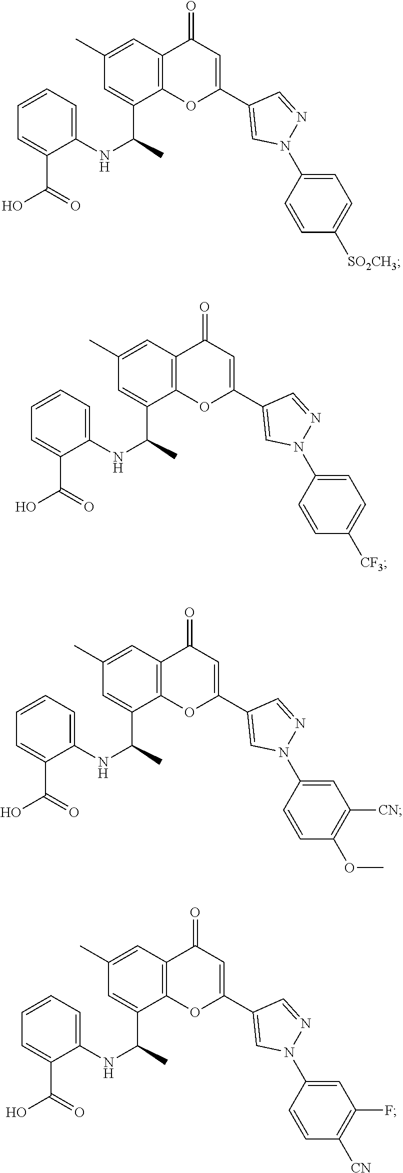 [SMILES: Cc1cc([C@@H](C)Nc2ccccc2C(=O)O)c2oc(-c3cnn(-c4ccc(S(C)(=O)=O)cc4)c3)cc(=O)c2c1 ; Cc1cc([C@@H](C)Nc2ccccc2C(=O)O)c2oc(-c3cnn(-c4ccc(C(F)(F)F)cc4)c3)cc(=O)c2c1 ; COc1ccc(-n2cc(-c3cc(=O)c4cc(C)cc([C@@H](C)Nc5ccccc5C(=O)O)c4o3)cn2)cc1C#N ; Cc1cc([C@@H](C)Nc2ccccc2C(=O)O)c2oc(-c3cnn(-c4ccc(C#N)c(F)c4)c3)cc(=O)c2c1] 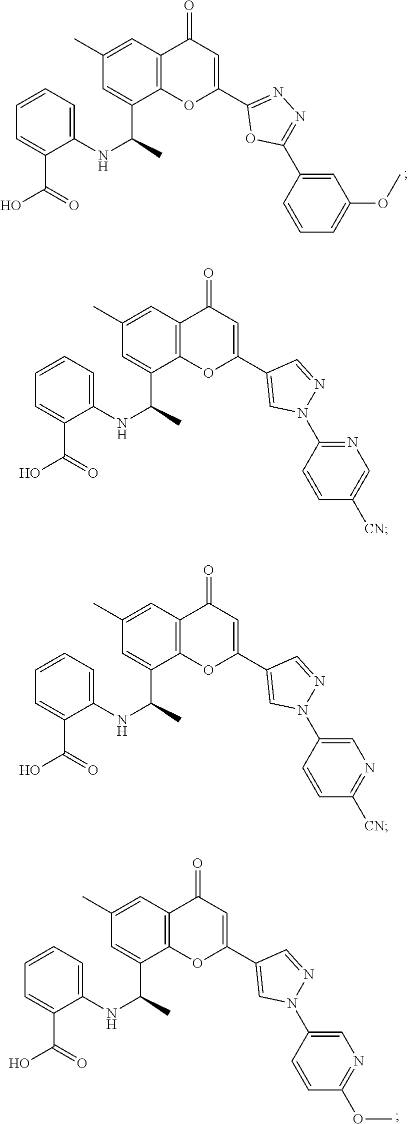 [SMILES: COc1cccc(-c2nnc(-c3cc(=O)c4cc(C)cc([C@@H](C)Nc5ccccc5C(=O)O)c4o3)o2)c1 ; Cc1cc([C@@H](C)Nc2ccccc2C(=O)O)c2oc(-c3cnn(-c4ccc(C#N)cn4)c3)cc(=O)c2c1 ; Cc1cc([C@@H](C)Nc2ccccc2C(=O)O)c2oc(-c3cnn(-c4ccc(C#N)nc4)c3)cc(=O)c2c1 ; COc1ccc(-n2cc(-c3cc(=O)c4cc(C)cc([C@@H](C)Nc5ccccc5C(=O)O)c4o3)cn2)cn1] 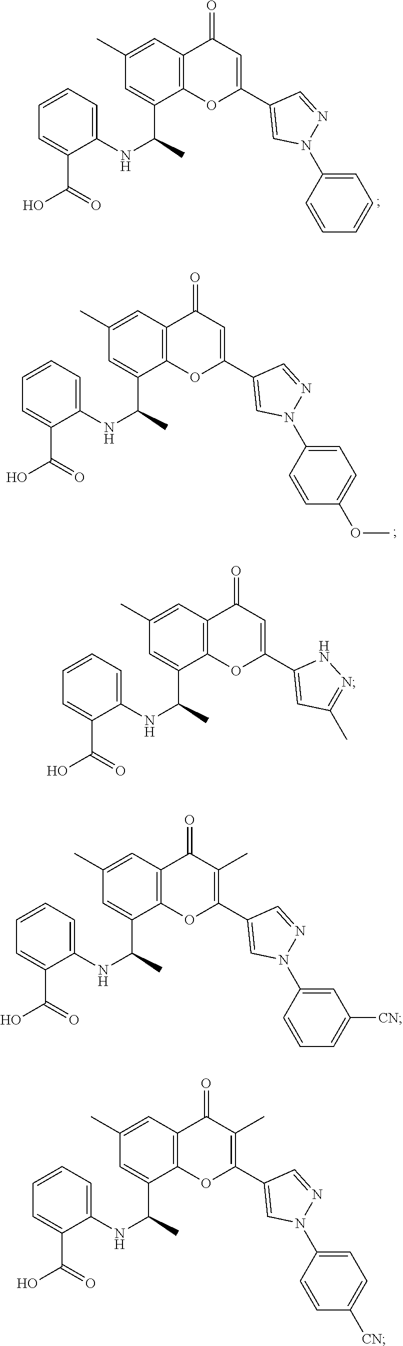 [SMILES: Cc1cc([C@@H](C)Nc2ccccc2C(=O)O)c2oc(-c3cnn(-c4ccccc4)c3)cc(=O)c2c1 ; COc1ccc(-n2cc(-c3cc(=O)c4cc(C)cc([C@@H](C)Nc5ccccc5C(=O)O)c4o3)cn2)cc1 ; Cc1cc([C@@H](C)Nc2ccccc2C(=O)O)c2oc(-c3cc(C)n[nH]3)cc(=O)c2c1 ; Cc1cc([C@@H](C)Nc2ccccc2C(=O)O)c2oc(-c3cnn(-c4cccc(C#N)c4)c3)c(C)c(=O)c2c1 ; Cc1cc([C@@H](C)Nc2ccccc2C(=O)O)c2oc(-c3cnn(-c4ccc(C#N)cc4)c3)c(C)c(=O)c2c1] 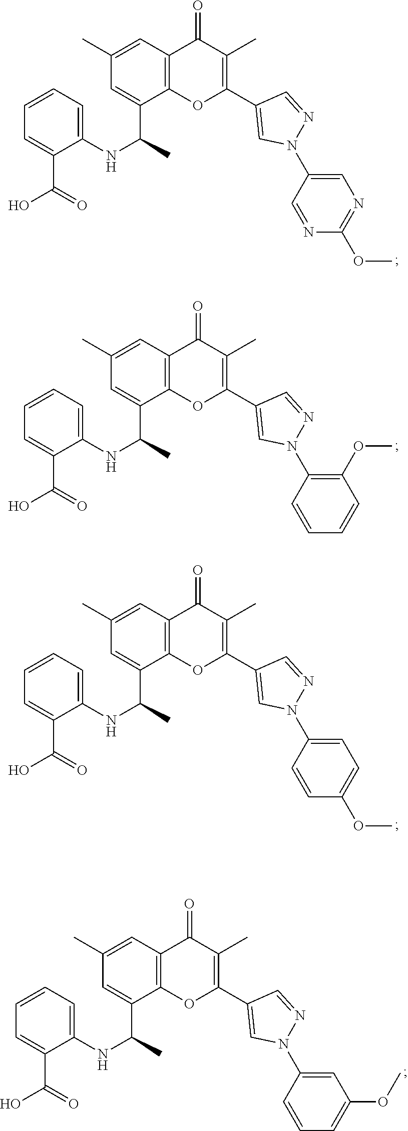 [SMILES: COc1ncc(-n2cc(-c3oc4c([C@@H](C)Nc5ccccc5C(=O)O)cc(C)cc4c(=O)c3C)cn2)cn1 ; COc1ccccc1-n1cc(-c2oc3c([C@@H](C)Nc4ccccc4C(=O)O)cc(C)cc3c(=O)c2C)cn1 ; COc1ccc(-n2cc(-c3oc4c([C@@H](C)Nc5ccccc5C(=O)O)cc(C)cc4c(=O)c3C)cn2)cc1 ; COc1cccc(-n2cc(-c3oc4c([C@@H](C)Nc5ccccc5C(=O)O)cc(C)cc4c(=O)c3C)cn2)c1] 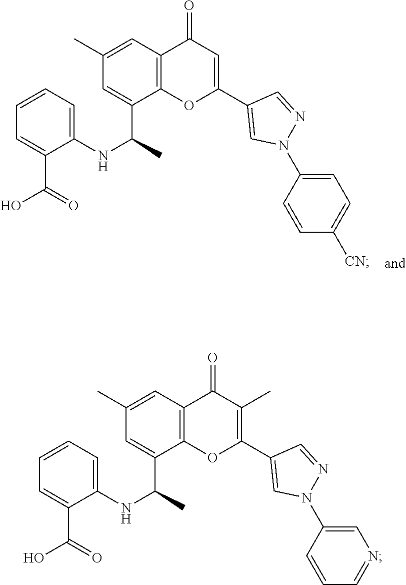 [SMILES: Cc1cc([C@@H](C)Nc2ccccc2C(=O)O)c2oc(-c3cnn(-c4ccc(C#N)cc4)c3)cc(=O)c2c1 ; Cc1cc([C@@H](C)Nc2ccccc2C(=O)O)c2oc(-c3cnn(-c4cccnc4)c3)c(C)c(=O)c2c1]
or a pharmaceutically acceptable salt thereof.

52. The compound of claim 1, selected from: 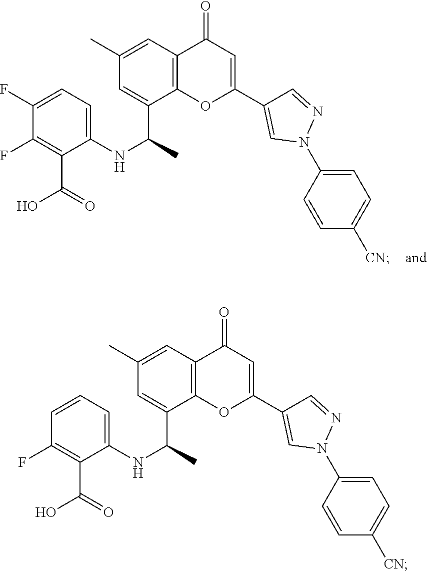 [SMILES: Cc1cc([C@@H](C)Nc2ccc(F)c(F)c2C(=O)O)c2oc(-c3cnn(-c4ccc(C#N)cc4)c3)cc(=O)c2c1 ; Cc1cc([C@@H](C)Nc2cccc(F)c2C(=O)O)c2oc(-c3cnn(-c4ccc(C#N)cc4)c3)cc(=O)c2c1]
or a pharmaceutically acceptable salt thereof.

53. The compound of claim 1, selected from: 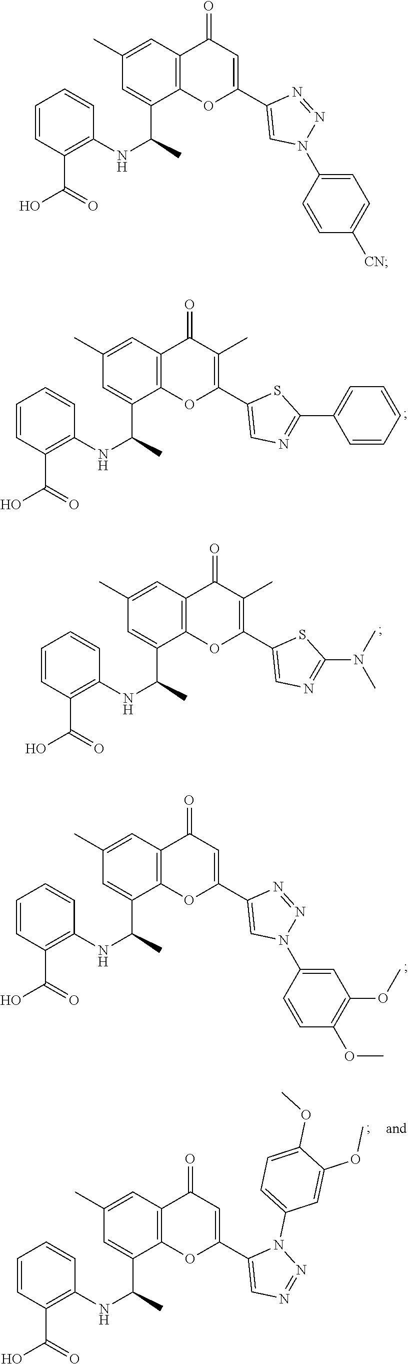 [SMILES: Cc1cc([C@@H](C)Nc2ccccc2C(=O)O)c2oc(-c3cn(-c4ccc(C#N)cc4)nn3)cc(=O)c2c1 ; Cc1cc([C@@H](C)Nc2ccccc2C(=O)O)c2oc(-c3cnc(-c4ccccc4)s3)c(C)c(=O)c2c1 ; Cc1cc([C@@H](C)Nc2ccccc2C(=O)O)c2oc(-c3cnc(N(C)C)s3)c(C)c(=O)c2c1 ; COc1ccc(-n2cc(-c3cc(=O)c4cc(C)cc([C@@H](C)Nc5ccccc5C(=O)O)c4o3)nn2)cc1OC ; COc1ccc(-n2nncc2-c2cc(=O)c3cc(C)cc([C@@H](C)Nc4ccccc4C(=O)O)c3o2)cc1OC] 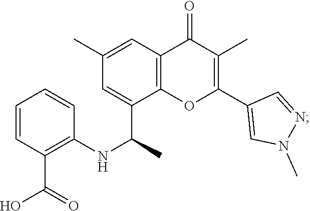 [SMILES: Cc1cc([C@@H](C)Nc2ccccc2C(=O)O)c2oc(-c3cnn(C)c3)c(C)c(=O)c2c1]
or a pharmaceutically acceptable salt thereof.

54. A pharmaceutical composition comprising a compound of claim 1, or a pharmaceutically acceptable salt thereof, and a pharmaceutically acceptable carrier.

55. A method of treating a disease or disorder associated with modulation of phosphoinositide 3-kinase (PI3K), comprising administering to a patient in need thereof a therapeutically effective amount of a compound of claim 1, or a pharmaceutically acceptable salt thereof.

56. (canceled)

57. (canceled)

58. The method of claim 55, wherein the disease or disorder is a cancer.

59. (canceled)

60. The method of claim 58, wherein the cancer is breast cancer.

61. The method of claim 58, wherein the cancer is hormone receptor-positive (HR+), human epidermal growth factor receptor 2-negative (HER2−) advanced or metastatic breast cancer.

62. (canceled)

63. A method of inhibiting phosphoinositide 3-kinase (PI3K), comprising administering to a patient in need thereof a therapeutically effective amount of a compound of claim 1, or a pharmaceutically acceptable salt thereof.

64. A method of treating cancer, the method comprising administering to a patient in need thereof a therapeutically effective amount of a compound of claim 1, or a pharmaceutically acceptable salt thereof.

65. (canceled)

66. The method of claim 64, wherein the cancer is breast cancer.

67. The method of claim 64, wherein the cancer is hormone receptor-positive (HR+), human epidermal growth factor receptor 2-negative (HER2−) advanced or metastatic breast cancer.

68.-81. (canceled)
